(19) United States
(12) Patent Application Publication
(10) Pub. No.: US 2023/0373952 A1
(43) Pub. Date: Nov. 23, 2023

(54) LACTAM COMPOUNDS AS Kv1.3 POTASSIUM SHAKER CHANNEL BLOCKERS

(71) Applicant: D.E. Shaw Research, LLC, Indianapolis, IN (US)

(72) Inventors: Fabrizio GIORDANETTO, New York, NY (US); Morten Østergaard JENSEN, Hellerup (DK); Vishwanath JOGINI, Hyderabad (IN); Roger John SNOW, Danbury, CT (US)

(21) Appl. No.: 18/247,521

(22) PCT Filed: Oct. 4, 2021
(86) PCT No.: PCT/US2021/053320

§ 371 Date: Mar. 31, 2023

Related U.S. Application Data
(60) Provisional application No. 63/088,171, filed on Oct. 6, 2020.

(51) Int. Cl. C07D 401/04; C07D 403/04; C07D 405/04; C07D 207/27; C07D 403/06; C07D 417/04; C07D 413/04; C07D 263/20; C07D 265/32; C07D 233/32

(52) U.S. Cl. CPC C07D 401/04; C07D 403/04; C07D 405/04; C07D 207/27; C07D 403/06; C07D 417/04; C07D 413/04; C07D 263/20; C07D 265/32; C07D 233/32

(57) ABSTRACT

A compound of Formula (I), or a pharmaceutically acceptable salt thereof, is described, where the substituents are as defined herein. Pharmaceutical compositions including the same and method of using the same are also described.

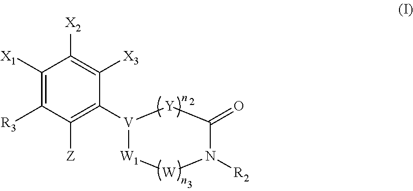

This application claims the benefit and priority of U.S. Provisional Application No. 63/088,171, filed Oct. 6, 2020, the entire contents of which is hereby incorporated by reference in its entirety.

This patent disclosure contains material that is subject to copyright protection. The copyright owner has no objection to the facsimile reproduction of the patent document or the patent disclosure as it appears in the U.S. Patent and Trademark Office patent file or records, but otherwise reserves any and all copyright rights.

### INCORPORATION BY REFERENCE

All documents cited herein are incorporated herein by reference in their entirety.

### FIELD OF THE INVENTION

The invention relates generally to the field of pharmaceutical science. More particularly, the invention relates to compounds and compositions useful as pharmaceuticals as potassium channel blockers.

### BACKGROUND

Voltage-gated Kv1.3 potassium (K⁺) channels are expressed in lymphocytes (T and B lymphocytes), the central nervous system, and other tissues, and regulate a large number of physiological processes, such as, but not limited to, neurotransmitter release, heart rate, insulin secretion, and neuronal excitability. Kv1.3 channels can regulate membrane potential and thereby indirectly influence calcium signaling in human effector memory T cells. Effector memory T cells are mediators of several conditions, including multiple sclerosis, type I diabetes mellitus, psoriasis, spondylitis, parodontitis, and rheumatoid arthritis. Upon activation, effector-memory T cells increase expression of the Kv1.3 channel. Amongst human B cells, naive and early memory B cells express small numbers of Kv1.3 channels when they are quiescent. In contrast, class-switched memory B cells express high numbers of Kv1.3 channels. Furthermore, the Kv1.3 channel promotes the calcium homeostasis required for T cell receptor-mediated cell activation, gene transcription, and proliferation. See Panyi, G., et al., 2004, Trends Immunol., 565-569. Blockade of Kv1.3 channels in effector memory T cells suppresses activities such as, but not limited to, calcium signaling, cytokine production (e.g., interferon-gamma or interleukin 2), and cell proliferation.

Autoimmune disease is a family of disorders resulting from tissue damage caused by attack from the body's own immune system. Such diseases may affect a single organ, as in, for example, multiple sclerosis and type I diabetes mellitus, or may involve multiple organs, as in, for example, rheumatoid arthritis and systemic lupus erythematosus. Treatment is generally palliative, with anti-inflammatory and immunosuppressive drugs, which can have severe side effects. A need for more effective therapies has led to a search for drugs that can selectively inhibit the function of effector memory T cells, known to be involved in the etiology of autoimmune diseases. These inhibitors are thought to be able to ameliorate autoimmune disease symptoms without compromising the protective immune response. Effector memory T cells express high numbers of the Kv1.3 channel and depend on these channels for their function. In vivo, Kv1.3 channel blockers paralyze effector memory T cells at the sites of inflammation and prevent their reactivation in inflamed tissues. Kv1.3 channel blockers do not affect the motility within lymph nodes of naive and central memory T cells. Suppressing the function of these cells by selectively blocking the Kv1.3 channel offers the potential for effective therapy of autoimmune diseases with minimal side effects.

Multiple sclerosis is caused by autoimmune damage to the central nervous system. Symptoms include, but are not limited to, muscle weakness and paralysis, which can severely affect quality of life for patients. Multiple sclerosis progresses rapidly and unpredictably and eventually leads to death. The Kv1.3 channel is also highly expressed in auto-reactive effector memory T cells from multiple sclerosis patients. See Wulff H., et al., 2003, J. Clin. Invest., 1703-1713; Rus H., et al., 2005, PNAS, 11094-11099. Animal models of multiple sclerosis have been successfully treated using blockers of the Kv1.3 channel.

Compounds which are selective Kv1.3 channel blockers are thus potential therapeutic agents as immunosuppressants or immune system modulators. The Kv1.3 channel is also considered as a therapeutic target for the treatment of obesity and for enhancing peripheral insulin sensitivity in patients with type II diabetes mellitus. These compounds can also be utilized in the prevention of graft rejection and the treatment of immunological (e.g., autoimmune) and inflammatory disorders.

Tubulointerstitial fibrosis is a progressive connective tissue deposition on the kidney parenchyma, leading to renal function deterioration, and is involved in the pathology of, for example, chronic kidney disease, chronic renal failure, nephritis, and inflammation in glomeruli, and is a common cause of end-stage renal failure. Overexpression of Kv1.3 channels in lymphocytes can promote their proliferation, leading to chronic inflammation and overstimulation of cellular immunity, which are involved in the underlying pathology of these renal diseases and are contributing factors in the progression of tubulointerstitial fibrosis. Inhibition of the lymphocyte Kv1.3 channel currents suppress proliferation of kidney lymphocytes and ameliorate the progression of renal fibrosis. See Kazama I., et al., 2015, Mediators Inflamm., 1-12.

Kv1.3 channels also play a role in gastroenterological disorders including, but not limited to, inflammatory bowel diseases, such as, but not limited to, ulcerative colitis and Crohn's disease. Ulcerative colitis is a chronic inflammatory bowel disease characterized by excessive T cell infiltration and cytokine production. Ulcerative colitis can impair quality of life and can lead to life-threatening complications. High levels of Kv1.3 channels in CD4- and CD8-positive T cells in the inflamed mucosa of ulcerative colitis patients have been associated with production of pro-inflammatory compounds in active ulcerative colitis. Kv1.3 channels are thought to serve as a marker of disease activity and pharmacological blockade might constitute a novel immunosuppressive strategy in ulcerative colitis. Present treatment regimens for ulcerative colitis, including, but not limited to, corticosteroids, salicylates, and anti-TNF-α reagents, are insufficient for many patients. See Hansen L. K., et al., 2014, J. Crohns Colitis, 1378-1391. Crohn's disease is a type of inflammatory bowel disease which may affect any part of the gastrointestinal tract. Crohn's disease is thought to be the result of intestinal inflammation due to a T cell-driven process initiated by normally safe bacteria. Thus, Kv1.3 channel inhibition can be utilized in treating the Crohn's disease.

In addition to T cells, Kv1.3 channels are also expressed in microglia, where the channel is involved in inflammatory cytokine and nitric oxide production and in microglia-mediated neuronal killing. In humans, strong Kv1.3 channel expression has been found in microglia in the frontal cortex of patients with Alzheimer's disease and on CD68⁺ cells in multiple sclerosis brain lesions. It has been suggested that Kv1.3 channel blockers might be able to preferentially target detrimental proinflammatory microglia functions. Kv1.3 channels are expressed on activated microglia in infarcted rodent and human brain. Higher Kv1.3 channel current densities are observed in acutely isolated microglia from the infarcted hemisphere than in microglia isolated from the contralateral hemisphere of a mouse model of stroke. See Chen Y. J., et al., 2017, Ann. Clin. Transl. Neurol., 147-161.

Expression of Kv1.3 channels is elevated in microglia of human Alzheimer's disease brains, suggesting that Kv1.3 channel is a pathologically relevant microglial target in Alzheimer's disease. See Rangaraju S., et al., 2015, J. Alzheimers Dis., 797-808. Soluble AβO enhances microglial Kv1.3 channel activity. Kv1.3 channels are required for AβO-induced microglial pro-inflammatory activation and neurotoxicity. Kv1.3 channel expression/activity is upregulated in transgenic Alzheimer's disease animals and human Alzheimer's disease brains. Pharmacological targeting of microglial Kv1.3 channels can affect hippocampal synaptic plasticity and reduce amyloid deposition in APP/PS1 mice. Thus, the Kv1.3 channel may be a therapeutic target for Alzheimer's disease.

Kv1.3 channel blockers could be also useful for ameliorating pathology in cardiovascular disorders such as, but not limited to, ischemic stroke, where activated microglia significantly contributes to the secondary expansion of the infarct.

Kv1.3 channel expression is associated with the control of proliferation in multiple cell types, apoptosis, and cell survival. These processes are crucial for cancer progression. In this context, Kv1.3 channels located in the inner mitochondrial membrane can interact with the apoptosis regulator Bax. See Serrano-Albarras, A., et al., 2018, Expert Opin. Ther. Targets, 101-105. Thus, inhibitors of Kv1.3 channels may be used as anticancer agents.

A number of peptide toxins with multiple disulfide bonds from spiders, scorpions, and anemones are known to block Kv1.3 channels. A few selective, potent peptide inhibitors of the Kv1.3 channel have been developed. A synthetic derivative of stichodactyla toxin (“shk”) with an unnatural amino acid (shk-186) is the most advanced peptide toxin. Shk has demonstrated efficacy in preclinical models and is currently in a phase I clinical trial for treatment of psoriasis. Shk can suppress proliferation of effector memory T cells, resulting in improved condition in animal models of multiple sclerosis. Unfortunately, shk also binds to the closely related Kvi channel subtype found in the central nervous system and the heart. Thus, there is a need for Kv1.3 channel-selective inhibitors to avoid potential cardio- and neurotoxicity. Additionally, small peptides like shk-186 are rapidly cleared from the body after administration, resulting in short circulating half-lives and frequent administration events. Thus, there is a need for the development of long-acting, selective Kv1.3 channel inhibitors for the treatment of chronic inflammatory diseases.

Thus, there remains a need for the development of novel Kv1.3 channel blockers as pharmaceutical agents.

### SUMMARY OF THE INVENTION

In one aspect, compounds useful as potassium channel blockers having a structure of Formula I

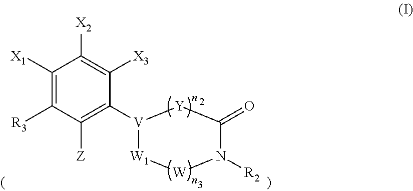

are described, where the various substituents are defined herein. The compounds of Formula I described herein can block Kv1.3 potassium (K⁺) channels and can be used in the treatment of a variety of disease conditions. Methods for synthesizing these compounds are also described herein. Pharmaceutical compositions and methods of using these compositions described herein are useful for treating conditions in vitro and in vivo. Such compounds, pharmaceutical compositions, and methods of treatment have a number of clinical applications, including, but not limited to, as pharmaceutically active agents and methods for treating cancer, an immunological disorder, a central nervous system disorder, an inflammatory disorder, a gastroenterological disorder, a metabolic disorder, a cardiovascular disorder, a kidney disease, or a combination thereof.

In one aspect, a compound of Formula I or a pharmaceutically acceptable salt thereof is described:

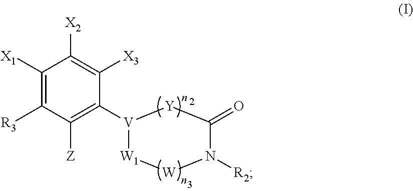

wherein:
X₁, X₂, and X₃ are each independently H, halogen, CN, alkyl, cycloalkyl, halogenated alkyl, or halogenated cycloalkyl;
or alternatively X₁ and X₂ and the carbon atoms they are connected to taken together form an optionally substituted 5- or 6-membered aryl;
or alternatively X₂ and X₃ and the carbon atoms they are connected to taken together form an optionally substituted 5- or 6-membered aryl;
Z is ORₐ;
R₃ is H, halogen, alkyl, cycloalkyl, saturated heterocycle, aryl, heteroaryl, CN, CF₃, OCF₃, ORₐ, SRₐ, NRₐR_{b}, or NRₐ(C═O)R_{b};
V is CR₁;
W₁ is CHR₁, O, or NR₄;
each occurrence of W is independently CHR₁, O, or NR₅;
each occurrence of Y is independently CHR₁, O, or NR₆;
each occurrence of R₁ is independently H, alkyl, halogen, or (CR₇R₈)ₚNRₐR_{b};
each occurrence of R₄, R₅, and R₆ is independently H, alkyl, heteroalkyl, cycloalkyl, cycloheteroalkyl, alkylaryl, aryl, or heteroaryl;
R₂ is H, alkyl, (CR₇R₈)ₚcycloalkyl, (CR₇R₈)ₚheteroalkyl, (CR₇R₈)ₚcycloheteroalkyl, (CR₇R₈)ₚaryl, (CR₇R₈)ₚheteroaryl, (CR₇R₈)ₚORₐ, (CR₇R₈)ₚNRₐR_{b}, (CR₇R₈)ₚ(C═O)ORₐ, (CR₇R₈)ₚNRₐ(C═O)R_{b}, or (CR₇R₈)ₚ(C═O)NRₐR_{b};
each occurrence of R₇ and R₈ is independently H, alkyl, cycloalkyl, aryl, or heteroaryl;
each occurrence of Rₐ and R_{b} is independently H, alkyl, cycloalkyl, heterocycle, aryl, or heteroaryl;
or alternatively Rₐ and R_{b} together with the atom that they are connected to form a 3-7-membered optionally substituted carbocycle or heterocycle;
the alkyl, cycloalkyl, heteroalkyl, cycloheteroalkyl, aryl, heteroaryl, carbocycle, and heterocycle of X₁, X₂, X₃, R₁, R₂, R₃, R₄, R₅, R₆, R₇, R₈, Rₐ, and R_{b}, where applicable, are each independently and optionally substituted by 1-4 substituents each independently selected from the group consisting of alkyl, cycloalkyl, halogenated alkyl, halogenated cycloalkyl, halogen, CN, R_{c}, (CR_{c}R_{d})ₚOR_{c}, (CR_{c}R_{d})ₚ(C═O)OR_{c}, (CR_{c}R_{d})ₚNR_{c}R_{d}, (CR_{c}R_{d})ₚ(C═O)NR_{c}R_{d}, (CR_{c}R_{d})ₚNR_{c}(C═O)R_{d}, and oxo where valence permits;
each occurrence of R_{c} and R_{d} is independently H, alkyl, cycloalkyl, heterocycle, aryl, or heteroaryl;
each heterocycle comprises 1-3 heteroatoms each independently selected from the group consisting of O, S, and N;
n₂ is an integer from 0-2;
n₃ is an integer from 0-2;
wherein the sum of n₂ and n₃ is 1 or 2; and
each occurrence of p is independently an integer from 0-4.

In any one of the embodiments described herein, W₁ is CHR₁ or NR₄.

In any one of the embodiments described herein, W₁ is CHR₁ or O.

In any one of the embodiments described herein, each occurrence of W is independently CHR₁ or NR₅.

In any one of the embodiments described herein, each occurrence of W is independently CHR₁ or O.

In any one of the embodiments described herein, each occurrence of Y is independently CHR₁ or O.

In any one of the embodiments described herein, each occurrence of Y is independently CHR₁ or NR₆.

In any one of the embodiments described herein, the compound has the structure of Formula Ia:

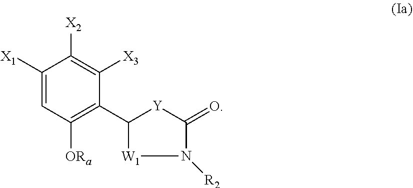

In any one of the embodiments described herein, the compound has the structure of Formula Ib:

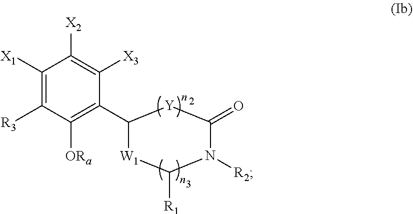

wherein n₂ is 1-2 and n₃ is 0-1; and wherein the sum of n₂ and n₃ is 2.

In any one of the embodiments described herein, each occurrence of R₁ is H.

In any one of the embodiments described herein, each occurrence of R₁ is independently alkyl or cycloalkyl.

In any one of the embodiments described herein, each occurrence of R₁ is independently H or (CR₇R₈)ₚNRₐR_{b}.

In any one of the embodiments described herein, each occurrence of R₁ is independently H, alkyl, or (CR₇R₈)ₚNRₐR_{b}.

In any one of the embodiments described herein, each occurrence of R₁ is independently H, CH₃, CH₂CH₃, NH₂, NHCH₃, or N(CH₃)₂.

In any one of the embodiments described herein, each occurrence of R₄, R₅, and R₆ is independently H, alkyl, heteroalkyl, cycloalkyl, or cycloheteroalkyl.

In any one of the embodiments described herein, each occurrence of R₄, R₅, and R₆ is independently aryl, alkylaryl, or heteroaryl.

In any one of the embodiments described herein, each occurrence of R₄, R₅, and R₆ is independently H or alkyl.

In any one of the embodiments described herein, each occurrence of R₄, R₅, and R₆ is H.

In any one of the embodiments described herein, R₂ is H, alkyl, or (CR₇R₈)ₚcycloalkyl.

In any one of the embodiments described herein, the cycloalkyl is selected from the group consisting of a cyclopropyl, cyclobutyl, and cyclopentyl group.

In any one of the embodiments described herein, R₂ is (CR₇R₈)ₚheteroalkyl or (CR₇R₈)ₚcycloheteroalkyl.

In any one of the embodiments described herein, the cycloheteroalkyl is selected from the group consisting of an azetidinyl, oxetanyl, pyrrolidinyl, tetrahydrofuranyl, tetrahydropyranyl, piperazinyl, piperazinonyl, and pyridinonyl group.

In any one of the embodiments described herein, R₂ is (CR₇R₈)ₚaryl or (CR₇R₈)ₚheteroaryl.

In any one of the embodiments described herein, the heteroaryl is selected from the group consisting of an isoxazolyl, isothiazolyl, pyridinyl, imidazolyl, thiazolyl, pyrazolyl, and triazolyl group.

In any one of the embodiments described herein, R₂ is (CR₇R₈)ₚORₐ or (CR₇R₈)ₚNRₐR_{b}.

In any one of the embodiments described herein, R₂ is (CR₇R₈)ₚ(C═O)ORₐ, (CR₇R₈)ₚNRₐ(C═O)R_{b}, or (CR₇R₈)ₚ(C═O)NRₐR_{b}.

In any one of the embodiments described herein, each occurrence of R₇ and R₈ is independently H or alkyl.

In any one of the embodiments described herein, each occurrence of R₇ and R₈ is independently H, cycloalkyl, aryl, or heteroaryl.

In any one of the embodiments described herein, each occurrence of Rₐ and R_{b} is independently H or alkyl.

In any one of the embodiments described herein, each occurrence of Rₐ and R_{b} is independently H, cycloalkyl, heterocycle, aryl, or heteroaryl.

In any one of the embodiments described herein, at least one occurrence of p is 0, 1, or 2.

In any one of the embodiments described herein, at least one occurrence of p is 3 or 4.

In any one of the embodiments described herein, V is CH and the structural moiety

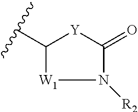

has the structure of

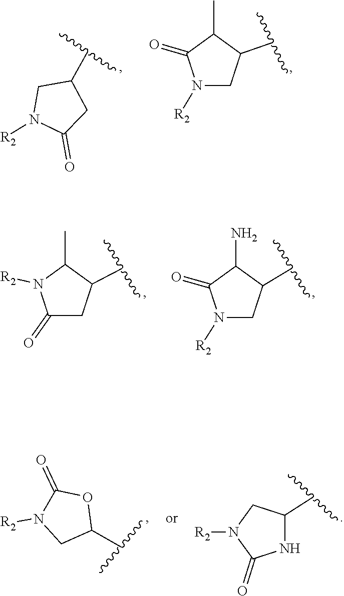

In any one of the embodiments described herein, V is CH and the structural moiety

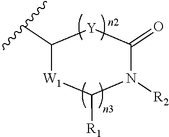

has the structure of

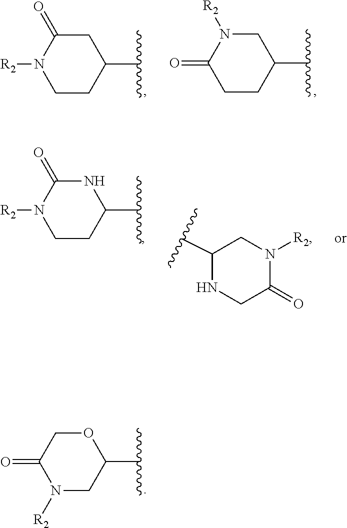

In any one of the embodiments described herein, R₂ is

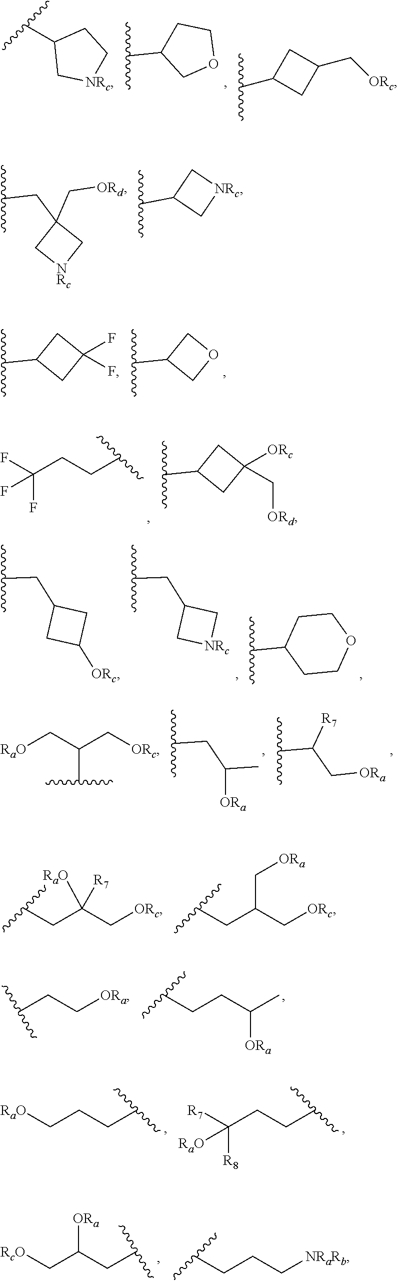

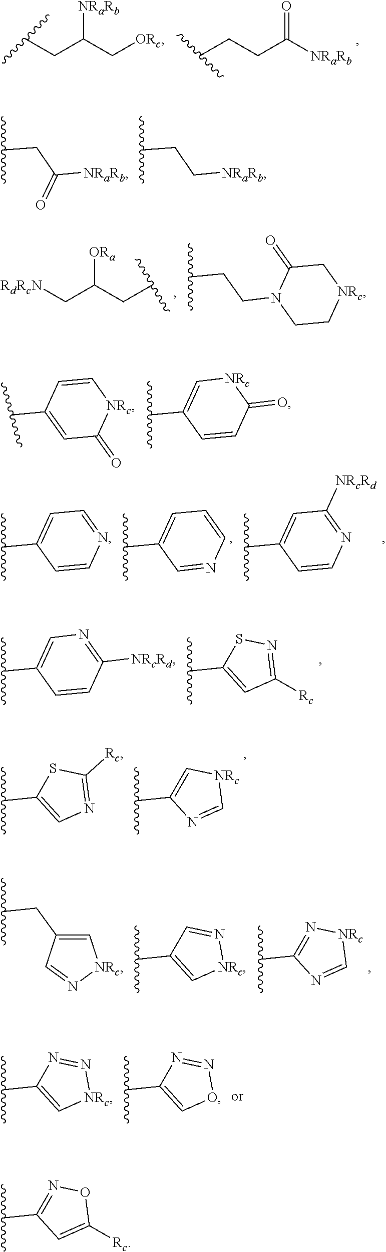

In any one of the embodiments described herein, R₂ is

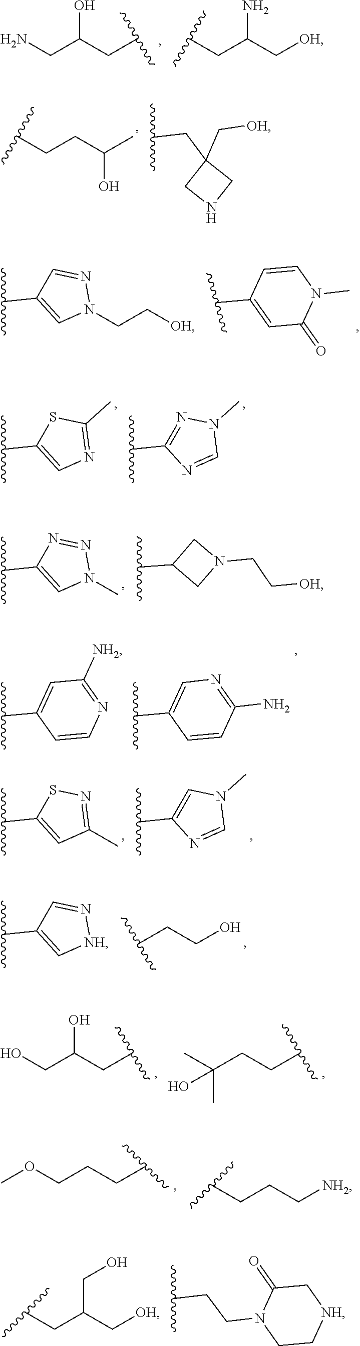

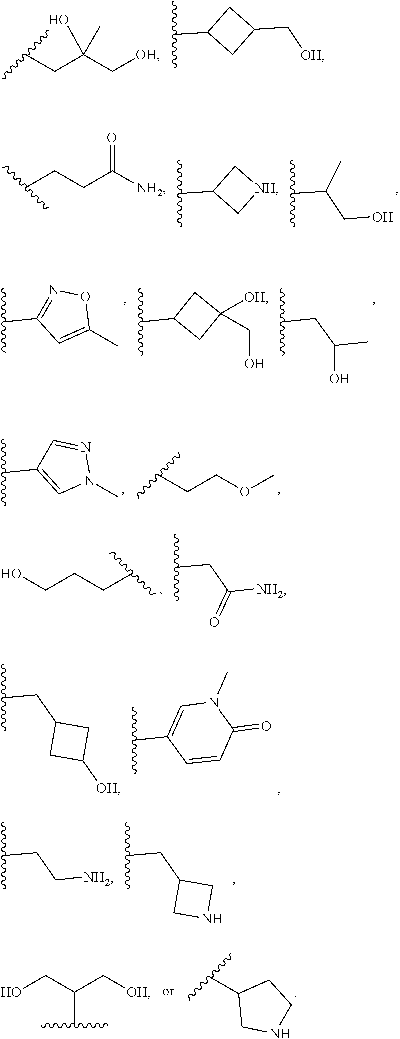

In any one of the embodiments described herein, the structural moiety

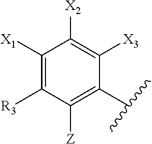

has the structure of

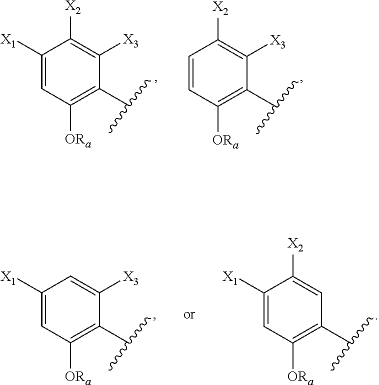

In any one of the embodiments described herein, X₁, X₂, and X₃ are each independently H, halogen, alkyl, or halogenated alkyl.

In any one of the embodiments described herein, X₁, X₂, and X₃ are each independently CN, cycloalkyl, or halogenated cycloalkyl.

In any one of the embodiments described herein, X₁, X₂, and X₃ are each independently H, F, Cl, Br, CH₃, CH₂F, CHF₂, or CF₃.

In any one of the embodiments described herein, X₁, X₂, and X₃ are each independently H or Cl.

In any one of the embodiments described herein, Z is OH or O(C₁₋₄ alkyl).

In any one of the embodiments described herein, Z is OH.

In any one of the embodiments described herein, R₃ is H, halogen, alkyl, or cycloalkyl.

In any one of the embodiments described herein, R₃ is saturated heterocycle, aryl, or heteroaryl.

In any one of the embodiments described herein, R₃ is CN, CF₃, OCF₃, ORₐ or SRₐ.

In any one of the embodiments described herein, R₃ is NRₐR_{b} or NRₐ(C═O)R_{b}.

In any one of the embodiments described herein, each occurrence of Rₐ and R_{b} is independently H or alkyl.

In any one of the embodiments described herein, R₃ is H, F, Cl, Br, C₁₋₄ alkyl, or CF₃.

In any one of the embodiments described herein, R₃ is H.

In any one of the embodiments described herein, the structural moiety

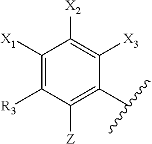

has the structure of

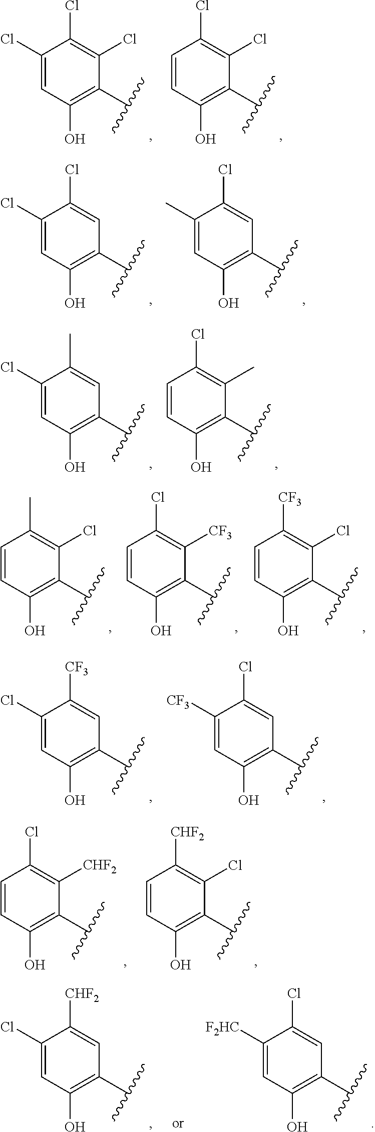

In any one of the embodiments described herein, the structural moiety

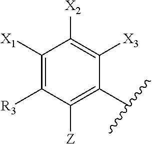

has the structure of

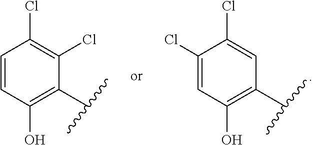 [SMILES: *c1c(O)ccc(Cl)c1Cl ; *c1cc(Cl)c(Cl)cc1O]

In any one of the embodiments described herein, the compound is selected from the group consisting of compounds 1-105 as shown in Table 1.

In another aspect, a pharmaceutical composition is described, including at least one compound according to any one of the embodiments described herein, or a pharmaceutically-acceptable salt thereof, and a pharmaceutically-acceptable carrier or diluent.

In yet another aspect, a method of treating a condition in a mammalian species in need thereof is described, including administering to the mammalian species a therapeutically effective amount of at least one compound according to any one of the embodiments described herein or a pharmaceutically-acceptable salt thereof, or a therapeutically effective amount of a pharmaceutical composition according to any one of the embodiments described herein, where the condition is selected from the group consisting of cancer, an immunological disorder, a central nervous system disorder, an inflammatory disorder, a gastroenterological disorder, a metabolic disorder, a cardiovascular disorder, and a kidney disease.

In any one of the embodiments described herein, the immunological disorder is transplant rejection or an autoimmune disease.

In any one of the embodiments described herein, the autoimmune disease is rheumatoid arthritis, multiple sclerosis, systemic lupus erythematosus, or type I diabetes mellitus.

In any one of the embodiments described herein, the central nervous system disorder is Alzheimer's disease.

In any one of the embodiments described herein, the inflammatory disorder is an inflammatory skin condition, arthritis, psoriasis, spondylitis, parodontitits, or an inflammatory neuropathy.

In any one of the embodiments described herein, the gastroenterological disorder is an inflammatory bowel disease.

In any one of the embodiments described herein, the metabolic disorder is obesity or type II diabetes mellitus.

In any one of the embodiments described herein, the kidney disease is chronic kidney disease, nephritis, or chronic renal failure.

In any one of the embodiments described herein, the condition is selected from the group consisting of cancer, transplant rejection, rheumatoid arthritis, multiple sclerosis, systemic lupus erythematosus, type I diabetes mellitus, Alzheimer's disease, inflammatory skin condition, inflammatory neuropathy, psoriasis, spondylitis, parodontitis, Crohn's disease, ulcerative colitis, obesity, type II diabetes mellitus, ischemic stroke, chronic kidney disease, nephritis, chronic renal failure, and a combination thereof.

In any one of the embodiments described herein, the mammalian species is human.

In still another aspect, a method of blocking Kv1.3 potassium channel in a mammalian species in need thereof is described, including administering to the mammalian species a therapeutically effective amount of at least one compound according to any one of the embodiments described herein or a pharmaceutically-acceptable salt thereof, or a therapeutically effective amount of a pharmaceutical composition according to any one of the embodiments described herein.

Any one of the embodiments described herein may be properly combined with any other embodiment disclosed herein. The combination of any one of the embodiments described herein with any other embodiments described herein is expressly contemplated. Specifically, the selection of one or more embodiments for one substituent group can be properly combined with the selection of one or more particular embodiments for any other substituent group. Such combination can be made in any one or more embodiments of the application described herein or any formula described herein.

### DETAILED DESCRIPTION OF THE INVENTION

### Definitions

The following are definitions of terms used in the present specification. The initial definition provided for a group or term herein applies to that group or term throughout the present specification individually or as part of another group, unless otherwise indicated. Unless otherwise defined, all technical and scientific terms used herein have the same meaning as commonly understood by one of ordinary skill in the art.

The terms “alkyl” and “alk” refer to a straight or branched chain alkane (hydrocarbon) radical containing from 1 to 12 carbon atoms, preferably 1 to 6 carbon atoms. Exemplary “alkyl” groups include methyl, ethyl, propyl, isopropyl, n-butyl, t-butyl, isobutyl pentyl, hexyl, isohexyl, heptyl, 4,4-dimethylpentyl, octyl, 2,2,4-trimethylpentyl, nonyl, decyl, undecyl, dodecyl, and the like. The term “(C₁-C₄)alkyl” refers to a straight or branched chain alkane (hydrocarbon) radical containing from 1 to 4 carbon atoms, such as methyl, ethyl, propyl, isopropyl, n-butyl, t-butyl, and isobutyl. “Substituted alkyl” refers to an alkyl group substituted with one or more substituents, preferably 1 to 4 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, where each occurrence of Rₐ is independently hydrogen, alkyl, cycloalkyl, alkenyl, cycloalkenyl, alkynyl, heterocycle, or aryl; each occurrence of R_{b}, Rₑ and R_{d} is independently hydrogen, alkyl, cycloalkyl, heterocycle, aryl, or said R_{b} and Rₑ together with the N to which they are bonded optionally form a heterocycle, and each occurrence of Rₑ is independently alkyl, cycloalkyl, alkenyl, cycloalkenyl, alkynyl, heterocycle, or aryl. In some embodiments, groups such as alkyl, cycloalkyl, alkenyl, alkynyl, cycloalkenyl, heterocycle, and aryl can themselves be optionally substituted.

The term “heteroalkyl” refers to a straight- or branched-chain alkyl group preferably having from 2 to 12 carbons, more preferably 2 to 10 carbons in the chain, one or more of which has been replaced by a heteroatom selected from the group consisting of S, O, P and N. Exemplary heteroalkyls include, but are not limited to, alkyl ethers, secondary and tertiary alkyl amines, alkyl sulfides, and the like. The group may be a terminal group or a bridging group.

The term “alkenyl” refers to a straight- or branched-chain hydrocarbon radical containing from 2 to 12 carbon atoms and at least one carbon-carbon double bond. Exemplary such groups include ethenyl or allyl. The term “C₂-C₆ alkenyl” refers to a straight or branched chain hydrocarbon radical containing from 2 to 6 carbon atoms and at least one carbon-carbon double bond, such as ethylenyl, propenyl, 2-propenyl, (E)-but-2-enyl, (Z)-but-2-enyl, 2-methy(E)-but-2-enyl, 2-methy(Z)-but-2-enyl, 2,3-dimethyl-but-2-enyl, (Z)-pent-2-enyl, (E)-pent-1-enyl, (Z)-hex-1-enyl, (E)-pent-2-enyl, (Z)-hex-2-enyl, (E)-hex-2-enyl, (Z)-hex-i-enyl, (E)-hex-1-enyl, (Z)-hex-3-enyl, (E)-hex-3-enyl, and (E)-hex-1,3-dienyl. “Substituted alkenyl” refers to an alkenyl group substituted with one or more substituents, preferably 1 to 4 substituents, at any available point of attachment. Exemplary substituents include, but are not limited to, one or more of the following groups: hydrogen, halogen, alkyl, halogenated alkyl (i.e., an alkyl group bearing a single halogen substituent or multiple halogen substituents such as CF₃ or CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₐ, C(═O)Rₐ, C(═O)NR_{b}R_{c}, OC(═O)Rₐ, OC(═O)NR_{b}R_{c}, NR_{b}C(═O)ORₑ, NRₐC(═O)NR_{b}R_{c}, NR_{d}S(═O)₂NR_{b}R_{c}, NRₐP(═O)₂NR_{b}R_{c}, NR_{b}C(═O)Rₐ, or NR_{b}P(═O)₂Rₑ, where each occurrence of Rₐ is independently hydrogen, alkyl, cycloalkyl, alkenyl, cycloalkenyl, alkynyl, heterocycle, or aryl; each occurrence of R_{b}, Rₑ and R_{d} is independently hydrogen, alkyl, cycloalkyl, heterocycle, aryl, or said R_{b} and Rₑ together with the N to which they are bonded optionally form a heterocycle; and each occurrence of Rₑ is independently alkyl, cycloalkyl, alkenyl, cycloalkenyl, alkynyl, heterocycle, or aryl. The exemplary substituents can themselves be optionally substituted.

The term “alkynyl” refers to a straight- or branched-chain hydrocarbon radical containing from 2 to 12 carbon atoms and at least one carbon to carbon triple bond. Exemplary groups include ethynyl. The term “C₂-C₆ alkynyl” refers to a straight- or branched-chain hydrocarbon radical containing from 2 to 6 carbon atoms and at least one carbon-carbon triple bond, such as ethynyl, prop-1-ynyl, prop-2-ynyl, but-1-ynyl, but-2-ynyl, pent-1-ynyl, pent-2-ynyl, hex-1-ynyl, hex-2-ynyl, or hex-3-ynyl. “Substituted alkynyl” refers to an alkynyl group substituted with one or more substituents, preferably 1 to 4 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, where each occurrence of Rₐ is independently hydrogen, alkyl, cycloalkyl, alkenyl, cycloalkenyl, alkynyl, heterocycle, or aryl; each occurrence of R_{b}, Rₑ and R_{d} is independently hydrogen, alkyl, cycloalkyl, heterocycle, aryl, or said R_{b} and Rₑ together with the N to which they are bonded optionally to form a heterocycle; and each occurrence of Rₑ is independently alkyl, cycloalkyl, alkenyl, cycloalkenyl, alkynyl, heterocycle, or aryl. The exemplary substituents can themselves be optionally substituted.

The term “cycloalkyl” refers to a fully saturated cyclic hydrocarbon group containing from 1 to 4 rings and 3 to 8 carbons per ring. “C₃-C₇ cycloalkyl” refers to cyclopropyl, cyclobutyl, cyclopentyl, cyclohexyl, or cycloheptyl. “Substituted cycloalkyl” refers to a cycloalkyl group substituted with one or more substituents, preferably 1 to 4 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, where each occurrence of Rₐ is independently hydrogen, alkyl, cycloalkyl, alkenyl, cycloalkenyl, alkynyl, heterocycle, or aryl; each occurrence of R_{b}, Rₑ and R_{d} is independently hydrogen, alkyl, cycloalkyl, heterocycle, aryl, or said R_{b} and Rₑ together with the N to which they are bonded optionally to form a heterocycle; and each occurrence of Rₑ is independently alkyl, cycloalkyl, alkenyl, cycloalkenyl, alkynyl, heterocycle, or aryl. The exemplary substituents can themselves be optionally substituted. Exemplary substituents also include spiro-attached or fused cyclic substituents, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

The term “heterocycloalkyl” or “cycloheteroalkyl” refers to a saturated or partially saturated monocyclic, bicyclic, or polycyclic ring containing at least one heteroatom selected from the group consisting of nitrogen, sulfur, and oxygen, preferably from 1 to 3 heteroatoms in at least one ring. Each ring is preferably from 3 to 10 membered, more preferably 4 to 7 membered. Examples of suitable heterocycloalkyl substituents include, but are not limited to, pyrrolidyl, tetrahydrofuryl, tetrahydrothiofuranyl, piperidyl, piperazyl, tetrahydropyranyl, morpholino, 1,3-diazepane, 1,4-diazepane, 1,4-oxazepane, and 1,4-oxathiapane. The group may be a terminal group or a bridging group.

The term “cycloalkenyl” refers to a partially unsaturated cyclic hydrocarbon group containing 1 to 4 rings and 3 to 8 carbons per ring. Exemplary such groups include cyclobutenyl, cyclopentenyl, cyclohexenyl, etc. “Substituted cycloalkenyl” refers to a cycloalkenyl group substituted with one more substituents, preferably 1 to 4 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₐ, C(═O)Rₐ, C(═O)NR_{b}R_{c}, OC(═O)Rₐ, OC(═O)NR_{b}R_{c}, NR_{b}C(═O)ORₑ, NRₐC(═O)NR_{b}R_{c}, NR_{d}S(═O)₂NR_{b}R_{c}, NRₐP(═O)₂NR_{b}R_{c}, NR_{b}C(═O)Rₐ, or NR_{b}P(═O)₂Rₑ, where each occurrence of Rₐ is independently hydrogen, alkyl, cycloalkyl, alkenyl, cycloalkenyl, alkynyl, heterocycle, or aryl; each occurrence of R_{b}, Rₑ, and R_{d} is independently hydrogen, alkyl, cycloalkyl, heterocycle, aryl, or said R_{b} and Rₑ together with the N to which they are bonded optionally form a heterocycle; and each occurrence of Rₑ is independently alkyl, cycloalkyl, alkenyl, cycloalkenyl, alkynyl, heterocycle, or aryl. The exemplary substituents can themselves be optionally substituted. Exemplary substituents also include spiro-attached or fused cyclic substituents, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

The term “aryl” refers to cyclic, aromatic hydrocarbon groups that have 1 to 5 aromatic rings, especially monocyclic or bicyclic groups such as phenyl, biphenyl, or naphthyl. Where containing two or more aromatic rings (bicyclic, etc.), the aromatic rings of the aryl group may be joined at a single point (e.g., biphenyl), or fused (e.g., naphthyl, phenanthrenyl, and the like). The term “fused aromatic ring” refers to a molecular structure having two or more aromatic rings where two adjacent aromatic rings have two carbon atoms in common. “Substituted aryl” refers to an aryl group substituted by one or more substituents, preferably 1 to 3 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, where each occurrence of Rₐ is independently hydrogen, alkyl, cycloalkyl, alkenyl, cycloalkenyl, alkynyl, heterocycle, or aryl; each occurrence of R_{b}, Rₑ and R_{d} is independently hydrogen, alkyl, cycloalkyl, heterocycle, aryl, or said R_{b} and Rₑ together with the N to which they are bonded optionally form a heterocycle; and each occurrence of Rₑ is independently alkyl, cycloalkyl, alkenyl, cycloalkenyl, alkynyl, heterocycle, or aryl. The exemplary substituents can themselves be optionally substituted. Exemplary substituents also include fused cyclic groups, especially fused cycloalkyl, fused cycloalkenyl, fused heterocycle, or fused aryl, where the aforementioned cycloalkyl, cycloalkenyl, heterocycle, and aryl substituents can themselves be optionally substituted.

The term “biaryl” refers to two aryl groups linked by a single bond. The term “biheteroaryl” refers to two heteroaryl groups linked by a single bond. Similarly, the term “heteroaryl-aryl” refers to a heteroaryl group and an aryl group linked by a single bond and the term “aryl-heteroaryl” refers to an aryl group and a heteroaryl group linked by a single bond. In certain embodiments, the numbers of the ring atoms in the heteroaryl and/or aryl rings are used to specify the sizes of the aryl or heteroaryl ring in the substituents. For example, 5,6-heteroaryl-aryl refers to a substituent in which a 5-membered heteroaryl is linked to a 6-membered aryl group. Other combinations and ring sizes can be similarly specified.

The term “carbocycle” or “carbon cycle” refers to a fully saturated or partially saturated cyclic hydrocarbon group containing from 1 to 4 rings and 3 to 8 carbons per ring, or cyclic, aromatic hydrocarbon groups that have 1 to 5 aromatic rings, especially monocyclic or bicyclic groups such as phenyl, biphenyl, or naphthyl. The term “carbocycle” encompasses cycloalkyl, cycloalkenyl, cycloalkynyl, and aryl as defined hereinabove. The term “substituted carbocycle” refers to carbocycle or carbocyclic groups substituted with one or more substituents, preferably 1 to 4 substituents, at any available point of attachment. Exemplary substituents include, but are not limited to, those described above for substituted cycloalkyl, substituted cycloalkenyl, substituted cycloalkynyl, and substituted aryl. Exemplary substituents also include spiro-attached or fused cyclic substituents at any available point or points of attachment, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle, and aryl substituents can themselves be optionally substituted.

The terms “heterocycle” and “heterocyclic” refer to fully saturated, or partially or fully unsaturated, including aromatic (i.e., “heteroaryl”) cyclic groups (for example, 3 to 7 membered monocyclic, 7 to 11 membered bicyclic, or 8 to 16 membered tricyclic ring systems) which have at least one heteroatom in at least one carbon atom-containing ring. Each ring of the heterocyclic group may independently be saturated, or partially or fully unsaturated. Each ring of the heterocyclic group containing a heteroatom may have 1, 2, 3, or 4 heteroatoms selected from the group consisting of nitrogen atoms, oxygen atoms, and sulfur atoms, where the nitrogen and sulfur heteroatoms may optionally be oxidized and the nitrogen heteroatoms may optionally be quaternized. (The term “heteroarylium” refers to a heteroaryl group bearing a quaternary nitrogen atom and thus a positive charge.) The heterocyclic group may be attached to the remainder of the molecule at any heteroatom or carbon atom of the ring or ring system. Exemplary monocyclic heterocyclic groups include azetidinyl, pyrrolidinyl, pyrrolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolodinyl, 2-oxoazepinyl, azepinyl, hexahydrodiazepinyl, 4-piperidonyl, pyridyl, pyrazinyl, pyrimidinyl, pyridazinyl, triazinyl, triazolyl, tetrazolyl, tetrahydropyranyl, morpholinyl, thiamorpholinyl, thiamorpholinyl sulfoxide, thiamorpholinyl sulfone, 1,3-dioxolane, and tetrahydro-1,1-dioxothienyl, and the like. Exemplary bicyclic heterocyclic groups include indolyl, indolinyl, isoindolyl, benzothiazolyl, benzoxazolyl, benzoxadiazolyl, benzothienyl, benzo[d][1,3]dioxolyl, dihydro-2H-benzo[b][1,4]oxazine, 2,3-dihydrobenzo[b][1,4]dioxinyl, quinuclidinyl, quinolinyl, tetrahydroisoquinolinyl, isoquinolinyl, benzimidazolyl, benzopyranyl, indolizinyl, benzofuryl, benzofurazanyl, dihydrobenzo[d]oxazole, chromonyl, coumarinyl, benzopyranyl, cinnolinyl, quinoxalinyl, indazolyl, pyrrolopyridyl, furopyridinyl (such as furo[2,3-c]pyridinyl, furo[3,2-b]pyridinyl] or furo[2,3-b]pyridinyl), dihydroisoindolyl, dihydroquinazolinyl (such as 3,4-dihydro-4-oxo-quinazolinyl), triazinylazepinyl, tetrahydroquinolinyl, and the like. Exemplary tricyclic heterocyclic groups include carbazolyl, benzidolyl, phenanthrolinyl, acridinyl, phenanthridinyl, xanthenyl, and the like.

“Substituted heterocycle” and “substituted heterocyclic” (such as “substituted heteroaryl”) refer to heterocycle or heterocyclic groups substituted with one or more substituents, preferably 1 to 4 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, where each occurrence of Rₐ is independently hydrogen, alkyl, cycloalkyl, alkenyl, cycloalkenyl, alkynyl, heterocycle, or aryl; each occurrence of R_{b}, Rₑ and R_{d} is independently hydrogen, alkyl, cycloalkyl, heterocycle, aryl, or said R_{b} and Rₑ together with the N to which they are bonded optionally form a heterocycle; and each occurrence of Rₑ is independently alkyl, cycloalkyl, alkenyl, cycloalkenyl, alkynyl, heterocycle, or aryl. The exemplary substituents can themselves be optionally substituted. Exemplary substituents also include spiro-attached or fused cyclic substituents at any available point or points of attachment, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

The term “oxo” refers to

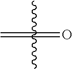 [SMILES: *=O]

substituent group, which may be attached to a carbon ring atom on a carboncycle or heterocycle. When an oxo substituent group is attached to a carbon ring atom on an aromatic group, e.g., aryl or heteroaryl, the bonds on the aromatic ring may be rearranged to satisfy the valence requirement. For instance, a pyridine with a 2-oxo substituent group may have the structure of

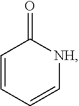

which also includes its tautomeric form of

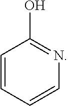

The term “alkylamino” refers to a group having the structure —NHR′, where R′ is hydrogen, alkyl or substituted alkyl, cycloalkyl or substituted cycloalkyl, as defined herein. Examples of alkylamino groups include, but are not limited to, methylamino, ethylamino, n-propylamino, iso-propylamino, cyclopropylamino, n-butylamino, t-butylamino, neopentylamino, n-pentylamino, hexylamino, cyclohexylamino, and the like.

The term “dialkylamino” refers to a group having the structure —NRR′, where R and R′ are each independently alkyl or substituted alkyl, cycloalkyl or substituted cycloalkyl, cycloalkenyl or substituted cyclolalkenyl, aryl or substituted aryl, heterocycle or substituted heterocycle, as defined herein. R and R′ may be the same or different in a dialkyamino moiety. Examples of dialkylamino groups include, but are not limited to, dimethylamino, methyl ethylamino, diethylamino, methylpropylamino, di(n-propyl)amino, di(iso-propyl)amino, di(cyclopropyl)amino, di(n-butyl)amino, di(t-butyl)amino, di(neopentyl)amino, di(n-pentyl)amino, di(hexyl)amino, di(cyclohexyl)amino, and the like. In certain embodiments, R and R′ are linked to form a cyclic structure. The resulting cyclic structure may be aromatic or non-aromatic. Examples of the resulting cyclic structure include, but are not limited to, aziridinyl, pyrrolidinyl, piperidinyl, morpholinyl, pyrrolyl, imidazolyl, 1,2,4-triazolyl, and tetrazolyl.

The terms “halogen” or “halo” refer to chlorine, bromine, fluorine, or iodine.

The term “substituted” refers to the embodiments in which a molecule, molecular moiety, or substituent group (e.g., alkyl, cycloalkyl, alkenyl, cycloalkenyl, alkynyl, heterocycle, or aryl group or any other group disclosed herein) is substituted with one or more substituents, where valence permits, preferably 1 to 6 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, alkyl, halogen-substituted alkyl, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, where each occurrence of Rₐ is independently hydrogen, alkyl, cycloalkyl, alkenyl, cycloalkenyl, alkynyl, heterocycle, or aryl; each occurrence of R_{b}, Rₑ and R_{d} is independently hydrogen, alkyl, cycloalkyl, heterocycle, aryl, or said R_{b} and Rₑ together with the N to which they are bonded optionally form a heterocycle; and each occurrence of Rₑ is independently alkyl, cycloalkyl, alkenyl, cycloalkenyl, alkynyl, heterocycle, or aryl. In the aforementioned exemplary substituents, groups such as alkyl, cycloalkyl, alkenyl, alkynyl, cycloalkenyl, heterocycle, and aryl can themselves be optionally substituted. The term “optionally substituted” refers to the embodiments in which a molecule, molecular moiety, or substituent group (e.g., alkyl, cycloalkyl, alkenyl, cycloalkenyl, alkynyl, heterocycle, or aryl group or any other group disclosed herein) may or may not be substituted with aforementioned one or more substituents.

Unless otherwise indicated, any heteroatom with unsatisfied valences is assumed to have hydrogen atoms sufficient to satisfy the valences.

The compounds of the present invention may form salts which are also within the scope of this invention. Reference to a compound of the present invention is understood to include reference to salts thereof, unless otherwise indicated. The term “salt(s)”, as employed herein, denotes acidic and/or basic salts formed with inorganic and/or organic acids and bases. In addition, when a compound of the present invention contains both a basic moiety, such as but not limited to a pyridine or imidazole, and an acidic moiety such as but not limited to a phenol or carboxylic acid, zwitterions (“inner salts”) may be formed and are included within the term “salt(s)” as used herein. Pharmaceutically-acceptable (i.e., non-toxic, physiologically-acceptable) salts are preferred, although other salts are also useful, e.g., in isolation or purification steps which may be employed during preparation. Salts of the compounds of the present invention may be formed, for example, by reacting a compound described herein with an amount of acid or base, such as an equivalent amount, in a medium such as one in which the salt precipitates, or in an aqueous medium followed by lyophilization.

The compounds of the present invention which contain a basic moiety, such as, but not limited to, an amine or a pyridine or imidazole ring, may form salts with a variety of organic and inorganic acids. Exemplary acid addition salts include acetates (such as those formed with acetic acid or trihaloacetic acid; for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, hydroxyethanesulfonates (e.g., 2-hydroxyethanesulfonates), lactates, maleates, methanesulfonates, naphthalenesulfonates (e.g., 2-naphthalenesulfonates), nicotinates, nitrates, oxalates, pectinates, persulfates, phenylpropionates (e.g., 3-phenylpropionates), phosphates, picrates, pivalates, propionates, salicylates, succinates, sulfates (such as those formed with sulfuric acid), sulfonates, tartrates, thiocyanates, toluenesulfonates such as tosylates, undecanoates, and the like.

The compounds of the present invention which contain an acidic moiety, such as, but not limited to, a phenol or carboxylic acid, may form salts with a variety of organic and inorganic bases. Exemplary basic salts include ammonium salts, alkali metal salts such as sodium, lithium and potassium salts, alkaline earth metal salts such as calcium and magnesium salts, salts with organic bases (for example, organic amines) such as benzathines, dicyclohexylamines, hydrabamines (formed with N,N-bis(dehydroabietyl) ethylenediamine), N-methyl-D-glucamines, N-methyl-D-glycamides, t-butyl amines, and salts with amino acids such as arginine, lysine, and the like. Basic nitrogen-containing groups may be quaternized with agents such as lower alkyl halides (e.g., methyl, ethyl, propyl, and butyl chlorides, bromides, and iodides), dialkyl sulfates (e.g., dimethyl, diethyl, dibutyl, and diamyl sulfates), long chain halides (e.g., decyl, lauryl, myristyl and stearyl chlorides, bromides, and iodides), aralkyl halides (e.g., benzyl and phenethyl bromides), and others.

Prodrugs and solvates of the compounds of the invention are also contemplated herein. The term “prodrug” as employed herein denotes a compound that, upon administration to a subject, undergoes chemical conversion by metabolic or chemical processes to yield a compound of the present invention, or a salt and/or solvate thereof. Solvates of the compounds of the present invention include, for example, hydrates.

Compounds of the present invention, and salts or solvates thereof, may exist in their tautomeric form (for example, as an amide or imino ether). All such tautomeric forms are contemplated herein as part of the present invention. As used herein, any depicted structure of the compound includes the tautomeric forms thereof.

All stereoisomers of the present compounds (for example, those which may exist due to asymmetric carbons on various substituents), including enantiomeric forms and diastereomeric forms, are contemplated within the scope of this invention. Individual stereoisomers of the compounds of the invention may, for example, be substantially free of other isomers (e.g., as a pure or substantially pure optical isomer having a specified activity), or may be admixed, for example, as racemates or with all other, or other selected, stereoisomers. The chiral centers of the present invention may have the S or R configuration as defined by the International Union of Pure and Applied Chemistry (IUPAC) 1974 Recommendations. The racemic forms can be resolved by physical methods, such as, for example, fractional crystallization, separation or crystallization of diastereomeric derivatives, or separation by chiral column chromatography. The individual optical isomers can be obtained from the racemates by any suitable method, including without limitation, conventional methods, such as, for example, salt formation with an optically active acid followed by crystallization.

Compounds of the present invention are, subsequent to their preparation, preferably isolated and purified to obtain a composition containing an amount by weight equal to or greater than 90%, for example, equal to or greater than 95%, equal to or greater than 99% of the compounds (“substantially pure” compounds), which is then used or formulated as described herein. Such “substantially pure” compounds of the present invention are also contemplated herein as part of the present invention.

All configurational isomers of the compounds of the present invention are contemplated, either in admixture or in pure or substantially pure form. The definition of compounds of the present invention embraces both cis (Z) and trans (E) alkene isomers, as well as cis and trans isomers of cyclic hydrocarbon or heterocyclic rings.

Throughout the specification, groups and substituents thereof may be chosen to provide stable moieties and compounds.

Definitions of specific functional groups and chemical terms are described in more detail herein. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in “Organic Chemistry”, Thomas Sorrell, University Science Books, Sausalito (1999).

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are all contemplated by the present invention. Those of ordinary skill in the art will readily appreciate that analogous ratios are contemplated for more complex isomer mixtures.

The present invention also includes isotopically labeled compounds, which are identical to the compounds disclosed herein, but for the fact that one or more atoms are replaced by an atom having an atomic mass or mass number different from the atomic mass or mass number usually found in nature. Examples of isotopes that can be incorporated into compounds of the present invention include isotopes of hydrogen, carbon, nitrogen, oxygen, phosphorous, sulfur, fluorine, and chlorine, such as ²H, ³H, ¹³C, ¹¹C, ¹⁴C, ¹⁵N, ¹⁸O, ¹⁷O, ³¹P, ³²P, ³⁵S, ¹⁸F, and ³⁶Cl, respectively. Compounds of the present invention, or an enantiomer, diastereomer, tautomer, or pharmaceutically-acceptable salt or solvate thereof, which contain the aforementioned isotopes and/or other isotopes of other atoms are within the scope of this invention. Certain isotopically labeled compounds of the present invention, for example, those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example, increased in vivo half-life or reduced dosage requirements, and hence may be preferred in some circumstances. Isotopically-labeled compounds can generally be prepared by carrying out the procedures disclosed in the Schemes and/or in the Examples below, by substituting a readily-available isotopically-labeled reagent for a non-isotopically-labeled reagent.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

It will be appreciated that the compounds, as described herein, may be substituted with any number of substituents or functional moieties. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. For purposes of this invention, heteroatoms such as nitrogen may have hydrogen substituents and/or any permissible substituents of organic compounds described herein which satisfy the valences of the heteroatoms. Furthermore, this invention is not intended to be limited in any manner by the permissible substituents of organic compounds. Combinations of substituents and variables envisioned by this invention are preferably those that result in the formation of stable compounds useful in the treatment, for example, of proliferative disorders. The term “stable,” as used herein, preferably refers to compounds which possess stability sufficient to allow manufacture and which maintain the integrity of the compound for a sufficient period of time to be detected and preferably for a sufficient period of time to be useful for the purposes detailed herein.

As used herein, the terms “cancer” and, equivalently, “tumor” refer to a condition in which abnormally replicating cells of host origin are present in a detectable amount in a subject. The cancer can be a malignant or non-malignant cancer. Cancers or tumors include, but are not limited to, biliary tract cancer; brain cancer; breast cancer; cervical cancer; choriocarcinoma; colon cancer; endometrial cancer; esophageal cancer; gastric (stomach) cancer; intraepithelial neoplasms; leukemias; lymphomas; liver cancer; lung cancer (e.g., small cell and non-small cell); melanoma; neuroblastomas; oral cancer; ovarian cancer; pancreatic cancer; prostate cancer; rectal cancer; renal (kidney) cancer; sarcomas; skin cancer; testicular cancer; thyroid cancer; as well as other carcinomas and sarcomas. Cancers can be primary or metastatic. Diseases other than cancers may be associated with mutational alternation of component of Ras signaling pathways and the compound disclosed herein may be used to treat these non-cancer diseases. Such non-cancer diseases may include: neurofibromatosis; Leopard syndrome; Noonan syndrome; Legius syndrome; Costello syndrome; cardio-facio-cutaneous syndrome; hereditary gingival fibromatosis type 1; autoimmune lymphoproliferative syndrome; and capillary malformation-arterovenous malformation.

As used herein, “effective amount” refers to any amount that is necessary or sufficient for achieving or promoting a desired outcome. In some instances, an effective amount is a therapeutically effective amount. A therapeutically effective amount is any amount that is necessary or sufficient for promoting or achieving a desired biological response in a subject. The effective amount for any particular application can vary depending on such factors as the disease or condition being treated, the particular agent being administered, the size of the subject, or the severity of the disease or condition. One of ordinary skill in the art can empirically determine the effective amount of a particular agent without necessitating undue experimentation.

As used herein, the term “subject” refers to a vertebrate animal. In one embodiment, the subject is a mammal or a mammalian species. In one embodiment, the subject is a human. In other embodiments, the subject is a non-human vertebrate animal, including, without limitation, non-human primates, laboratory animals, livestock, racehorses, domesticated animals, and non-domesticated animals.

### Compounds

Novel compounds as Kv1.3 potassium channel blockers are described. Applicants have surprisingly discovered that the compounds disclosed herein exhibit potent Kv1.3 potassium channel-inhibiting properties. Additionally, Applicants have surprisingly discovered that the compounds disclosed herein selectively block the Kv1.3 potassium channel and do not block the hERG channel and, thus, have desirable cardiovascular safety profiles.

In one aspect, a compound of Formula I or a pharmaceutically acceptable salt thereof is described:

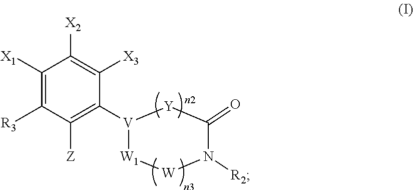

where:
X₁, X₂, and X₃ are each independently H, halogen, CN, alkyl, cycloalkyl, halogenated alkyl, or halogenated cycloalkyl;
or alternatively X₁ and X₂ and the carbon atoms they are connected to taken together form an optionally substituted 5- or 6-membered aryl;
or alternatively X₂ and X₃ and the carbon atoms they are connected to taken together form an optionally substituted 5- or 6-membered aryl;
Z is ORₐ;
R₃ is H, halogen, alkyl, cycloalkyl, saturated heterocycle, aryl, heteroaryl, CN, CF₃, OCF₃, ORₐ, SRₐ, NRₐR_{b}, or NRₐ(C═O)R_{b};
V is CR₁;
W₁ is CHR₁, O, or NR₄;
each occurrence of W is independently CHR₁, O, or NR₅;
each occurrence of Y is independently CHR₁, O, or NR₆;
each occurrence of R₁ is independently H, alkyl, halogen, or (CR₇R₈)ₚNRₐR_{b}; each occurrence of R₄, R₅, and R₆ is independently H, alkyl, heteroalkyl, cycloalkyl, cycloheteroalkyl, alkylaryl, aryl, or heteroaryl;
R₂ is H, alkyl, (CR₇R₈)ₚcycloalkyl, (CR₇R₈)ₚheteroalkyl, (CR₇R₈)ₚcycloheteroalkyl, (CR₇R₈)ₚaryl, (CR₇R₈)ₚheteroaryl, (CR₇R₈)ₚORₐ, (CR₇R₈)ₚNRₐR_{b}, (CR₇R₈)ₚ(C═O)ORₐ, (CR₇R₈)ₚNRₐ(C═O)R_{b}, or (CR₇R₈)ₚ(C═O)NRₐR_{b};
each occurrence of R₇ and R₈ is independently H, alkyl, cycloalkyl, aryl, or heteroaryl;
each occurrence of Rₐ and R_{b} is independently H, alkyl, cycloalkyl, heterocycle, aryl, or heteroaryl;
or alternatively Rₐ and R_{b} together with the atom that they are connected to form a 3-7-membered optionally substituted carbocycle or heterocycle;
the alkyl, cycloalkyl, heteroalkyl, cycloheteroalkyl, aryl, heteroaryl, carbocycle, and heterocycle of X₁, X₂, X₃, R₃, R₁, R₄, R₅, R₆, R₂, R₇, R₈, Rₐ, and R_{b} are each independently and optionally substituted by 1-4 substituents each independently selected from the group consisting of alkyl, cycloalkyl, halogenated alkyl, halogenated cycloalkyl, halogen, CN, Rₑ, (CR_{c}R_{d})ₚOR_{c}, (CR_{c}R_{d})ₚ(C═O)OR_{C}, (CR_{c}R_{d})ₚNR_{c}R_{d}, (CR_{c}R_{d})ₚ(C═O)NR_{c}R_{d}, (CR_{c}R_{d})ₚNR_{c}(C═O)Rₐ, and oxo where valence permits;
each occurrence of Rₑ and R_{d} is independently H, alkyl, cycloalkyl, heterocycle, aryl, or heteroaryl;
each heterocycle comprises 1-3 heteroatoms each independently selected from the group consisting of O, S, and N;
n₂ is an integer from 0-2;
n₃ is an integer from 0-2;
   wherein the sum of n₂ and n₃ is 1 or 2; and
each occurrence of p is independently an integer from 0-4.

In some embodiments, n₂ is 1 and n₃ is 0. In some embodiments, n₂ is 0 and n₃ is 1. In some embodiments, n₂ is 1 and n₃ is 1. In some embodiments, n₂ is 2 and n₃ is 0. In some embodiments, n₂ is 0 and n₃ is 2.

In some embodiments, V is CR₁, wherein R₁ is H, halogen, or alkyl. In some embodiments, V is CR₁, wherein R₁ is alkyl. In some embodiments, V is CR₁, wherein R₁ is (CR₇R₈)ₚNRₐR_{b}. In some embodiments, V is CR₁, wherein R₁ is H or alkyl. In certain embodiments, the alkyl is a C₁-C₄ alkyl group. Non-limiting examples of C₁-C₄ alkyl groups include methyl, ethyl, propyl, isopropyl, butyl, isobutyl, sec-butyl, and tert-butyl. In some embodiments, V is CH.

In some embodiments, the compound has the structure of Formula Ia:

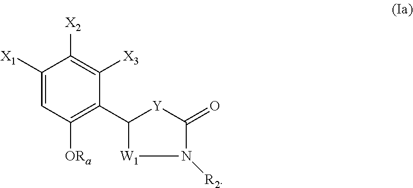

In some embodiments, the compound has the structure of Formula Ib:

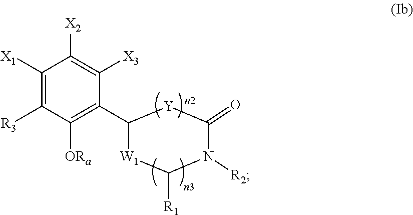

where n₂ is 1-2 and n₃ is 0-1; and where the sum of n₂ and n₃ is 2.

In some embodiments, W₁ is CHR₁ or NR₄. In some embodiments, W₁ is CHR₁ or O. In some embodiments, W₁ is O. In some embodiments, W₁ is CHR₁. In some embodiments, W₁ is NR₄.

In some embodiments, each occurrence of W is independently CHR₁ or NR₅. In some embodiments, each occurrence of W is independently CHR₁ or O. In some embodiments, at least one occurrence of W is O. In some embodiments, W is CHR₁. In some embodiments, W is NR₄.

In some embodiments, each occurrence of Y is independently CHR₁ or O. In some embodiments, each occurrence of Y is independently CHR₁ or NR₆. In some embodiments, at least one occurrence of Y is O. In some embodiments, Y is CHR₁. In some embodiments, Y is NR₄.

In some embodiments, each occurrence of R₁ is independently H, halogen, or alkyl. In some embodiments, each occurrence of R₁ is independently H or (CR₇R₈)ₚNRₐR_{b}. In some embodiments, each occurrence of R₁ is independently H, alkyl, or (CR₇R₈)ₚNRₐR_{b}. In some embodiments, each occurrence of R₁ is independently H, CH₃, CH₂CH₃, NH₂, NHCH₃, or N(CH₃)₂. In some embodiments, each occurrence of R₁ is H.

In some embodiments, W₁ is CHR₁, where R₁ is H, halogen, or alkyl. In some embodiments, W₁ is CHR₁, where R₁ is H or (CR₇R₈)ₚNRₐR_{b}. In some embodiments, W₁ is CHR₁, where R₁ is H, alkyl, or (CR₇R₈)ₚNRₐR_{b}. In some embodiments, W₁ is CHR₁, where R₁ is H, CH₃, CH₂CH₃, NH₂, NHCH₃, or N(CH₃)₂.

In some embodiments, R₄ is H, alkyl, heteroalkyl, cycloalkyl, or cycloheteroalkyl. In some embodiments, R₄ is aryl, alkylaryl, or heteroaryl. In some embodiments, R₄ is H or alkyl. In some embodiments, R₄ is H.

In some embodiments, W₁ is NR₄. In some embodiments, W₁ is NR₄, where R₄ is H, alkyl, heteroalkyl, cycloalkyl, or cycloheteroalkyl. In some embodiments, W₁ is NR₄, where R₄ is aryl, alkylaryl, or heteroaryl. In some embodiments, W₁ is NR₄, where R₄ is H or alkyl. In some embodiments, W₁ is NR₄, where R₄ is H.

In some embodiments, at least one occurrence of W is CHR₁, where R₁ is H, halogen, or alkyl. In some embodiments, at least one occurrence of W is CHR₁, where R₁ is H or (CR₇R₈)ₚNRₐR_{b}. In some embodiments, at least one occurrence of W is CHR₁, where R₁ is H, alkyl, or (CR₇R₈)ₚNRₐR_{b}. In some embodiments, at least one occurrence of W is CHR₁, where R₁ is H, CH₃, CH₂CH₃, NH₂, NHCH₃, or N(CH₃)₂.

In some embodiments, each occurrence of R₅ is independently H, alkyl, heteroalkyl, cycloalkyl, or cycloheteroalkyl. In some embodiments, each occurrence of R₅ is independently aryl, alkylaryl, or heteroaryl. In some embodiments, each occurrence of R₅ is H or alkyl. In some embodiments, each occurrence of R₅ is H.

In some embodiments, at least one occurrence of W is NR₅. In some embodiments, at least one occurrence of W is NR₅, where R₅ is H, alkyl, heteroalkyl, cycloalkyl, or cycloheteroalkyl. In some embodiments, at least one occurrence of W is NR₅, where R₅ is aryl, alkylaryl, or heteroaryl. In some embodiments, at least one occurrence of W is NR₅, where R₅ is H or alkyl. In some embodiments, at least one occurrence of W is NR₅, where R₅ is H.

In some embodiments, at least one occurrence of Y is CHR₁, where R₁ is H, halogen, or alkyl. In some embodiments, at least one occurrence of Y is CHR₁, where R₁ is H or (CR₇R₈)ₚNRₐR_{b}. In some embodiments, at least one occurrence of Y is CHR₁, where R₁ is H, alkyl, or (CR₇R₈)ₚNRₐR_{b}. In some embodiments, at least one occurrence of Y is CHR₁, where R₁ is H, CH₃, CH₂CH₃, NH₂, NHCH₃, or N(CH₃)₂.

In some embodiments, each occurrence of R₆ is independently H, alkyl, heteroalkyl, cycloalkyl, or cycloheteroalkyl. In some embodiments, each occurrence of R₆ is independently aryl, alkylaryl, or heteroaryl. In some embodiments, each occurrence of R₆ is H or alkyl. In some embodiments, each occurrence of R₆ is H.

In some embodiments, at least one occurrence of Y is NR₆. In some embodiments, at least one occurrence of Y is NR₆, where R₆ is H, alkyl, heteroalkyl, cycloalkyl, or cycloheteroalkyl. In some embodiments, at least one occurrence of Y is NR₆, where R₆ is aryl, alkylaryl, or heteroaryl. In some embodiments, at least one occurrence of Y is NR₆, where R₆ is H or alkyl. In some embodiments, at least one occurrence of Y is NR₆, where R₆ is H.

In some embodiments, R₂ is H, alkyl, or (CR₇R₈)ₚcycloalkyl. Non-limiting examples of cycloalkyl groups include optionally substituted cyclopropyl, optionally substituted cyclobutyl, optionally substituted cyclopentyl, or optionally substituted cyclohexyl. In some embodiments, R₂ is (CR₇R₈)ₚheteroalkyl or (CR₇R₈)ₚcycloheteroalkyl. Non-limiting examples of cycloheteroalkyl groups include optionally substituted azetidinyl, optionally substituted oxetanyl, optionally substituted pyrrolidinyl, optionally substituted tetrahydrofuranyl, optionally substituted tetrahydropyranyl, optionally substituted piperazinyl, optionally substituted piperazinonyl, and optionally substituted pyridinonyl. In some embodiments, R₂ is (CR₇R₈)ₚaryl or (CR₇R₈)ₚheteroaryl. Non-limiting examples of heteroaryl groups include optionally substituted isoxazolyl, optionally substituted isothiazolyl, optionally substituted pyridinyl, optionally substituted imidazolyl, optionally substituted thiazolyl, optionally substituted pyrazolyl, and optionally substituted triazolyl. In some embodiments, R₂ is (CR₇R₈)ₚORₐ or (CR₇R₈)ₚNRₐR_{b}. In some embodiments, R₂ is (CR₇R₈)ₚ(C═O)ORₐ, (CR₇R₈)ₚNRₐ(C═O)R_{b}, or (CR₇R₈)ₚ(C═O)NRₐR_{b}. In some embodiments, R₂ is (CR₇R₈)ₚNRₐ(C═O)R_{b}.

In some embodiments, each occurrence of R₇ and R₈ is independently H or alkyl. In some embodiments, each occurrence of R₇ and R₈ is H. In some embodiments, each occurrence of R₇ and R₈ is alkyl. In certain embodiments, the alkyl is a C₁-C₄ alkyl group, such as, but not limited to, methyl, ethyl, propyl, isopropyl, butyl, isobutyl, sec-butyl, and tert-butyl. In some embodiments, each occurrence of R₇ and R₈ is independently H, cycloalkyl, aryl, or heteroaryl. In some embodiments, each occurrence of R₇ and R₈ is independently H or cycloalkyl.

In some embodiments, each occurrence of Rₐ and R_{b} is independently H or alkyl. In some embodiments, each occurrence of Rₐ and R_{b} is H. In some embodiments, each occurrence of Rₐ and R_{b} is alkyl. In certain embodiments, the alkyl is a C₁-C₄ alkyl group, such as, but not limited to, methyl, ethyl, propyl, isopropyl, butyl, isobutyl, sec-butyl, and tert-butyl. In some embodiments, each occurrence of Rₐ and R_{b} is independently cycloalkyl, heterocycle, aryl, or heteroaryl. In some embodiments, each occurrence of Rₐ and R_{b} is independently H or cycloalkyl.

In some embodiments, at least one occurrence of p is 0, 1, or 2. In some embodiments, at least one occurrence of p is 0. In some embodiments, at least one occurrence of p is 1. In some embodiments, at least one occurrence of p is 2. In some embodiments, at least one occurrence of p is 3 or 4. In some embodiments, at least one occurrence of p is 3. In some embodiments, at least one occurrence of p is 4.

In some embodiments, V is CH and the structural moiety

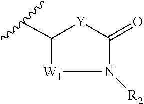

has the structure of

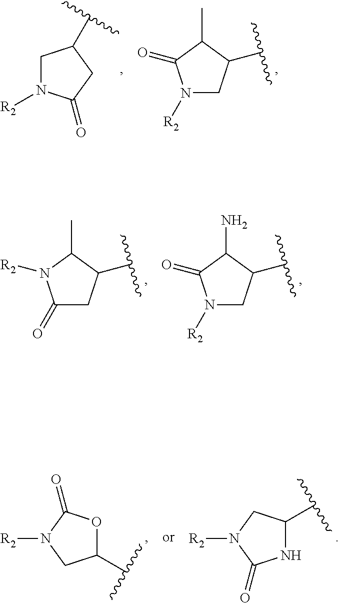

In some embodiments, V is CH and the structural moiety

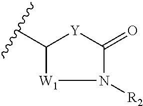

has the structure of

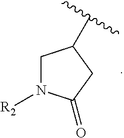

In some embodiments, V is CH and the structural moiety

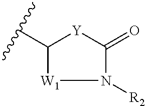

has the structure of

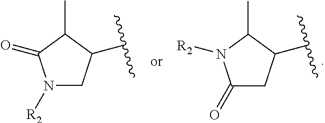

In some embodiments, V is CH and the structural moiety

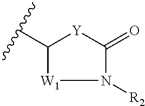

has the structure of

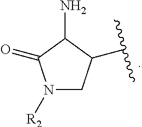

In some embodiments, V is CH and the structural moiety

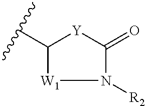

has the structure of

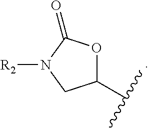

In some embodiments, V is CH and the structural moiety

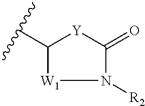

has the structure of

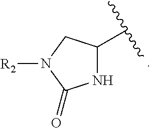

In some embodiments, V is CH and the structural moiety

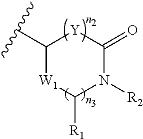

has the structure of

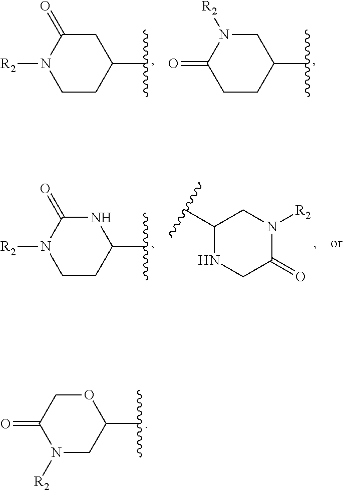

In some embodiments, V is CH and the structural moiety

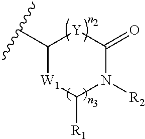

has the structure of

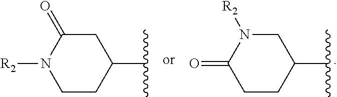

In some embodiments, V is CH and the structural moiety

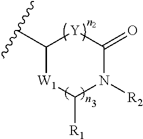

has the structure of

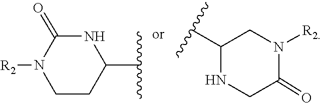

In some embodiments, V is CH and the structural moiety

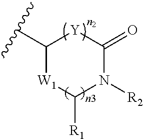

has the structure of

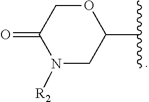

In some embodiments, R₂ is

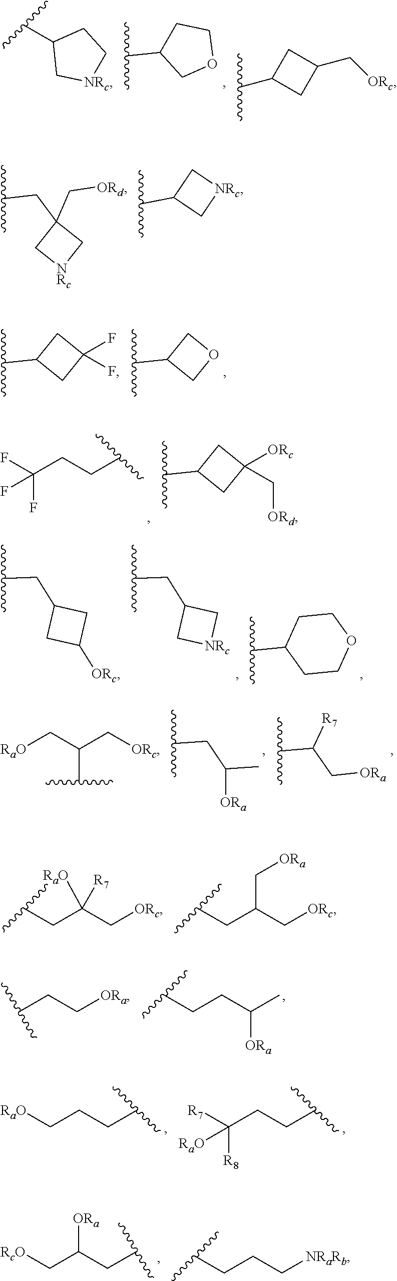

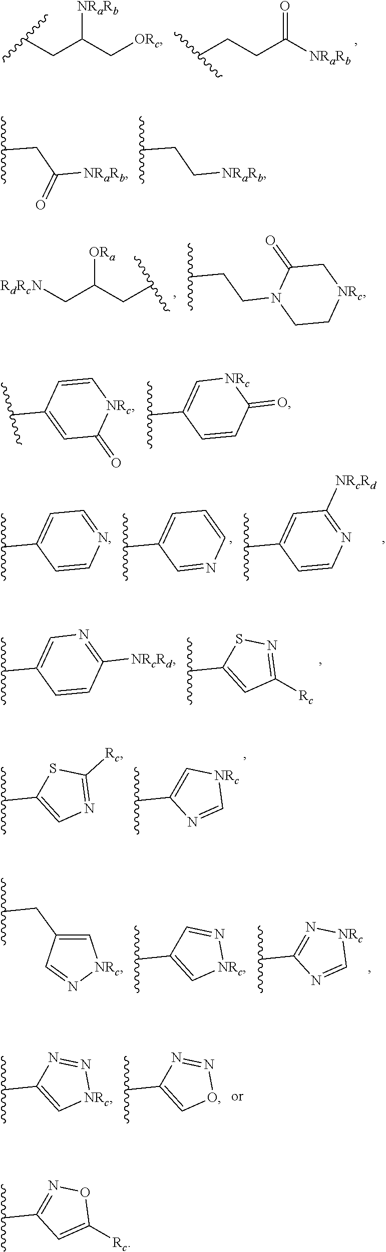

In some embodiments, R₂ is

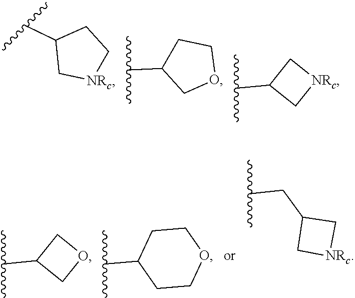

In some embodiments, R₂ is

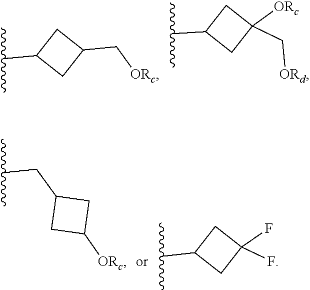

In some embodiments, R₂ is

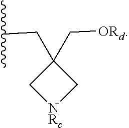

In some embodiments, R₂ is

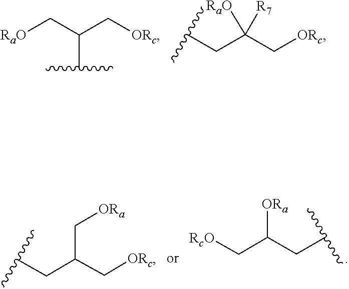

In some embodiments, R₂ is

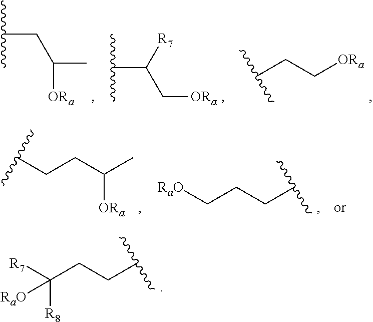

In some embodiments, R₂ is

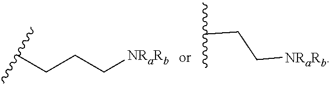

In some embodiments, R₂ is

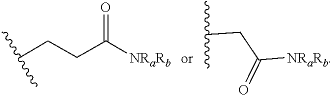

In some embodiments, R₂ is

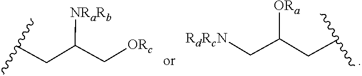

In some embodiments, R₂ is

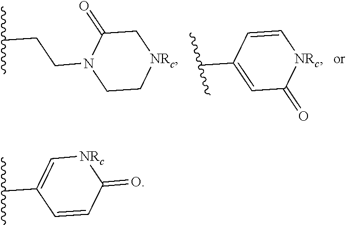

In some embodiments, R₂ is

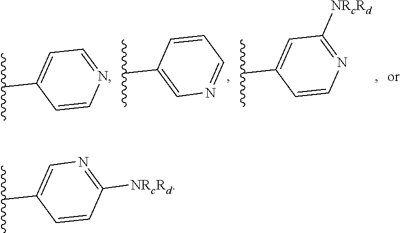

In some embodiments, R₂ is

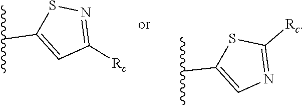

In some embodiments, R₂ is

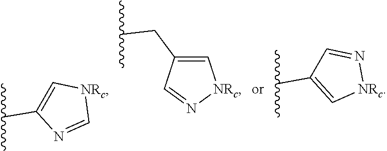

In some embodiments, R₂ is

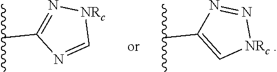

In some embodiments, R₂ is

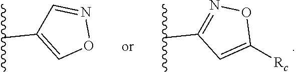

In some embodiments, each occurrence of Rₐ and R_{b} is independently H or alkyl. In some embodiments, each occurrence of Rₐ and R_{b} is independently H or O(C₁₋₄ alkyl). Non-limiting examples of C₁₋₄ alkyl include methyl, ethyl, propyl, isopropyl, butyl, isobutyl, sec-butyl, and tert-butyl. In some embodiments, each occurrence of Rₐ and R_{b} is independently H, cycloalkyl, heterocycle, aryl, or heteroaryl. In some embodiments, each occurrence of Rₐ and R_{b} is independently H or cycloalkyl.

In some embodiments, each occurrence of R_{c} and R_{d} is independently H or alkyl. In some embodiments, each occurrence of R_{c} and R_{d} is independently H or C₁-C₄ alkyl. Non-limiting examples of C₁-C₄ alkyl include methyl, ethyl, propyl, isopropyl, butyl, isobutyl, sec-butyl, and tert-butyl. In some embodiments, each occurrence of R_{c} and R_{d} is independently H, cycloalkyl, heterocycle, aryl, or heteroaryl. In some embodiments, each occurrence of R_{c} and R_{d} is independently H or cycloalkyl

In some embodiments R₂ is

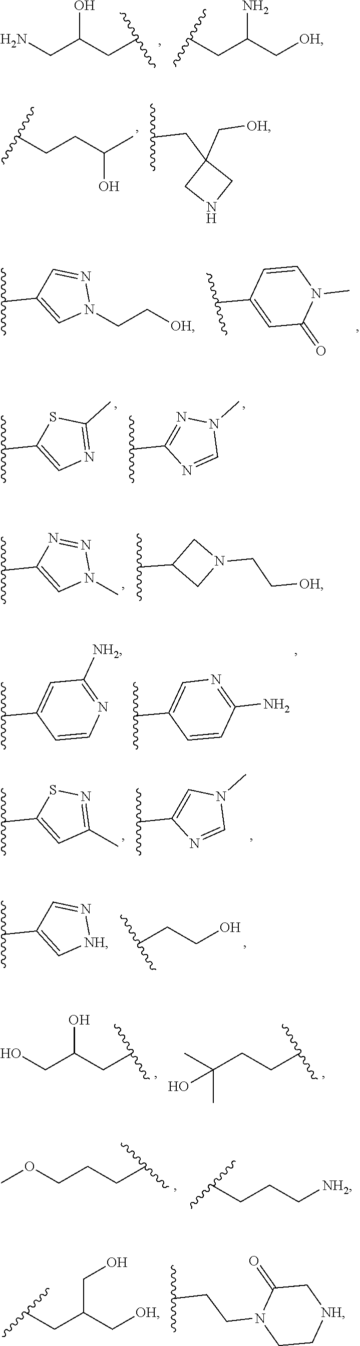

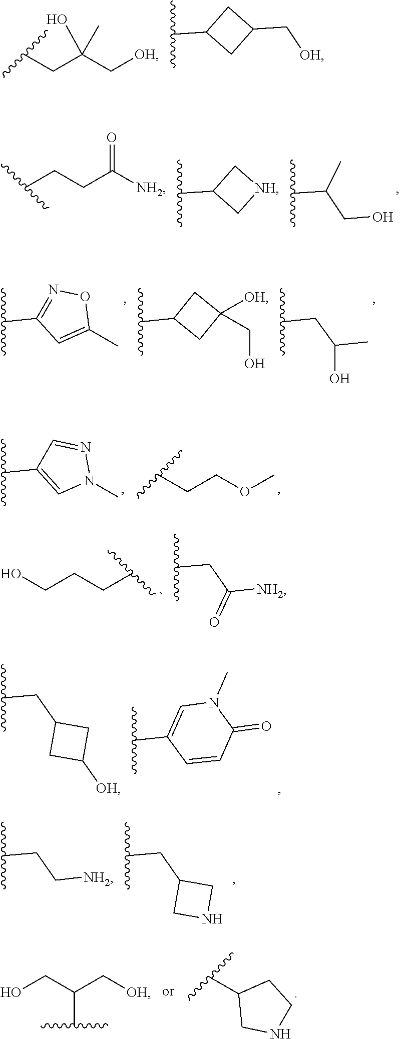

In some embodiments, R₂ is

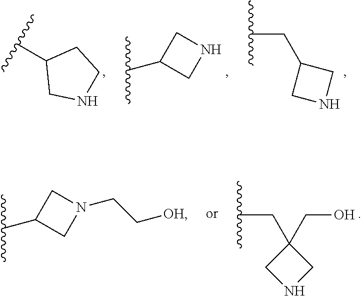

In some embodiments, R₂ is

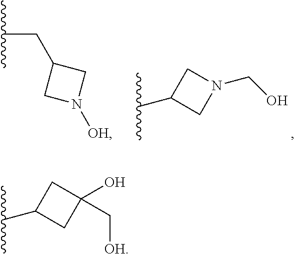 [SMILES: *CC1CN(O)C1 ; *C1CN(CO)C1 ; *C1CC(O)(CO)C1]

In some embodiments, R₂ is

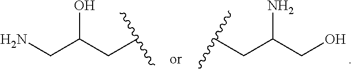 [SMILES: *CC(O)CN ; *CC(N)CO]

In some embodiments, R₂ is

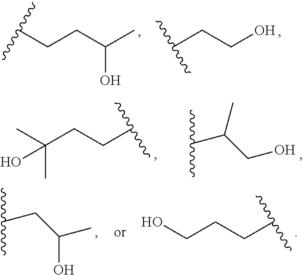

In some embodiments, R₂ is

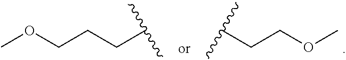

In some embodiments, R₂ is

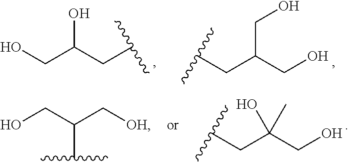

In some embodiments, R₂ is

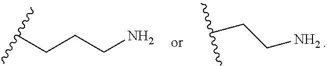

In some embodiments, R₂ is

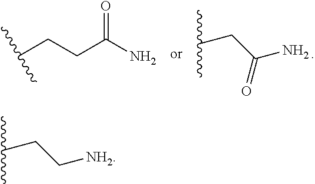

In some embodiments, R₂ is

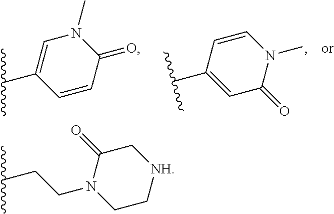

In some embodiments, R₂ is

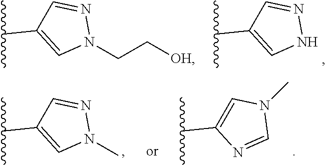

In some embodiments, R₂ is

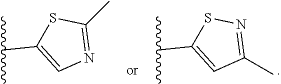

In some embodiments, R₂ is

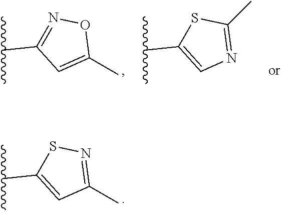

In some embodiments, R₂ is

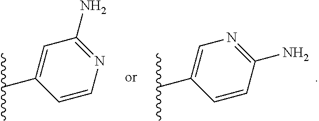 [SMILES: *c1ccnc(N)c1 ; *c1ccc(N)nc1]

In some embodiments, R₂ is

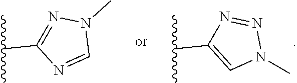

In some embodiments, the structural moiety

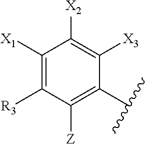

has the structure of

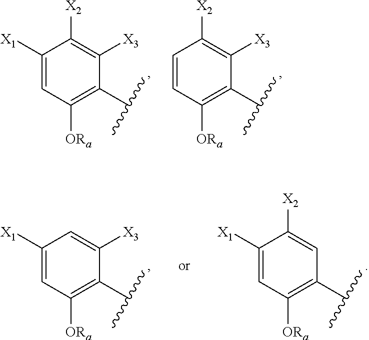

In some embodiments, the structural moiety

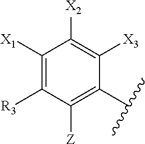

has the structure of

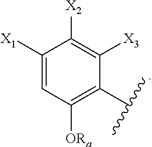

In some embodiments, the structural moiety

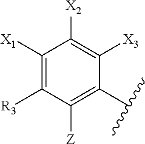

has the structure of

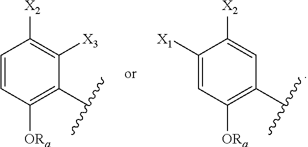

In some embodiments, the structural moiety

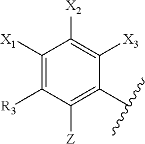

has the structure of

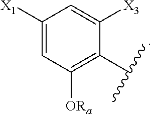

In some embodiments, X₁ is H, halogen, alkyl, or halogenated alkyl. In some embodiments, X₁ is H, F, Cl, Br, CH₃, CH₂F, CHF₂, or CF₃. In some embodiments, X₁ is H or Cl. In some embodiments, X₁ is H. In some embodiments, X₁ is Cl. In some embodiments, X₁ is CN, cycloalkyl, or halogenated cycloalkyl. In some embodiments, X₁ is CN. In some embodiments, X₁ is cycloalkyl or halogenated cycloalkyl.

In some embodiments, X₂ is H, halogen, alkyl, or halogenated alkyl. In some embodiments, X₂ is H, F, Cl, Br, CH₃, CH₂F, CHF₂, or CF₃. In some embodiments, X₂ is H or Cl. In some embodiments, X₂ is H. In some embodiments, X₂ is Cl. In some embodiments, X₂ is CN, cycloalkyl, or halogenated cycloalkyl. In some embodiments, X₂ is CN. In some embodiments, X₂ is cycloalkyl or halogenated cycloalkyl.

In some embodiments, X₃ is H, halogen, alkyl, or halogenated alkyl. In some embodiments, X₃ is H, F, Cl, Br, CH₃, CH₂F, CHF₂, or CF₃. In some embodiments, X₃ is H or Cl. In some embodiments, X₃ is H. In some embodiments, X₃ is Cl. In some embodiments, X₃ is CN, cycloalkyl, or halogenated cycloalkyl. In some embodiments, X₃ is CN. In some embodiments, X₃ is cycloalkyl or halogenated cycloalkyl.

In some embodiments, Z is ORₐ, where Rₐ is H, alkyl, cycloalkyl, heterocycle, aryl, or heteroaryl. In some embodiments, Z is ORₐ, where Rₐ is H or O(C₁₋₄ alkyl). Non-limiting examples of C₁₋₄ alkyl include methyl, ethyl, propyl, isopropyl, butyl, isobutyl, sec-butyl, and tert-butyl. In some embodiments, Z is OH, OCH₃, or OCH₂CH₃. In some embodiments, Z is OH.

In some embodiments, R₃ is H, halogen, alkyl, or cycloalkyl. In some embodiments, R₃ is saturated heterocycle, aryl, or heteroaryl. In some embodiments, R₃ is CN, CF₃, OCF₃, ORₐ or SRₐ, where Rₐ is H, alkyl, cycloalkyl, heterocycle, aryl, or heteroaryl. In some embodiments, R₃ is CN, CF₃, OCF₃, ORₐ or SRₐ, where Rₐ is H or alkyl. In some embodiments, R₃ is NRₐR_{b} or NRₐ(C═O)R_{b}, where Rₐ and R_{b} are each independently H, alkyl, cycloalkyl, heterocycle, aryl, or heteroaryl. In some embodiments, R₃ is NRₐR_{b} or NRₐ(C═O)R_{b}, where Rₐ and R_{b} are each independently H or alkyl. In some embodiments, R₃ is H, F, Cl, Br, C₁₋₄ alkyl, or CF₃. Non-limiting examples of C₁₋₄ alkyl include methyl, ethyl, propyl, isopropyl, butyl, isobutyl, sec-butyl, and tert-butyl. In some embodiments, R₃ is H.

In some embodiments, the structural moiety

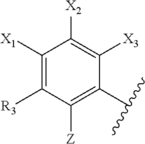

has the structure of

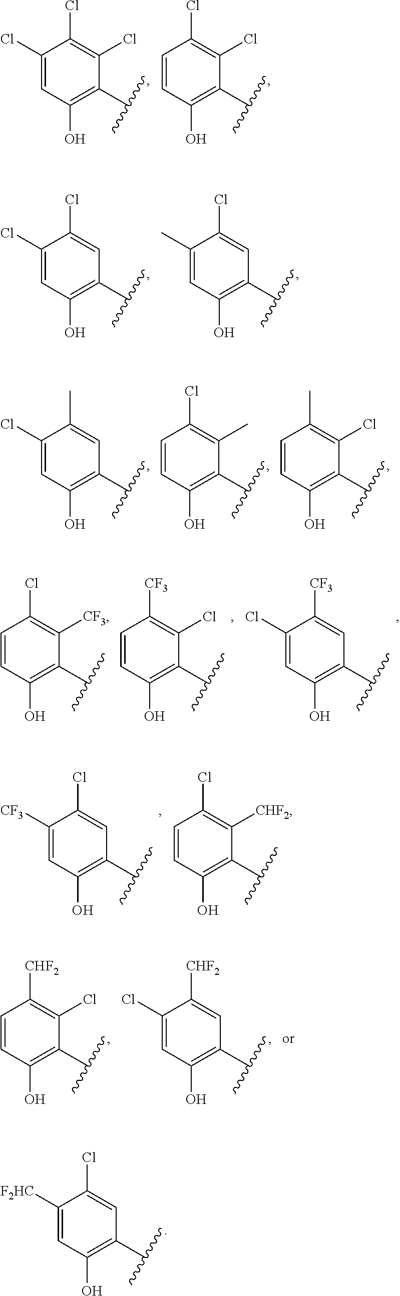

In some embodiments, the structural moiety

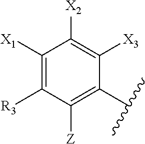

has the structure of

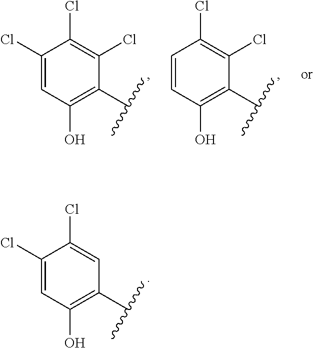

In some embodiments, the structural moiety

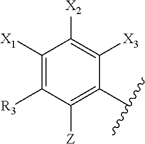

has the structure of

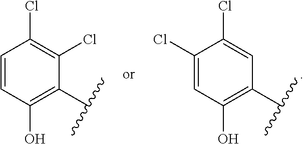 [SMILES: *c1c(O)ccc(Cl)c1Cl ; *c1cc(Cl)c(Cl)cc1O]

In some embodiments, the structural moiety

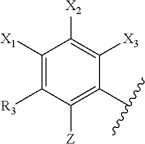

has the structure of

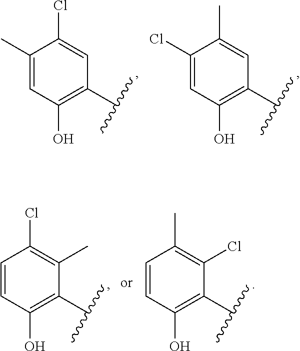

In some embodiments, the structural moiety

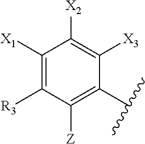

has the structure of

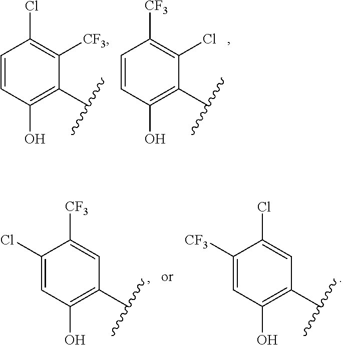

In some embodiments, the structural moiety

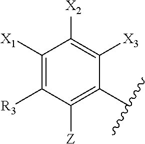

has the structure of

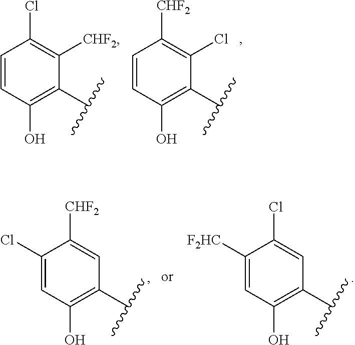

In some embodiments, the alkyl, cycloalkyl, heteroalkyl, cycloheteroalkyl, aryl, heteroaryl, carbocycle, and heterocycle of X₁, X₂, X₃, R₁, R₂, R₃, R₄, R₅, R₆, R₇, R₈, Rₐ, and R_{b}, where applicable, are each independently and optionally substituted by 1-4 substituents each independently selected from the group consisting of alkyl, cycloalkyl, halogenated alkyl, halogenated cycloalkyl, and halogen where valence permits. In some embodiments, the alkyl, cycloalkyl, heteroalkyl, cycloheteroalkyl, aryl, heteroaryl, carbocycle, and heterocycle of X₁, X₂, X₃, R₁, R₂, R₃, R₄, R₅, R₆, R₇, R₈, Rₐ, and R_{b}, where applicable, are each independently and optionally substituted by 1-4 substituents each independently selected from the group consisting of CN, Rₑ, (CR_{c}R_{d})ₚOR_{c}, and (CR_{c}R_{d})ₚNR_{c}R_{d} where valence permits. In some embodiments, the alkyl, cycloalkyl, heteroalkyl, cycloheteroalkyl, aryl, heteroaryl, carbocycle, and heterocycle of X₁, X₂, X₃, R₁, R₂, R₃, R₄, R₅, R₆, R₇, R₈, Rₐ, and R_{b}, where applicable, are each independently and optionally substituted by 1-4 substituents each independently selected from the group consisting of (CR_{c}R_{d})ₚ(C═O)OR_{c}, (CR_{c}R_{d})ₚ(C═O)NR_{c}R_{d}, (CR_{c}R_{d})ₚNR_{c}(C═O)R_{d}, and oxo where valence permits.

In certain embodiments, the compound is selected from the group consisting of compounds 1-105 as shown in Table 1.

### Abbreviations

ACN Acetonitrile
Boc Tert-butyloxycarbonyl
CDI Carbonyldiimidazole
DBU 1,8-Diazabicyclo[5.4.0]undec-7-ene
DCE 1,2-Dichloroethane
DCM Dichloromethane
DIEA Diisopropylethylamine
DMEDA 1,2-Dimethylethylenediamine
DMEM Dulbecco's Modified Eagle Medium
DMF Dimethyl formamide
DPPA Diphenylphosphoryl azide
EA Ethyl acetate
EGTA Ethylene glycol-bis(3-aminoethyl ether)-N,N,N′,N′-tetraacetic acid
ESI Electrospray ionization
FBS Fetal bovine serum
MOM Methoxymethyl acetal
MsCl Methanesulfonyl chloride
NMO N-Methylmorpholine N-oxide
PE Petroleum ether
SEMCl 2-(Trimethylsilyl)ethoxymethyl chloride
SFC Supercritical fluid chromatography
TBSCl Tert-butyldimethylsilyl chloride
TEA Triethylamine
TFA Trifluoroacetic acid
THF Tetrahydrofuran
TMEDA Tetramethylethylenediamine

### Methods of Preparation

Following are general synthetic schemes for manufacturing compounds of the present invention. These schemes are illustrative and are not meant to limit the possible techniques one skilled in the art may use to manufacture the compounds disclosed herein. Different methods will be evident to those skilled in the art. Additionally, the various steps in the synthesis may be performed in an alternate sequence or order to give the desired compound(s). For example, the following reactions are illustrations, but not limitations of the preparation of some of the starting materials and compounds disclosed herein.

Schemes 1-11 below describe synthetic routes which may be used for the synthesis of compounds of the present invention, e.g., compounds having a structure of Formula I or a precursor thereof. Various modifications to these methods may be envisioned by those skilled in the art to achieve similar results to that of the inventions given below. In the embodiments below, the synthetic route is described using compounds having the structure of Formula I or a precursor thereof as examples. The general synthetic routes described in Schemes 1-11 and examples described in the Example section below illustrate methods used for the preparation of the compounds described herein.

As shown in Scheme 1 below, the core of certain compounds of Formula I can be synthesized from a suitable substituted bromo or iodo benzene I-1a that is converted to the corresponding boronic acid I-2 by metalation with, for example, n-butyl lithium and reaction with a trialkyl borate (e.g., trimethyl borate). Alternatively, with certain protecting groups (“PG”) (e.g., MOM or SEM), direct deprotonation of benzene I-1b with, for example, n-butyl lithium and reaction with a trialkyl borate (e.g., trimethyl borate) can afford the boronic acid I-2.

Also as shown in Scheme 1 below, for six-membered lactams, boronic acid I-2 can be reacted with 5,6-dihydropyran-2-one in the presence of a rhodium catalyst (e.g., [Rh(COD)Cl]₂) and a base (e.g., K₃PO₄) in an inert solvent (e.g., dioxane) to give lactone I-3. Reaction of lactone I-3 with an amine (e.g., RNH₂) and a Lewis acid (e.g., trimethylaluminum) results in ring-opening of the lactone to hydroxy amide I-4. Depending on the PG used, it may be necessary to re-protect or change PG. The alcohol in I-4 is then converted to a leaving group such as mesylate or tosylate (I-5) and cyclized using a base (e.g., sodium hydride) in a polar solvent (e.g., DMF) to give lactam I-6. Removal of the PG yields 6-member lactam I-7.

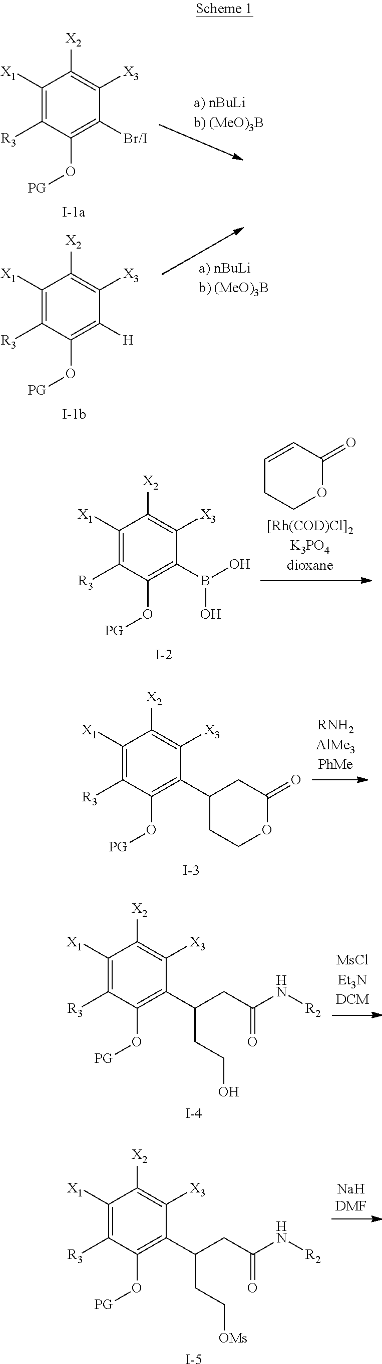

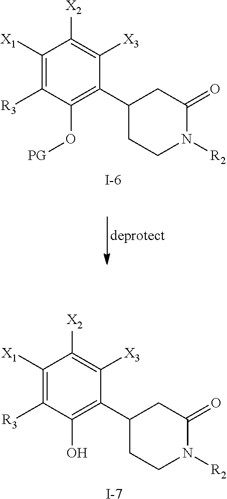

As shown in Scheme 2 below, an alternative approach to 6-member lactams where Y=CR₁ or NR₆ starts from an aromatic heterocycle such as I-8, containing an amide in the ring. N-Alkylation with R₂X gives I-9 that undergoes Suzuki coupling with boronic acid I-2 in the presence of a catalyst (e.g., Pd(dppf)) and a base (e.g., sodium carbonate) in a solvent (e.g., dioxane). The resulting biaryl I-10 is reduced by hydrogenation over a catalyst (e.g., palladium or platinum) to give I-11. Removal of the PG gives I-12.

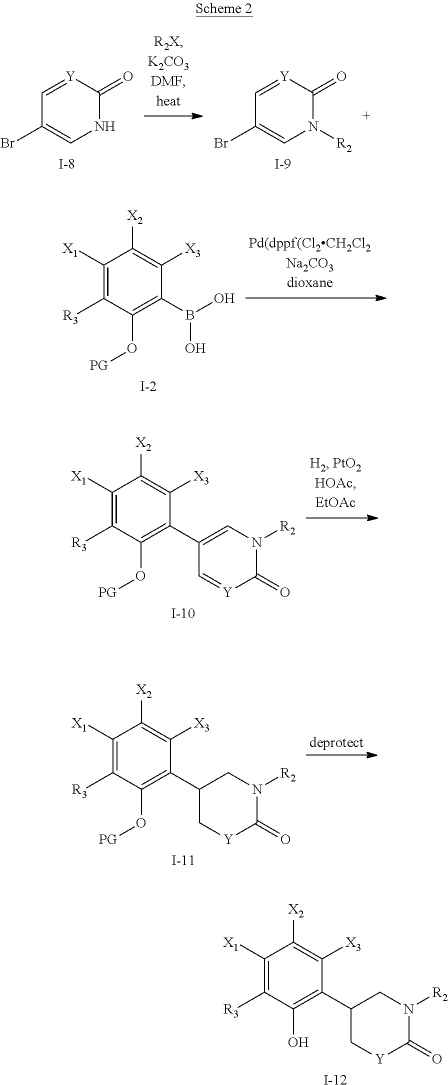

A variation of the preceding route (Scheme 3 below) uses a thiomethyl substituted heterocycle that is coupled with boronic acid I-2 using a palladium catalyst (e.g., XPhos Pd) and a base (e.g., potassium phosphate) to form biaryl I-13. Hydrolysis of the thiomethyl ether in I-13 converts it to I-14 that is then alkylated on nitrogen with a suitable alkylating agent R₂X, where X is a halogen or a sulfonate and a base (e.g., potassium carbonate), to form I-15. Hydrogenation of I-15 over a platinum or palladium catalyst yields the saturated heterocycle I-16, that is then deprotected to give I-17.

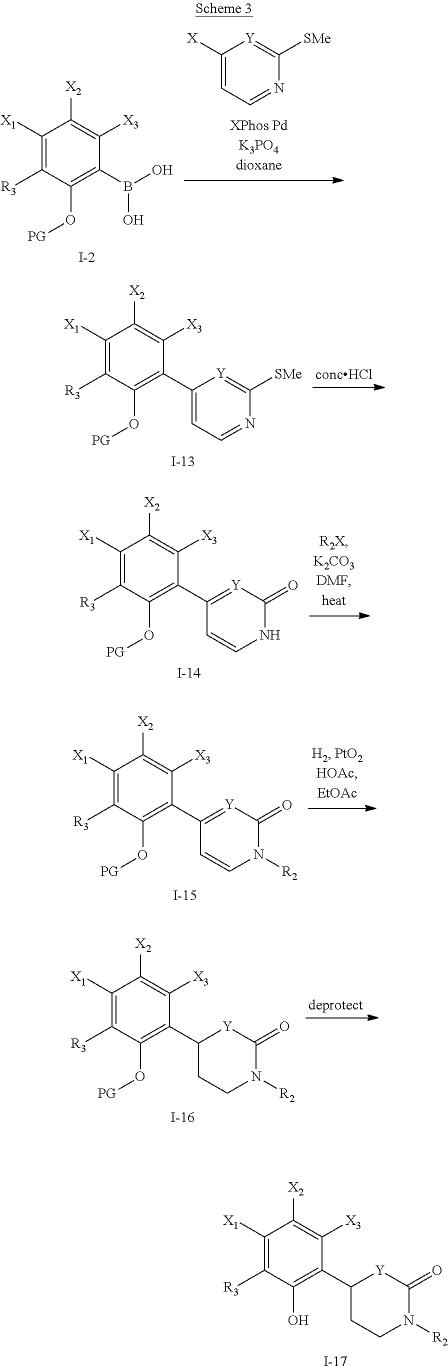

As shown in Scheme 4 below, five-membered lactams may be prepared by Michael addition of a nitroalkane to an unsaturated ester. A suitable substituted phenol is first protected with a PG to form I-1b. Preferably, PG is an ether-containing group that can direct ortho lithiation of the benzene ring (e.g., SEM or MOM). Treatment of I-1b with an alkyl lithium (e.g., n-butyl lithium) in an ether solvent (e.g., THF) at low temperature followed by addition of a formamide (e.g., DMF) yields the aldehyde I-18. Reaction of I-18 with an ester (e.g., EA) and a base (e.g., sodium hydride) gives the unsaturated ester I-19. There are other methods known in the art that may be used to prepare I-18, such as, but not limited to, Villsmeier formylation, and methods to convert I-18 to I-19 (e.g., Wittig or Horner-Wadsworth Emmons reactions). Unsaturated ester I-19 undergoes Michael addition of a nitroalkane R₁NO₂ in the presence of a base (e.g., DBU) and solvent (e.g., a nitroalkane) to give I-20. Reduction of the nitro group in I-20 using zinc in acetic acid provides the amino ester I-21 that can be stored in the open chain form as an amine salt (e.g., the trifluoroacetate). Treatment of the salt I-21 with a mild base (e.g., potassium carbonate) in methanol results in cyclization to the lactam I-22. One way to obtain N-substituted lactams is by reductive amination of amino ester I-21 with the appropriate aldehyde or ketone to give the N-substituted amine I-23 that cyclizes to I-24 on treatment with a base (e.g., lithium hydroxide). Alternatively, lactam I-22 can be alkylated with R₂X and a base (e.g., sodium hydride) in a solvent (e.g., THF). For R₂ groups containing a hydroxyl group, lactam I-22 is reacted with an epoxide and a base (e.g., cesium carbonate) in an alcohol solvent (e.g., isopropanol). Removal of all PG from I-24 yields lactam I-25.

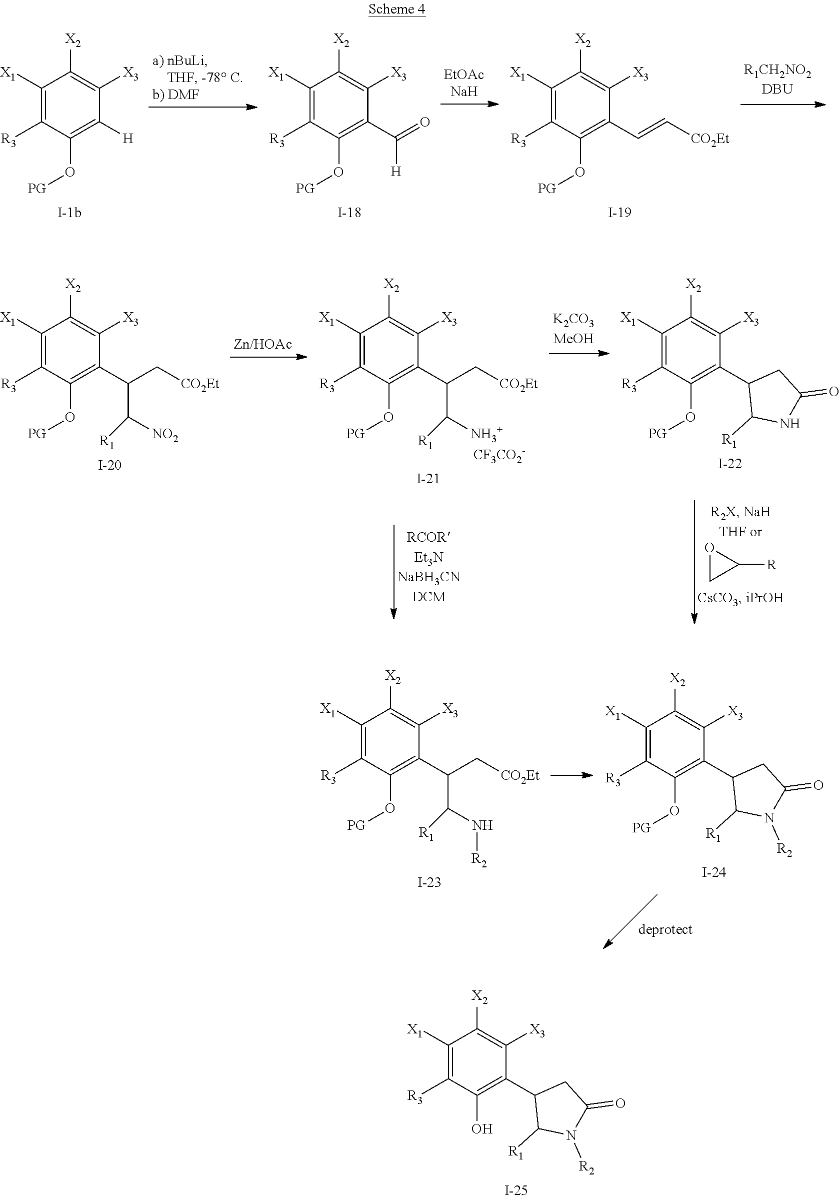

An alternative approach that provides an enantioselective synthesis of lactam I-22 and also gives access to lactams substituted at C3 is shown in Scheme 5 below. Reaction of aldehyde I-18 with nitromethane and a base (e.g., potassium carbonate) forms the nitroalcohol I-26. Elimination of water to form the nitrostyrene I-27 may be carried out using Burgess reagent in a hydrocarbon solvent (e.g., toluene). The nitrostyrene allows an enantioselective synthesis of I-29. Using diethyl malonate and the N-benzyl cyclohexanediamine nickel catalyst I-28 according to the method described in Evans et al., J. Am. Chem. Soc., 2007:11583-11592 provides I-29 enriched in the S enantiomer. Lactams with carbon substituents at C3 are obtained in racemic form using a substituted malonate R₁CH(CO₂Et)₂ and a base (e.g., potassium carbonate) in a polar solvent (e.g., DMF) to give I-29 (R₁=alkyl). Reduction of I-29 with zinc in acetic acid gives the amine salt I-30. Treatment of I-30 with a base (e.g., lithium hydroxide) in a solvent (e.g., methanol) results in cyclization to the lactam and hydrolysis of the ester to give the carboxylic acid I-31. Heating I-31 in an inert solvent (e.g., toluene) causes decarboxylation to form lactam I-32. The N-substituted lactam I-33 is formed either by N-alkylation of I-32 in the same way as lactam I-22 (Scheme 4) or by reductive amination of I-30 as described for I-21 (Scheme 4), followed by the same cyclization, hydrolysis, and decarboxylation sequence as for I-30. Removal of all PG yields I-34.

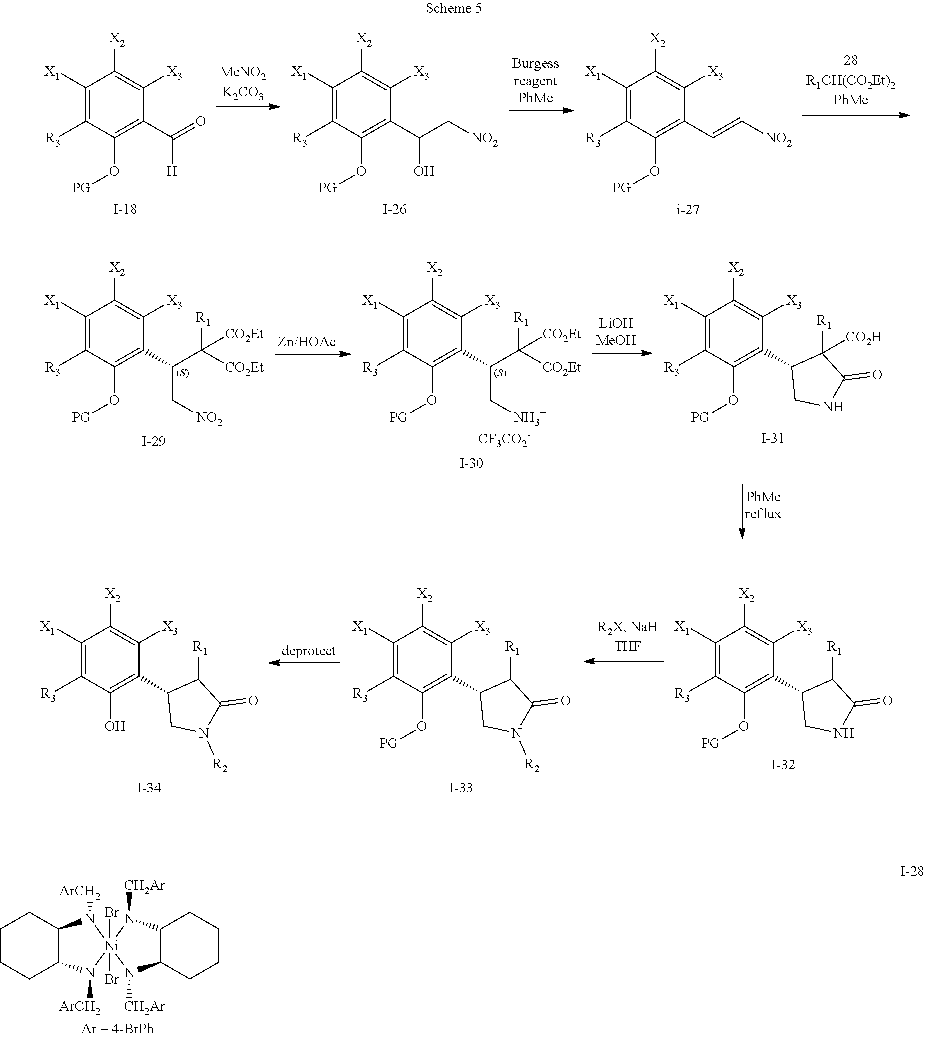

As shown in Scheme 6 below, lactams where R₂ is aryl can be made by an Ullmann reaction of the lactam I-22 or I-32 with a bromoarene, copper(I) iodide, potassium carbonate, and TMEDA, and heating in a solvent (e.g., dioxane) to give I-24a, which is deprotected to give I-25a.

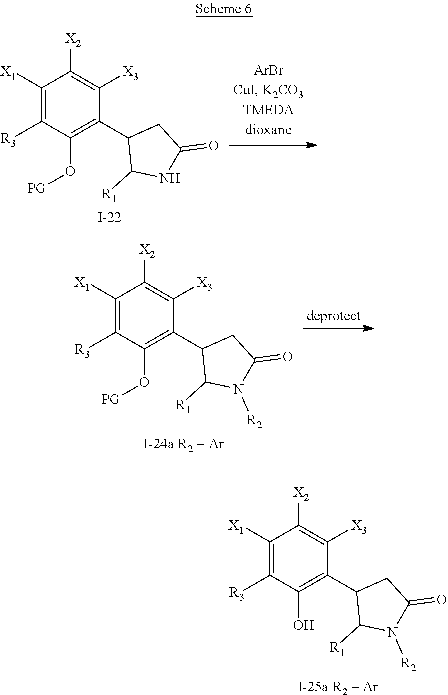

An alternative synthesis of lactam I-24 in which the substituent R₂ is introduced as an amine is shown in Scheme 7 below. Homologation of aldehyde I-18 using a methoxymethyl Wittig reagent gives the enol ether I-35 that is hydrolyzed to aldehyde I-36 with aqueous acid. Aldehyde I-36 is converted to an enamine by refluxing with a secondary amine (e.g., diisobutylamine) in a solvent (e.g., toluene). The enamine is then alkylated with ethyl bromoacetate and the iminium salt hydrolyzed to yield the ester aldehyde I-37. Reductive amination of aldehyde I-37 with an amine R₂NH₂ and a reducing agent (e.g., sodium triacetoxyborohydride) forms the substituted amine that cyclizes under the reaction conditions to give lactam I-24, which is then deprotected to give I-25.

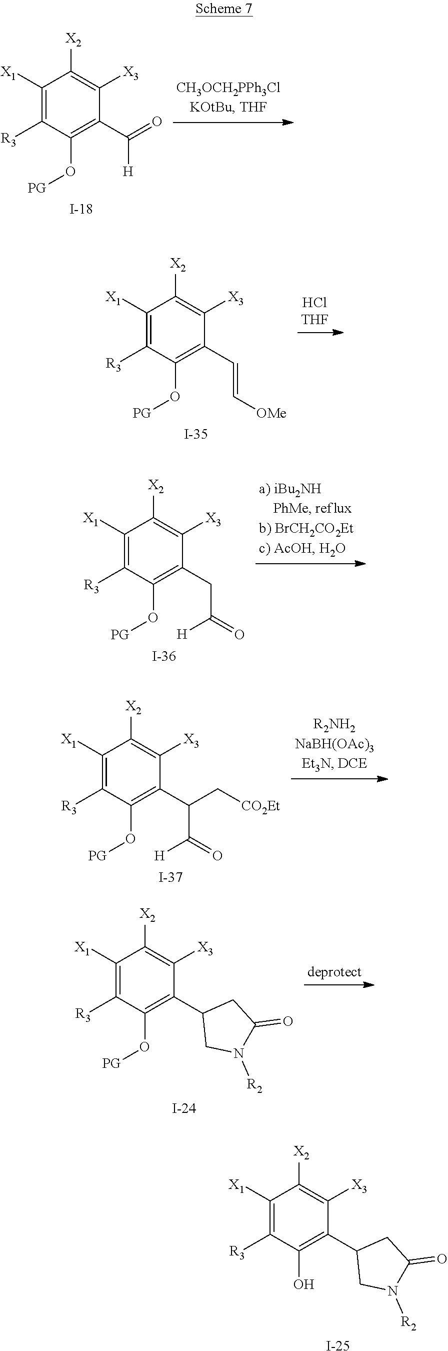

As shown in Scheme 8 below, lactams where R₁ is an amine attached to C3 can be prepared from amino ester I-30. Reductive amination of I-30 with an appropriate aldehyde or ketone using a reducing agent (e.g., sodium triacetoxyborohydride) followed by cyclization and ester hydrolysis with a base (e.g., lithium hydroxide) provides the N-substituted lactam carboxylic acid I-31a. Curtius reaction of I-31a with diphenyl phosphoryl azide and trapping with an alcohol (e.g., benzyl alcohol) forms the CBz-protected amine that can be deprotected to the free amine with, e.g., hydrogen bromide in acetic acid.

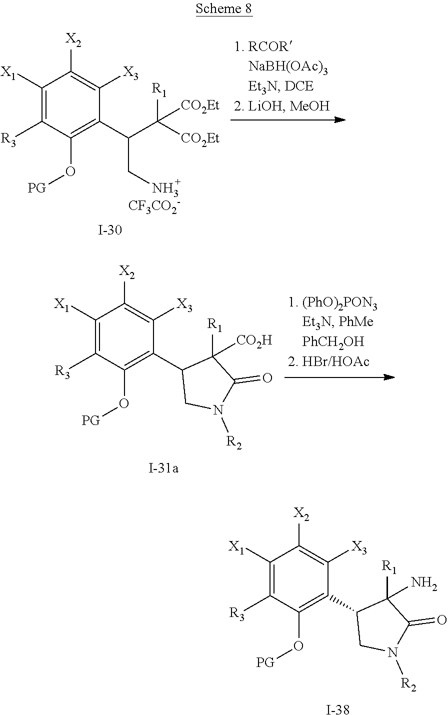

Compounds where W═O can be obtained from nitroalcohol I-26 as shown in Scheme 9 below. Reduction of the nitro group with zinc and acetic acid gives the amino alcohol I-39. Reductive amination of I-39 with an appropriate aldehyde or ketone and a reducing agent (e.g., sodium cyanoborohydride) provides the N-substituted amine I-40. To form the 5-membered ring, I-40 is reacted with carbonyl diimidazole to give I-41. For the 6-membered ring, I-40 is acylated on nitrogen with chloroacetyl chloride, and the resulting chloroamide is cyclized to give I-42 by treatment with a base (e.g., potassium hydroxide) in an alcohol solvent (e.g., isopropanol).

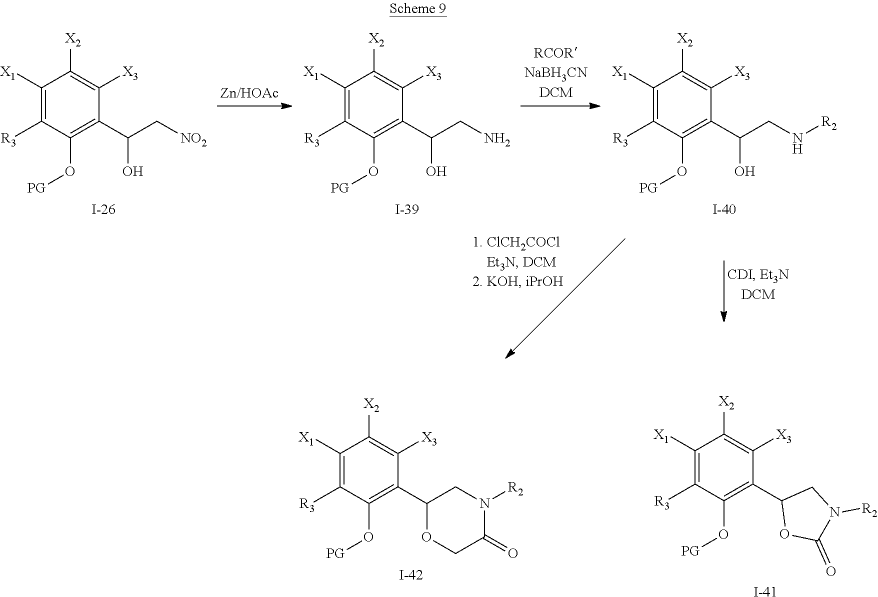

When W═N, the 6-membered ring can be prepared from nitrostyrene I-27, as shown in Scheme 10 below. Michael addition of ethyl glycinate in the presence of an amine base (e.g., diisopropylethylamine) yields I-43. The amine is protected with, e.g., a Boc group, and the nitro group is then reduced with zinc and acetic acid to form the amine I-44. Reductive amination of I-44 with an appropriate aldehyde or ketone and a reducing agent (e.g., sodium triacetoxyborohydride) gives the N-substituted amine that cyclizes under the reaction conditions to give piperazinone I-45. Removal of all PG by methods known in the art yields I-46.

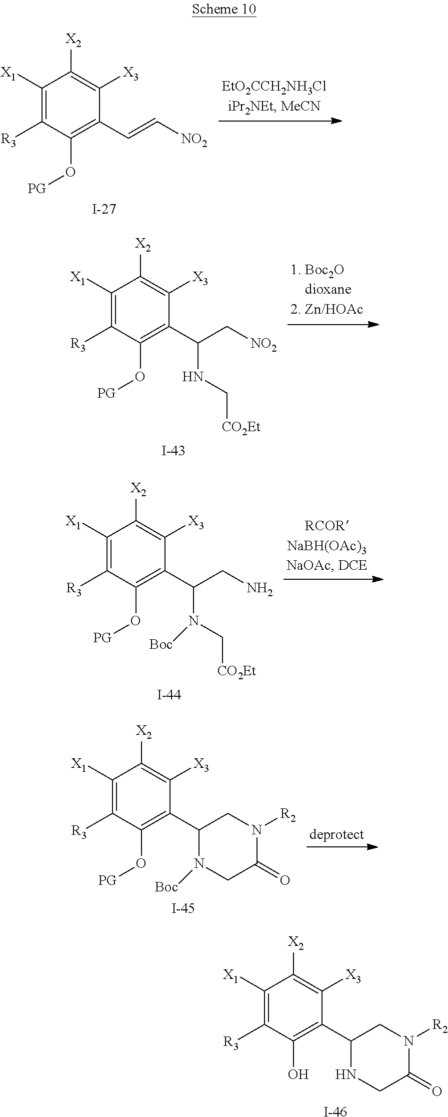

When W═N, the 5-membered ring can be prepared by the method shown in Scheme 11 below. Aldehyde I-18 is reacted with (S)-t-butyl sulfinimide and a Lewis acid (e.g., titanium tetraethoxide) in an ether solvent (e.g., THF) to form the sulfinyl imine I-47. Addition of a nitroalkane R₁CH₂NO₂ to I-47, catalyzed by a base (e.g., potassium carbonate) gives I-48. It is known from Garcia-Munoz et al., Tet. Asymm., 2014, 25:362-372 that addition of nitroalkanes to optically pure (S)-sulfinyl imines creates the S stereochemistry at the newly formed amine, so the configuration of I-48 is S,S as shown. Reduction of the nitro group with zinc and acetic acid gives the amine I-49. Reductive amination with an appropriate aldehyde or ketone introduces the R₂ substituent in I-50. Removal of the sulfinimide by hydrolysis with dilute acid (e.g., HCl) and an alcohol cosolvent (e.g., methanol) provides the diamine I-51. Treatment of I-51 with carbonyl diimidazole gives the cyclic urea I-52. Removal of PG by methods known in the art yields I-53.

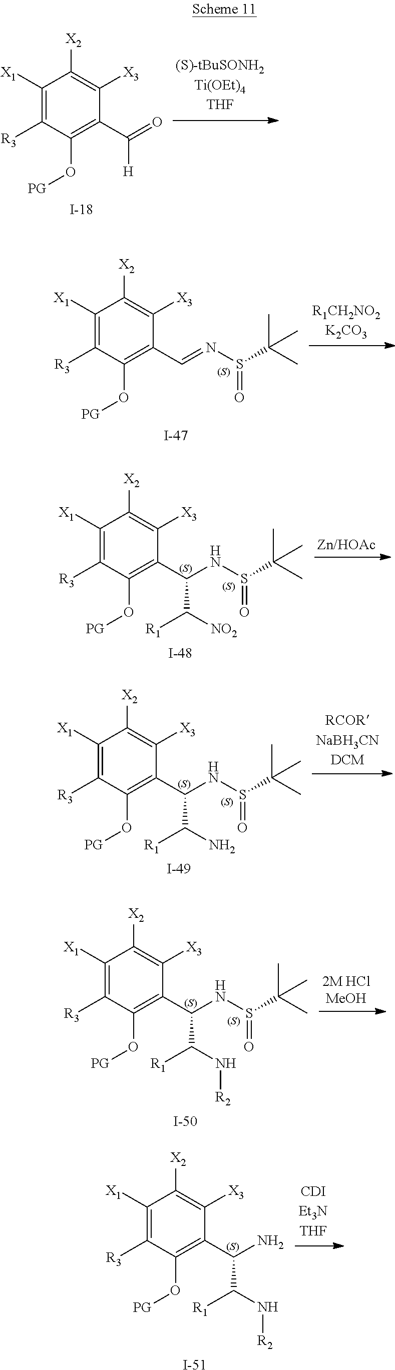

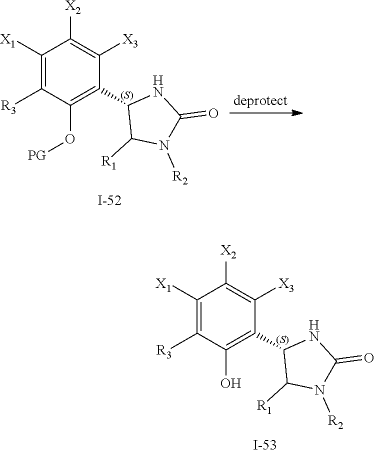

The reactions described above in Schemes 1-11 can be carried out in a suitable solvent. Suitable solvents include, but are not limited to, acetonitrile, methanol, ethanol, dichloromethane, dichloroethane, dioxane, DMF, THF, MTBE, or toluene. The reactions described in Schemes 1-11 may be conducted under inert atmosphere, e.g., under nitrogen or argon, or the reaction may be carried out in a sealed tube. The reaction mixture may be heated in a microwave or heated to an elevated temperature. Suitable elevated temperatures include, but are not limited to, 40, 50, 60, 80, 90, 100, 110, 120° C. or higher or the refluxing/boiling temperature of the solvent used. The reaction mixture may alternatively be cooled in a cold bath at a temperature lower than room temperature, e.g., 0, −10, −20, −30, −40, −50, −78, or −90° C. The reaction may be worked up by removing the solvent or partitioning of the organic solvent phase with one or more aqueous phases, each optionally containing NaCl, NaHCO₃, or NH₄Cl. The solvent in the organic phase can be removed by vacuum evaporation and the resulting residue may be purified using a silica gel column or HPLC.

### Pharmaceutical Compositions

This invention also provides a pharmaceutical composition including at least one of the compounds as described herein or a pharmaceutically-acceptable salt or solvate thereof, and a pharmaceutically-acceptable carrier.

In yet another aspect, the present invention provides a pharmaceutical composition including at least one compound selected from the group consisting of compounds of Formula I as described herein and a pharmaceutically-acceptable carrier or diluent.

In certain embodiments, the composition is in the form of a hydrate, solvate or pharmaceutically-acceptable salt. The composition can be administered to the subject by any suitable route of administration, including, without limitation, oral and parenteral.

The phrase “pharmaceutically-acceptable carrier” as used herein means a pharmaceutically-acceptable material, composition or vehicle, such as a liquid or solid filler, diluent, excipient, solvent, or encapsulating material, involved in carrying or transporting the subject pharmaceutical agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose, and sucrose; starches, such as corn starch and potato starch; cellulose and its derivatives, such as sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as butylene glycol; polyols, such as glycerin, sorbitol, mannitol, and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. The term “carrier” denotes an organic or inorganic ingredient, natural or synthetic, with which the active ingredient is combined to facilitate the application. The components of the pharmaceutical compositions also are capable of being comingled with the compounds of the present invention, and with each other, in a manner such that there is no interaction which would substantially impair the desired pharmaceutical efficiency.

As set out above, certain embodiments of the present pharmaceutical agents may be provided in the form of pharmaceutically-acceptable salts. The term “pharmaceutically-acceptable salt,” in this respect, refers to the relatively non-toxic, inorganic, and organic acid salts of compounds of the present invention. These salts can be prepared in situ during the final isolation and purification of the compounds of the invention, or by separately reacting a purified compound of the invention in its free base form with a suitable organic or inorganic acid, and isolating the salt thus formed. Representative salts include hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts, and the like. See, e.g., Berge et al., (1977) “Pharmaceutical Salts”, J. Pharm. Sci. 66:1-19.

The pharmaceutically-acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, butionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic, and organic base addition salts of compounds of the present invention. These salts can likewise be prepared in situ during the final isolation and purification of the compounds, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary, or tertiary amine. Representative alkali or alkaline earth salts include the lithium, sodium, potassium, calcium, magnesium, and aluminum salts, and the like. Representative organic amines useful for the formation of base addition salts include ethylamine, diethylamine, ethylenediamine, ethanolamine, diethanolamine, piperazine, and the like. See, e.g., Berge et al. (supra).

Wetting agents, emulsifiers, and lubricants, such as sodium lauryl sulfate, magnesium stearate, and polyethylene oxide-polybutylene oxide copolymer, as well as coloring agents, release agents, coating agents, sweetening, flavoring and perfuming agents, preservatives, and antioxidants can also be present in the compositions.

Formulations of the present invention include those suitable for oral, nasal, topical (including buccal and sublingual), rectal, vaginal, and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated and the particular mode of administration. The amount of active ingredient, which can be combined with a carrier material to produce a single dosage form will generally be that amount of the compound which produces a therapeutic effect. Generally, out of 100%, this amount will range from about 1% to about 99% of active ingredient, preferably from about 5% to about 70%, most preferably from about 10% to about 30%.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia), and/or as mouthwashes and the like, each containing a predetermined amount of a compound of the present invention as an active ingredient. A compound of the present invention may also be administered as a bolus, electuary, or paste.

In solid dosage forms of the invention for oral administration (capsules, tablets, pills, dragees, powders, granules, and the like), the active ingredient is mixed with one or more pharmaceutically-acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose, and/or acacia; humectants, such as glycerol; disintegrating agents, such as agar-agar, calcium carbonate, potato or tapioca starch, alginic acid, certain silicates, sodium carbonate, and sodium starch glycolate; solution retarding agents, such as paraffin; absorption accelerators, such as quaternary ammonium compounds; wetting agents, such as, for example, cetyl alcohol, glycerol monostearate, and polyethylene oxide-polybutylene oxide copolymer; absorbents, such as kaolin and bentonite clay; lubricants, such as talc, calcium stearate, magnesium stearate, solid polyethylene glycols, sodium lauryl sulfate, and mixtures thereof; and coloring agents. In the case of capsules, tablets and pills, the pharmaceutical compositions may also include buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxybutylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills, and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxybutylmethyl cellulose in varying proportions, to provide the desired release profile, other polymer matrices, liposomes, and/or microspheres. They may be sterilized by, for example, filtration through a bacteria-retaining filter, or by incorporating sterilizing agents in the form of sterile solid compositions, which can be dissolved in sterile water or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions, which can be used include polymeric substances and waxes. The active ingredient can also be in micro-encapsulated form, if appropriate, with one or more of the above-described excipients.

Liquid dosage forms for oral administration of the compounds of the invention include pharmaceutically-acceptable emulsions, microemulsions, solutions, suspensions, syrups, and elixirs. In addition to the active ingredient, the liquid dosage forms may contain inert diluents commonly used in the art, such as, for example, water or other solvents, solubilizing agents and emulsifiers, such as ethyl alcohol, isobutyl alcohol, ethyl carbonate, ethyl acetate, benzyl alcohol, benzyl benzoate, butylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Additionally, cyclodextrins, e.g., hydroxybutyl-β-cyclodextrin, may be used to solubilize compounds.

Besides inert diluents, the oral compositions can also include adjuvants such as wetting agents, emulsifying and suspending agents, sweetening, flavoring, coloring, perfuming, and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar, and tragacanth, and mixtures thereof.

Dosage forms for the topical or transdermal administration of a compound of this invention include powders, sprays, ointments, pastes, creams, lotions, gels, solutions, patches, and inhalants. The active compound may be mixed under sterile conditions with a pharmaceutically-acceptable carrier, and with any preservatives, buffers, or propellants which may be required.

The ointments, pastes, creams, and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and butane.

Transdermal patches have the added advantage of providing controlled delivery of a compound of the present invention to the body. Such dosage forms can be made by dissolving, or dispersing the pharmaceutical agents in the proper medium. Absorption enhancers can also be used to increase the flux of the pharmaceutical agents of the invention across the skin. The rate of such flux can be controlled, by either providing a rate-controlling membrane or dispersing the compound in a polymer matrix or gel.

Ophthalmic formulations, eye ointments, powders, solutions, and the like, are also contemplated as being within the scope of this invention.

Pharmaceutical compositions of this invention suitable for parenteral administration include one or more compounds of the invention in combination with one or more pharmaceutically-acceptable sterile isotonic aqueous or nonaqueous solutions, dispersions, suspensions, or emulsions; or sterile powders which may be reconstituted into sterile injectable solutions or dispersions just prior to use, which may contain antioxidants, buffers, bacteriostats, or solutes which render the formulation isotonic with the blood of the intended recipient or suspending or thickening agents.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally-administered drug form is accomplished by dissolving or suspending the drug in an oil vehicle. One strategy for depot injections includes the use of polyethylene oxide-polypropylene oxide copolymers where the vehicle is fluid at room temperature and solidifies at body temperature.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly (orthoesters) and poly (anhydrides). Depot-injectable formulations are also prepared by entrapping the drug in liposomes or microemulsions, which are compatible with body tissue.

When the compounds of the present invention are administered as pharmaceuticals, to humans and animals, they can be given per se or as a pharmaceutical composition containing, for example, 0.1% to 99.5% (more preferably, 0.5% to 90%) of active ingredient in combination with a pharmaceutically-acceptable carrier.

The compounds and pharmaceutical compositions of the present invention can be employed in combination therapies, that is, the compounds and pharmaceutical compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, the compound of the present invention may be administered concurrently with another anticancer agents).

The compounds of the invention may be administered intravenously, intramuscularly, intraperitoneally, subcutaneously, topically, orally, or by other acceptable means. The compounds may be used to treat arthritic conditions in mammals (e.g., humans, livestock, and domestic animals), racehorses, birds, lizards, and any other organism which can tolerate the compounds.

The invention also provides a pharmaceutical pack or kit including one or more containers filled with one or more of the ingredients of the pharmaceutical compositions of the invention. Optionally associated with such container(s) can be a notice in the form prescribed by a governmental agency regulating the manufacture, use, or sale of pharmaceuticals or biological products, which notice reflects approval by the agency of manufacture, use, or sale for human administration.

### Administration to a Subject

In yet another aspect, the present invention provides a method for treating a condition in a mammalian species in need thereof, the method including administering to the mammalian species a therapeutically effective amount of at least one compound selected from the group consisting of compounds of Formula I, or a pharmaceutically-acceptable salt thereof, or a pharmaceutical composition thereof, where the condition is selected from the group consisting of cancer, an immunological disorder, a central nervous system disorder, an inflammatory disorder, a gastroenterological disorder, a metabolic disorder, a cardiovascular disorder, and a kidney disease.

In some embodiments, the cancer is selected from the group consisting of biliary tract cancer, brain cancer, breast cancer, cervical cancer, choriocarcinoma, colon cancer, endometrial cancer, esophageal cancer, gastric (stomach) cancer, intraepithelial neoplasms, leukemias, lymphomas, liver cancer, lung cancer, melanoma, neuroblastomas, oral cancer, ovarian cancer, pancreatic cancer, prostate cancer, rectal cancer, renal (kidney) cancer, sarcomas, skin cancer, testicular cancer, and thyroid cancer.

In some embodiments, the inflammatory disorder is an inflammatory skin condition, arthritis, psoriasis, spondylitis, parodontitits, or an inflammatory neuropathy. In some embodiments, the gastroenterological disorder is an inflammatory bowel disease such as Crohn's disease or ulcerative colitis.

In some embodiments, the immunological disorder is transplant rejection or an autoimmune disease (e.g., rheumatoid arthritis, multiple sclerosis, systemic lupus erythematosus, or type I diabetes mellitus). In some embodiments, the central nervous system disorder is Alzheimer's disease.

In some embodiments, the metabolic disorder is obesity or type II diabetes mellitus. In some embodiments, the cardiovascular disorder is an ischemic stroke. In some embodiments, the kidney disease is chronic kidney disease, nephritis, or chronic renal failure.

In some embodiments, the mammalian species is human.

In some embodiments, the condition is selected from the group consisting of cancer, transplant rejection, rheumatoid arthritis, multiple sclerosis, systemic lupus erythematosus, type I diabetes mellitus, Alzheimer's disease, inflammatory skin condition, inflammatory neuropathy, psoriasis, spondylitis, parodontitis, inflammatory bowel disease, obesity, type II diabetes mellitus, ischemic stroke, chronic kidney disease, nephritis, chronic renal failure, and a combination thereof.

In yet another aspect, a method of blocking Kv1.3 potassium channel in a mammalian species in need thereof is described, including administering to the mammalian species a therapeutically effective amount of at least one compound of Formula I, or a pharmaceutically-acceptable salt thereof, or a pharmaceutical composition thereof.

In some embodiments, the compounds described herein is selective in blocking the Kv1.3 potassium channels with minimal or no off-target inhibition activities against other potassium channels, or against calcium or sodium channels. In some embodiments, the compounds described herein do not block the hERG channels and therefore have desirable cardiovascular safety profiles.

Some aspects of the invention involve administering an effective amount of a composition to a subject to achieve a specific outcome. The small molecule compositions useful according to the methods of the present invention thus can be formulated in any manner suitable for pharmaceutical use.

The formulations of the invention are administered in pharmaceutically-acceptable solutions, which may routinely contain pharmaceutically-acceptable concentrations of salt, buffering agents, preservatives, compatible carriers, adjuvants, and optionally other therapeutic ingredients.

For use in therapy, an effective amount of the compound can be administered to a subject by any mode allowing the compound to be taken up by the appropriate target cells. “Administering” the pharmaceutical composition of the present invention can be accomplished by any means known to the skilled artisan. Specific routes of administration include, but are not limited to, oral, transdermal (e.g., via a patch), parenteral injection (subcutaneous, intradermal, intramuscular, intravenous, intraperitoneal, intrathecal, etc.), or mucosal (intranasal, intratracheal, inhalation, intrarectal, intravaginal, etc.). An injection can be in a bolus or a continuous infusion.

For example the pharmaceutical compositions according to the invention are often administered by intravenous, intramuscular, or other parenteral means. They can also be administered by intranasal application, inhalation, topically, orally, or as implants; even rectal or vaginal use is possible. Suitable liquid or solid pharmaceutical preparation forms are, for example, aqueous or saline solutions for injection or inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops, or preparations with protracted release of active compounds in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of present methods for drug delivery, see Langer R (1990) Science 249:1527-33.

The concentration of compounds included in compositions used in the methods of the invention can range from about 1 nM to about 100 μM. Effective doses are believed to range from about 10 picomole/kg to about 100 micromole/kg.

The pharmaceutical compositions are preferably prepared and administered in dose units. Liquid dose units are vials or ampoules for injection or other parenteral administration. Solid dose units are tablets, capsules, powders, and suppositories. For treatment of a patient, different doses may be necessary depending on activity of the compound, manner of administration, purpose of the administration (i.e., prophylactic or therapeutic), nature and severity of the disorder, age, and body weight of the patient. The administration of a given dose can be carried out both by single administration in the form of an individual dose unit or else several smaller dose units. Repeated and multiple administration of doses at specific intervals of days, weeks, or months apart are also contemplated by the invention.

The compositions can be administered per se (neat) or in the form of a pharmaceutically-acceptable salt. When used in medicine the salts should be pharmaceutically acceptable, but non-pharmaceutically-acceptable salts can conveniently be used to prepare pharmaceutically-acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium, or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v); and thimerosal (0.004-0.02% w/v).

Compositions suitable for parenteral administration conveniently include sterile aqueous preparations, which can be isotonic with the blood of the recipient. Among the acceptable vehicles and solvents are water, Ringer's solution, phosphate buffered saline, and isotonic sodium chloride solution. In addition, sterile, fixed oils are conventionally employed as a solvent or suspending medium. For this purpose, any bland fixed mineral or non-mineral oil may be employed including synthetic mono- or diglycerides. In addition, fatty acids such as oleic acid find use in the preparation of injectables. Carrier formulations suitable for subcutaneous, intramuscular, intraperitoneal, intravenous, etc. administrations can be found in, e.g., Remington's Pharmaceutical Sciences, Mack Publishing Company, Easton, PA.

The compounds useful in the invention can be delivered in mixtures of more than two such compounds. A mixture can further include one or more adjuvants in addition to the combination of compounds.

A variety of administration routes is available. The particular mode selected will depend, of course, upon the particular compound selected, the age and general health status of the subject, the particular condition being treated, and the dosage required for therapeutic efficacy. The methods of this invention, generally speaking, can be practiced using any mode of administration that is medically acceptable, meaning any mode that produces effective levels of response without causing clinically unacceptable adverse effects. Preferred modes of administration are discussed above.

The compositions can conveniently be presented in unit dosage form and can be prepared by any of the methods well known in the art of pharmacy. All methods include the step of bringing the compounds into association with a carrier which constitutes one or more accessory ingredients. In general, the compositions are prepared by uniformly and intimately bringing the compounds into association with a liquid carrier, a finely divided solid carrier, or both, and then, if necessary, shaping the product.

Other delivery systems can include time-release, delayed release, or sustained-release delivery systems. Such systems can avoid repeated administrations of the compounds, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, e.g., U.S. Pat. No. 5,075,109. Delivery systems also include non-polymer systems that are: lipids including sterols such as cholesterol, cholesterol esters and fatty acids, or neutral fats such as mono-di- and tri-glycerides; hydrogel release systems; silastic systems; peptide-based systems; wax coatings; compressed tablets using conventional binders and excipients; partially fused implants; and the like. Specific examples include, but are not limited to: (a) erosional systems in which an agent of the invention is contained in a form within a matrix, such as those described in U.S. Pat. Nos. 4,452,775, 4,675,189, and 5,736,152, and (b) diffusional systems in which an active component permeates at a controlled rate from a polymer, such as described in U.S. Pat. Nos. 3,854,480, 5,133,974 and 5,407,686. In addition, pump-based hardware delivery systems can be used, some of which are adapted for implantation.

### Assay for Determining the Effectiveness of Kv1.3 Potassium Channel Blockers

In some embodiments, the compounds as described herein are tested for their activities against Kv1.3 potassium channel. In some embodiments, the compounds as described herein are tested for their Kv1.3 potassium channel electrophysiology. In some embodiments, the compounds as described herein are tested for their hERG electrophysiology.

### EQUIVALENTS

The representative examples which follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art. The following examples contain important additional information, exemplification, and guidance which can be adapted to the practice of this invention in its various embodiments and equivalents thereof.

### EXAMPLES

Examples 1-9 describe various intermediates used in the synthesis of representative compounds of Formula I disclosed herein.

### Example 1. Intermediate 1 (ethyl (2E)-3-(2,3-dichloro-6-[[2-(trimethylsilyl)ethoxy]methoxy]phenyl)prop-2-enoate)

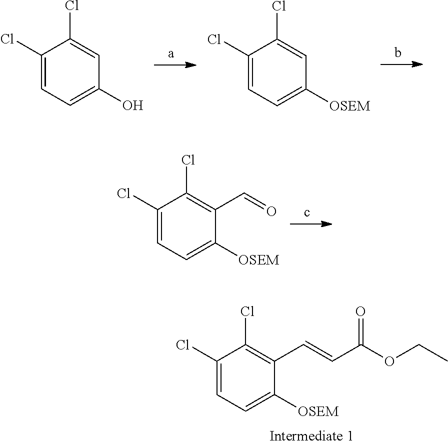

Step a:

To a stirred mixture of 3,4-dichlorophenol (200 g, 1.23 mol) and K₂CO₃ (339 g, 2.45 mol) in DMF (1 L) was added SEMCl (245 g, 1.47 mol) in portions at 0° C. The resulting mixture was stirred for 16 h, diluted with water (3 L), and extracted with EA (3×3 L). The combined organic layers were washed with brine (3×1 L) and dried over anhydrous Na₂SO₄. After filtration, the filtrate was concentrated under reduced pressure. The residue was purified by silica gel column chromatography, eluting with PE/EA (100/1) to afford [2-(3,4-dichlorophenoxymethoxy)ethyl]trimethylsilane as a colorless oil (250 g, 69%): ¹H NMR (400 MHz, CDCl₃) δ 7.35 (d, J=8.8 Hz, 1H), 7.19 (d, J=2.8 Hz, 1H), 6.92 (dd, J=8.9, 2.8 Hz, 1H), 5.21 (s, 2H), 3.79-3.73 (m, 2H), 1.00-0.95 (m, 2H), 0.03 (s, 9H).

Step b:

To a solution of [2-(3,4-dichlorophenoxymethoxy)ethyl]trimethylsilane (120 g, 409 mmol) in THE (1.50 L) was added n-BuLi (164 mL, 409 mmol, 2.5 M in hexane) dropwise over 30 min at −78° C. The resulting solution was stirred for 1 h and DMF (59.8 g, 818 mmol) was added dropwise over 20 min at −78° C., and then stirred for a further 1 h. The reaction mixture was quenched with saturated aqueous NH₄Cl (1 L) and extracted with EA (3×1 L). The combined organic layers were washed with brine (3×1 L) and dried over anhydrous Na₂SO₄. After filtration, the filtrate was concentrated under reduced pressure. The residue was purified by silica gel column chromatography, eluting with PE/EA (12/1) to afford 2,3-dichloro-6-[[2-(trimethylsilyl)ethoxy]methoxy]benzaldehyde as a light-yellow solid (107 g, 81%): ¹H NMR (300 MHz, CDCl₃) δ 10.46 (s, 1H), 7.55 (d, J=9.0 Hz, 1H), 7.17 (d, J=9.0 Hz, 1H), 5.31 (s, 2H), 3.83-3.68 (m, 2H), 1.01-0.90 (m, 2H), 0.01 (s, 9H).

Step c:

To a stirred mixture of NaH (1.50 g, 62.6 mmol, 60% in oil) in EA (100 mL) was added 2,3-dichloro-6-[[2-(trimethylsilyl)ethoxy]methoxy]benzaldehyde (10.0 g, 31.1 mmol) at 0° C. under nitrogen atmosphere. The resulting reaction mixture was stirred for 16 h, quenched with water (100 mL), and extracted with EA (3×100 mL). The combined organic layers were washed with brine (2×100 mL) and dried over anhydrous Na₂SO₄. After filtration, the filtrate was concentrated under reduced pressure. The residue was purified by silica gel column chromatography, eluting with PE/EA (10/1) to afford Intermediate 1 (ethyl (2E)-3-(2,3-dichloro-6-[[2-(trimethylsilyl)ethoxy]methoxy]phenyl)prop-2-enoate) as a light-yellow oil (8.80 g, 57.8%): ¹H NMR (300 MHz, CDCl₃) δ 7.96 (d, J=16.2 Hz, 1H), 7.37 (d, J=9.0 Hz, 1H), 7.11 (d, J=9.1 Hz, 1H), 6.79 (d, J=16.2 Hz, 1H), 5.29 (s, 2H), 4.30 (q, J=7.1 Hz, 2H), 3.81-3.67 (m, 2H), 1.36 (t, J=7.1 Hz, 3H), 1.02-0.90 (m, 2H), 0.01 (s, 9H).

### Example 2. Intermediate 2 (ethyl 4-amino-3-(2,3-dichloro-6-[[2-(trimethylsilyl)ethoxy]methoxy]phenyl)butanoate)

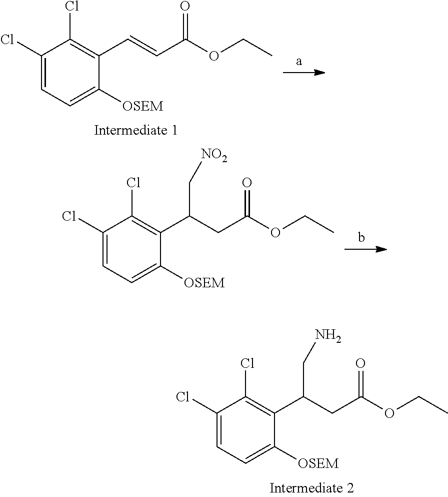

Step a:

To a stirred solution of ethyl (2E)-3-(2,3-dichloro-6-[[2-(trimethylsilyl)ethoxy]methoxy]phenyl)prop-2-enoate (Intermediate 1, Example 1) (14.0 g, 35.8 mmol) in CH₃NO₂ (140 mL) was added DBU (6.54 g, 42.9 mmol) at room temperature under nitrogen atmosphere. The reaction mixture was stirred at 60° C. for 16 h, poured into water (100 mL), and extracted with EA (3×80 mL). The combined organic layers were washed with brine (3×50 mL) and dried over anhydrous Na₂SO₄. After filtration, the filtrate was concentrated under reduced pressure. The residue was purified by silica gel column chromatography, eluting with PE/EA (10/1) to afford ethyl 3-(2,3-dichloro-6-[[2-(trimethylsilyl)ethoxy]methoxy]phenyl)-4-nitrobutanoate as a light-yellow oil (10.0 g, 56%): LCMS (ESI) calculated (“calc'd”) for C₁₈H₂₇Cl₂NO₆Si [M+Na]⁺: 474, 476 (3:2) found 474, 476 (3:2); ¹H NMR (300 MHz, CDCl₃) δ 7.34 (d, J=9.0 Hz, 1H), 7.07 (d, J=9.0 Hz, 1H), 5.27 (s, 2H), 4.94-4.82 (m, 3H), 4.10 (q, J=7.1 Hz, 2H), 3.78 (td, J=8.1, 1.3 Hz, 2H), 2.94-2.85 (m, 2H), 1.20 (t, J=7.2 Hz, 3H), 1.01-0.92 (m, 2H), 0.03 (s, 9H).

Step b:

To a stirred solution of ethyl 3-(2,3-dichloro-6-[[2-(trimethylsilyl)ethoxy]methoxy]phenyl)-4-nitrobutanoate (10.0 g, 19.9 mmol) in AcOH (36 mL) was added Zn (19.5 g, 299 mmol) in portions under nitrogen atmosphere. The reaction mixture was stirred for 4 h and filtered. The filter cake was washed with EA (3×30 mL) and the filtrate concentrated under reduced pressure. The residue was purified by reverse phase chromatography, eluting with 55% ACN in water (plus 0.05% TFA) to afford Intermediate 2 (ethyl 4-amino-3-(2,3-dichloro-6-[[2-(trimethylsilyl)ethoxy]methoxy]phenyl)butanoate) as an off-white solid (6.00 g, 51%): LCMS (ESI) calc'd for C₁₈H₂₉Cl₂NO₄Si [M+H]⁺: 422, 424 (3:2) found 422, 424 (3:2); ¹H NMR (400 MHz, CDCl₃) δ 8.27 (brs, 3H), 7.32 (d, J=9.0, 2.3 Hz, 1H), 7.08 (d, J=9.0 Hz, 1H), 5.34-5.17 (m, 2H), 4.32-4.21 (m, 1H), 4.13-4.01 (m, 2H), 3.81-3.69 (m, 2H), 3.43 (d, J=58.6 Hz, 2H), 3.14-2.76 (m, 2H), 1.17 (t, J=6.5 Hz, 3H), 1.00-0.86 (m, 2H), 0.02 (s, 9H).

### Example 3. Intermediate 3 (4-(2,3-dichloro-6-[[2-(trimethylsilyl)ethoxy]methoxy]phenyl)pyrrolidin-2-one)

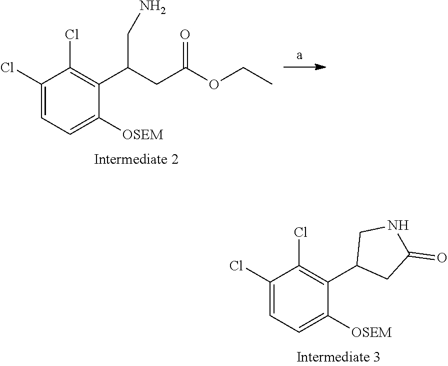

Step a:

A solution of ethyl 4-amino-3-(2,3-dichloro-6-[[2-(trimethylsilyl)ethoxy]methoxy]phenyl)butanoate (Intermediate 2, Example 2) (4.00 g, 7.69 mmol) and K₂CO₃ (3.19 g, 23.1 mmol) in MeOH (40 mL) was stirred at room temperature for 2 h. The resulting mixture was diluted with water (50 mL) and extracted with EA (3×50 mL). The combined organic layers were washed with brine (3×50 mL) and dried over anhydrous Na₂SO₄. After filtration, the filtrate was concentrated under reduced pressure. The residue was purified by reverse flash chromatography, eluting with 85% ACN in water (plus 10 mM NH₄HCO₃) to afford Intermediate 3 (4-(2,3-dichloro-6-[[2-(trimethylsilyl)ethoxy]methoxy]phenyl)pyrrolidin-2-one) as a light-yellow oil (2.40 g, 75%): LCMS (ESI) calc'd for C₁₆H₂₃Cl₂NO₃Si [M+Na]⁺: 398, 400 (3:2) found 398, 400 (3:2); ¹H NMR (400 MHz, CDCl₃) δ 7.34 (d, J=9.0 Hz, 1H), 7.11 (d, J=9.0 Hz, 1H), 5.27 (s, 2H), 4.64-4.50 (m, 1H), 3.78-3.71 (m, 2H), 3.63 (dt, J=29.9, 8.8 Hz, 2H), 2.78 (dd, J=17.0, 8.3 Hz, 1H), 2.61 (dd, J=17.0, 10.8 Hz, 1H), 0.98-0.92 (m, 2H), 0.02 (s, 9H).

### Example 4. Intermediate 4 ((2-[3,4-dichloro-2-[(E)-2-nitroethenyl]phenoxymethoxy]ethyl)trimethylsilane)

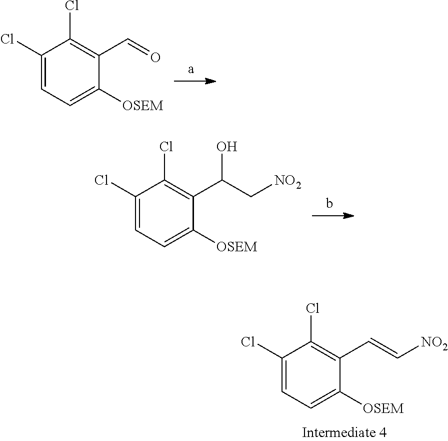

Step a:

To a stirred solution of 2,3-dichloro-6-[[2-(trimethylsilyl)ethoxy]methoxy]benzaldehyde (Example 1, step b) (15.0 g, 46.7 mmol) in CH₃NO₂ (200 mL) was added K₂CO₃ (16.1 g, 117 mmol) at room temperature. The resulting reaction mixture was stirred for 30 min, diluted with water (100 mL), and extracted with EA (3×80 mL). The combined organic layers were washed with brine (3×50 mL) and dried over anhydrous Na₂SO₄. After filtration, the filtrate was concentrated under reduced pressure. The residue was purified by silica gel column chromatography, eluting with PE/EA (5/1) to afford 1-(2,3-dichloro-6-[[2-(trimethylsilyl)ethoxy]methoxy]phenyl)-2-nitroethanol as a light-orange oil (16.0 g, 90%): ¹H NMR (400 MHz, CDCl₃) δ 7.43 (d, J=8.9 Hz, 1H), 7.14 (d, J=9.0 Hz, 1H), 6.06 (s, 1H), 5.36 (s, 2H), 4.90 (dd, J=12.2, 9.7 Hz, 1H), 4.58 (dd, J=12.2, 3.7 Hz, 1H), 4.16-4.12 (m, 1H), 3.84-3.76 (m, 2H), 1.03-0.96 (m, 2H), 0.04 (s, 9H).

Step b:

To a stirred solution of 1-(2,3-dichloro-6-[[2-(trimethylsilyl)ethoxy]methoxy]phenyl)-2-nitroethanol (15.0 g, 39.2 mmol) in toluene (150 mL) was added Burgess reagent (28.1 g, 118 mmol) at room temperature under nitrogen atmosphere. The resulting reaction mixture was stirred at 60° C. for 2 h and concentrated under reduced pressure. The residue was purified by silica gel column chromatography, eluting with PE/EA (12/1) to afford Intermediate 4 ((2-[3,4-dichloro-2-[(E)-2-nitroethenyl]phenoxymethoxy]ethyl)trimethylsilane) as a light-yellow solid (12.0 g, 84%): ¹H NMR (400 MHz, CDCl₃) δ 8.50 (d, J=13.6 Hz, 1H), 8.05 (d, J=13.6 Hz, 1H), 7.51 (d, J=9.1 Hz, 1H), 7.20 (d, J=9.1 Hz, 1H), 5.39 (s, 2H), 3.83-3.75 (m, 2H), 1.01-0.93 (m, 2H), 0.03 (s, 9H).

### Example 5. Intermediate 5 (1,3-diethyl 2-[(1S)-2-amino-1-(2,3-dichloro-6-[[2-(trimethylsilyl)ethoxy]methoxy]phenyl)ethyl]propanedioate trifluoroacetic acid salt)

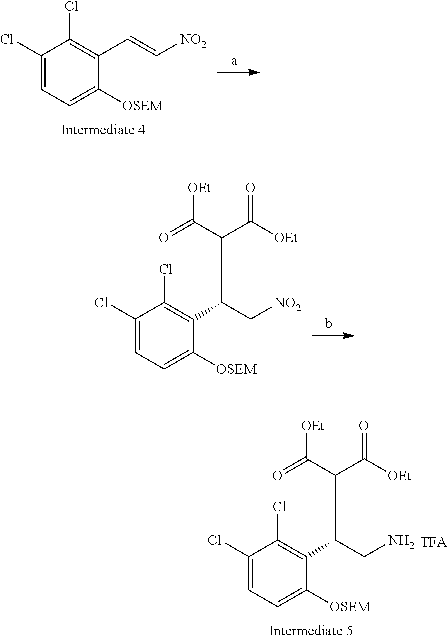

Step a:

To a stirred solution of (2-[3,4-dichloro-2-[(E)-2-nitroethenyl]phenoxymethoxy]ethyl)trimethylsilane (Intermediate 4, Example 4) (13.0 g, 35.7 mmol) and diethyl malonate (6.86 g, 42.8 mmol) in toluene (130 mL) was added bis[(1R,2R)—N¹,N²-bis(phenylmethyl)-1,2-cyclohexanediamine-κN¹,κN²]dibromonickel (5.73 g, 7.13 mmol) at room temperature under nitrogen atmosphere. The resulting reaction mixture was stirred for 16 h, diluted with water (100 mL), and extracted with EA (3×100 mL). The combined organic layers were washed with brine (3×100 mL) and dried over anhydrous Na₂SO₄. After filtration, the filtrate was concentrated under reduced pressure. The residue was purified by silica gel column chromatography, eluting with PE/EA (3/2) to afford 1,3-diethyl 2-[(1S)-1-(2,3-dichloro-6-[[2-(trimethylsilyl)ethoxy]methoxy]phenyl)-2-nitroethyl]propanedioate as a light-yellow oil (16.0 g, 85%): LCMS (ESI) calc'd for C₂₁H₃₁Cl₂NO₈Si [M+Na]⁺: 546, 548 (3:2) found 546, 548 (3:2); ¹H NMR (300 MHz, CDCl₃) δ 7.35 (d, J=9.0 Hz, 1H), 7.11 (d, J=9.1 Hz, 1H), 5.28 (s, 2H), 5.10-4.97 (m, 1H), 4.90 (dd, J=12.3, 4.7 Hz, 1H), 4.38-4.15 (m, 4H), 4.03-3.73 (m, 4H), 1.40-1.21 (m, 6H), 1.01 (t, J=7.2 Hz, 2H), 0.05 (s, 9H).

Step b:

To a stirred solution of 1,3-diethyl 2-[(1S)-1-(2,3-dichloro-6-[[2-(trimethylsilyl)ethoxy]methoxy]phenyl)-2-nitroethyl]propanedioate (16.0 g, 30.5 mmol) in AcOH (160 mL) was added Zn (29.9 g, 458 mmol) in portions at room temperature under nitrogen atmosphere. The resulting reaction mixture was stirred for 16 h and filtered. The filter cake was washed with EA (3×50 mL) and the filtrate concentrated under reduced pressure. The residue was purified by reverse phase chromatography, eluting with 40% ACN in water (plus 0.05% TFA) to afford Intermediate 5 (1,3-diethyl 2-[(1S)-2-amino-1-(2,3-dichloro-6-[[2-(trimethylsilyl)ethoxy]methoxy]phenyl)ethyl]propanedioate trifluoroacetic acid salt) as a light-yellow oil (13.0 g, 86%): LCMS (ESI) calc'd for C₂₁H₃₃Cl₂NO₆Si [M+H]⁺: 494, 496 (3:2) found 494, 496 (3:2); ¹H NMR (300 MHz, CDCl₃) δ 7.36 (d, J=9.0 Hz, 1H), 7.11 (d, J=9.0 Hz, 1H), 5.28 (s, 2H), 5.06-4.92 (m, 1H), 4.32-4.07 (m, 4H), 3.94 (d, J=8.7 Hz, 1H), 3.80-3.63 (m, 3H), 3.60-3.48 (m, 1H), 1.35-1.23 (m, 6H), 1.03-0.90 (m, 2H), 0.03 (s, 9H).

### Example 6. Intermediate 6 ((4S)-4-(2,3-dichloro-6-[[2-(trimethylsilyl)ethoxy]methoxy]phenyl)pyrrolidin-2-one)

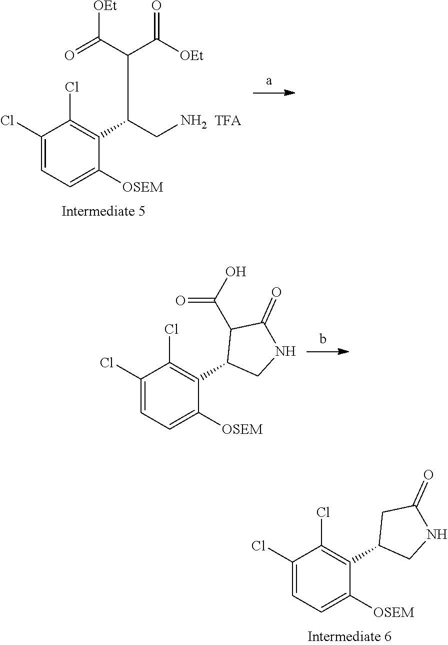

Step a:

A solution of 1,3-diethyl 2-[(1S)-2-amino-1-(2,3-dichloro-6-[[2-(trimethylsilyl)ethoxy]methoxy]phenyl)ethyl]propanedioate trifluoroacetic acid salt (Intermediate 5, Example 5) (13.0 g, 26.3 mmol) and LiOH (1.89 g, 78.9 mmol) in MeOH (130 mL) and H₂O (10 mL) was stirred at room temperature for 16 h. The resulting mixture was acidified with saturated aqueous citric acid to pH 3 followed by extraction with EA (2×150 mL). The combined organic layers were washed with brine (2×150 mL) and dried over anhydrous Na₂SO₄. After filtration, the filtrate was concentrated under reduced pressure to afford (4S)-4-(2,3-dichloro-6-[[2-(trimethylsilyl)ethoxy]methoxy]phenyl)-2-oxopyrrolidine-3-carboxylic acid as a light-yellow oil (10.0 g, crude), which was used directly in the next step without purification: LCMS (ESI) calc'd for C₁₇H₂₃Cl₂NO₅Si [M−H]⁻: 418, 420 (3:2) found 418, 420 (3:2); ¹H NMR (400 MHz, CDCl₃) δ 7.37 (d, J=9.0 Hz, 1H), 7.11 (d, J=9.0 Hz, 1H), 6.98 (s, 1H), 5.29 (d, J=1.4 Hz, 2H), 4.17-4.10 (m, 1H), 3.79-3.71 (m, 3H), 3.71-3.55 (m, 2H), 0.99-0.92 (m, 2H), 0.02 (s, 9H).

Step b:

A solution of (4S)-4-(2,3-dichloro-6-[[2-(trimethylsilyl)ethoxy]methoxy]phenyl)-2-oxopyrrolidine-3-carboxylic acid (10.0 g, 23.8 mmol) in toluene (100 mL) was stirred at 120° C. for 4 h. The resulting mixture was concentrated under reduced pressure. The residue was purified by reverse phase chromatography, eluting with 55% ACN in water (plus 0.05% TFA) to afford the desired product. The product was purified by Prep SFC with following conditions: Column: CHIRALPAK IH, 3×25 cm, 5 μm; Mobile Phase A: CO₂, Mobile Phase B: MeOH (plus 0.1% 2M NH₃-MeOH); Flow rate: 70 mL/min; Gradient: 35% B; Detector: UV 220 nm; Retention Time: 8.63 min. The fractions containing the desired product were collected and concentrated under reduced pressure to afford Intermediate 6 ((4S)-4-(2,3-dichloro-6-[[2-(trimethylsilyl)ethoxy]methoxy]phenyl)pyrrolidin-2-one) as a light-yellow oil (1.00 g, 11%): LCMS (ESI) calc'd for C₁₆H₂₃Cl₂NO₃Si [M+H]⁺: 376, 378 (3:2) found 376, 378 (3:2); ¹H NMR (400 MHz, CDCl₃) δ 7.34 (d, J=8.9 Hz, 1H), 7.10 (d, J=9.0 Hz, 1H), 5.27 (s, 2H), 4.63-4.49 (m, 1H), 3.75 (t, J=8.2 Hz, 2H), 3.63 (dt, J=29.0, 8.9 Hz, 2H), 2.83-2.55 (m, 2H), 0.96 (t, J=8.2 Hz, 2H), 0.02 (s, 9H).

### Example 7. Intermediate 7 (ethyl 3-[2,3-dichloro-6-(prop-2-en-1-yloxy)phenyl]-4-oxobutanoate)

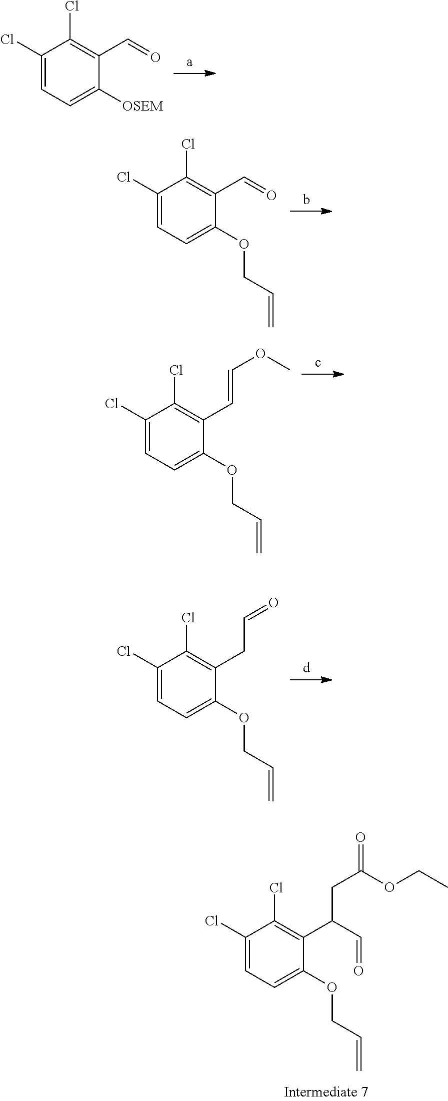

Step a:

To a stirred solution of 2,3-dichloro-6-[[2-(trimethylsilyl)ethoxy]methoxy]benzaldehyde (10.0 g, 31.1 mmol) (Example 1, step b) was added TFA (20.0 mL) in DCM (40 mL). The reaction mixture was stirred at room temperature for 1 h and concentrated under reduced pressure. To the residue was added K₂CO₃ (12.9 g, 93.4 mmol) and allyl bromide (5.65 g, 46.7 mmol) in DMF (50 mL). The resulting reaction mixture was stirred for 3 h, diluted with water (50 mL), and extracted with EA (3×70 mL). The combined organic layers were washed with brine (5×50 mL) and dried over anhydrous Na₂SO₄. After filtration, the filtrate was concentrated under reduced pressure. The residue was purified by silica gel column chromatography, eluting with PE/EA (10/1) to afford 2,3-dichloro-6-(prop-2-en-1-yloxy)benzaldehyde as a light-yellow solid(5.40 g, 68%): ¹H NMR (300 MHz, CDCl₃) δ 10.51 (s, 1H), 7.57 (d, J=9.0 Hz, 1H), 6.90 (d, J=9.1 Hz, 1H), 6.14-5.91 (m, 1H), 5.55-5.42 (m, 1H), 5.42-5.31 (m, 1H), 4.71-4.62 (m, 2H).

Step b:

To a stirred solution of (methoxymethyl)triphenylphosphanium chloride (22.3 g, 64.9 mmol) in THE (100 mL) was added t-BuOK (64.9 mL, 64.9 mmol, 1 M in THF) dropwise at −10° C. under nitrogen atmosphere. The resulting mixture was stirred at for 30 min and 2,3-dichloro-6-(prop-2-en-1-yloxy)benzaldehyde (5.00 g, 21.64 mmol) was added over 2 min at −10° C. The reaction mixture was stirred at room temperature for 1 h, quenched with saturated aqueous NH₄Cl (100 mL) at 0° C., and extracted with EA (3×150 mL). The combined organic layers were washed with brine (3×100 mL) and dried over anhydrous Na₂SO₄. After filtration, the filtrate was concentrated under reduced pressure. The residue was purified by silica gel column chromatography, eluting with PE/EA (10/1) to afford 1,2-dichloro-3-[(E)-2-methoxyethenyl]-4-(prop-2-en-1-yloxy)benzene as a light-yellow oil (4.80 g, 86%): ¹H NMR (300 MHz, CDCl₃) δ 7.54 (d, J=12.8 Hz, 1H), 7.17 (d, J=8.9 Hz, 1H), 6.74 (d, J=8.8 Hz, 1H), 6.14-5.95 (m, 2H), 5.48-5.28 (m, 2H), 4.60-4.51 (m, 2H), 3.74 (s, 3H).

Step c:

To a stirred solution of 1,2-dichloro-3-[(E)-2-methoxyethenyl]-4-(prop-2-en-1-yloxy)benzene (4.80 g, 18.5 mmol) in THE (25 mL) was added HCl (25 mL, 4 M) at room temperature. The resulting mixture was stirred at 50° C. for 16 h, diluted with water (50 mL), and extracted with EA (3×50 mL). The combined organic layers were washed with brine (3×50 mL) and dried over anhydrous Na₂SO₄. After filtration, the filtrate was concentrated under reduced pressure. The residue was purified by silica gel column chromatography, eluting with PE/EA (5/1) to afford 2-[2,3-dichloro-6-(prop-2-en-1-yloxy)phenyl]acetaldehyde as a light-yellow oil (4.10 g, 81%): LCMS (ESI) calc'd for C₁₁H₁₀Cl₂O₂ [M−H]⁻: 243, 245 (3:2) found 243, 245 (3:2); ¹H NMR (300 MHz, CDCl₃) δ 9.70 (t, J=1.4 Hz, 1H), 7.38 (d, J=8.9 Hz, 1H), 6.80 (d, J=8.9 Hz, 1H), 6.08-5.90 (m, 1H), 5.43-5.25 (m, 2H), 4.62-4.51 (m, 2H), 4.00 (d, J=1.4 Hz, 2H).

Step d:

To a stirred solution of 2-[2,3-dichloro-6-(prop-2-en-1-yloxy)phenyl]acetaldehyde (4.10 g, 16.7 mmol) in toluene (40 mL) was added bis(2-methylpropyl)amine (3.24 g, 25.1 mmol) at room temperature under nitrogen atmosphere. The resulting mixture was stirred at 110° C. for 2 h and concentrated under reduced pressure. The residue was mixed with ACN (20 mL) and ethyl 2-bromoacetate (4.19 g, 25.1 mmol) was added at room temperature. The resulting reaction mixture was stirred at 90° C. for 16 h. The mixture was cooled to room temperature and AcOH (5.00 mL) and H₂O (15 mL) were added. The resulting reaction mixture was stirred at 40° C. for 2 h, diluted with water (30 mL), and extracted with EA (3×60 mL). The combined organic layers were washed with brine (3×50 mL) and dried over anhydrous Na₂SO₄. After filtration, the filtrate was concentrated under reduced pressure. The residue was purified by silica gel column chromatography, eluting with PE/EA (10/1) to afford Intermediate 7 (ethyl 3-[2,3-dichloro-6-(prop-2-en-1-yloxy)phenyl]-4-oxobutanoate) as a light-yellow oil (4.00 g, 72%): LCMS (ESI) calc'd for C₁₅H₁₆Cl₂O₄ [M+H]⁺: 331, 333 (3:2) found 331, 333 (3:2); ¹H NMR (300 MHz, CDCl₃) δ 9.59 (s, 1H), 7.40 (d, J=9.0 Hz, 1H), 6.79 (d, J=9.0 Hz, 1H), 6.02-5.85 (m, 1H), 5.39-5.27 (m, 2H), 4.66 (dd, J=7.8, 5.6 Hz, 1H), 4.56-4.50 (m, 2H), 4.19-4.10 (m, 2H), 3.24 (dd, J=16.2, 7.8 Hz, 1H), 2.50 (dd, J=16.2, 5.6 Hz, 1H), 1.25 (t, J=7.2 Hz, 3H).

### Example 8. Intermediate 8 (ethyl-(3R,4R)-rel-4-amino-3-(2,3-dichloro-6-[[2-(trimethylsilyl)ethoxy]methoxy]phenyl)pentanoate) and Intermediate 9 (ethyl-(3R,4S)-rel-4-amino-3-(2,3-dichloro-6-[[2-(trimethylsilyl)ethoxy]methoxy]phenyl)pentanoate)

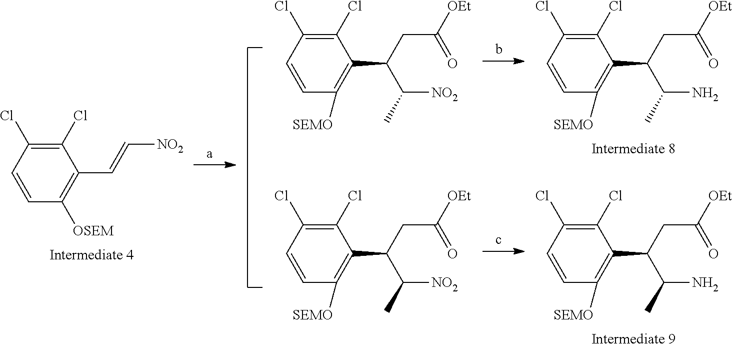

Step a:

To a stirred solution of ethyl (2E)-3-(2,3-dichloro-6-[[2-(trimethylsilyl)ethoxy]methoxy]phenyl)prop-2-enoate (Intermediate 4, Example 4) (1.80 g, 4.60 mmol) in C₂H₅NO₂ (18 mL) was added DBU (1.05 g, 6.90 mmol) at room temperature under nitrogen atmosphere. The mixture was stirred at 60° C. for 5, diluted with water (50 mL), and extracted with EA (2×50 mL). The combined organic layers were washed with brine (3×50 mL) and dried over anhydrous Na₂SO₄. After filtration, the filtrate was concentrated under reduced pressure. The residue was purified by silica gel column chromatography, eluting with PE/EA (10/1) to afford ethyl-(3R,4R)-rel-3-(2,3-dichloro-6-[[2-(trimethylsilyl)ethoxy]methoxy]phenyl)-4-nitropentanoate a light-yellow oil (0.95 g, 44%) and ethyl-(3R,4S)-rel-3-(2,3-dichloro-6-[[2-(trimethylsilyl)ethoxy]methoxy]phenyl)-4-nitropentanoate as a light-yellow oil (0.57 g, 27%): LCMS (ESI) calc'd for C₁₉H₂₉Cl₂NO₆Si [M+Na]⁺: 488, 490 (3:2) found 488, 490 (3:2). Ethyl-(3R,4R)-rel-3-(2,3-dichloro-6-[[2-(trimethylsilyl)ethoxy]methoxy]phenyl)-4-nitropentanoate: ¹H NMR (400 MHz, CDCl₃) δ 7.37 (d, J=9.0 Hz, 1H), 7.08 (d, J=9.0 Hz, 1H), 5.30 (s, 2H), 5.26-5.17 (m, 1H), 4.71-4.62 (m, 1H), 3.99-3.96 (m, 2H), 3.84-3.76 (m, 2H), 3.13 (dd, J=15.2, 10.2 Hz, 1H), 2.64 (dd, J=15.2, 4.8 Hz, 1H), 1.37 (d, J=6.7 Hz, 3H), 1.10 (t, J=7.2 Hz, 3H), 1.04-0.94 (m, 2H), 0.04 (s, 9H). Ethyl-(3R,4S)-rel-3-(2,3-dichloro-6-[[2-(trimethylsilyl)ethoxy]methoxy]phenyl)-4-nitropentanoate: ¹H NMR (400 MHz, CDCl₃) δ 7.32 (d, J=9.0 Hz, 1H), 7.09 (d, J=9.0 Hz, 1H), 5.34-5.27 (m, 1H), 5.26 (s, 2H), 4.62-4.55 (m, 1H), 4.10-4.00 (m, 2H), 3.87-3.77 (m, 2H), 2.98-2.79 (m, 2H), 1.67 (d, J=6.6 Hz, 3H), 1.15 (t, J=7.1 Hz, 3H), 1.04-0.95 (m, 2H), 0.05 (s, 9H).

Step b:

A mixture of ethyl-(3R,4R)-rel-3-(2,3-dichloro-6-[[2-(trimethylsilyl)ethoxy]methoxy]phenyl)-4-nitropentanoate (1.20 g, 2.57 mmol) and Zn (3.37 g, 51.52 mmol) in AcOH (10 mL) was stirred at room temperature for 16 h. The mixture was filtered and the filter cake washed with MeOH (2×10 mL). The filtrate was concentrated under reduced pressure and the residue purified by reverse phase chromatography, eluting with 40% ACN in water (plus 0.05% TFA) to afford Intermediate 8 (ethyl-(3R,4R)-rel-4-amino-3-(2,3-dichloro-6-[[2-(trimethylsilyl)ethoxy]methoxy]phenyl)pentanoate) as an off-white solid (1.10 g, 78%): LCMS (ESI) calc'd for C₁₉H₃₁Cl₂NO₄Si [M+H]⁺: 436, 438 (3:2) found 436, 438 (3:2); ¹H NMR (400 MHz, CDCl₃) δ 8.22 (brs, 3H), 7.36 (d, J=9.0 Hz, 1H), 7.11 (d, J=9.1 Hz, 1H), 5.29-5.26 (m, 2H), 4.24-4.00 (m, 4H), 3.82-3.71 (m, 2H), 3.30 (dd, J=16.7, 6.2 Hz, 1H), 2.89 (dd, J=16.4, 5.6 Hz, 1H), 1.20 (d, J=6.1 Hz, 3H), 1.16 (t, J=7.1 Hz, 3H), 1.01-0.91 (m, 2H), 0.02 (s, 9H).

Step c:

A mixture of ethyl-(3R,4S)-rel-3-(2,3-dichloro-6-[[2-(trimethylsilyl)ethoxy]methoxy]phenyl)-4-nitropentanoate (0.770 g, 1.65 mmol) and Zn (2.16 g, 32.97 mmol) in AcOH (7 mL) was stirred for 16 h at room temperature. The mixture was filtered and the filter cake washed with MeOH (2×10 mL). The filtrate was concentrated under reduced pressure and the residue purified by reverse phase chromatography, eluting with 40% ACN in water (plus 0.05% TFA) to afford Intermediate 9 (ethyl-(3R,4S)-rel-4-amino-3-(2,3-dichloro-6-[[2-(trimethylsilyl)ethoxy]methoxy]phenyl)pentanoate) as an off-white solid (0.430 g, 47%): LCMS (ESI) calc'd for C₁₉H₃₁Cl₂NO₄Si [M+H]⁺: 436, 438 (3:2) found 436,438 (3:2); ¹H NMR (400 MHz, CDCl₃) δ 7.40 (d, J=9.0 Hz, 1H), 7.12 (d, J=9.0 Hz, 1H), 5.32 (dd, J=52.2, 6.9 Hz, 2H), 4.33-4.30 (m, 1H), 4.15-4.00 (m, 3H), 3.82-3.73 (m, 2H), 3.07-2.87 (m, 2H), 1.40 (d, J=6.1 Hz, 3H), 1.16 (t, J=7.1 Hz, 3H), 1.03-0.91 (m, 2H), 0.02 (s, 9H).

### Example 9. Intermediate 10 (2-([2-[(tert-butyldimethylsilyl)oxy]ethyl]amino)-1-(2,3-dichloro-6-[[2-(trimethylsilyl)ethoxy]methoxy]phenyl)ethanol)

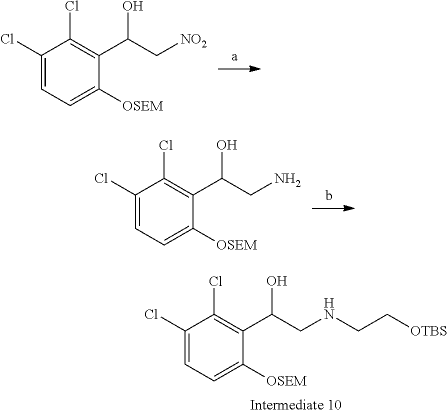

Step a:

To a stirred solution of 1-(2,3-dichloro-6-[[2-(trimethylsilyl)ethoxy]methoxy]phenyl)-2-nitroethanol (6.00 g, 15.7 mmol) (Example 4, step a) in AcOH (60 mL) was added Zn (10.3 g, 157 mmol) at room temperature under nitrogen atmosphere. The resulting mixture was stirred for 16 h and filtered. The filter cake was washed with MeOH (2×20 mL) and the filtrate was concentrated under reduced pressure. The residue was purified by reverse phase chromatography, eluting with 50% ACN in water (plus 0.05% TFA) to afford 2-amino-1-(2,3-dichloro-6-[[2-(trimethylsilyl)ethoxy]methoxy]phenyl)ethanol as a yellow oil (4.50 g, 81%): LCMS (ESI) calc'd for C₁₄H₂₃Cl₂NO₃Si [M+H]⁺: 352, 354 (3:2) found 352, 354 (3:2); ¹H NMR (300 MHz, CDCl₃) δ 7.35 (d, J=9.0 Hz, 1H), 7.10 (d, J=9.0 Hz, 1H), 5.35-5.28 (m, 2H), 5.25-5.14 (m, 1H), 3.83-3.71 (m, 2H), 3.10 (t, J=11.1 Hz, 1H), 2.95 (d, J=12.7 Hz, 1H), 1.02-0.91 (m, 2H), 0.02 (s, 9H).

Step b:

To a solution of 2-amino-1-(2,3-dichloro-6-[[2-(trimethylsilyl)ethoxy]methoxy]phenyl)ethanol (1.20 g, 3.41 mmol) and 2-[(tert-butyldimethylsilyl)oxy]acetaldehyde (0.590 g, 3.41 mmol) in DCM (15 mL) was added NaBH₃CN (0.430 g, 6.85 mmol) at room temperature. The reaction mixture was stirred for 2 h, quenched with saturated aqueous NH₄Cl (50 mL), and extracted with EA (3×50 mL). The combined organic layers were washed with brine (3×50 mL) and dried over anhydrous Na₂SO₄. After filtration, the filtrate was concentrated under reduced pressure. The residue was purified by reverse phase chromatography, eluting with 55% ACN in water (plus 0.05% TFA) to afford Intermediate 10 (2-([2-[(tert-butyldimethylsilyl)oxy]ethyl]amino)-1-(2,3-dichloro-6-[[2-(trimethylsilyl)ethoxy]methoxy]phenyl)ethanol) as a yellow oil (0.600 g, 35%): LCMS (ESI) calc'd for C₂₂H₄₁Cl₂NO₄Si₂ [M+H]⁺: 510, 512 (3:2) found 510, 512 (3:2); ¹H NMR (300 MHz, CDCl₃) δ 7.39-7.33 (m, 1H), 7.15-7.06 (m, 1H), 5.38-5.26 (m, 4H), 3.95-3.71 (m, 5H), 3.44-3.24 (m, 1H), 3.10-2.87 (m, 2H), 1.04-0.93 (m, 2H), 0.92 (s, 9H), 0.11 (s, 6H), 0.03 (s, 9H).

Examples 10-27 describe the syntheses and/or characterization data of representative compounds of Formula I disclosed herein.

### Example 10. Compound 1 (4-(2,3-dichloro-6-hydroxyphenyl)-1-[pyrrolidin-3-yl]piperidin-2-one isomer 1), Compound 2 (4-(2,3-dichloro-6-hydroxyphenyl)-1-[pyrrolidin-3-yl]piperidin-2-one isomer 2), Compound 3 (4-(2,3-dichloro-6-hydroxyphenyl)-1-[pyrrolidin-3-yl]piperidin-2-one isomer 3), and Compound 4 (4-(2,3-dichloro-6-hydroxyphenyl)-1-[pyrrolidin-3-yl]piperidin-2-one isomer 4)

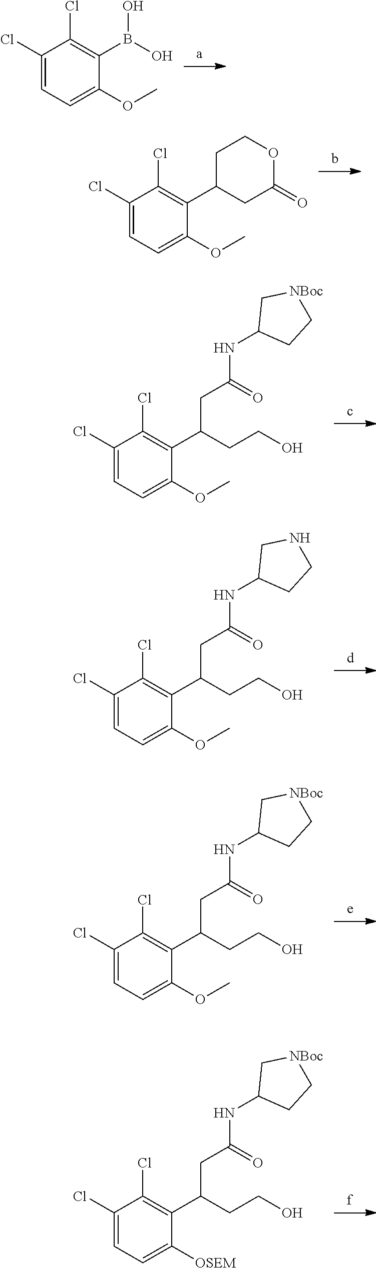

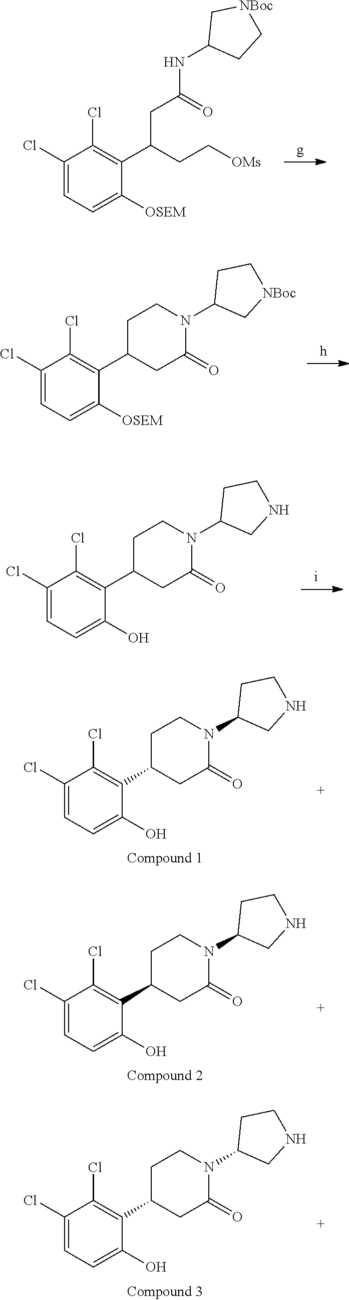

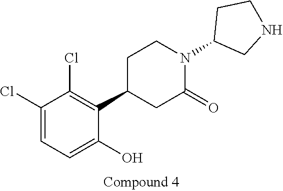 [SMILES: O=C1C[C@@H](c2c(O)ccc(Cl)c2Cl)CCN1[C@@H]1CCNC1]

Step a:

To a stirred mixture of 5,6-dihydropyran-2-one (1.00 g, 10.2 mmol) and 2,3-dichloro-6-methoxyphenylboronic acid (3.37 g, 15.3 mmol) in 1,4-dioxane (15 mL) were added K₃PO₄ (4.33 g, 20.4 mmol) and [Rh(COD)Cl]₂ (0.500 g, 1.02 mmol) at room temperature. The reaction mixture was stirred at 80° C. for 5 h and filtered. The filter cake was washed with EA (3×10 mL) and the filtrate was concentrated under reduced pressure. The residue was purified by reverse phase chromatography, eluting with 50% ACN in water (plus 10 mM NH₄HCO₃) to afford 4-(2,3-dichloro-6-methoxyphenyl)pyran-2-one as a yellow oil (1.50 g, 53%): LCMS (ESI) calc'd for C₁₂H₁₂Cl₂O₃ [M+H]⁺: 275, 277 (3:2) found 275, 277 (3:2); ¹H NMR (400 MHz, CDCl₃) δ 7.37 (d, J=8.9 Hz, 1H), 6.81 (d, J=8.9 Hz, 1H), 4.53-4.41 (m, 1H), 4.41-4.29 (m, 1H), 4.19-4.04 (m, 1H), 3.83 (d, J=1.0 Hz, 3H), 2.94-2.74 (m, 2H), 2.23-2.02 (m, 2H).

Step b:

To a stirred mixture of tert-butyl 3-aminopyrrolidine-1-carboxylate (2.03 g, 10.9 mmol) in toluene (15 mL) was added AlMe₃ (4.91 mL, 9.82 mmol) dropwise at room temperature under nitrogen atmosphere. The reaction mixture was stirred for 1 h, then a solution of 4-(2,3-dichloro-6-methoxyphenyl)pyran-2-one (1.50 g, 5.45 mmol) in THE (2 mL) was added dropwise. The reaction mixture was stirred for 2 h, quenched with water (10 mL), basified to pH 8 with saturated aqueous Na₂CO₃ (20 mL), and extracted with EA (3×20 mL). The combined organic layers were washed with brine (2×20 mL) and dried over anhydrous Na₂SO₄. After filtration, the filtrate was concentrated under reduced pressure. The residue was purified by reverse phase chromatography, eluting with 50% ACN in water (plus 10 mM NH₄HCO₃) to afford tert-butyl 3-[3-(2,3-dichloro-6-methoxyphenyl)-5-hydroxypentanamido]pyrrolidine-1-carboxylate as a light-yellow oil (2.00 g, 72%): LCMS (ESI) calc'd for C₂₁H₃₀Cl₂N₂O₅ [M+H]⁺: 461, 463 (3:2) found 461, 463 (3:2); ¹H NMR (400 MHz, CD₃OD) δ 7.37 (dd, J=8.9, 2.5 Hz, 1H), 6.94 (dd, J=9.3, 2.5 Hz, 1H), 4.47-4.38 (m, 1H), 4.25-4.17 (m, 1H), 4.17-4.08 (m, 1H), 3.88 (d, J=2.3 Hz, 3H), 3.70-3.61 (m, 1H), 3.53-3.36 (m, 4H), 3.28 (dd, J=11.4, 4.9 Hz, 1H), 2.81-2.62 (m, 2H), 2.29-2.14 (m, 1H), 2.06-1.90 (m, 2H), 1.48 (d, J=2.8 Hz, 9H).

Step c:

To a stirred mixture of tert-butyl 3-[3-(2,3-dichloro-6-methoxyphenyl)-5-hydroxypentanamido]pyrrolidine-1-carboxylate (1.00 g, 2.17 mmol) in DCM (10 mL) was added BBr₃ (1 mL, 10.6 mmol) dropwise at 0° C. The reaction mixture was stirred at 40° C. for 2 h, quenched with MeOH (3 mL), and concentrated under reduced pressure. The residue was purified by reverse phase chromatography, eluting with 50% ACN in water (plus 10 mM NH₄HCO₃) to afford 3-(2,3-dichloro-6-hydroxyphenyl)-5-hydroxy-N-(pyrrolidin-3-yl)pentanamide as a colorless oil (0.300 g, 34%): LCMS (ESI) calc'd for C₁₅H₂₀Cl₂N₂O₃ [M+H]⁺: 347, 349 (3:2) found 347, 349 (3:2); ¹H NMR (400 MHz, CD₃OD) δ 7.20 (d, J=8.8 Hz, 1H), 6.71 (d, J=8.8 Hz, 1H), 4.23-4.03 (m, 2H), 3.58-3.39 (m, 2H), 3.102.92 (m, 2H), 2.922.79 (m, 2H), 2.752.59 (m, 1H), 2.522.25 (m, 2H), 2.121.89 (m, 2H), 1.72-1.42 (m, 1H).

Step d:

To a stirred mixture of 3-(2,3-dichloro-6-hydroxyphenyl)-5-hydroxy-N-(pyrrolidin-3-yl)pentanamide (0.280 g, 0.81 mmol) and TEA (82.0 mg, 0.80 mmol) in MeOH (3 mL) was added Boc₂O (0.530 g, 2.42 mmol) at room temperature. The reaction mixture was stirred for 1 h, diluted with water (20 mL), and extracted with EA (3×20 mL). The combined organic layers were washed with brine (2×20 mL) and dried over anhydrous Na₂SO₄. After filtration, the filtrate was concentrated under reduced pressure to afford tert-butyl 3-[3-(2,3-dichloro-6-hydroxyphenyl)-5-hydroxypentanamido]pyrrolidine-1-carboxylate as a colorless oil (0.270 g, crude), which was used directly in the next step without purification: LCMS (ESI) calc'd for C₂₀H₂₈Cl₂N₂O₅ [M+H]⁺: 447, 449 (3:2) found 447, 449 (3:2).

Step e:

To a stirred mixture of tert-butyl 3-[3-(2,3-dichloro-6-hydroxyphenyl)-5-hydroxypentanamido]pyrrolidine-1-carboxylate (0.270 g, 0.60 mmol) and K₂CO₃ (0.250 g, 1.81 mmol) in DMF (3 mL) was added SEMCl (0.300 g, 1.81 mmol) dropwise at room temperature. The reaction mixture was stirred for 16 h, diluted with water (20 mL), and extracted with EA (3×20 mL). The combined organic layers were washed with brine (2×20 mL) and dried over anhydrous Na₂SO₄. After filtration, the filtrate was concentrated under reduced pressure. The residue was purified by reverse phase chromatography, eluting with 70% ACN in water (plus 10 mM NH₄HCO₃) to afford tert-butyl 3-[3-(2,3-dichloro-6-[[2-(trimethylsilyl)ethoxy]methoxy]phenyl)-5-hydroxypentanamido]pyrrolidine-1-carboxylate as a colorless oil (0.190 g, 40% overall two steps): LCMS (ESI) calc'd for C₂₆H₄₂Cl₂N₂O₆Si [M+H]⁺: 577, 579 (3:2) found 577, 579 (3:2); ¹H NMR (400 MHz, CD₃OD) δ 7.37-7.32 (m, 1H), 7.17-7.03 (m, 1H), 5.40-5.26 (m, 2H), 4.27-4.09 (m, 2H), 3.83 (t, J=8.5 Hz, 2H), 3.56-3.39 (m, 4H), 3.22-3.06 (m, 1H), 3.05-2.88 (m, 1H), 2.79-2.67 (m, 2H), 2.26-2.13 (m, 1H), 2.13-1.91 (m, 2H), 1.87-1.61 (m, 1H), 1.48 (d, J=2.3 Hz, 9H), 0.98 (t, J=8.0 Hz, 2H), 0.03 (s, 9H).

Step f:

To a stirred mixture of tert-butyl-3-[3-(2,3-dichloro-6-[[2-(trimethylsilyl)ethoxy]methoxy]phenyl)-5-hydroxypentanamido]pyrrolidine-1-carboxylate (0.190 g, 0.33 mmol) and TEA (67.0 mg, 0.66 mmol) in DCM (2 mL) was added MsCl (75.0 mg, 0.66 mmol) dropwise at 0° C. The reaction mixture was stirred at room temperature for 3 h, diluted with water (20 mL), and extracted with EA (3×20 mL). The combined organic layers were washed with brine (2×20 mL) and dried over anhydrous Na₂SO₄. After filtration, the filtrate was concentrated under reduced pressure to afford tert-butyl-3-[3-(2,3-dichloro-6-[[2-(trimethylsilyl)ethoxy]methoxy]phenyl)-5-(methanesulfonyloxy)pentanamido]pyrrolidine-1-carboxylate as a yellow oil (0.210 g, crude), which was used directly in the next step without purification: LCMS (ESI) calc'd for C₂₇H₄₄Cl₂N₂O₈SSi [M+H]⁺: 655, 657 (3:2) found 655, 657 (3:2).

Step g:

To a stirred mixture of tert-butyl-3-[3-(2,3-dichloro-6-[[2-(trimethylsilyl)ethoxy]methoxy]phenyl)-5-(methanesulfonyloxy)pentanamido]pyrrolidine-1-carboxylate (0.210 g, 0.32 mmol) in DMF (3 mL) was added NaH (12.0 mg, 0.48 mmol, 60% in oil) at room temperature under nitrogen atmosphere. The reaction mixture was stirred for 3 h, quenched with saturated aqueous NH₄Cl (2 mL), diluted with water (20 mL), and extracted with EA (3×20 mL). The combined organic layers were washed with brine (2×20 mL) and dried over anhydrous Na₂SO₄. After filtration, the filtrate was concentrated under reduced pressure. The residue was purified by silica gel chromatography, eluting with PE/EA (2/3) to afford tert-butyl-3-[4-(2,3-dichloro-6-[[2-(trimethylsilyl)ethoxy]methoxy]phenyl)-2-oxopiperidin-1-yl]pyrrolidine-1-carboxylate as a colorless oil (0.110 g, 69% overall two steps): LCMS (ESI) calc'd for C₂₆H₄₀Cl₂N₂O₅Si [M+H]⁺: 559, 561 (3:2) found 559, 561 (3:2); ¹H NMR (400 MHz, CD₃OD) δ 7.38 (d, J=9.0 Hz, 1H), 7.16 (d, J=9.1 Hz, 1H), 5.30 (d, J=2.3 Hz, 2H), 5.20-5.04 (m, 1H), 4.05-3.94 (m, 1H), 3.85-3.73 (m, 2H), 3.65-3.52 (m, 2H), 3.50-3.34 (m, 2H), 3.27-3.23 (m, 1H), 3.01-2.90 (m, 1H), 2.63-2.52 (m, 1H), 2.50-2.36 (m, 1H), 2.23-2.07 (m, 2H), 2.07-1.93 (m, 2H), 1.49 (d, J=1.3 Hz, 9H), 1.00-0.92 (m, 2H), 0.02 (s, 9H).

Step h:

To a stirred mixture of tert-butyl-3-[4-(2,3-dichloro-6-[[2-(trimethylsilyl)ethoxy]methoxy]phenyl)-2-oxopiperidin-1-yl]pyrrolidine-1-carboxylate (0.110 g, 0.20 mmol) in DCM (1 ml) was added TFA (0.5 mL) at 0° C. The reaction mixture was stirred at room temperature for 1 h and concentrated under reduced pressure. The residue was purified with Prep-HPLC Column: X Select CSH Prep C18 OBD Column, 19×250 mm, 5 μm; Mobile Phase A: water (plus 0.05% TFA), Mobile Phase B: ACN; Flow rate: 20 mL/min; Gradient: 20% B to 40% B in 6.5 min; Detector: UV 210 nm; Retention Time: 6.45 min. The fractions containing the desired product were collected and concentrated under reduced pressure to afford 4-(2,3-dichloro-6-hydroxyphenyl)-1-(pyrrolidin-3-yl)-piperidin-2-one as a white solid (27.0 mg, 29%): LCMS (ESI) calc'd for C₁₅H₁₈Cl₂N₂O₂ [M+H]⁺: 329, 331 (3:2) found 329, 331 (3:2); ¹H NMR (400 MHz, CD₃OD) δ 7.25 (dd, J=8.7, 0.8 Hz, 1H), 6.75 (d, J=8.8 Hz, 1H), 4.49-4.35 (m, 1H), 3.99-3.87 (m, 1H), 3.76-3.64 (m, 1H), 3.64-3.37 (m, 4H), 3.27-3.09 (m, 2H), 2.72-2.53 (m, 1H), 2.53-2.40 (m, 2H), 2.40-2.19 (m, 1H), 2.03-1.86 (m, 1H).

Step i:

4-(2,3-dichloro-6-hydroxyphenyl)-1-(pyrrolidin-3-yl)-piperidin-2-one (27.0 mg, 0.08 mmol) was separated by Prep Chiral HPLC with the following conditions: Column: CHIRALPAK IG, 3×25 cm, 5 μm; Mobile Phase A: Hex (plus 8 mmol/L NH₃ MeOH)-HPLC, Mobile Phase B: EtOH-HPLC; Flow rate: 40 mL/min; Gradient: 15% B to 15% B in 28 min; Detector UV 220/254 nm; Retention Time 1: 13.33 min; Retention Time 2: 15.84 min; Retention Time 3:22.11 min. The faster-eluting isomer at 13.33 min was further purified by reverse phase chromatography, eluting with 35% ACN in water (plus 0.05% TFA) to afford Compound 1 (4-(2,3-dichloro-6-hydroxyphenyl)-1-[pyrrolidin-3-yl]piperidin-2-one isomer 1) as a white solid (2.90 mg, 8%): LCMS (ESI) calc'd for C₁₅H₁₈Cl₂N₂O₂ [M+H]⁺: 329, 331 (3:2) found 329, 331 (3:2); ¹H NMR (400 MHz, CD₃OD) δ 7.25 (d, J=8.8 Hz, 1H), 6.76 (d, J=8.8 Hz, 1H), 4.49-4.37 (m, 1H), 3.99-3.85 (m, 1H), 3.74-3.63 (m, 1H), 3.62-3.49 (m, 3H), 3.45 (dd, J=12.4, 8.8 Hz, 1H), 3.30-3.21 (m, 1H), 3.15 (dd, J=17.5, 10.3 Hz, 1H), 2.69-2.56 (m, 1H), 2.54-2.39 (m, 2H), 2.37-2.24 (m, 1H), 2.00-1.89 (m, 1H). The middle-eluting peak at 15.84 min was separated by Prep Chiral HPLC with the following conditions: Column: Chiral pak IC, 2×25 cm, 5 m; Mobile Phase A: MTBE (plus 0.3% IPA)-HPLC, Mobile Phase B: EtOH-HPLC; Flow rate: 20 mL/min; Gradient: 20% B to 20% B in 11 min; Detector UV 254/220 nm; Retention Time 1: 7.32 min; Retention Time 2: 9.90 min. The faster-eluting isomer at 7.32 min was obtained as Compound 2 (4-(2,3-dichloro-6-hydroxyphenyl)-1-[pyrrolidin-3-yl]piperidin-2-one isomer 2) as an off-white solid (1.70 mg, 6.30%): LCMS (ESI) calc'd for C₁₅H₁₈Cl₂N₂O₂ [M+H]⁺: 329, 331 (3:2) found 329, 331 (3:2); ¹H NMR (400 MHz, CD₃OD) δ 7.24 (d, J=8.8 Hz, 1H), 6.75 (d, J=8.8 Hz, 1H), 4.73-4.53 (m, 1H), 3.90 (d, J=6.1 Hz, 1H), 3.50 (dd, J=7.8, 4.0 Hz, 2H), 3.43-3.36 (m, 2H), 3.28-3.22 (m, 1H), 3.16 (dd, J=17.4, 10.7 Hz, 1H), 3.11-3.00 (m, 1H), 2.71-2.56 (m, 1H), 2.51-2.40 (m, 1H), 2.36-2.23 (m, 1H), 2.11-2.01 (m, 1H), 1.98-1.88 (m, 1H). The slower-eluting isomer at 9.90 min was obtained as Compound 3 (4-(2,3-dichloro-6-hydroxyphenyl)-1-[pyrrolidin-3-yl]piperidin-2-one isomer 3) as an off-white solid (1.80 mg, 6.67%): LCMS (ESI) calc'd for C₁₅H₁₈Cl₂N₂O₂ [M+H]⁺: 329, 331 (3:2) found 329, 331 (3:2); ¹H NMR (400 MHz, CD₃OD) δ 7.24 (d, J=8.8 Hz, 1H), 6.75 (d, J=8.9 Hz, 1H), 4.74-4.57 (m, 1H), 3.90 (d, J=6.1 Hz, 1H), 3.50 (dd, J=7.9, 4.0 Hz, 2H), 3.43-3.35 (m, 2H), 3.27-3.22 (m, 1H), 3.21-3.11 (m, 1H), 3.11-2.98 (m, 1H), 2.67-2.55 (m, 1H), 2.50-2.41 (m, 1H), 2.33-2.23 (m, 1H), 2.10-1.99 (m, 1H), 1.99-1.88 (m, 1H). The last-eluting isomer at 22.11 min was further purified by reverse phase chromatography, eluting with 35% ACN in water (plus 0.05% TFA) to afford Compound 4 (4-(2,3-dichloro-6-hydroxyphenyl)-1-[pyrrolidin-3-yl]piperidin-2-one isomer 4) as a white solid (3.60 mg, 10%): LCMS (ESI) calc'd for C₁₅H₁₈Cl₂N₂O₂ [M+H]⁺: 329, 331 (3:2) found 329, 331 (3:2). ¹H NMR (400 MHz, CD₃OD) δ 7.25 (d, J=8.8 Hz, 1H), 6.76 (d, J=8.8 Hz, 1H), 4.49-4.37 (m, 1H), 3.99-3.85 (m, 1H), 3.74-3.63 (m, 1H), 3.62-3.49 (m, 3H), 3.45 (dd, J=12.4, 8.8 Hz, 1H), 3.30-3.21 (m, 1H), 3.15 (dd, J=17.5, 10.3 Hz, 1H), 2.69-2.56 (m, 1H), 2.54-2.39 (m, 2H), 2.37-2.24 (m, 1H), 2.00-1.89 (m, 1H).

### Example 11. Compound 5 (5-(2,3-dichloro-6-hydroxyphenyl)-1-[pyrrolidin-3-yl]piperidin-2-one isomer 1), Compound 6 (5-(2,3-dichloro-6-hydroxyphenyl)-1-[pyrrolidin-3-yl]piperidin-2-one isomer 2), and Compound 7 (5-(2,3-dichloro-6-hydroxyphenyl)-1-[pyrrolidin-3-yl]piperidin-2-one isomer 3)

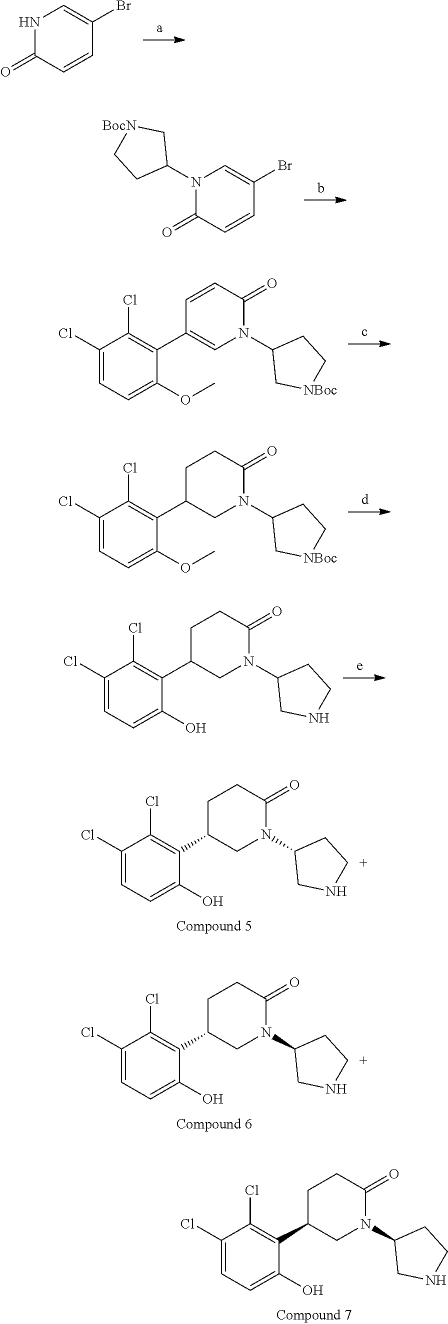

Step a:

To a stirred mixture of 5-bromo-1H-pyridin-2-one (1.00 g, 5.75 mmol) in DMF (13.0 mL) was added K₂CO₃ (1.58 g, 11.5 mmol) at room temperature. The reaction mixture was stirred for 20 min and tert-butyl-3-bromopyrrolidine-1-carboxylate (2.58 g, 10.3 mmol) was added. The reaction mixture was stirred at 100° C. for 2 h, diluted with water (80 mL), and extracted with EA (3×60 mL). The combined organic layers were washed with brine (2×50 mL) and dried over anhydrous Na₂SO₄. After filtration, the filtrate was concentrated under reduced pressure. The residue was purified by reverse phase chromatography, eluting with 50% ACN in water (plus 0.05% TFA) to afford tert-butyl-3-(5-bromo-2-oxopyridin-1-yl)pyrrolidine-1-carboxylate (0.280 g, 12%): LCMS (ESI) calc'd for C₁₄H₁₉BrN₂O₃ [M+H]⁺: 343, 345 (1:1) found 343, 345 (1:1); ¹H NMR (400 MHz, CD₃OD) δ 7.74 (s, 1H), 7.60 (dd, J=9.6, 2.6 Hz, 1H), 6.53 (dd, J=9.6, 2.6 Hz, 1H), 5.37-5.23 (m, 1H), 3.92-3.69 (m, 1H), 3.65-3.55 (m, 1H), 3.55-3.46 (m, 2H), 2.50-2.20 (m, 2H), 1.50 (s, 9H).

Step b:

To a stirred mixture of 2,3-dichloro-6-methoxyphenylboronic acid (0.350 g, 1.60 mmol), tert-butyl-3-(5-bromo-2-oxopyridin-1-yl)pyrrolidine-1-carboxylate (0.220 g, 0.64 mmol), and Na₂CO₃ (0.200 g, 1.92 mmol) in 1,4-dioxane (2 mL) and H₂O (0.50 mL) was added Pd(dppf)Cl₂ CH₂Cl₂ (26.0 mg, 0.03 mmol) at room temperature under nitrogen atmosphere. The reaction mixture was stirred at 80° C. for 16 h under nitrogen atmosphere. After cooling to room temperature, the mixture was diluted with water (50 mL) and extracted with EA (3×30 mL). The combined organic layers were washed with brine (2×20 mL) and dried over anhydrous Na₂SO₄. After filtration, the filtrate was concentrated under reduced pressure. The residue was purified by reverse phase chromatography, eluting with 75% ACN in water (plus 0.05% TFA) to afford tert-butyl-3-[5-(2,3-dichloro-6-methoxyphenyl)-2-oxopyridin-1-yl]pyrrolidine-1-carboxylate as a yellow oil (0.250 g, 80%): LCMS (ESI) calc'd for C₂₁H₂₄Cl₂N₂O₄ [M+H]⁺: 439, 441 (3:2) found 439, 441 (3:2).

Step c:

To a stirred mixture of tert-butyl-3-[5-(2,3-dichloro-6-methoxyphenyl)-2-oxopyridin-1-yl]pyrrolidine-1-carboxylate (70.0 mg, 0.16 mmol) in AcOH (3 mL) and EA (3 mL) was added PtO₂ (10.0 mg, 0.04 mmol) at room temperature. The reaction mixture was degassed under reduced pressure and purged with hydrogen three times followed by stirring at 30° C. for 24 h under hydrogen atmosphere (1.5 atm). The mixture was filtered and the filter cake washed with EA (3×10 mL). The filtrate was concentrated under reduced pressure. The residue was purified by reverse phase chromatography, eluting with 78% ACN in water (plus 0.05% TFA) to afford tert-butyl-3-[5-(2,3-dichloro-6-methoxyphenyl)-2-oxopiperidin-1-yl]pyrrolidine-1-carboxylate as a colorless oil (70.0 mg, 84%): LCMS (ESI) calc'd for C₂₁H₂₈Cl₂N₂O₄ [M+H]⁺: 443, 445 (3:2) found 443, 445 (3:2); ¹H NMR (400 MHz, CD₃OD) δ 7.44 (d, J=9.0 Hz, 1H), 7.02 (d, J=9.0 Hz, 1H), 5.12-4.98 (m, 1H), 4.00-3.91 (m, 1H), 3.89 (s, 3H), 3.80 (t, J=10.5 Hz, 1H), 3.60-3.45 (m, 3H), 3.29-3.21 (m, 2H), 2.68-2.43 (m, 2H), 2.19-2.02 (m, 2H), 1.93-1.79 (m, 2H), 1.44 (d, J=5.0 Hz, 9H).

Step d:

To a stirred mixture of tert-butyl-3-[5-(2,3-dichloro-6-methoxyphenyl)-2-oxopiperidin-1-yl]pyrrolidine-1-carboxylate (30.0 mg, 0.07 mmol) in DCM (2 mL) was added BBr₃ (0.03 mL, 0.32 mmol) at 0° C. The reaction mixture was stirred at room temperature for 1 h, quenched with MeOH (2 mL) at 0° C., and concentrated under reduced pressure. The residue was purified with prep-HPLC with the following conditions: X Bridge Prep Phenyl OBD Column, 5 μm, 19×250 mm; Mobile Phase A: Water (plus 10 mmol/L NH₄HCO₃), Mobile Phase B: ACN; Flow rate: 20 mL/min; Gradient: 40% to 60% in 6.5 min; Detector: UV 254/220 nm; Retention time: 6.45 min. The fractions containing the desired product were collected and concentrated under reduced pressure to afford 5-(2,3-dichloro-6-hydroxyphenyl)-1-(pyrrolidin-3-yl)piperidin-2-one as an off-white solid (20.1 mg, 88%): LCMS (ESI) calc'd for C₁₅H₁₈Cl₂N₂O₂ [M+H]⁺: 329, 331 (3:2) found 329, 331 (3:2); ¹H NMR (300 MHz, CD₃OD) δ 7.23 (d, J=8.8 Hz, 1H), 6.74 (d, J=8.8 Hz, 1H), 4.93-4.86 (m, 1H), 4.08-3.77 (m, 2H), 3.29-3.18 (m, 1H), 3.15-2.95 (m, 2H), 2.95-2.78 (m, 2H), 2.74-2.54 (m, 2H), 2.54-2.41 (m, 1H), 2.22-1.99 (m, 1H), 1.93-1.76 (m, 2H).

Step e:

The product 5-(2,3-dichloro-6-hydroxyphenyl)-1-(pyrrolidin-3-yl)piperidin-2-one (17.0 mg, 0.05 mmol) was purified by Prep Chiral HPLC with the following conditions: Column: CHIRAL IC, 2×25 cm, 5 μm; Mobile Phase A: Hex (plus 0.5% 2M NH₃-MeOH)-HPLC, Mobile Phase B: EtOH-HPLC; Flow rate: 20 mL/min; Gradient: 8% to 8% in 35 min; Detector: UV 254/220 nm; Retention time 1: 19.90 min; Retention time 2: 26.65 min; Retention time 3: 30.28 min. The faster-eluting isomer at 19.90 min was obtained as Compound 5 (5-(2,3-dichloro-6-hydroxyphenyl)-1-[pyrrolidin-3-yl]piperidin-2-one isomer 1) as an off-white solid (1.00 mg, 5.88%): LCMS (ESI) calc'd for C₁₅H₁₈Cl₂N₂O₂ [M+H]⁺: 329, 331 (3:2) found 329, 331 (3:2); ¹H NMR (400 MHz, CD₃OD) δ 7.26 (d, J=8.8 Hz, 1H), 6.77 (d, J=8.8 Hz, 1H), 4.76-4.63 (m, 1H), 4.09-3.79 (m, 2H), 3.31-3.24 (m, 2H), 3.24-3.07 (m, 2H), 3.05-2.96 (m, 1H), 2.76-2.55 (m, 2H), 2.55-2.41 (m, 1H), 2.31-2.16 (m, 1H), 2.09-1.94 (m, 1H), 1.94-1.76 (m, 1H). The second peak at 26.65 min was obtained as Compound 6 (5-(2,3-dichloro-6-hydroxyphenyl)-1-[pyrrolidin-3-yl]piperidin-2-one isomer 2) as a light-yellow oil (1.00 mg, 5.88%): LCMS (ESI) calc'd for C₁₅H₁₈Cl₂N₂O₂ [M+H]⁺: 329, 331 (3:2) found 329, 331 (3:2); ¹H NMR (400 MHz, CD₃OD) δ 7.27 (d, J=8.8 Hz, 1H), 6.78 (d, J=8.8 Hz, 1H), 4.34 (dd, J=11.9, 7.1 Hz, 1H), 4.15-4.09 (m, 1H), 4.05-3.87 (m, 1H), 3.73-3.61 (m, 1H), 3.58 (dd, J=12.3, 4.3 Hz, 1H), 3.46-3.36 (m, 2H), 3.27-3.16 (m, 1H), 2.77-2.64 (m, 1H), 2.61-2.40 (m, 3H), 2.30-2.19 (m, 1H), 1.91-1.80 (m, 1H). The last-eluting isomer at 30.28 min was obtained as Compound 7 (5-(2,3-dichloro-6-hydroxyphenyl)-1-[pyrrolidin-3-yl]piperidin-2-one isomer 3) as an off-white solid (1 mg, 5.88%): LCMS (ESI) calc'd for C₁₅H₁₈Cl₂N₂O₂ [M+H]⁺: 329, 331 (3:2) found 329, 331 (3:2); ¹H NMR (400 MHz, CD₃OD) δ 7.26 (d, J=8.8 Hz, 1H), 6.77 (d, J=8.8 Hz, 1H), 4.67-4.52 (m, 1H), 4.10-4.01 (m, 1H), 3.99-3.82 (m, 1H), 3.48-3.39 (m, 1H), 3.31-3.25 (m, 3H), 3.14-3.03 (m, 1H), 2.77-2.65 (m, 1H), 2.65-2.56 (m, 1H), 2.53-2.43 (m, 1H), 2.34-2.25 (m, 1H), 2.19-2.09 (m, 1H), 1.92-1.80 (m, 1H).

### Example 12. Compound 8 (4-(2,3-dichloro-6-hydroxyphenyl)-1-[pyrrolidin-3-yl]-tetrahydropyrimidin-2(1H)-one isomer 1), Compound 9 (4-(2,3-dichloro-6-hydroxyphenyl)-1-[pyrrolidin-3-yl]-tetrahydropyrimidin-2(1H)-one isomer 2), Compound 10 (4-(2,3-dichloro-6-hydroxyphenyl)-1-[pyrrolidin-3-yl]-tetrahydropyrimidin-2(1H)-one isomer 3), and Compound 11 (4-(2,3-dichloro-6-hydroxyphenyl)-1-[pyrrolidin-3-yl]-tetrahydropyrimidin-2(1H)-one isomer 4)

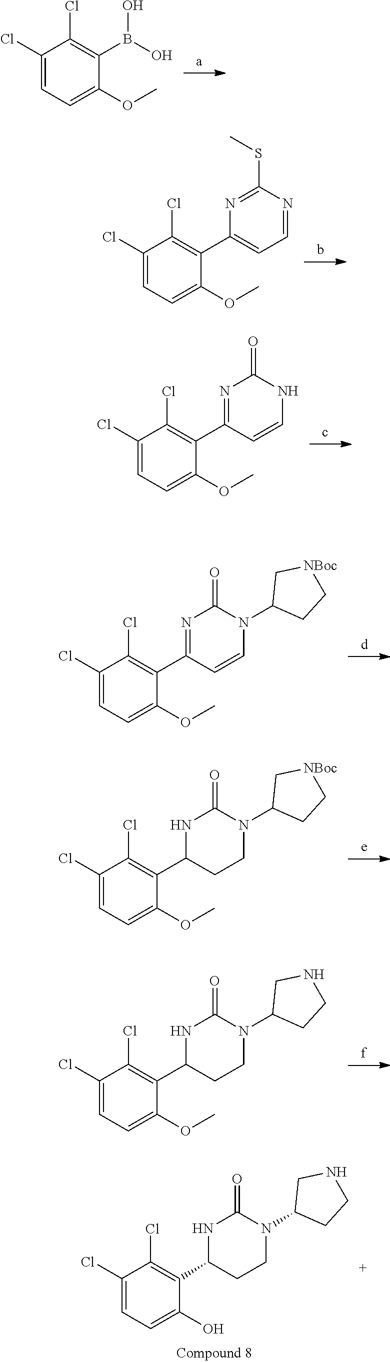

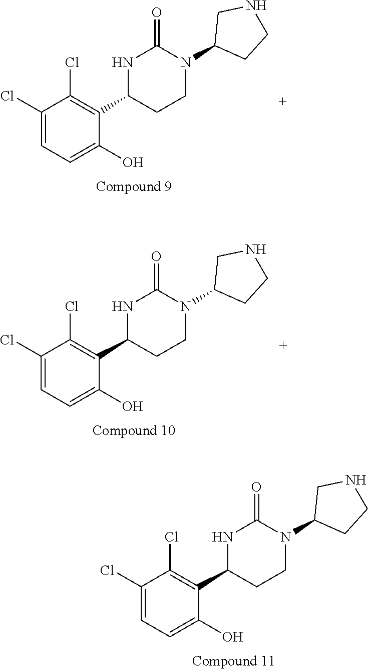 [SMILES: O=C1N[C@@H](c2c(O)ccc(Cl)c2Cl)CCN1[C@@H]1CCNC1 ; O=C1N[C@H](c2c(O)ccc(Cl)c2Cl)CCN1[C@H]1CCNC1 ; O=C1N[C@H](c2c(O)ccc(Cl)c2Cl)CCN1[C@@H]1CCNC1]

Step a:

To a stirred mixture of 4-chloro-2-(methylsulfanyl)pyrimidine (0.500 g, 3.11 mmol), 2,3-dichloro-6-methoxyphenylboronic acid (0.830 g, 3.74 mmol), and K₃PO₄ (1.32 g, 6.23 mmol) in 1,4-dioxane (4 mL) and H₂O (1 mL) were added XPhos Pd G3 (0.260 g, 0.31 mmol) and XPhos (0.150 g, 0.31 mmol) at room temperature under nitrogen atmosphere. The resulting mixture was stirred at 90° C. for 3 h. After cooling to room temperature, the resulting mixture was diluted with water (50 mL) and extracted with EA (3×30 mL). The combined organic layers were washed with brine (3×30 mL) and dried over anhydrous Na₂SO₄. After filtration, the filtrate was concentrated under reduced pressure. The residue was purified by silica gel column chromatography, eluting with PE/EA (5/1) to afford 4-(2,3-dichloro-6-methoxyphenyl)-2-(methylsulfanyl)pyrimidine as a yellow solid (0.800 g, 85%): LCMS (ESI) calc'd for C₁₂H₁₀Cl₂N₂OS [M+H]⁺: 301, 303 (3:2) found 301, 303 (3:2); ¹H NMR (400 MHz, CHCl₃) δ 8.63 (d, J=5.1 Hz, 1H), 7.51 (d, J=9.0 Hz, 1H), 6.98 (d, J=5.1 Hz, 1H), 6.89 (d, J=9.0 Hz, 1H), 3.77 (s, 3H), 2.62 (s, 3H).

Step b:

A solution of 4-(2,3-dichloro-6-methoxyphenyl)-2-(methylsulfanyl) pyrimidine (0.700 g, 2.32 mmol) in concentrated HCl (7 mL) was stirred at 100° C. for 16 h under nitrogen atmosphere. The resulting mixture was concentrated under reduced pressure. The residue was purified by reverse phase chromatography, eluting with 40% ACN in water (plus 0.05% TFA) to afford 4-(2,3-dichloro-6-methoxyphenyl)-1H-pyrimidin-2-one as a yellow solid (0.500 g, 80%): LCMS (ESI) calc'd for C₁₁H₈Cl₂N₂O₂ [M+H]⁺: 271, 273 (3:2) found 271, 273 (3:2); ¹H NMR (400 MHz, CDCl₃) δ 10.45-10.40 (brs, 1H), 8.52 (s, 1H), 7.56 (d, J=8.9 Hz, 1H), 6.91 (d, J=9.0 Hz, 1H), 6.67 (d, J=4.6 Hz, 1H), 3.82 (s, 3H).

Step c:

To a stirred solution of 4-(2,3-dichloro-6-methoxyphenyl)-1H-pyrimidin-2-one (0.500 g, 1.97 mmol) and tert-butyl-3-bromopyrrolidine-1-carboxylate (0.980 g, 0.01 mmol) in DMF (10 mL) was added K₂CO₃ (0.820 g, 0.02 mmol) at room temperature. The reaction mixture was stirred at 100° C. for 16 h under nitrogen atmosphere. After cooling to room temperature, the mixture was diluted with water (50 mL) and extracted with EA (3×30 mL). The combined organic layers were washed with brine (5×20 mL) and dried over anhydrous Na₂SO₄. After filtration, the filtrate was concentrated under reduced pressure. The residue was purified by silica gel column chromatography, eluting with DCM/MeOH (10/1) to afford tert-butyl-3-[4-(2,3-dichloro-6-methoxyphenyl)-2-oxopyrimidin-1-yl]pyrrolidine-1-carboxylate as a light-yellow solid (0.200 g, 25%): LCMS (ESI) calc'd for C₂₀H₂₃Cl₂N₃O₄ [M+H]⁺: 440, 442 (3:2) found 440, 442 (3:2); ¹H NMR (400 MHz, CHCl₃) δ 7.72 (d, J=6.8 Hz, 1H), 7.49 (d, J=8.9 Hz, 1H), 6.86 (d, J=9.0 Hz, 1H), 6.39 (d, J=6.8 Hz, 1H), 5.39-5.32 (m, 1H), 3.95-3.84 (m, 2H), 3.79 (s, 3H), 3.68-3.56 (m, 2H), 2.55-2.41 (m, 1H), 2.34-2.24 (m, 1H), 1.51 (s, 9H).

Step d:

To a solution of tert-butyl-3-[4-(2,3-dichloro-6-methoxyphenyl)-2-oxopyrimidin-1-yl]pyrrolidine-1-carboxylate (0.150 g, 0.34 mmol) in AcOH (2 mL) and EA (2 mL) was added PtO₂ (0.150 g, 0.68 mmol) at room temperature. The reaction mixture was stirred for 16 h under hydrogen atmosphere (1.5 atm) and filtered. The filter cake was washed with MeOH (5×3 mL) and the filtrate was concentrated under reduced pressure. The residue was purified by reverse phase chromatography, eluting with 45% ACN in water (plus 0.05% TFA) to afford tert-butyl-3-[4-(2,3-dichloro-6-methoxyphenyl)-2-oxotetrahydropyrimidin-1(2H)-yl]pyrrolidine-1-carboxylate as a yellow solid (70.0 mg, 46%): LCMS (ESI) calc'd for C₂₀H₂₇Cl₂N₃O₄ [M+H]⁺: 444, 446 (3:2) found 444, 446 (3:2); ¹H NMR (400 MHz, CDCl₃) δ 7.41 (d, J=9.0 Hz, 1H), 6.83 (d, J=8.9 Hz, 1H), 5.33-5.28 (m, 1H), 5.24-5.08 (m, 1H), 3.88-3.74 (m, 3H), 3.66-3.42 (m, 3H), 3.42-2.98 (m, 3H), 2.35-2.15 (m, 2H), 2.15-1.88 (m, 2H), 1.49 (s, 9H).

Step e:

To a stirred mixture of tert-butyl-3-[4-(2,3-dichloro-6-methoxyphenyl)-2-oxotetrahydropyrimidin-1(2H)-yl]pyrrolidine-1-carboxylate (70.0 mg, 0.16 mmol) in DCM (1 mL) was added BBr₃ (0.5 mL) at room temperature under nitrogen atmosphere. The reaction mixture was stirred for 2 h, quenched with MeOH (2 mL) at 0° C., and concentrated under reduced pressure. The residue was purified by Prep-HPLC with the following conditions: Column: X Select CSH Prep C18 OBD Column, 19×250 mm, 5 m; Mobile Phase A: water (plus 0.05% TFA), Mobile Phase B: ACN; Flow rate: 20 mL/min; Gradient: 24% B to 27% B in 6.5 min; Detector: UV 210 nm; Retention Time: 6.45 min. The fractions containing the desired product were collected and concentrated under reduced pressure to afford 4-(2,3-dichloro-6-hydroxyphenyl)-1-(pyrrolidin-3-yl)-tetrahydropyrimidin-2(1H)-one as a red solid (20.2 mg, 34%): LCMS (ESI) calc'd for C₁₄H₁₇Cl₂N₃O₂ [M+H]⁺: 330, 332 (3:2) found 330, 332 (3:2); ¹H NMR (400 MHz, CD₃OD) δ 8.56 (s, 1H), 7.30 (d, J=8.8 Hz, 1H), 6.80 (d, J=8.8 Hz, 1H), 5.32-5.20 (m, 1H), 4.51-4.39 (m, 1H), 3.68-3.59 (m, 1H), 3.55-3.36 (m, 4H), 3.25-3.12 (m, 1H), 2.49-2.20 (m, 3H), 2.18-2.05 (m, 1H).

Step f:

4-(2,3-dichloro-6-hydroxyphenyl)-1-(pyrrolidin-3-yl)-1,3-diazinan-2-one (16.0 mg, 0.04 mmol) was separated by Prep Chiral HPLC with the following conditions: Column: CHIRALPAK IH, 2×25 cm, 5 m; Mobile Phase A: Hex (plus 0.5% 2M NH₃-MeOH)-HPLC, Mobile Phase B: EtOH-HPLC; Flow rate: 20 mL/min; Gradient: 30% B to 30% B in 24 min; Detector: UV 220/254 nm; Retention Time 1: 7.44 min; Retention Time 2: 16.65 min. The faster-eluting peak at 7.44 min was separated by Prep Chiral HPLC with the following conditions: Column: CHIRALPAK IG, 2×25 cm, 5 m; Mobile Phase A: Hex (plus 0.5% 2M NH₃-MeOH)-HPLC, Mobile Phase B: EtOH-HPLC; Flow rate: 20 mL/min; Gradient: 30% B to 30% B in 52 min; Detector: UV 220/254 nm; Retention Time 1: 14.91 min; Retention Time 2: 46.25 min. The faster-eluting isomer at 14.91 min was obtained as Compound 8 (4-(2,3-dichloro-6-hydroxyphenyl)-1-[pyrrolidin-3-yl]-tetrahydropyrimidin-2(1H)-one isomer 1) as a light-yellow solid (3.80 mg, 23%): LCMS (ESI) calc'd for C₁₄H₁₇Cl₂N₃O₂ [M+H]⁺: 330, 332 (3:2) found 330, 332 (3:2); ¹H NMR (400 MHz, CD₃OD) δ 8.56 (s, 1H), 7.30 (d, J=8.8 Hz, 1H), 6.79 (d, J=8.8 Hz, 1H), 5.26 (dd, J=8.2, 5.5 Hz, 1H), 4.51-4.36 (m, 1H), 3.64-3.53 (m, 1H), 3.51-3.34 (m, 4H), 3.23-3.13 (m, 1H), 2.44-2.32 (m, 2H), 2.32-2.19 (m, 1H), 2.16-2.06 (m, 1H). The slower-eluting isomer at 46.25 min was obtained as Compound 9 (4-(2,3-dichloro-6-hydroxyphenyl)-1-[pyrrolidin-3-yl]-tetrahydropyrimidin-2(1H)-one isomer 2) as a light-yellow solid (0.500 mg, 3%): LCMS (ESI) calc'd for C₁₄H₁₇Cl₂N₃O₂ [M+H]⁺: 330, 332 (3:2) found 330, 332 (3:2); ¹H NMR (400 MHz, CD₃OD) δ 8.55 (s, 1H), 7.30 (d, J=8.8 Hz, 1H), 6.79 (d, J=8.8 Hz, 1H), 5.28 (dd, J=8.9, 5.4 Hz, 1H), 4.53-4.41 (m, 1H), 3.67-3.57 (m, 1H), 3.53-3.36 (m, 4H), 3.25-3.11 (m, 1H), 2.49-2.34 (m, 2H), 2.28-2.17 (m, 1H), 2.14-1.99 (m, 1H). The slower-eluting peak at 16.65 min was separated by Prep Chiral HPLC with the following conditions: Column: CHIRALPAK IC, 2×25 cm, 5 μm; Mobile Phase A: Hex (0.5% 2M NH₃-MeOH)-HPLC, Mobile Phase B: EtOH-HPLC; Flow rate: 20 mL/min; Gradient: 20% B to 20% B in 17 min; Detector: UV 220/254 nm; Retention Time 1: 10.56 min; Retention Time 2: 14.00 min. The faster-eluting isomer at 10.58 min was obtained as Compound 10 (4-(2,3-dichloro-6-hydroxyphenyl)-1pyrrolidin-3-yl]-tetrahydropyrimidin-2(1H)-one isomer 3) as an off-white solid (3.50 mg, 21%): LCMS (ESI) calc'd for C₁₄H₁₇Cl₂N₃O₂ [M+H]⁺: 330, 332 (3:2) found 330, 332 (3:2); ¹H NMR (400 MHz, CD₃OD) δ 8.57 (s, 1H), 7.29 (d, J=8.8 Hz, 1H), 6.78 (d, J=8.8 Hz, 1H), 5.27 (dd, J=8.5, 5.5 Hz, 1H), 4.65-4.56 (m, 1H), 3.48-3.36 (m, 3H), 3.30-3.21 (m, 2H), 3.13-3.04 (m, 1H), 2.42-2.22 (m, 2H), 2.22-2.04 (m, 2H). The slower-eluting isomer at 14.00 min was obtained as Compound 11 (4-(2,3-dichloro-6-hydroxyphenyl)-1-[pyrrolidin-3-yl]-tetrahydropyrimidin-2(1H)-one isomer 4) as an off-white solid (0.500 mg, 3.13%): LCMS (ESI) calc'd for C₁₄H₁₇Cl₂N₃O₂ [M+H]⁺: 330, 332 (3:2) found 330, 332 (3:2); ¹H NMR (400 MHz, CD₃OD) δ 8.56 (s, 1H), 7.30 (d, J=8.8 Hz, 1H), 6.79 (d, J=8.8 Hz, 1H), 5.28 (dd, J=8.9, 5.4 Hz, 1H), 4.54-4.43 (m, 1H), 3.64-3.54 (m, 1H), 3.51-3.36 (m, 4H), 3.22-3.13 (m, 1H), 2.50-2.33 (m, 2H), 2.25-2.14 (m, 1H), 2.12-2.04 (m, 1H).

### Example 13. Compound 12 (4-(2,3-dichloro-6-hydroxyphenyl)-[1,3-bipyrrolidin]-2-one isomer 1), Compound 13 (4-(2,3-dichloro-6-hydroxyphenyl)-[1,3-bipyrrolidin]-2-one isomer 2), Compound 14 (4-(2,3-dichloro-6-hydroxyphenyl)-[1,3-bipyrrolidin]-2-one isomer 3), and Compound 15 (4-(2,3-dichloro-6-hydroxyphenyl)-[1,3-bipyrrolidin]-2-one isomer 4)

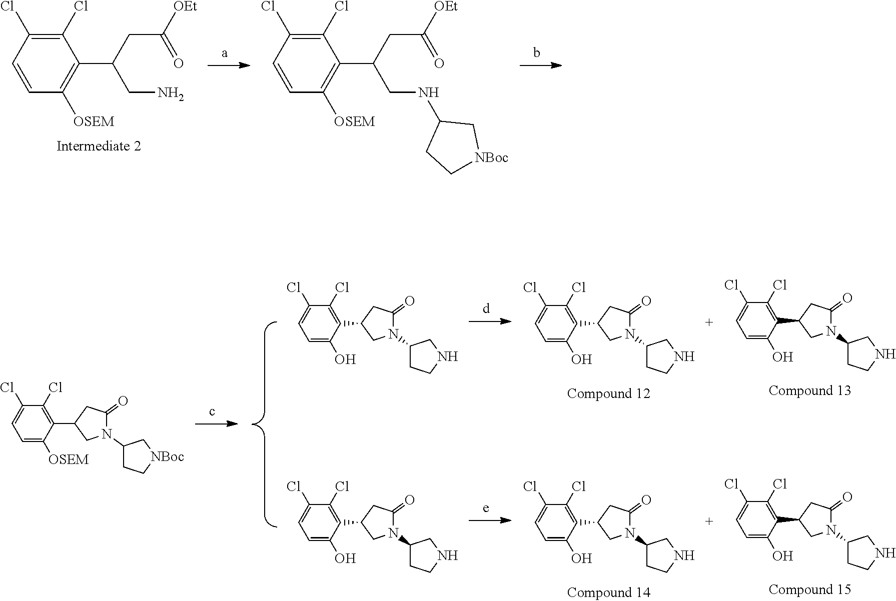

Step a:

To a stirred solution of ethyl 4-amino-3-(2,3-dichloro-6-[[2-(trimethylsilyl)ethoxy]methoxy]phenyl)butanoate (Intermediate 2, Example 2) (0.250 g, 0.59 mmol) in DCM (4 mL) were added TEA (0.120 g, 1.19 mmol) and tert-butyl-3-oxopyrrolidine-1-carboxylate (0.110 g, 0.59 mmol) at room temperature. The reaction mixture was stirred for 1 h and NaBH₃CN (75.0 mg, 1.20 mmol) was added. The reaction mixture was stirred for 5 h, quenched with water (15 mL), and extracted with EA (2×20 mL). The combined organic layers were washed with brine (2×20 mL) and dried over anhydrous Na₂SO₄. After filtration, the filtrate was concentrated under reduced pressure. The residue was purified by reverse phase chromatography, eluting with 75% ACN in water (plus 0.05% TFA) to afford tert-butyl-3-[[2-(2,3-dichloro-6-[[2-(trimethylsilyl)ethoxy]methoxy]phenyl)-4-ethoxy-4-oxobutyl]amino]pyrrolidine-1-carboxylate as a light-yellow oil (0.200 g, 57%): LCMS (ESI) calc'd for C₂₇H₄₄Cl₂N₂O₆Si [M+H]⁺: 591, 593 (3:2) found 591, 593 (3:2); ¹H NMR (300 MHz, CD₃OD) δ 7.50 (d, J=9.1 Hz, 1H), 7.22 (d, J=9.1 Hz, 1H), 5.46-5.29 (m, 2H), 4.45-4.26 (m, 1H), 4.10 (q, J=6.9 Hz, 2H), 3.97-3.72 (m, 4H), 3.68-3.49 (m, 3H), 3.48-3.38 (m, 2H), 3.13-2.87 (m, 2H), 2.49-2.31 (m, 1H), 2.19-1.96 (m, 1H), 1.48 (d, J=1.5 Hz, 9H), 1.17 (t, J=7.1 Hz, 3H), 1.00 (t, J=8.1 Hz, 2H), 0.06 (s, 9H).

Step b:

To a stirred solution of tert-butyl-3-[[2-(2,3-dichloro-6-[[2-(trimethylsilyl)ethoxy]methoxy]phenyl)-4-ethoxy-4-oxobutyl]amino]pyrrolidine-1-carboxylate (0.200 g, 0.34 mmol) in EtOH (3 mL) was added LiOH H₂O (28.0 mg, 0.68 mmol) at room temperature. The reaction mixture was stirred for 4 h, diluted with water (20 mL), and extracted with EA (2×20 mL). The combined organic layers were washed with brine (2×20 mL) and dried over anhydrous Na₂SO₄. After filtration, the filtrate was concentrated under reduced pressure. The residue was purified by reverse phase chromatography, eluting with 85% ACN in water (plus 0.05% TFA) to afford tert-butyl-4-(2,3-dichloro-6-[[2-(trimethylsilyl)ethoxy]methoxy]phenyl)-2-oxo-[1,3′-bipyrrolidine]-1′-carboxylate as a light-yellow oil (0.140 g, 76%): LCMS (ESI) calc'd for C₂₅H₃₈Cl₂N₂O₅Si [M+H]⁺: 545, 547 (3:2) found 545, 547 (3:2); ¹H NMR (300 MHz, CD₃OD) δ 7.41 (d, J=8.9 Hz, 1H), 7.18 (d, J=8.9 Hz, 1H), 5.39-5.21 (m, 2H), 4.76-4.65 (m, 1H), 4.57-4.40 (m, 1H), 3.91-3.70 (m, 3H), 3.66-3.46 (m, 3H), 3.46-3.36 (m, 2H), 2.88-2.59 (m, 2H), 2.26-2.02 (m, 2H), 1.57-1.36 (m, 9H), 0.97 (t, J=8.0 Hz, 2H), 0.06 (s, 9H).

Step c:

To a stirred solution of tert-butyl-4-(2,3-dichloro-6-[[2-(trimethylsilyl)ethoxy]methoxy]phenyl)-2-oxo-[1,3-bipyrrolidine]-1-carboxylate (0.130 g, 0.24 mmol) in DCM (2 mL) was added TFA (0.50 mL, 6.73 mmol) at room temperature. The reaction mixture was stirred for 1 h and concentrated under reduced pressure. The residue was purified by Prep-HPLC with the following conditions: Column: X Select CSH Prep C18 OBD Column, 19×250 mm, 5 μm; Mobile Phase A: Water (plus 0.1% FA), Mobile Phase B: ACN; Flow rate: 20 mL/min; Gradient: 25% B to 35% B in 6.5 min; Detector: UV 210 nm; Retention time 1: 6.45 min; Retention time 2: 6.84 min. The faster-eluting diastereoisomer at 6.45 min was obtained 4-(2,3-dichloro-6-hydroxyphenyl)-[1,3′-bipyrrolidin]-2-one diastereoisomer 1 as a light-pink solid (17.4 mg, 20%): LCMS (ESI) calc'd for C₁₄H₁₆Cl₂N₂O₂ [M+H]⁺: 315, 317 (3:2) found 315, 317 (3:2); ¹H NMR (300 MHz, CD₃OD) δ 8.55 (s, 1H), 7.28 (d, J=8.8 Hz, 1H), 6.80 (d, J=8.8 Hz, 1H), 4.72-4.58 (m, 1H), 4.53-4.36 (m, 1H), 3.85-3.80 (m, 1H), 3.76-3.65 (m, 1H), 3.59-3.40 (m, 3H), 3.36-3.26 (m, 1H), 2.90-2.64 (m, 2H), 2.40-2.15 (m, 2H). The slower-eluting diastereoisomer at 6.84 min was obtained 4-(2,3-dichloro-6-hydroxyphenyl)-[1,3′-bipyrrolidin]-2-one diastereoisomer 2 as a light-pink solid (19.0 mg, 22%): LCMS (ESI) calc'd for C₁₄H₁₆Cl₂N₂O₂ [M+H]⁺: 315, 317 (3:2) found 315, 317 (3:2); ¹H NMR (300 MHz, CD₃OD) δ 8.54 (s, 1H), 7.28 (d, J=8.7 Hz, 1H), 6.80 (d, J=8.6 Hz, 1H), 4.66-4.52 (m, 1H), 4.52-4.36 (m, 1H), 3.85-3.80 (m, 1H), 3.74-3.63 (m, 1H), 3.61-3.40 (m, 3H), 3.39-3.24 (m, 1H), 2.88-2.64 (m, 2H), 2.42-2.14 (m, 2H).

Step d:

4-(2,3-dichloro-6-hydroxyphenyl)-[1,3-bipyrrolidin]-2-one diastereoisomer 1 (15.0 mg, 0.04 mmol) was separated by Prep Chiral HPLC with the following conditions: Column: CHIRALPAK IG, 2×25 cm, 5 μm; Mobile Phase A: Hex (plus 7M NH₃ MeOH)-HPLC, Mobile Phase B: EtOH-HPLC; Flow rate: 20 mL/min; Gradient: 20% B to 20% B in 13 min; Detector: UV 220/254 nm; Retention time 1: 8.54 min; Retention time 2: 11.21 min. The faster-eluting enantiomer at 8.54 min was obtained as Compound 12 (4-(2,3-dichloro-6-hydroxyphenyl)-[1,3-bipyrrolidin]-2-one isomer 1) as an off-white solid (3.10 mg, 23%): LCMS (ESI) calc'd for C₁₄H₁₆Cl₂N₂O₂ [M+H]⁺: 315, 317 (3:2) found 315, 317 (3:2); ¹H NMR (400 MHz, CD₃OD) δ 7.27 (d, J=8.7 Hz, 1H), 6.78 (d, J=8.8 Hz, 1H), 4.72-4.58 (m, 1H), 4.48-4.31 (m, 1H), 3.80-3.78 (m, 1H), 3.73-3.66 (m, 1H), 3.24-3.11 (m, 2H), 3.10-2.96 (m, 2H), 2.82 (dd, J=17.1, 7.7 Hz, 1H), 2.71 (dd, J=17.1, 10.9 Hz, 1H), 2.19-2.06 (m, 1H), 2.06-1.94 (m, 1H). The slower-eluting enantiomer at 11.21 min was obtained as Compound 13 (4-(2,3-dichloro-6-hydroxyphenyl)-[1,3-bipyrrolidin]-2-one isomer 2) as an off-white solid (3.50 mg, 27%): LCMS (ESI) calc'd for C₁₄H₁₆C₁₂N₂O₂ [M+H]⁺: 315, 317 (3:2), found 315, 317 (3:2); ¹H NMR (400 MHz, CD₃OD) δ 7.27 (d, J=8.7 Hz, 1H), 6.78 (d, J=8.8 Hz, 1H), 4.72-4.58 (m, 1H), 4.48-4.31 (m, 1H), 3.80-3.78 (m, 1H), 3.73-3.66 (m, 1H), 3.24-3.11 (m, 2H), 3.10-2.96 (m, 2H), 2.82 (dd, J=17.1, 7.7 Hz, 1H), 2.71 (dd, J=17.1, 10.9 Hz, 1H), 2.19-2.06 (m, 1H), 2.06-1.94 (m, 1H).

Step e:

4-(2,3-dichloro-6-hydroxyphenyl)-[1,3-bipyrrolidin]-2-one diastereoisomer 2 (15.0 mg, 0.04 mmol) was separated by Prep Chiral HPLC with the following conditions: Column: CHIRALPAK IG, 2×25 cm, 5 μm; Mobile Phase A: Hex (plus 7M NH₃ MeOH)-HPLC, Mobile Phase B: EtOH-HPLC; Flow rate: 20 mL/min; Gradient: 15% B to 15% B in 17 min; Detector: UV 220/254 nm; Retention time 1: 8.54 min; Retention time 2: 11.21 min. The faster-eluting enantiomer at 8.54 min was obtained as Compound 14 (4-(2,3-dichloro-6-hydroxyphenyl)-[1,3-bipyrrolidin]-2-one isomer 3) as an off-white solid (2.70 mg, 20%): LCMS (ESI) calc'd for C₁₄H₁₆Cl₂N₂O₂ [M+H]⁺: 315, 317 (3:2), found 315, 317 (3:2); ¹H NMR (400 MHz, CD₃OD) δ 7.27 (d, J=8.8 Hz, 1H), 6.78 (d, J=8.8 Hz, 1H), 4.68-4.56 (m, 1H), 4.48-4.31 (m, 1H), 3.83-3.79 (m, 1H), 3.72-3.60 (m, 1H), 3.23-3.08 (m, 2H), 3.08-2.95 (m, 2H), 2.81-2.69 (m, 2H), 2.21-2.06 (m, 1H), 2.05-1.93 (m, 1H). The slower-eluting enantiomer at 11.21 min was obtained as Compound 15 (4-(2,3-dichloro-6-hydroxyphenyl)-[1,3-bipyrrolidin]-2-one isomer 4) as an off-white solid (3.50 mg, 27%): LCMS (ESI) calc'd for C₁₄H₁₆Cl₂N₂O₂ [M+H]⁺: 315, 317 (3:2) found 315, 317 (3:2); ¹H NMR (400 MHz, CD₃OD) δ 7.27 (d, J=8.8 Hz, 1H), 6.78 (d, J=8.8 Hz, 1H), 4.68-4.56 (m, 1H), 4.48-4.31 (m, 1H), 3.83-3.79 (m, 1H), 3.72-3.60 (m, 1H), 3.23-3.08 (m, 2H), 3.08-2.95 (m, 2H), 2.81-2.69 (m, 2H), 2.21-2.06 (m, 1H), 2.05-1.93 (m, 1H).

### Example 14. Compound 16 ((4S)-4-(2,3-dichloro-6-hydroxyphenyl)-1-[1-(2-hydroxyethyl)azetidin-3-yl]pyrrolidin-2-one)

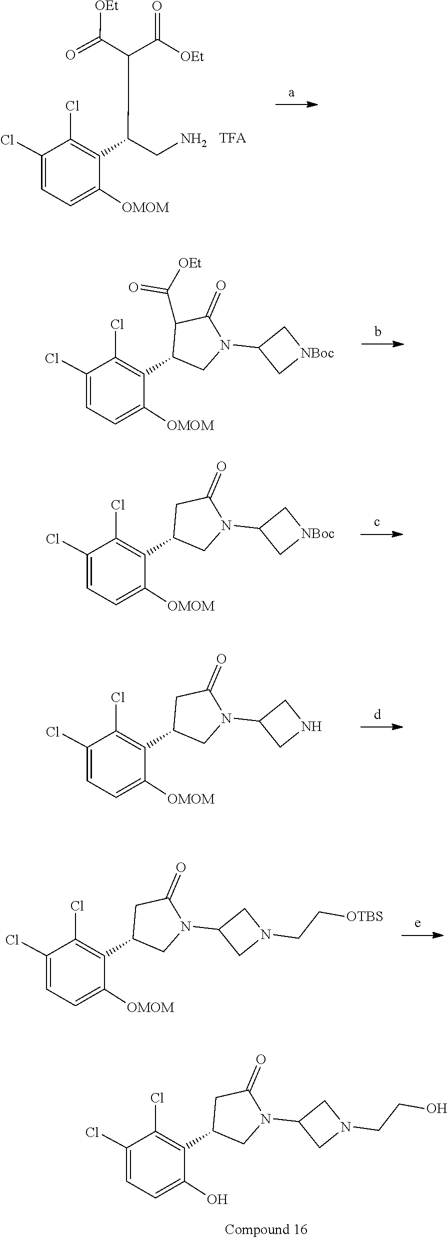

Step a:

A mixture of 1,3-diethyl 2-[(LS)-2-amino-1-[2,3-dichloro-6-(methoxymethoxy)phenyl]ethyl]propanedioate trifluoroacetic acid salt (1.00 g, 1.91 mmol), tert-butyl 3-oxoazetidine-1-carboxylate (0.490 g, 2.87 mmol), NaBH(OAc)₃ (1.22 g, 5.74 mmol), and NaOAc (0.150 g, 1.91 mmol) in DCE (20 mL) was stirred at 60° C. for 1 h. The resulting mixture was quenched with water (20 mL) at room temperature and extracted with EA (3×20 mL). The combined organic layers were washed with brine (2×20 mL) and dried over anhydrous Na₂SO₄. After filtration, the filtrate was concentrated under reduced pressure. The residue was purified by reverse phase chromatography, eluting with 40% ACN in water (plus 10 mM NH₄HCO₃) to afford ethyl (4S)-1-[1-(tert-butoxycarbonyl)azetidin-3-yl]-4-[2,3-dichloro-6-(methoxymethoxy)phenyl]-2-oxopyrrolidine-3-carboxylate as an off-white solid (0.570 g, 57%): LCMS (ESI) calc'd for C₂₃H₃₀Cl₂N₂O₇ [M+Na]⁺: 539, 541 (3:2) found 539, 541 (3:2); ¹H NMR (300 MHz, CDCl₃) δ 7.38 (d, J=9.0 Hz, 1H), 7.08 (d, J=9.0 Hz, 1H), 5.20 (s, 2H), 5.13-4.99 (m, 1H), 4.98-4.84 (m, 1H), 4.31-4.13 (m, 4H), 4.11-3.84 (m, 4H), 3.72 (dd, J=9.0, 7.1 Hz, 1H), 3.48 (s, 3H), 1.45 (s, 9H), 1.30 (t, J=7.1 Hz, 3H).

Step b:

A mixture of ethyl (4S)-1-[1-(tert-butoxycarbonyl)azetidin-3-yl]-4-[2,3-dichloro-6-(methoxymethoxy)phenyl]-2-oxopyrrolidine-3-carboxylate (0.560 g, 1.09 mmol) and LiOH H₂O (0.320 g, 7.68 mmol) in MeOH (5 mL) and H₂O (5 mL) was stirred at room temperature for 1 h. The resulting mixture was acidified to pH 4-5 with citric acid and extracted with EA (3×30 mL). The combined organic layers were washed with brine (2×30 mL) and dried over anhydrous Na₂SO₄. After filtration, the filtrate was concentrated under reduced pressure. The residue was dissolved in toluene (12 mL) and stirred at 110° C. for 16 h. The resulting mixture was cooled to room temperature and concentrated under reduced pressure. The residue was purified by reverse phase chromatography, eluting with 47% ACN in water (plus 10 mM NH₄HCO₃) to afford tert-butyl 3-[(4S)-4-[2,3-dichloro-6-(methoxymethoxy)phenyl]-2-oxopyrrolidin-1-yl]azetidine-1-carboxylate as an off-white solid (0.390 g, 80%): LCMS (ESI) calc'd for C₂₀H₂₆Cl₂N₂O₅ [M+H]⁺: 445, 447 (3:2) found 445, 447 (3:2); ¹H NMR (400 MHz, CDCl₃) δ 7.35 (d, J=9.0 Hz, 1H), 7.07 (d, J=9.0 Hz, 1H), 5.24-5.12 (m, 2H), 5.12-5.05 (m, 1H), 4.54-4.44 (m, 1H), 4.19 (dt, J=15.3, 8.7 Hz, 2H), 4.04 (dd, J=9.4, 5.4 Hz, 1H), 4.00 (dd, J=9.2, 5.4 Hz, 1H), 3.92-3.89 (m, 1H), 3.75 (dd, J=9.2, 6.7 Hz, 1H), 3.47 (s, 3H), 2.88-2.73 (m, 2H), 1.45 (s, 9H).

Step c:

A solution of tert-butyl 3-[(4S)-4-[2,3-dichloro-6-(methoxymethoxy)phenyl]-2-oxopyrrolidin-1-yl]azetidine-1-carboxylate (0.390 g, 0.87 mmol) in DCM (5 mL) and TFA (0.5 mL) was stirred at room temperature for 4 h. The resulting mixture was basified to pH 8 with saturated aqueous NaHCO₃(20 mL) and extracted with EA (3×30 mL). The combined organic layers were washed with brine (2×30 mL) and dried over anhydrous Na₂SO₄. After filtration, the filtrate was concentrated under reduced pressure. The residue was purified by reverse phase chromatography, eluting with 23% ACN in water (plus 0.05% TFA) to afford (4S)-1-(azetidin-3-yl)-4-[2,3-dichloro-6-(methoxymethoxy)phenyl]pyrrolidin-2-one as a yellow oil (0.170 g, 59%): LCMS (ESI) calc'd for C₁₅H₁₈Cl₂N₂O₃ [M+H]⁺: 345, 347 (3:2) found 345,347 (3:2); ¹H NMR (300 MHz, CDCl₃) δ 7.35 (dd, J=9.0, 1.4 Hz, 1H), 7.07 (dd, J=9.0, 1.4 Hz, 1H), 5.25-5.07 (m, 3H), 4.54-4.38 (m, 1H), 4.01-3.88 (m, 5H), 3.77 (dd, J=9.2, 6.8 Hz, 1H), 3.47 (d, J=1.8 Hz, 3H), 2.83-2.73 (m, 2H).

Step d:

To a solution of (4S)-1-(azetidin-3-yl)-4-[2,3-dichloro-6-(methoxymethoxy)phenyl]pyrrolidin-2-one (0.170 g, 0.51 mmol) and K₂CO₃ (0.210 g, 1.54 mmol) in ACN (5 mL) was added (2-bromoethoxy)(tert-butyl)dimethylsilane (0.240 g, 1.03 mmol) at room temperature. The reaction mixture was stirred at 80° C. for 3 h, cooled to room temperature, and concentrated under reduced pressure. The residue was purified by reverse phase chromatography, eluting with 80% ACN in water (plus 10 mM NH₄HCO₃) to afford (4S)-1-(1-[2-[(tert-butyldimethylsilyl)oxy]ethyl]azetidin-3-yl)-4-[2,3-dichloro-6-(methoxymethoxy)phenyl]pyrrolidin-2-one as a yellow oil (0.100 g, 41%): LCMS (ESI) calc'd for C₂₃H₃₆Cl₂N₂O₄Si [M+H]⁺: 503, 505 (3:2) found 503, 505 (3:2); ¹H NMR (400 MHz, CDCl₃) δ 7.34 (d, J=9.0 Hz, 1H), 7.06 (d, J=9.0 Hz, 1H), 5.24-5.09 (m, 2H), 4.95-4.81 (m, 1H), 4.53-4.36 (m, 1H), 3.90-3.87 (m, 1H), 3.74-3.63 (m, 5H), 3.46 (s, 3H), 3.41-3.26 (m, 2H), 2.87-2.69 (m, 2H), 2.63 (t, J=5.6 Hz, 2H), 0.89 (s, 9H), 0.06 (s, 6H).

Step e:

To a solution of (4S)-1-(1-[2-[(tert-butyldimethylsilyl)oxy]ethyl]azetidin-3-yl)-4-[2,3-dichloro-6-(methoxymethoxy)phenyl]pyrrolidin-2-one (0.100 g, 0.19 mmol) in MeOH (0.5 mL) was added HCl (4 M, 1.5 mL) at room temperature. The reaction mixture was stirred for 3 h and concentrated under reduced pressure. The residue was purified by reverse phase chromatography, eluting with 23% ACN in water (plus 0.05% TFA) to afford the product. The product was purified by Prep Chiral HPLC with the following conditions: Column: CHIRALPAK IC, 2×25 cm, 5 μm; Mobile Phase A: Hex (plus 0.5% 2 M NH₃-MeOH), Mobile Phase B: EtOH; Flow rate: 20 mL/min; Gradient: 15% B to 15% B in 16 min; Detector: UV 220/254 nm; Retention time: 10.48 min. The fractions containing the desired product were collected and concentrated under reduced pressure to afford Compound 16 ((4S)-4-(2,3-dichloro-6-hydroxyphenyl)-1-[1-(2-hydroxyethyl)azetidin-3-yl]pyrrolidin-2-one) as an off-white solid (32.0 mg, 46%): LCMS (ESI) calc'd for C₁₅H₁₈Cl₂N₂O₃ [M+H]⁺: 345, 347 (3:2) found 345, 347 (3:2); ¹H NMR (300 MHz, CD₃OD) δ 7.27 (d, J=8.8 Hz, 1H), 6.79 (d, J=8.8 Hz, 1H), 4.83-4.73 (m, 1H), 4.51-4.34 (m, 1H), 3.91 (t, J=9.5 Hz, 1H), 3.80 (dd, J=9.3, 6.8 Hz, 1H), 3.71-3.68 (m, 2H), 3.58 (t, J=5.7 Hz, 2H), 3.46-3.42 (m, 2H), 2.89-2.75 (m, 1H), 2.73-2.62 (m, 3H).

The compounds in Table A below were prepared in analogous fashion to Compound 16.

TABLE A

| Compound | | | |
|---|---|---|---|
| Number | Structure | Chcmical Name | MS: (M + H⁺) & ¹H NMR |
| 17 | 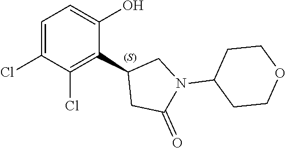 | (4S)-4-(2,3-dichloro- 6-hydroxyphenyl)-1- (pyran-4- yl)pyrrolidin-2-one | [M + H]⁺: 330, 332 (3:2); ¹H NMR (400 MHz, CD₃OD) δ 7.27 (d, J = 8.8 Hz, 1H), 6.78 (d, J = 8.7 Hz, 1H), 4.46-4.33 (m, 1H), 4.26-4.17 (m, 1H), 4.06-3.98 (m, 2H), 3.79-3.73 (m, 1H), 3.66 (dd, J = 9.3, 6.7 Hz, 1H), 3.57-3.47 (m, 2H), 2.82 (dd, J = 17.0, 7.7 Hz, |
| | | | 1H), 2.76-2.68 (m, 1H), |
| | | | 1.92-1.78 (m, 2H), |
| | | | 1.36-1.28 (m, 2H). |
| 18 | 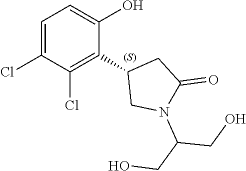 | (4S)-4-(2,3-dichloro- 6-hydroxyphenyl)-1- (1,3- dihydroxypropan-2- yl)pyrrolidin-2-one | [M + H]⁺: 320, 322 (3:2); ¹H NMR (400 MHz, CD₃OD) δ 7.26 (d, J = 8.8 Hz, 1H), 6.78 (d, J = 8.8 Hz, 1H), 4.51-4.37 (m, 1H), 4.22-4.15 (m, 1H), 3.85-3.69 (m, 6H), 2.92 (dd, J = 16.9, 8.0 Hz, 1H), 2.75-2.63 (m, 1H). |
| 19 | 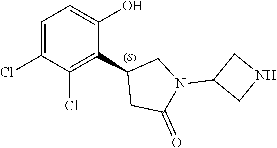 | (4S)-1-(azetidin-3- yl)-4-(2,3-dichloro-6- hydroxyphenyl)pyrro- lidin-2-one | [M + H]⁺: 301, 303 (3:2); ¹H NMR (400 MHz, CD₃OD) δ 7.29 (d, J = 8.8 Hz, 1H), 6.80 (d, J = 8.8 Hz, 1H), 4.99-4.87 (m, 1H), 4.59-4.42 (m, 3H), 4.39-4.24 (m, 2H), 3.96-3.86 (m, 1H), 3.79 (dd, J = 9.0, 6.6 Hz, 1H), 2.93-2.69 (m, 2H). |
| 20 | 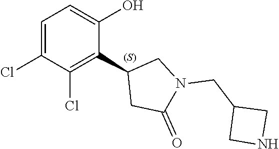 | (4S)-1-(azetidin-3- ylmethyl)-4-(2,3- dichloro-6- hydroxyphenyl)pyrro- lidin-2-one | [M + H]⁺: 315, 317 (3:2); ¹H NMR (400 MHz, CD₃OD) δ 7.24 (d, J = 8.8 Hz, 1H), 6.77 (d, J = 8.8 Hz, 1H), 4.50-4.32 (m, 1H), 3.87-3.57 (m, 6H), 3.52-3.34 (m, 2H), 3.21-2.99 (m, 1H), 2.86-2.63 (m, 2H). |
| 21 | 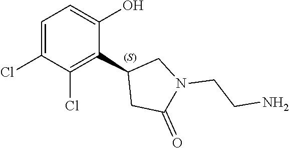 | (4S)-1-(2- aminoethyl)-4-(2,3- dichloro-6- hydroxyphenyl)pyrro- lidin-2-one | [M + H]⁺: 289, 291 (3:2); ¹H NMR (400 MHz, CD₃OD) δ 7.26 (d, J = 8.8 Hz, 1H), 6.77 (d, J = 8.8 Hz, 1H), 4.50-4.41 (m, 1H), 3.81-3.76 (m, 1H), 3.67 (dd, J = 9.4, 6.4 Hz, 1H), 3.52-3.43 (m, 2H), 2.91 (t, J = 6.3 Hz, 2H), 2.86-2.70 (m, 2H). |
| 22 | 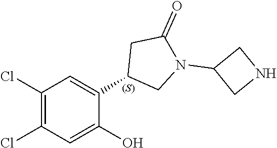 | (4S)-1-(azetidin-3- yl)-4-(4,5-dichloro-2- hydroxyphenyl)pyrro- lidin-2-one | [M + H]⁺: 301, 303 (3:2); ¹H NMR (400 MHz, CD₃OD) δ 7.32 (s, 1H), 6.98 (s, 1H), 4.96-4.87 (m, 1H), 4.55-4.47 (m, 2H), 4.33-4.24 (m, 2H), 4.00- 3.94 (m, 1H), 3.89-3.80 (m, 1H), 3.63 (dd, J = 9.0, 7.0 Hz, 1H), 2.88 (dd, J = |
| | | | 17.0, 9.2 Hz, 1H), 2.70 (dd, |
| | | | J = 17.0, 8.4 Hz, 1H). |
| 23 | 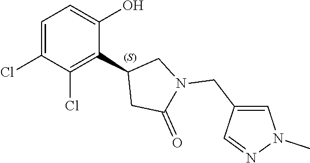 | (4S)-4-(2,3-dichloro- 6-hydroxyphenyl)-1- [(1-methylpyrazol-4- yl)methyl]pyrrolidin- 2-one | [M + H]⁺: 340, 342 (3:2); ¹H NMR (400 MHz, CD₃OD) δ 7.57 (s, 1H), 7.44 (s, 1H), 7.23 (d, J = 8.7 Hz, 1H), 6.74 (d, J = 8.8 Hz, 1H), 4.53-4.26 (m, 3H), 3.87 (s, 3H), 3.62 (d, J = 8.5 Hz, 2H), 2.86 (dd, J = 17.0, 7.9 Hz, 1H), 2.68 (dd, J = 17.0, 9.0 Hz, 1H). |
| 24 | 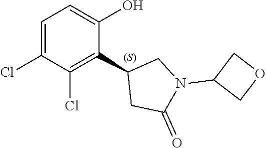 | (S)-4-(2,3-dichloro-6- hydroxyphenyl)-1- (oxetan-3- yl)pyrrolidin-2-one | [M + H]⁺: 302, 304 (3:2); ¹H NMR (300 MHz, CD₃OD) δ 7.28 (d, J = 8.8 Hz, 1H), 6.80 (d, J = 8.8 Hz, 1H), 5.37-5.25 (m, 1H), 4.97-4.79 (m, 4H), 4.55-4.83 (m, 1H), 4.10-4.01 (m, 1H), 3.94 (dd, J = 9.2, 6.7 Hz, 1H), 2.90-2.65 (m, 2H). |
| 25 | 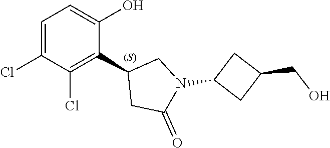 | (S)-4-(2,3-dichloro-6- hydroxyphenyl)-1-(3- (hydroxymethyl)cyclo- butyl)pyrrolidin-2- one isomer 1 | [M + H]⁺: 330, 332 (3:2); ¹H NMR (400 MHz, CD₃OD) δ 7.27 (d, J = 8.8 Hz, 1H), 6.79 (d, J = 8.7 Hz, 1H), 4.79-4.71 (m, 1H), 4.49-4.36 (m, 1H), 3.93-3.87 (m, 1H), 3.81 (dd, J = 9.4, 6.9 Hz, 1H), 3.65 (d, J = 6.8 Hz, 2H), 2.84 (dd, J = 17.0, 7.8 Hz, 1H), 2.69 (dd, |
| | | | J = 17.0, 10.8 Hz, 1H), |
| | | | 2.50-2.31 (m, 3H), |
| | | | 2.13-2.03 (m, 2H). |
| 26 | 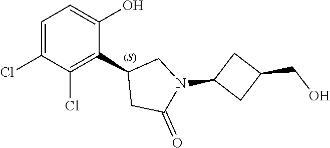 | (S)-4-(2,3-dichloro-6- hydroxyphenyl)-1-(3- (hydroxymethyl)cyclo- butyl)pyrrolidin-2- one isomer 2 | [M + H]⁺: 330, 332 (3:2); ¹H NMR (400 MHz, CD₃OD) δ 7.27 (d, J = 8.8 Hz, 1H), 6.78 (d, J = 8.8 Hz, 1H), 4.60-4.49 (m, 1H), 4.45-4.34 (m, 1H), 3.85-3.79 (m, 1H), 3.75 (dd, J = 9.4, 7.0 Hz, 1H), 3.51 (d, J = 5.5 Hz, 2H), 2.85 (dd, J = 17.0, 7.8 Hz, 1H), 2.69 (dd, |
| | | | J = 17.0, 10.9 Hz, 1H), |
| | | | 2.28-2.17 (m, |
| | | | 3H), 2.13-2.03 (m, 2H). |
| 27 | 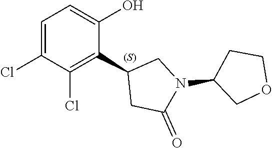 | (4S)-4-(2,3-dichloro- 6-hydroxyphenyl)-1- [oxolan-3- yl]pyrrolidin-2-one isomer 1 | [M + H]⁺: 316, 318 (3:2); ¹H NMR (400 MHz, CD₃OD) δ 7.27 (d, J = 8.8 Hz, 1H), 6.78 (d, J = 8.8 Hz, 1H), 4.89-4.80 (m, 1H), 4.45-4.36 (m, 1H), 4.03-3.95 (m, 1H), 3.90 (dd, J = 9.7, 3.3 Hz, 1H), 3.86- 3.70 (m, 3H), 3.66 (dd, J = 9.5, 6.6 Hz, 1H), 2.84-2.65 |
| | | | (m, 2H), 2.30-2.20 (m, |
| | | | 1H), 2.08-1.98 (m, 1H). |
| 28 | 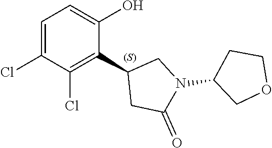 | (4S)-4-(2,3-dichloro- 6-hydroxyphenyl)-1- [oxolan-3- yl]pyrrolidin-2-one isomer 2 | [M + H]⁺: 316, 318 (3:2); ¹H NMR (300 MHz, CD₃OD) δ 7.26 (d, J = 8.8 Hz, 1H), 6.78 (d, J = 8.8 Hz, 1H), 4.89-4.80 (m, 1H), 4.45-4.35 (m, 1H), 4.05-3.98 (m, 1H), 3.88-3.73 (m, 4H), 3.67 (dd, J = 9.5, 6.5 Hz, 1H), 2.84-2.66 (m, 2H), 2.31-2.22 (m, 1H), 2.09-2.00 (m, 1H). |

### Example 15. Compound 29 ((4S)-4-(2,3-dichloro-6-hydroxyphenyl)-1-(2-hydroxyethyl)pyrrolidin-2-one)

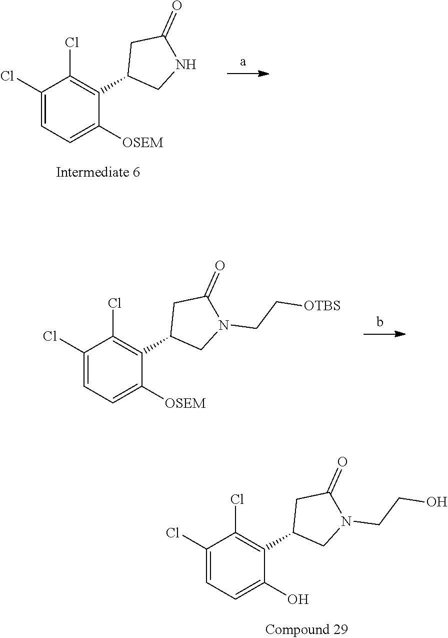

To a stirred mixture of (4S)-4-(2,3-dichloro-6-[[2-(trimethylsilyl)ethoxy]methoxy]phenyl)pyrrolidin-2-one (Intermediate 6, Example 6) (0.160 g, 0.42 mmol) in THE (2 mL) was added NaH (68.0 mg, 1.70 mmol, 60% in oil) in portions at room temperature. The reaction mixture was stirred for 20 min and (2-bromoethoxy)(tert-butyl)dimethylsilane (0.410 g, 1.70 mmol) was added. The resulting mixture was stirred at 60° C. for 16 h, quenched with saturated aqueous NH₄Cl (20 mL) at 0° C., and extracted with EA (3×20 mL). The combined organic layers were washed with brine (2×20 mL) and dried over anhydrous Na₂SO₄. After filtration, the filtrate was concentrated under reduced pressure. The residue was purified by Prep-TLC (PE/EA=3/1) to afford (S)-1-[2-[(tert-butyldimethylsilyl)oxy]ethyl]-4-(2,3-dichloro-6-[[2-(trimethylsilyl)ethoxy]methoxy]phenyl)pyrrolidin-2-one as a light-yellow oil (0.240 g, 95%): LCMS (ESI) calc'd for C₂₄H₄₁Cl₂NO₄Si₂ [M+H]⁺: 534, 536 (3:2) found 534, 536 (3:2); ¹H NMR (400 MHz, CDCl₃) δ 7.33 (d, J=9.0 Hz, 1H), 7.09 (d, J=9.0 Hz, 1H), 5.25-5.21 (m, 2H), 4.45-4.35 (m, 1H), 3.86-3.68 (m, 5H), 3.703.62 (m, 2H), 3.343.25 (m, 1H), 2.82 (dd, J=16.9, 8.1 Hz, 1H), 2.68 (dd, J=16.9, 10.8 Hz, 1H), 0.980.93 (m, 2H), 0.92 (s, 9H), 0.09 (d, J=3.3 Hz, 6H), 0.02 (s, 9H).

Step b:

To a stirred solution of (S)-1-[2-[(tert-butyldimethylsilyl)oxy]ethyl]-4-(2,3-dichloro-6-[[2-(trimethylsilyl)ethoxy]methoxy]phenyl)pyrrolidin-2-one (0.200 g, 0.37 mmol) in 1,4-dioxane (2 mL) was added HCl (6 M, 1 mL) at room temperature. The reaction mixture was stirred for 2 h and concentrated under reduced pressure. The residue was purified by Prep Chiral HPLC with the following conditions: Column: CHIRALPAK IH, 2.0×25 cm, 5 m; Mobile Phase A: Hex (plus 0.2% DEA)-HPLC, Mobile Phase B: EtOH-HPLC; Flow rate: 20 mL/min; Gradient: 20% B to 20% B in 22 min; Detector: UV 220/254 nm; Retention time: 15.71 min. The fractions containing the desired product were collected and concentrated under reduced pressure to afford Compound 29 ((S)-4-(2,3-dichloro-6-hydroxyphenyl)-1-(2-hydroxyethyl)pyrrolidin-2-one) as an off-white solid (69.1 mg, 61%): LCMS (ESI) calc'd for C₁₂H₁₃Cl₂NO₃ [M+H]⁺: 290, 292 (3:2) found 290, 292 (3:2); ¹H NMR (400 MHz, CD₃OD) δ 7.26 (d, J=8.8 Hz, 1H), 6.78 (d, J=8.8 Hz, 1H), 4.50-4.37 (m, 1H), 3.86-3.82 (m, 1H), 3.80-3.70 (m, 3H), 3.62-3.52 (m, 1H), 3.40-3.35 (m, 1H), 2.87 (dd, J=16.9, 7.9 Hz, 1H), 2.68 (dd, J=16.9, 10.8 Hz, 1H).

The compounds in Table B below were prepared in analogous fashion to Compound 29.

TABLE B

| Compound | | | |
|---|---|---|---|
| Number | Structure | Chemcial Name | MS: (M + H)⁺ & ¹H NMR |
| 30 | 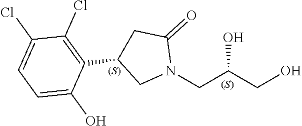 | (4S)-4-(2,3-dichloro-6- hydroxyphenyl)-1-[(2S)- 2,3- dihydroxypropyl]pyrroli- din-2-one | [M + H]⁺: 320, 322 (3:2); ¹H NMR (400 MHz, CD₃OD) δ 7.27 (d, J = 8.7 Hz, 1H), 6.78 (d, J = 8.7 Hz, 1H), 4.51-4.37 (m, 1H), 3.92-3.79 (m, 2H), 3.78 (dd, J = 9.5, 7.1 Hz, 1H), 3.61- 3.50 (m, 2H), 3.45 (dd, J = 6.0, 2.3 Hz, 2H), 2.87 (dd, J = 1 7.0, |
| | | | 7.8 Hz, 1H), 2.70 (dd, J = 17.0, |
| | | | 10.8 Hz, 1H). |
| 31 | 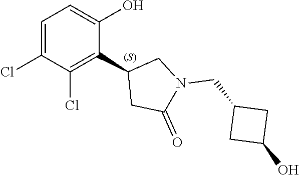 | (S)-4-(2,3-dichloro-6- hydroxyphenyl)-1-((3- hydroxycyclobutyl)meth- yl)pyrrolidin-2-one isomer 1 | [M + H]⁺: 330, 332 (3:2); ¹H NMR (400 MHz, CD₃OD) δ 7.25 (d, J = 8.8 Hz, 1H), 6.77 (d, J = 8.8 Hz, 1H), 4.48-4.33 (m, 2H), 3.74-3.62 (m, 2H), 3.53 (dd, J = 13.6, 8.7 Hz, 1H), 3.34- 3.18 (m, 1H), 2.87 (dd, J = 17.0, 7.9 Hz, 1H), 2.68 (dd, J = 17.0, 10.9 Hz, 1H), 2.59-2.48 (m, 1H), 2.20-2.01 (m, 3H), 1.18 (dd, J = 17.4, 6.4 Hz, 1H). |
| 32 | 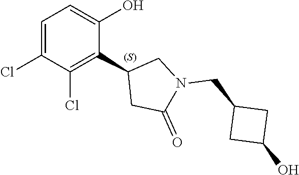 | (S)-4-(2,3-dichloro-6- hydroxyphenyl)-1-((3- hydroxycyclobutyl)meth- yl)pyrrolidin-2-one isomer 2 | [M + H ]⁺: 330, 332 (3:2); ¹H NMR (400 MHz, CD₃OD) δ 7.26 (d, J = 8.8 Hz, 1H), 6.78 (d, J = 8.8 Hz, 1H), 4.46-4.33 (m, 1H), 4.10-4.02 (m, 1H), 3.76- 3.63 (m, 2H), 3.49 (dd, J = 13.6, 7.4 Hz, 1H), 3.27 (dd, J = 13.6, 7.1 Hz, 1H), 2.84 (dd, J = 16.9, 7.9 Hz, 1H), 2.66 (dd, J = 17.0, 10.9 Hz, 1H), 2.48-2.38 (m, 2H), 2.12-2.01 (m, 1H), 1.73-1.62 (m, |
| | | | 2H). |
| 33 | 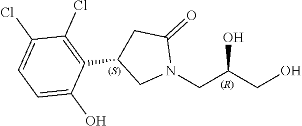 | (4S)-4-(2,3-dichloro-6- hydroxyphenyl)-1-[(2R)- 2,3- dihydroxypropyl]pyrroli- din-2-one | [M + H]⁺: 320, 322 (3:2); ¹H NMR (400 MHz, CD₃OD) δ 7.27 (d, J = 8.7 Hz, 1H), 6.79 (d, J = 8.7 Hz, 1H), 4.50-4.37 (m, 1H), 3.91-3.79 (m, 3H), 3.59- 3.49 (m, 3H), 3.36-3.33 (m, 1H), 2.91 (dd, J = 17.0, 8.0 Hz, 1H), 2.67 (dd, J = 17.0, 10.8 Hz, 1H). |
| 34 | 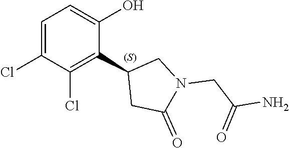 | 2-[(4S)-4-(2,3-dichloro- 6-hydroxyphenyl)-2- oxopyrrolidin-1- yl]acetamide | [M + H]⁺: 303, 305 (3:2); ¹H NMR (400 MHz, CD₃OD) δ 7.27 (d, J = 8.8 Hz, 1H), 6.79 (d, J = 8.8 Hz, 1H), 4.57-4.45 (m, 1H), 4.18 (d, J = 16.8 Hz, 1H), 3.92-3.75 (m, 2H), 3.70 (dd, J = 9.2, 6.8 Hz, 1H), 2.87 (dd, J = 17.0, 7.7 Hz, 1H), 2.74 (dd, J = 17.1, 10.8 Hz, 1H). |
| 35 | 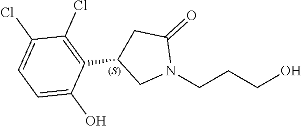 | (4S)-4-(2,3-dichloro-6- hydroxyphenyl)-1-(3- hydroxypropyl)pyrrolidin- 2-one | [M + H]⁺: 304, 306 (3:2); ¹H NMR (400 MHz, CD₃OD) δ 7.26 (d, J = 8.8 Hz, 1H), 6.78 (d, J = 8.8 Hz, 1H), 4.49-4.37 (m, 1H), 3.73 (d, J = 8.4 Hz, 2H), 3.68-3.46 (m, 3H), 3.41-3.33 (m, 1H), 2.85 (dd, J = 17.0, 7.8 Hz, 1H), 2.68 (dd, J = 17.0, 10.9 Hz, |
| | | | 1H), 1.85-1.75 (m, 2H). |
| 36 | 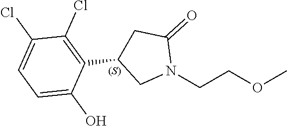 | (4S)-4-(2,3-dichloro-6- hydroxyphenyl)-1-(2- methoxyethyl)pyrrolidin- 2-one | [M + H]⁺: 304, 306 (3:2); ¹H NMR (400 MHz, CD₃OD) δ 7.26 (d, J = 8.7 Hz, 1H), 6.78 (d, J = 8.8 Hz, 1H), 4.46-4.36 (m, 1H), 3.78 (d, J = 8.4 Hz, 2H), 3.61-3.54 (m, 2H), 3.58-3.45 (m, 2H), 3.37 (s, 3H), 2.87 (dd, J = 17.0, 8.0 Hz, 1H), 2.66 (dd, J = |
| | | | 16.9, 10.8 Hz, 1H). |
| 37 | 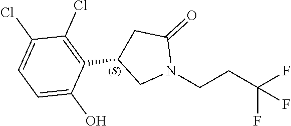 | (4S)-4-(2,3-dichloro-6- hydroxyphenyl)-1- (3,3,3- trifluoropropyl)pyrrolidin- 2-one | [M + H]⁺: 342, 344 (3:2); ¹H NMR (400 MHz, CD₃OD) δ 7.27 (d, J = 8.8 Hz, 1H), 6.78 (d, J = 8.8 Hz, 1H), 4.47-4.37 (m, 1H), 3.80-3.52 (m, 4H), 2.83 (dd, J = 17.0, 7.7 Hz, 1H), 2.68 (dd, J = 17.0, 10.8 Hz, 1H), 2.57-2.46 (m, 2H). |
| 38 | 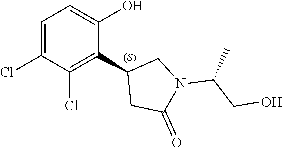 | (4S)-4-(2,3-dichloro-6- hydroxyphenyl)-1-[1- hydroxypropan-2- yl]pyrrolidin-2-one isomer 1 | [M + H]⁺: 304, 306 (3:2); ¹H NMR (400 MHz, CD₃OD) δ 7.26 (d, J = 8.8 Hz, 1H), 6.78 (d, J = 8.7 Hz, 1H), 4.44-4.35 (m, 1H), 4.25-4.15 (m, 1H), 3.80- 3.63 (m, 3H), 3.57 (dd, J = 11.2, 6.0 Hz, 1H), 2.92 (dd, J = 16.9, 8.1 Hz, 1H), 2.66 (dd, J = 16.8, 10.9 Hz, 1H), 1.23 (d, J = 6.9 |
| | | | Hz, 3H). |
| 39 | 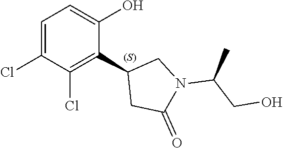 | (4S)-4-(2,3-dichloro-6- hydroxyphenyl)-1-[1- hydroxypropan-2- yl]pyrrolidin-2-one isomer 2 | [M + H]⁺: 304, 306 (3:2); ¹H NMR (400 MHz, CD₃OD) δ 7.26 (d, J = 8.7 Hz, 1H), 6.78 (d, J = 8.8 Hz, 1H), 4.48-4.38 (m, 1H), 4.35-4.26 (m, 1H), 3.76- 3.69 (m, 1H), 3.66 (dd, J = 9.2, 6.8 Hz, 1H), 3.60 (d, J = 6.7 Hz, 2H), 2.83 (dd, J = 17.0, 7.7 Hz, 1H), 2.78-2.66 (m, 1H), 1.15 (d, |
| | | | J = 6.9 Hz, 3H). |
| 40 | 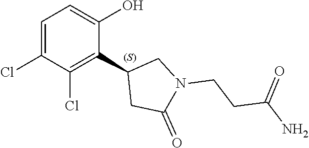 | (S)-3-(4-(2,3-dichloro-6- hydroxyphenyl)-2- oxopyrrolidin-1- yl)propanamide | [M + H]⁺: 317, 319 (3:2); ¹H NMR (400 MHz, CD₃OD) δ 7.25 (d, J = 8.7 Hz, 1H), 6.77 (d, J = 8.8 Hz, 1H), 4.46-4.31 (m, 1H), 3.82-3.70 (m, 2H), 3.65- 3.54 (m, 2H), 2.83 (dd, J = 17.0, 7.9 Hz, 1H), 2.65 (dd, J = 17.0, 10.8 Hz, 1H), 2.50 (t, J = 7.1 Hz, 2H). |
| 41 | 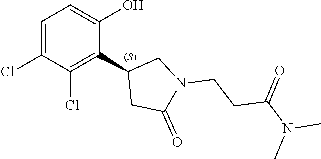 | (S)-3-(4-(2,3-dichloro-6- hydroxyphenyl)-2- oxopyrrolidin-1-yl)-N,N- dimethylpropanamide | [M + H]⁺: 345, 347 (3:2); ¹H NMR (400 MHz, CD₃OD) δ 7.24 (d, J = 8.8 Hz, 1H), 6.76 (d, J = 8.8 Hz, 1H), 4.49-4.31 (m, 1H), 3.82-3.71 (m, 2H), 3.65- 3.57 (m, 2H), 3.09 (s, 3H), 2.94 (s, 3H), 2.83 (dd, J = 16.9, 7.8 Hz, 1H), 2.75-2.57 (m, 3H). |
| 42 | 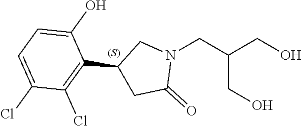 | (4S)-4-(2,3-dichloro-6- hydroxyphenyl)-1-[3- hydroxy-2- (hydroxymethyl)propyl] pyrrolidin-2-one | [M + H]⁺: 334, 336 (3:2); ¹H NMR (400 MHz, CD₃OD) δ 7.27 (d, J = 8.8 Hz, 1H), 6.79 (d, J = 8.8 Hz, 1H), 4.51-4.37 (m, 1H), 3.81-3.72 (m, 2H), 3.68- 3.55 (m, 4H), 3.49 (dd, J = 14.0, 7.7 Hz, 1H), 3.36 (d, J = 6.4 Hz, 1H), 2.88 (dd, J = 17.1, 7.7 Hz, |
| | | | 1H), 2.71 (dd, J = 17.0, 10.9 Hz, |
| | | | 1H), 2.08-1.96 (m, 1H). |
| 43 | 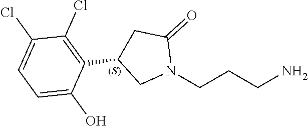 | (4S)-1-(3-aminopropyl)- 4-(2,3-dichloro-6- hydroxyphenyl)pyrrolidin- 2-one | [M + H]⁺: 303, 305 (3:2); ¹H NMR (400 MHz, CD₃OD) δ 7.28 (d, J = 8.7 Hz, 1H), 6.80 (d, J = 8.8 Hz, 1H), 4.53-4.44 (m, 1H), 3.80-3.70 (m, 2H), 3.59- 3.50 (m, 1H), 3.48-3.40 (m, 1H), 3.07-2.93 (m, 2H), 2.86 (dd, J = 17.1, 7.6 Hz, 1H), 2.75 (dd, J = |
| | | | 17.2, 10.9 Hz, 1H), 2.02-1.89 |
| | | | (m, 2H). |
| 44 | 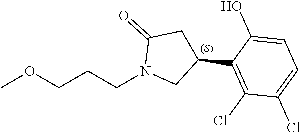 | (S)-4-(2,3-dichloro-6- hydroxyphenyl)-1-(3- methoxypropyl)pyrrolidin- 2-one | [M + H]⁺: 318, 320 (3:2); ¹H NMR (400 MHz, CD₃OD) δ 7.27 (d, J = 8.7 Hz, 1H), 6.79 (d, J = 8.8 Hz, 1H), 4.48-4.35 (m, 1H), 3.75-3.66 (m, 2H), 3.55- 3.43 (m, 3H), 3.39-3.36 (m, 1H), 3.35 (s, 3H), 2.86 (dd, J = 17.0, 7.9 Hz, 1H), 2.67 (dd, J = 16.9, 10.8 Hz, 1H), 1.91-1.80 (m, 2H). |
| 45 | 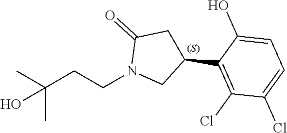 | (S)-4-(2,3-dichloro-6- hydroxyphenyl)-1-(3- hydroxy-3- methylbutyl)pyrrolidin- 2-one | [M + H]⁺: 332, 334 (3:2); ¹H NMR (400 MHz, CD₃OD) δ 7.26 (d, J = 8.8 Hz, 1H), 6.78 (d, J = 8.8 Hz, 1H), 4.48-4.34 (m, 1H), 3.73 (d, J = 8.4 Hz, 2H), 3.57-3.49 (m, 1H), 3.45-3.38 (m, 1H), 2.83 (dd, J = 16.9, 7.8 Hz, 1H), 2.67 (dd, J = 16.9, 10.8 Hz, |
| | | | 1H), 1.79-1.71 (m, 2H), 1.25 (s, |
| | | | 6H). |
| 46 | 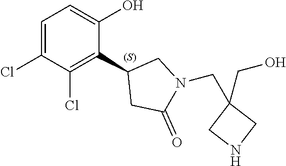 | (S)-4-(2,3-dichloro-6- hydroxyphenyl)-1-((3- (hydroxymethyl)azetidin- 3-yl)methyl)pyrrolidin- 2-one | [M + H]⁺: 345, 347 (3:2); ¹H NMR (400 MHz, CD₃OD) δ 7.25 (d, J = 8.8 Hz, 1H), 6.77 (d, J = 8.8 Hz, 1H), 4.51-4.46 (m, 1H), 3.81-3.45 (m, 10H), 2.86 (dd, J = 17.1, 7.5 Hz, 1H), 2.71 (dd, J = 17.1, 11.0 Hz, 1H). |
| 47 | 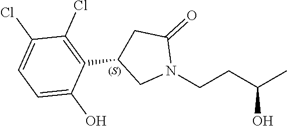 | (4S)-4-(2,3-dichloro-6- hydroxyphenyl)-1-[3- hydroxybutyl]pyrrolidin- 2-one isomer 1 | [M + H]⁺: 318, 320 (3:2); ¹H NMR (400 MHz, CD₃OD) δ 7.26 (d, J = 8.8 Hz, 1H), 6.78 (d, J = 8.8 Hz, 1H), 4.49-4.35 (m, 1H), 3.87-3.75 (m, 1H), 3.76- 3.69 (m, 2H), 3.53-3.37 (m, 2H), 2.85 (dd, J = 16.9, 7.8 Hz, 1H), 2.67 (dd, J = 16.9, 10.9 Hz, 1H), |
| | | | 1.83-1.58 (m, 2H), 1.22 (d, J = |
| | | | 6.2 Hz, 3H)). |
| 48 | 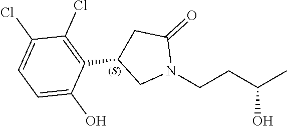 | (4S)-4-(2,3-dichloro-6- hydroxyphenyl)-1-[3- hydroxybutyl]pyrrolidin- 2-one isomer 2 | [M + H]⁺: 318, 320 (3:2); ¹H NMR (400 MHz, CD₃OD) δ 7.26 (d, J = 8.8 Hz, 1H), 6.79 (d, J = 8.8 Hz, 1H), 4.49-4.37 (m, 1H), 3.86-3.76 (m, 1H), 3.74- 3.69 (m, 2H), 3.54-3.54 (m, 1H), 3.36-3.28 (m, 1H), 2.84 (dd, J = 17.0, 7.8 Hz, 1H), 2.69 (dd, J = |
| | | | 17.0, 10.9 Hz, 1H), 1.83-1.53 |
| | | | (m, 2H), 1.22 (d, J = 6.2 Hz, |
| | | | 3H). |
| 49 | 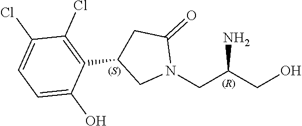 | (4S)-1-[(2R)-2-amino-3- hydroxypropyl]-4-(2,3- dichloro-6- hydroxyphenyl)pyrrolidin- 2-one | [M + H]⁺: 319, 321 (3:2); ¹H NMR (400 MHz, DMSO-d₆) δ 10.57 (s, 1H), 7.96-7.93 (brs, 3H), 7.36 (d, J = 8.8 Hz, 1H), 6.86 (d, J = 8.8 Hz, 1H), 5.33 (t, J = 4.7 Hz, 1H), 4.33-4.23 (m, 1H), 3.71-3.43 (m, 6H), 3.18 (dd, J = 13.9, 6.2 Hz, 1H), 2.66 |
| | | | (dd, J = 1.67, 8.3 Hz, 1H), 2.57- |
| | | | 2.52 (m, 1H). |
| 50 | 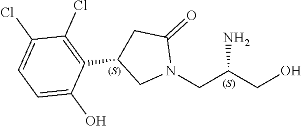 | (4S)-1-[(2S)-2-amino-3- hydroxypropyl]-4-(2,3- dichloro-6- hydroxyphenyl)pyrrolidin- 2-one | [M + H]⁺: 319, 321 (3:2); ¹H NMR (400 MHz, DMSO-d₆) δ 10.57 (s, 1H), 7.92-7.87 (brs, 3H), 7.35 (d, J = 8.8 Hz, 1H), 6.86 (d, J = 8.8 Hz, 1H), 5.33 (t, J = 4.8 Hz, 1H), 4.34-4.24 (m, 1H), 3.70-3.58 (m, 3H), 3.54- 3.43 (m, 3H), 3.19 (dd, J = 14.1, |
| | | | 5.5 Hz, 1H), 2.64 (dd, J = 16.7, |
| | | | 8.2 Hz, 1H), 2.59-2.51 (m, 1H). |
| 51 | 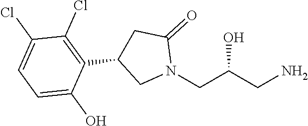 [SMILES: NC[C@@H](O)CN1C[C@H](c2c(O)ccc(Cl)c2Cl)CC1=O] | (4S)-1-[3-amino-2- hydroxypropyl]-4-(2,3- dichloro-6- hydroxyphenyl)pyrrolidin- 2-one isomer 1 | [M + H]⁺: 319, 321 (3:2); ¹H NMR (400 MHz, CD₃OD) δ 7.25 (d, J = 8.8 Hz, 1H), 6.77 (d, J = 8.8 Hz, 1H), 4.49-4.40 (m, 1H), 3.91-3.71 (m, 3H), 3.49- 3.35 (m, 2H), 2.91-2.75 (m, 2H), 2.75-2.63 (m, 2H). |
| 52 | 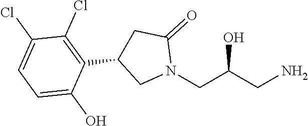 [SMILES: NC[C@H](O)CN1C[C@H](c2c(O)ccc(Cl)c2Cl)CC1=O] | (4S)-1-[3-amino-2- hydroxypropyl]-4-(2,3- dichloro-6- hydroxyphenyl)pyrrolidin- 2-one isomer 2 | [M + H]⁺: 319, 321 (3:2); ¹H NMR (400 MHz, CD₃OD) δ 7.25 (d, J = 8.8 Hz, 1H), 6.77 (d, J = 8.8 Hz, 1H), 4.50-4.36 (m, 1H), 3.89-3.81 (m, 3H), 3.45 (dd, J = 14.0, 5.0 Hz, 1H), 3.41- 3.34 (m, 1H), 2.92 (dd, J = 17.0, 8.0 Hz, 1H), 2.78 (dd, J = 13.2, |
| | | | 4.5 Hz, 1H), 2.70-2.63 (m, 2H). |

### Example 16. Compound 53 ((S)-4-(2,3-dichloro-6-hydroxyphenyl)-1-((R)-2-hydroxypropyl)pyrrolidin-2-one)

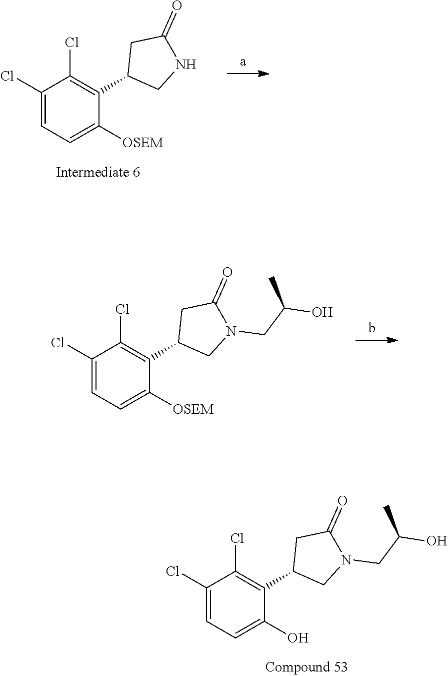

Step a:

To a stirred solution of (4S)-4-(2,3-dichloro-6-[[2-(trimethylsilyl)ethoxy]methoxy]phenyl)pyrrolidin-2-one (Intermediate 6, Example 6) (0.200 g, 0.53 mmol) and (R)-propylene oxide (62.0 mg, 1.06 mmol) in i-PrOH (2 mL) was added Cs₂CO₃ (0.340 g, 1.06 mmol) at room temperature. The reaction mixture was stirred at 100° C. for 16 h, diluted with water (20 mL), and extracted with EA (3×20 mL). The combined organic layers were washed with brine (3×20 mL) and dried over anhydrous Na₂SO₄. After filtration, the filtrate was concentrated under reduced pressure. The residue was purified by reverse phase chromatography, eluted 60% with ACN in water (plus 0.05% TFA) to afford (4S)-4-(2,3-dichloro-6-[[2-(trimethylsilyl)ethoxy]methoxy]phenyl)-1-[(2R)-2-hydroxypropyl]pyrrolidin-2-one as a yellow oil (0.200 g, 78%): LCMS (ESI) calc'd for C₁₉H₂₉Cl₂NO₄Si [M+H]⁺: 434, 436 (3:2) found 434, 436 (3:2); ¹H NMR (400 MHz, CDCl₃) δ 7.34 (d, J=9.0 Hz, 1H), 7.10 (d, J=9.0 Hz, 1H), 5.31-5.21 (m, 2H), 4.52-4.40 (m, 1H), 4.18-4.03 (m, 1H), 3.85-3.60 (m, 4H), 3.58-3.38 (m, 1H), 3.28 (dd, J=29.4, 14.1 Hz, 1H), 2.90-2.74 (m, 2H), 1.26 (d, J=6.5 Hz, 3H), 0.99-0.91 (m, 2H), 0.02 (d, J=2.0 Hz, 9H).

Step b:

To a stirred solution of (4S)-4-(2,3-dichloro-6-[[2-(trimethylsilyl)ethoxy]methoxy]phenyl)-1-[(2R)-2-hydroxypropyl]pyrrolidin-2-one (0.200 g, 0.46 mmol) in DCM (2 mL) was added TFA (0.50 mL) at room temperature. The reaction mixture was stirred for 1 h and concentrated under reduced pressure. The residue was purified by Prep Chiral HPLC with the following conditions: Column: CHIRALPAK IG, 3×25 cm, 5 μm; Mobile Phase A: Hex (plus 0.5% 2M NH₃-MeOH)-HPLC, Mobile Phase B: EtOH-HPLC; Flow rate: 40 mL/min; Gradient: 10% to 10% in 18 min; Detector: UV 254/220 nm; Retention time: 15.93 min. The fractions containing the desired product were collected and concentrated under reduced pressure to afford Compound 53 ((4S)-4-(2,3-dichloro-6-hydroxyphenyl)-1-[(2R)-2-hydroxypropyl] pyrrolidin-2-one) as an off-white solid (40.0 mg, 28%): LCMS (ESI) calc'd for C₁₃H₁₅Cl₂NO₃ [M+H]⁺: 304, 306 (3:2) found 304, 306 (3:2); ¹H NMR (400 MHz, CD₃OD) δ 7.26 (d, J=8.8 Hz, 1H), 6.78 (d, J=8.8 Hz, 1H), 4.49-4.38 (m, 1H), 4.07-3.97 (m, 1H), 3.85-3.74 (m, 2H), 3.40 (dd, J=13.8, 7.1 Hz, 1H), 3.31-3.25 (m, 1H), 2.88 (dd, J=16.9, 8.0 Hz, 1H), 2.68 (dd, J=16.9, 10.9 Hz, 1H), 1.21 (d, J=6.3 Hz, 3H).

The compounds in Table C below were prepared in analogous fashion to Compound 53.

TABLE C

| Compound | | | |
|---|---|---|---|
| Number | Structure | Chemical Name | MS: (M + H)⁺ & ¹H NMR |
| 54 | 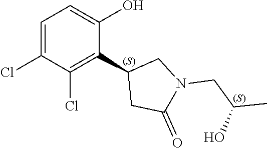 | (4S)-4-(2,3-dichloro-6- hydroxyphenyl)-1-[(2S)- 2- hydroxypropyl]pyrrolidin- 2-one | [M + H]⁺: 304, 306 (3:2); ¹H NMR (400 MHz, CD₃OD) δ 7.26 (d, J = 8.8 Hz, 1H), 6.78 (d, J = 8.8 Hz, 1H), 4.50-4.36 (m, 1H), 4.09-4.00 (m, 1H), 3.89- 3.79 (m, 2H), 3.40 (dd, J = 13.8, 4.4 Hz, 1H), 3.24 (dd, J = 13.8, 7.2 Hz, 1H), 2.91 (dd, J = 16.9, 8.1 Hz, 1H), 2.66 (dd, J = 16.9, 10.8 Hz, 1H), 1.20 (d, J = 6.3 |
| | | | Hz, 3H). |
| 55 | 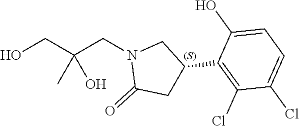 | (4S)-4-(2,3-dichloro-6- hydroxyphenyl)-1-(2,3- dihydroxy-2- methylpropyl)pyrrolidin- 2-one | [M + H]⁺: 334, 336 (3:2); ¹H NMR (400 MHz, CD₃OD) δ 7.26 (d, J = 8.8 Hz, 1H), 6.78 (d, J = 8.8 Hz, 1H), 4.49-4.36 (m, 1H), 3.93-3.82 (m, 2H), 3.51- 3.38 (m, 4H), 2.98-2.87 (m, 1H), 2.75-2.63 (m, 1H), 1.20 (s, 3H). |

### Example 17. Compound 56 ((4S)-4-(2,3-dichloro-6-hydroxyphenyl)-1-(1-methylpyrazol-4-yl)pyrrolidin-2-one)

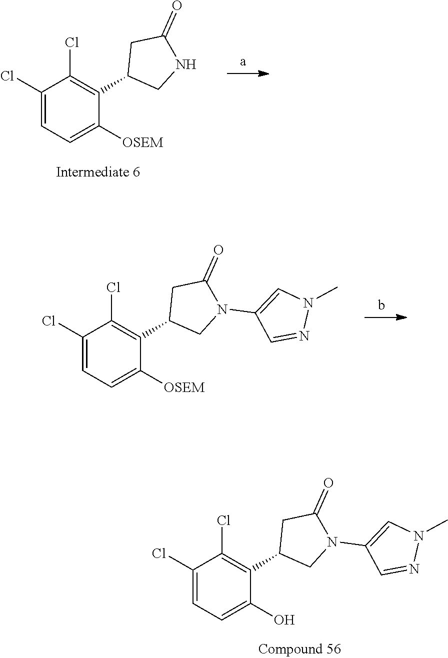

Step a:

To a stirred solution of (4S)-4-(2,3-dichloro-6-[[2-(trimethylsilyl)ethoxy]methoxy]phenyl)pyrrolidin-2-one (Intermediate 6, Example 6) (50.0 mg, 0.13 mmol) and 4-bromo-1-methylpyrazole (32.0 mg, 0.20 mmol) in 1,4-dioxane (1 mL) were added CuI (5.06 mg, 0.03 mmol), DMEDA (2.00 mg, 0.03 mmol), and K₂CO₃ (55.0 mg, 0.40 mmol) at room temperature under nitrogen atmosphere. The reaction mixture was stirred at 100° C. for 16 h under nitrogen atmosphere. After cooling to room temperature, the resulting mixture was diluted with water (20 mL) and extracted with EA (2×20 mL). The combined organic layers were washed with brine (2×20 mL) and dried over anhydrous Na₂SO₄. After filtration, the filtrate was concentrated under reduced pressure. The residue was purified by reverse phase chromatography, eluting with 55% ACN in water (plus 0.05% TFA) to afford (4S)-4-(2,3-dichloro-6-[[2-(trimethylsilyl)ethoxy]methoxy]phenyl)-1-(1-methylpyrazol-4-yl)pyrrolidin-2-one as a light-yellow oil (60.0 mg, 99%): LCMS (ESI) calc'd for C₂₀H₂₇Cl₂N₃O₃Si [M+H]⁺: 456, 458 (3:2) found 456, 458 (3:2).

Step b:

A solution of (4S)-4-(2,3-dichloro-6-[[2-(trimethylsilyl)ethoxy]methoxy]phenyl)-1-(1-methylpyrazol-4-yl)pyrrolidin-2-one (60.0 mg, 0.13 mmol) and TFA (0.5 mL) in DCM (2 mL) was stirred at room temperature for 1 h. The resulting mixture was concentrated under reduced pressure. The residue was purified by reverse phase chromatography, eluting with 30% ACN in water (plus 0.05% TFA) to afford the product. The product was purified by Prep Chiral HPLC with the following conditions: Column: CHIRALPAK IG, 20×250 mm, 5 μm; Mobile Phase A: Hex (plus 0.5% 2 M NH₃-MeOH)-HPLC, Mobile Phase B: EtOH-HPLC; Flow rate: 40 mL/min; Gradient: 50% B to 50% B in 14 min; Detector: UV 220/254 nm; Retention time 1: 8.54 min; Retention time 2: 11.46 min. The fractions containing the desired product were collected and concentrated under reduced pressure to afford Compound 56 ((4S)-4-(2,3-dichloro-6-hydroxyphenyl)-1-(1-methylpyrazol-4-yl)pyrrolidin-2-one) as an off-white solid (12.0 mg, 27%): LCMS (ESI) calc'd for C₁₄H₁₃Cl₂N₃O₂ [M+H]⁺: 326, 328 (3:2) found 326, 328 (3:2); ¹H NMR (400 MHz, CD₃OD) δ 7.98 (s, 1H), 7.70 (s, 1H), 7.29 (d, J=8.8 Hz, 1H), 6.80 (d, J=8.7 Hz, 1H), 4.65-4.55 (m, 1H), 4.10-3.95 (m, 2H), 3.90 (s, 3H), 3.03-2.78 (m, 2H).

The compounds in Table D below were prepared in analogous fashion to Compound 56.

TABLE D

| Compound | | | |
|---|---|---|---|
| Number | Structure | Chemical Name | MS: (M + H)⁺ & ¹H NMR |
| 57 | 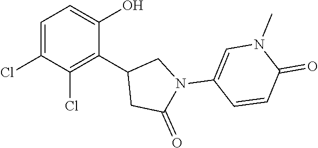 [SMILES: Cn1cc(N2CC(c3c(O)ccc(Cl)c3Cl)CC2=O)ccc1=O] | 5-[4-(2,3-dichloro-6- hydroxyphenyl)-2- oxopyrrolidin-1-yl]-1- methylpyridin-2-one | [M + H]⁺: 353, 355 (3:2); ¹H NMR (400 MHz, CD₃OD) δ 7.97 (d, J = 2.8 Hz, 1H), 7.88 (dd, J = 9.7, 2.9 Hz, 1H), 7.30 (d, J = 8.8 Hz, 1H), 6.82 (d, J = 8.8 Hz, 1H), 6.61 (d, J = 9.6 Hz, 1H), 4.62- 4.49 (m, 1H), 4.12-3.98 (m, 2H), 3.61 (s, 3H), 2.99 (dd, J = 17.3, 7.6 Hz, 1H), 2.87 (dd, J = 17.3, |
| | | | 10.8 Hz, 1H). |
| 58 | 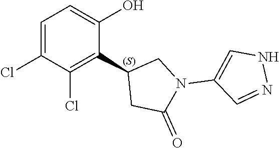 | (S)-4-(2,3-dichloro-6- hydroxyphenyl)-1-(1H- pyrazol-4-yl)pyrrolidin-2- one | [M + H]⁺: 312, 314 (3:2); H NMR (400 MHz, CD₃OD) δ 7.85 (s, 2H), 7.38 (d, J = 8.8 Hz, 1H), 6.86 (d, J = 8.8 Hz, 1H), 4.50- 4.34 (m, 1H), 4.02-3.94 (m, 1H), 3.79 (dd, J = 9.3, 6.9 Hz, 1H), 2.75 (d, J = 9.2 Hz, 2H). |
| 59 | 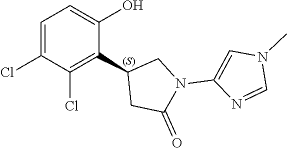 | (S)-4-(2,3-dichloro-6- hydroxyphenyl)-1-(1- methyl-1H-imidazol-4- yl)pyrrolidin-2-one | [M + H]⁺: 326, 328 (3:2); ¹H NMR (400 MHz, DMSO-d₆) δ 10.48 (s, 1H), 7.46-7.33 (m, 3H), 6.85 (d, J = 8.8 Hz, 1H), 4.45- 4.31 (m, 1H), 4.16-4.10 (m, 1H), 3.66 (s, 3H), 3.96-3.89 (m, 1H), 2.82-2.73 (m, 2H). |
| 60 | 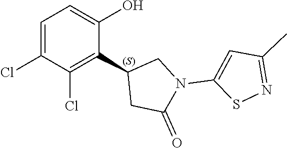 | (S)-4-(2,3-dichloro-6- hydroxyphenyl)-1-(3- methylisothiazol-5- yl)pyrrolidin-2-one | [M + H]⁺: 343, 345 (3:2); ¹H NMR (400 MHz, CD₃OD) δ 7.30 (d, J = 8.8 Hz, 1H), 6.79 (d, J = 8.8 Hz, 1H), 6.70 (s, 1H), 4.76- 4.64 (m, 1H), 4.31-4.23 (m, 1H), 4.05 (dd, J = 9.6, 6.3 Hz, 1H), 3.11-2.89 (m, 2H), 2.42 (s, 3H). |
| 61 | 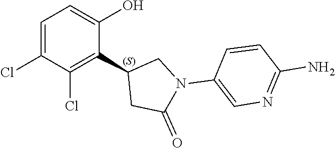 | (S)-1-(6-aminopyridin-3- yl)-4-(2,3-dichloro-6- hydroxyphenyl)pyrrolidin- 2-one | [M + H]⁺: 338, 340 (3:2); ¹H NMR (400 MHz, CD₃OD) δ 8.08 (dd, J = 2.7, 0.8 Hz, 1H), 7.70 (dd, J = 8.9, 2.7 Hz, 1H), 7.29 (d, J = 8.7 Hz, 1H), 6.81 (d, J = 8.8 Hz, 1H), 6.64 (dd, J = 9.0, 0.8 Hz, 1H), 4.62-4.49 (m, 1H), 4.13-4.01 (m, 2H), 2.98 (dd, J = 17.2, 7.6 Hz, 1H), 2.88 (dd, J = 17.3, 10.8 |
| | | | Hz, 1H). |
| 62 | 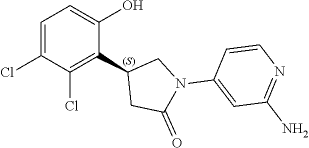 | (S)-1-(2-aminopyridin-4- yl)-4-(2,3-dichloro-6- hydroxyphenyl)pyrrolidin- 2-one | [M + H]⁺: 338, 340 (3:2); ¹H NMR (400 MHz, CD₃OD) δ 7.84 (d, J = 6.1 Hz, 1H), 7.29 (d, J = 8.8 Hz, 1H), 7.08 (dd, J = 6.1, 2.0 Hz, 1H), 6.95 (d, J = 1.9 Hz, 1H), 6.80 (d, J = 8.8 Hz, 1H), 4.60- 4.47 (m, 1H), 4.18-4.04 (m, 2H), 3.09 (dd, J = 17.5, 8.0 Hz, 1H), 2.91 (dd, J = 17.5, 10.8 Hz, 1H). |
| 63 | 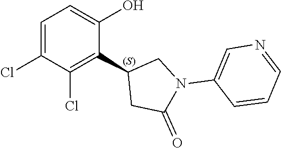 | (S)-4-(2,3-dichloro-6- hydroxyphenyl)-1- (pyridin-3-yl)pyrrolidin-2- one | [M + H]⁺: 323, 325 (3:2); ¹H NMR (400 MHz, CD₃OD) δ 8.97 (d, J = 2.6 Hz, 1H), 8.36 (d, J = 4.8 Hz, 1H), 8.20-8.14 (m, 1H), 7.49 (dd, J = 8.4, 4.8 Hz, 1H), 7.30 (d, J = 8.8 Hz, 1H), 6.81 (d, J = 8.8 Hz, 1H), 4.65-4.55 (m, 1H), 4.26-4.15 (m, 2H), 3.07 (dd, J = 17.4, 7.7 Hz, 1H), 2.95 (dd, J = |
| | | | 17.4, 10.8 Hz, 1H). |
| 64 | 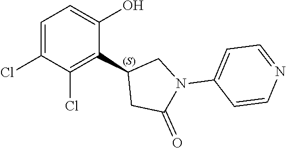 | (S)-4-(2,3-dichloro-6- hydroxyphenyl)-1- (pyridin-4-yl)pyrrolidin-2- one | [M + H]⁺: 323, 325 (3:2); ¹H NMR (400 MHz, CD₃OD) δ 8.50- 8.44 (m, 2H), 7.87-7.81 (m, 2H), 7.30 (d, J = 8.8 Hz, 1H), 6.80 (d, J = 8.8 Hz, 1H), 4.63-4.53 (m, 1H), 4.25-4.20 (m, 1H), 4.13 (dd, J = 9.4, 7.0 Hz, 1H), 3.11 (dd, J = 17.6, 7.8 Hz, 1H), 2.96 (dd, J = 17.6, 10.8 Hz, 1H). |
| 65 | 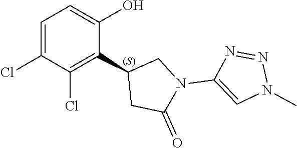 | (S)-4-(2,3-dichloro-6- hydroxyphenyl)-1-(1- methyl-1H-1,2,3-triazol-4- yl)pyrrolidin-2-one | [M + H]⁺: 327, 329 (3:2); ¹H NMR (300 MHz, CD₃OD) δ 8.30 (s, 1H), 7.30 (d, J = 8.8 Hz, 1H), 6.81 (d, J = 8.8 Hz, 1H), 4.78- 4.55 (m, 1H), 4.39-4.01 (m, 5H), 3.18-2.76 (m, 2H). |
| 66 | 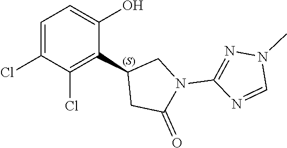 | (S)-4-(2,3-dichloro-6- hydroxyphenyl)-1-(1- methyl-1H-1,2,4-triazol-3- yl)pyrrolidin-2-one | [M + H]⁺: 327, 329 (3:2); ¹H NMR (300 MHz, CD₃OD) δ 8.30 (s, 1H), 7.28 (d, J = 8.8 Hz, 1H), 6.80 (d, J = 8.8 Hz, 1H), 4.63- 4.51 (m, 1H), 4.22-4.14 (m, 2H), 3.90 (s, 3H), 3.13-3.03 (m, 1H), 2.93-2.81 (m, 1H). |
| 67 | 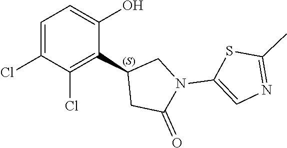 | (S)-4-(2,3-dichloro-6- hydroxyphenyl)-1-(2- methylthiazol-5- yl)pyrrolidin-2-one | [M + H]⁺: 343, 335 (3:2); ¹H NMR (300 MHz, CD₃OD) δ 7.33- 7.24 (m, 2H), 6.79 (d, J = 8.8 Hz, 1H), 4.70-4.55 (m, 1H), 4.25-4.14 (m, 1H), 4.02 (dd, J = 9.4, 6.5 Hz, 1H), 2.95 (d, J = 9.0 Hz, 2H), 2.64 (s, 3H). |
| 68 | 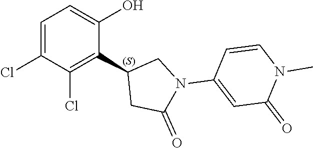 | (S)-4-(4-(2,3-dichloro-6- hydroxyphenyl)-2- oxopyrrolidin-1-yl)-1- methylpyridin-2(1H)-one | [M + H]⁺: 353, 355 (3:2); ¹H NMR (400 MHz, DMSO-d₆) δ 10.57 (s, 1H), 7.64 (d, J = 7.5 Hz, 1H), 7.36 (d, J = 8.7 Hz, 1H), 7.05 (d, J = 7.4 Hz, 1H), 6.83 (d, J = 8.7 Hz, 1H), 6.33 (s, 1H), 4.37-4.26 (m, 1H), 4.06-4.00 (m, 1H), 3.82 (dd, J = 9.7, 6.4 Hz, 1H), 3.37 (s, 3H), 2.91-2.77 (m, |
| | | | 2H). |
| 69 | 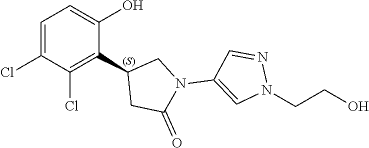 | (S)-4-(2,3-dichloro-6- hydroxyphenyl)-1-(1-(2- hydroxyethyl)-1H-pyrazol- 4-yl)pyrrolidin-2-one | [M + H]⁺: 356, 358 (3:2); ¹H NMR (300 MHz, DMSO-d₆ + D₂O) δ 7.99 (d, J = 0.8 Hz, 1H), 7.63 (d, J = 0.8 Hz, 1H), 7.36 (d, J = 8.8 Hz, 1H), 6.85 (d, J = 8.8 Hz, 1H), 4.51-4.33 (m, 1H), 4.11 (t, J = 5.5 Hz, 2H), 4.00-3.94 (m, 1H), 3.82-3.66 (m, 2H), 3.69-3.61 (m, 1H), 2.74 (d, J = 8.9 Hz, 2H). |

### Example 18. Compound 70 ((4S)-4-(2,3-dichloro-6-hydroxyphenyl)-1-[3-hydroxy-3-(hydroxymethyl)cyclobutyl]pyrrolidin-2-one isomer 3) and Compound 71 ((4S)-4-(2,3-dichloro-6-hydroxyphenyl)-1-[3-hydroxy-3-(hydroxymethyl)cyclobutyl]pyrrolidin-2-one isomer 4)

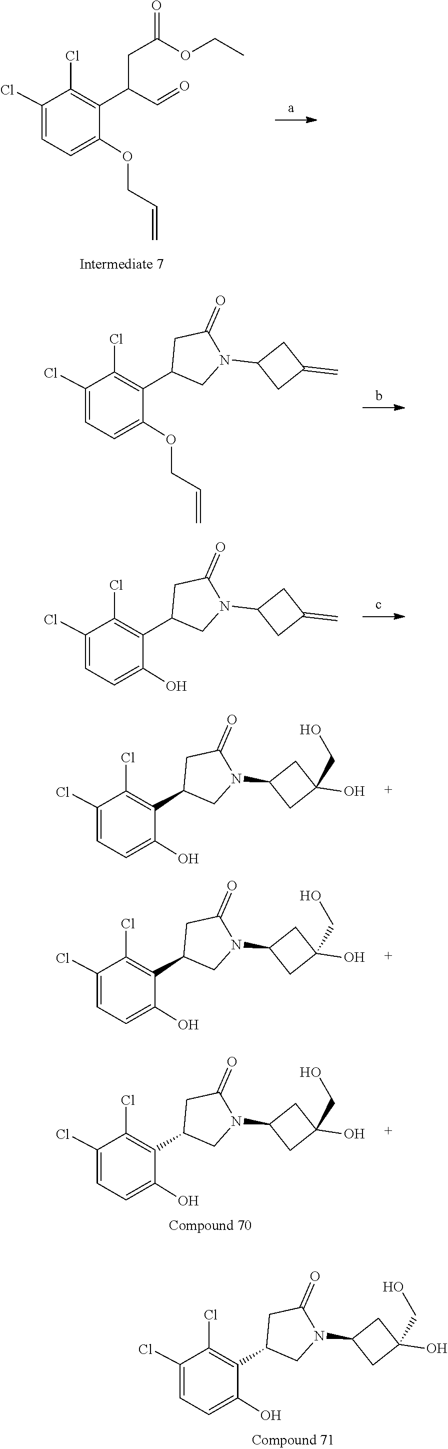

Step a:

To a stirred solution of ethyl 3-[2,3-dichloro-6-(prop-2-en-1-yloxy)phenyl]-4-oxobutanoate (Intermediate 7, Example 7) (0.300 g, 0.91 mmol) and 3-methylenecyclobutan-1-amine hydrochloride salt (0.108 g, 1.00 mmol) in DCE (5 mL) were added TEA (0.280 g, 2.72 mmol) and NaBH(OAc)₃ (0.580 g, 2.72 mmol) at room temperature. The reaction mixture was stirred for 16 h, quenched with saturated aqueous NH₄Cl (30 mL), and extracted with EA (3×30 mL). The combined organic layers were washed with brine (3×20 mL) and dried over anhydrous Na₂SO₄. After filtration, the filtrate was concentrated under reduced pressure. The residue was purified by reverse phase chromatography, eluting with 70% ACN in water (plus 0.05% TFA) to afford 4-[2,3-dichloro-6-(prop-2-en-1-yloxy)phenyl]-1-(3-methylidenecyclobutyl)pyrrolidin-2-one (0.220 g, 69%) as a light-yellow oil: LCMS (ESI) calc'd for C₁₈H₁₉Cl₂NO₂ [M+H]⁺: 352, 354 (3:2) found 352, 354 (3:2); ¹H NMR (300 MHz, CDCl₃) δ 7.33 (d, J=8.9 Hz, 1H), 6.78 (d, J=8.9 Hz, 1H), 6.07-5.91 (m, 1H), 5.39-5.26 (m, 2H), 4.90-4.74 (m, 3H), 4.63-4.48 (m, 2H), 4.48-4.34 (m, 1H), 3.80-3.64 (m, 2H), 2.99-2.77 (m, 5H), 2.71 (dd, J=17.1, 10.8 Hz, 1H).

Step b:

To a stirred solution of 4-[2,3-dichloro-6-(prop-2-en-1-yloxy)phenyl]-1-(3-methylidenecyclobutyl)pyrrolidin-2-one (0.220 g, 0.63 mmol) and Pd(PPh₃)₄ (72.2 mg, 0.06 mmol) in THE (3 mL) was added NaBH₄ (35.4 mg, 0.94 mmol) at room temperature. The reaction mixture was stirred for 2 h, quenched with saturated aqueous NH₄Cl (20 mL), and extracted with EA (3×20 mL). The combined organic layers were washed with brine (3×20 mL) and dried over anhydrous Na₂SO₄. After filtration, the filtrate was concentrated under reduced pressure. The residue was purified by reverse phase chromatography, eluting with 60% ACN in water (plus 0.05% TFA) to afford 4-(2,3-dichloro-6-hydroxyphenyl)-1-(3-methylidenecyclobutyl)pyrrolidin-2-one as a colorless oil (80.0 mg, 37%): LCMS (ESI) calc'd for C₁₅H₁₅Cl₂NO₂ [M+H]⁺: 312, 314 (3:2) found 312, 314 (3:2); ¹H NMR (400 MHz, CDCl₃) δ 7.25 (d, J=8.7 Hz, 1H), 6.92 (d, J=8.7 Hz, 1H), 4.90-4.81 (m, 2H), 4.73-4.61 (m, 1H), 4.39-4.24 (m, 1H), 3.89-3.86 (m, 1H), 3.69 (dd, J=9.6, 5.6 Hz, 1H), 3.01-2.88 (m, 2H), 2.86-2.72 (m, 3H), 1.77-1.70 (m, 1H).

Step c:

To a stirred mixture of 4-(2,3-dichloro-6-hydroxyphenyl)-1-(3-methylidenecyclobutyl)pyrrolidin-2-one (80.0 mg, 0.26 mmol) in THE (0.8 mL), acetone (0.8 mL), and H₂O (0.8 mL) was added NMO (45.0 mg, 0.38 mmol) and K₂OsO₄ 2H₂O (18.9 mg, 0.05 mmol) at room temperature. The reaction mixture was stirred for 2 h, quenched with saturated aqueous Na₂S₂O₃ (20 mL), and extracted with EA (3×20 mL). The combined organic layers were washed with brine (3×20 mL) and dried over anhydrous Na₂SO₄. After filtration, the filtrate was concentrated under reduced pressure. The residue was purified by Prep-HPLC with the following conditions: Column: Sun Fire Prep C18 OBD Column, 19×150 mm, 5 μm, 10 nm; Mobile Phase A: Water (plus 0.05% TFA), Mobile Phase B: ACN; Flow rate: 20 mL/min; Gradient: 28% B to 45% B in 4.3 min; Detector: UV 210 nm; Retention time: 4.20 min. The fractions containing the desired product were collected and concentrated under reduced pressure to afford 4-(2,3-dichloro-6-hydroxyphenyl)-1-(3-hydroxy-3-(hydroxymethyl)cyclobutyl)pyrrolidin-2-one as an off-white solid. 4-(2,3-dichloro-6-hydroxyphenyl)-1-(3-hydroxy-3-(hydroxymethyl)cyclobutyl)pyrrolidin-2-one was separated by Prep Chiral HPLC with the following conditions: Column: (R,R)Whelk-O 1, 21.1×250 mm, 5 μm; Mobile Phase A: Hex (plus 0.5% 2 M NH₃-MeOH)-HPLC, Mobile Phase B: EtOH-HPLC; Flow rate: 20 mL/min; Gradient: 10% B to 10% B in 40 min; Detector: UV 220/254 nm; Retention time 1: 22.74 min; Retention time 2: 23.73 min; Retention time 3:25.46 min; Retention time 4: 26.64 min. The third-eluting isomer at 25.46 min was obtained as Compound 70 ((4S)-4-(2,3-dichloro-6-hydroxyphenyl)-1-[3-hydroxy-3-(hydroxymethyl)cyclobutyl]pyrrolidin-2-one isomer 3) as an off-white solid (6.10 mg, 7%): LCMS (ESI) calc'd for C₁₅H₁₇Cl₂NO₄ [M+H]⁺: 346, 348 (3:2) found 346, 348 (3:2); ¹H NMR (300 MHz, CD₃OD) δ 7.26 (d, J=8.8 Hz, 1H), 6.78 (d, J=8.8 Hz, 1H), 4.98-4.90 (m, 1H), 4.47-4.30 (m, 1H), 3.88-3.68 (m, 2H), 3.40 (s, 2H), 2.85 (dd, J=17.0, 7.8 Hz, 1H), 2.69 (dd, J=17.0, 10.8 Hz, 1H), 2.60-2.43 (m, 2H), 2.21-2.03 (m, 2H). The forth-eluting isomer at 26.64 min was obtained as Compound 71 ((4S)-4-(2,3-dichloro-6-hydroxyphenyl)-1-[3-hydroxy-3-(hydroxymethyl)cyclobutyl]pyrrolidin-2-one isomer 4) (15.6 mg, 17%) as an off-white solid: LCMS (ESI) calc'd for C₁₅H₁₇Cl₂NO₄ [M+H]⁺: 346, 348 (3:2) found 346, 348 (3:2); ¹H NMR (300 MHz, CD₃OD) δ 7.27 (d, J=8.8 Hz, 1H), 6.79 (d, J=8.8 Hz, 1H), 4.49-4.34 (m, 1H), 4.29-4.25 (m, 1H), 3.92-3.73 (m, 2H), 3.54 (s, 2H), 2.85 (dd, J=17.0, 7.8 Hz, 1H), 2.71 (dd, J=17.1, 10.8 Hz, 1H), 2.52-2.38 (m, 2H), 2.36-2.20 (m, 2H).

The compounds in Table E below were prepared in analogous fashion to Compounds 70 and 71.

TABLE E

| Compound | | | |
|---|---|---|---|
| Number | Structure | Chemical Name | MS: (M + H)⁺ & ¹H NMR |
| 72 | 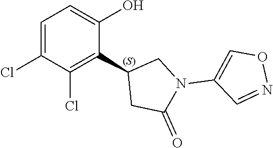 | (4S)-4-(2,3-dichloro-6- hydroxyphenyl)-1- (isoxazol-4-yl)pyrrolidin- 2-one | [M + H]⁺: 313, 315 (3:2); ¹H NMR (300 MHz, DMSO-d₆) δ 10.57 (s, 1H), 9.20 (s, 1H), 9.01 (s, 1H), 7.39 (d, J = 8.8 Hz, 1H), 6.86 (d, J = 8.8 Hz, 1H), 4.56-4.38 (m, 1H), 4.08-4.01 (m, 1H), 3.80 (dd, J = 9.2, 6.6 Hz, 1H), 2.92-2.68 (m, 2H). |
| 73 | 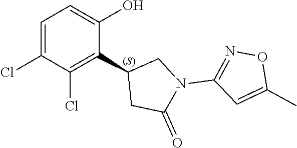 | (S)-4-(2,3-dichloro-6- hydroxyphenyl)-1-(5- methylisoxazol-3- yl)pyrrolidin-2-one | [M + H]⁺: 327, 329 (3:2); ¹H NMR (400 MHz, CD₃OD) δ 7.29 (d, J = 8.8 Hz, 1H), 6.88 (s, 1H), 6.80 (d, J = 8.8 Hz, 1H), 4.65-4.53 (m, 1H), 4.17-4.09 (m, 1H), 4.01 (dd, J = 10.0, 6.8 Hz, 1H), 2.99 (d, J = 17.6, 7.6 Hz, 1H), 2.89 (d, J = 17.6, 10.7 Hz, 1H), 2.44 (s, 3H). |
| 74 | 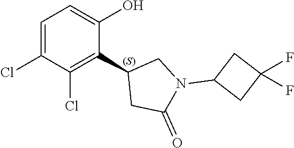 | (S)-4-(2,3-dichloro-6- hydroxyphenyl)-1-(3,3- difluorocyclobutyl)pyrrolidin- 2-one | [M + H]⁺: 336, 338 (3:2); ¹H NMR (300 MHz, DMSO-d₆) δ 10.49 (s, 1H), 7.37 (d, J = 8.8 Hz, 1H), 6.88 (d, J = 8.8 Hz, 1H), 4.53- 4.39 (m, 1H), 4.29-4.17 (m, 1H), 3.79-3.70 (m, 1H), 3.59-3.52 (m, 1H), 2.96-2.75 (m, 4H), 2.67-2.59 (m, 2H). |
| 75 | 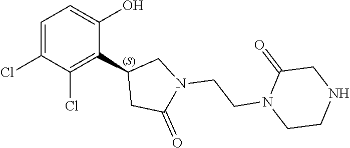 | 1-[2-[(4S)-4-(2,3-dichloro- 6-hydroxyphenyl)-2- oxopyrrolidin-1- yl]ethyl]piperazin-2-one | [M + H]⁺: 372, 374 (3:2); ¹H NMR (300 MHz, CD₃OD) δ 7.26 (d, J = 8.8 Hz, 1H), 6.78 (d, J = 8.8 Hz, 1H), 4.46-4.33 (m, 1H), 3.96- 3.69 (m, 4H), 3.57-3.28 (m, 6H), 3.10-3.00 (m, 2H), 2.89 (dd, J = 16.9, 8.2 Hz, 1H), 2.61 (dd, J = 16.9, 10.7 Hz, 1H). |

### Example 19. Compound 76 ((4R,5R)-1-(azetidin-3-yl)-4-(2,3-dichloro-6-hydroxyphenyl)-5-methylpyrrolidin-2-one) and Compound 77 ((4S,5S)-1-(azetidin-3-yl)-4-(2,3-dichloro-6-hydroxyphenyl)-5-methylpyrrolidin-2-one)

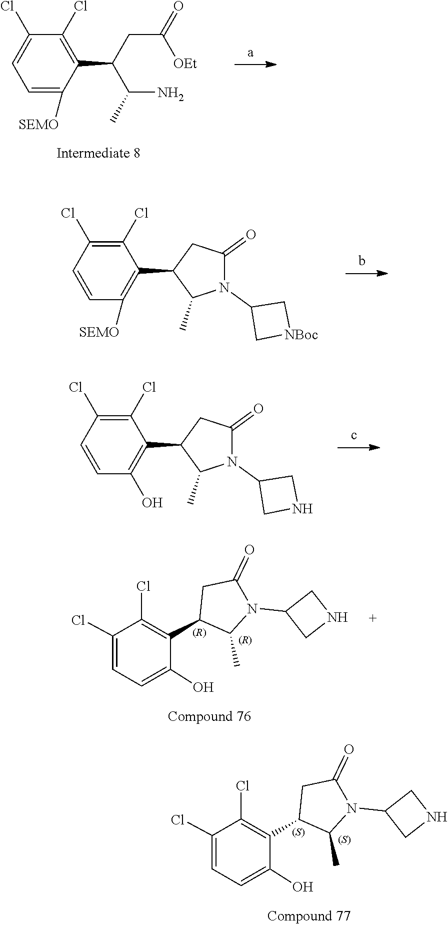

Step a:

A mixture of ethyl-(3R,4R)-rel-4-amino-3-(2,3-dichloro-6-[[2-(trimethylsilyl)ethoxy]methoxy]phenyl)pentanoate (Intermediate 8, Example 8) (0.200 g, 0.36 mmol), tert-butyl 3-oxoazetidine-1-carboxylate (0.190 g, 1.10 mmol), TEA (0.110 g, 1.10 mmol), and NaBH(AcO)₃ (0.230 g, 1.10 mmol) in DCE (4 mL) was stirred at 80° C. for 2 h. The resulting mixture was quenched with saturated aqueous NH₄Cl (20 mL) and extracted with DCM (2×20 mL). The combined organic layers were washed with brine (2×20 mL) and dried over anhydrous Na₂SO₄. After filtration, the filtrate was concentrated under reduced pressure. The residue was purified by reverse phase chromatography, eluting with 50% ACN in water (plus 0.05% TFA) to afford tert-butyl 3-[(2R,3R)-rel-3-(2,3-dichloro-6-[[2-(trimethylsilyl)ethoxy]methoxy]phenyl)-2-methyl-5-oxopyrrolidin-1-yl]azetidine-1-carboxylate (a mixture of trans isomers) as a light-yellow oil (0.190 g, 96%): LCMS (ESI) calc'd for C₂₅H₃₈Cl₂N₂O₅Si [M+H]⁺: 545, 547 (3:2) found 545, 547 (3:2); H NMR (400 MHz, CDCl₃) δ 7.36 (d, J=9.0 Hz, 1H), 7.11 (d, J=9.0 Hz, 1H), 5.29-5.15 (m, 2H), 4.72-4.60 (m, 1H), 4.34 (dd, J=9.4, 6.0 Hz, 1H), 4.26-4.17 (m, 3H), 4.07-3.97 (m, 2H), 3.72 (dd, J=8.8, 7.4 Hz, 2H), 2.88 (d, J=8.7 Hz, 2H), 1.47 (s, 9H), 1.43 (d, J=5.7 Hz, 3H), 0.95 (dd, J=8.8, 7.4 Hz, 2H), 0.02 (s, 9H).

Step b:

A solution of tert-butyl 3-[(2R,3R)-rel-3-(2,3-dichloro-6-[[2-(trimethylsilyl)ethoxy]methoxy]phenyl)-2-methyl-5-oxopyrrolidin-1-yl]azetidine-1-carboxylate (a mixture of trans isomers) (0.180 g, 0.33 mmol) and TFA (0.50 mL) in DCM (2 mL) was stirred at room temperature for 1 h. The resulting mixture was concentrated under reduced pressure. The residue was purified by Prep-HPLC with the following conditions: Column: X Bridge Shield RP18 OBD Column, 30×150 mm, 5 μm; Mobile Phase A: Water (plus 0.05% TFA), Mobile Phase B: ACN; Flow rate: 60 mL/min; Gradient: 15% B to 45% B in 8 min; Detector: UV 220 nm; Retention time: 7.23 min. The fractions containing the desired product were collected and concentrated under reduced pressure to afford (4R,5R)-rel-1-(azetidin-3-yl)-4-(2,3-dichloro-6-hydroxyphenyl)-5-methylpyrrolidin-2-one as an off-white solid (76.0 mg, 52%): LCMS (ESI) calc'd for C₁₄H₁₆C₁₂N₂O₂ [M+H]⁺: 315, 317 (3:2) found 315, 317 (3:2); ¹H NMR (400 MHz, CD₃OD) δ 7.28 (d, J=8.7 Hz, 1H), 6.80 (d, J=8.8 Hz, 1H), 4.76-4.66 (m, 2H), 4.52-4.31 (m, 3H), 4.05-3.87 (m, 2H), 2.86-2.82 (m, 2H), 1.30 (d, J=6.1 Hz, 3H).

Step c:

(4R,5R)-rel-1-(azetidin-3-yl)-4-(2,3-dichloro-6-hydroxyphenyl)-5-methylpyrrolidin-2-one (76.0 mg, 0.18 mmol) was separated by Chiral Prep HPLC with the following conditions: Column: CHIRALPAK IH, 2.0×25 cm, 5 μm; Mobile Phase A: Hex (plus 0.2% IPA)-HPLC, Mobile Phase B: EtOH-HPLC; Flow rate: 20 mL/min; Gradient: 20% B to 20% B in 20 min; Detector: UV 220/254 nm; Retention time 1: 8.68 min; Retention time 2: 16.63 min. The faster-eluting enantiomer at 8.68 min was isolated. The product was purified by Prep-HPLC with the following conditions: Column: Sun Fire Prep C18 OBD Column, 19×150 mm, 5 μm 10 nm; Mobile Phase A: Water (plus 0.05% TFA), Mobile Phase B: ACN; Flow rate: 20 mL/min; Gradient: 25% B to 65% B in 4.3 min; Detector: UV 210 nm; Retention Time: 4.20 min. The fractions containing the desired product were collected and concentrated under reduced pressure to afford Compound 76 ((4R,5R)-1-(azetidin-3-yl)-4-(2,3-dichloro-6-hydroxyphenyl)-5-methylpyrrolidin-2-one) as an off-white solid (8.30 mg, 11%): LCMS (ESI) calc'd for C₁₄H₁₆Cl₂N₂O₂ [M+H]⁺: 315, 317 (3:2) found 315, 317 (3:2); ¹H NMR (400 MHz, CD₃OD) δ 7.29 (d, J=8.8 Hz, 1H), 6.79 (d, J=8.8 Hz, 1H), 4.76-4.68 (m, 2H), 4.48-4.31 (m, 3H), 4.05-3.91 (m, 2H), 2.94-2.73 (m, 2H), 1.30 (d, J=6.1 Hz, 3H). The slower-eluting enantiomer at 16.63 min was isolated. The product was purified by Prep-HPLC with the following conditions: Column: Sun Fire Prep C18 OBD Column, 19×150 mm, 5 μm 10 nm; Mobile Phase A: Water (plus 0.05% TFA), Mobile Phase B: ACN; Flow rate: 20 mL/min; Gradient: 25% B to 65% B in 4.3 min; Detector: UV 210 nm; Retention Time: 4.20 min. The fractions containing the desired product were collected and concentrated under reduced pressure to afford Compound 77 ((4S,5S)-1-(azetidin-3-yl)-4-(2,3-dichloro-6-hydroxyphenyl)-5-methylpyrrolidin-2-one) as an off-white solid (14.2 mg, 19%): LCMS (ESI) calc'd for C₁₄H₁₆Cl₂N₂O₂ [M+H]⁺: 315, 317 (3:2) found 315, 317 (3:2); ¹H NMR (400 MHz, CD₃OD) δ 7.28 (d, J=8.8 Hz, 1H), 6.79 (d, J=8.8 Hz, 1H), 4.76-4.67 (m, 2H), 4.50-4.31 (m, 3H), 4.03-3.90 (m, 2H), 2.86-2.83 (m, 2H), 1.29 (d, J=6.1 Hz, 3H).

### Example 20. Compound 78 ((4R,5R)-4-(2,3-dichloro-6-hydroxyphenyl)-1-[(2S)-2,3-dihydroxypropyl]-5-methylpyrrolidin-2-one) and Compound 79 ((4S,5S)-4-(2,3-dichloro-6-hydroxyphenyl)-1-[(2S)-2,3-dihydroxypropyl]-5-methylpyrrolidin-2-one)

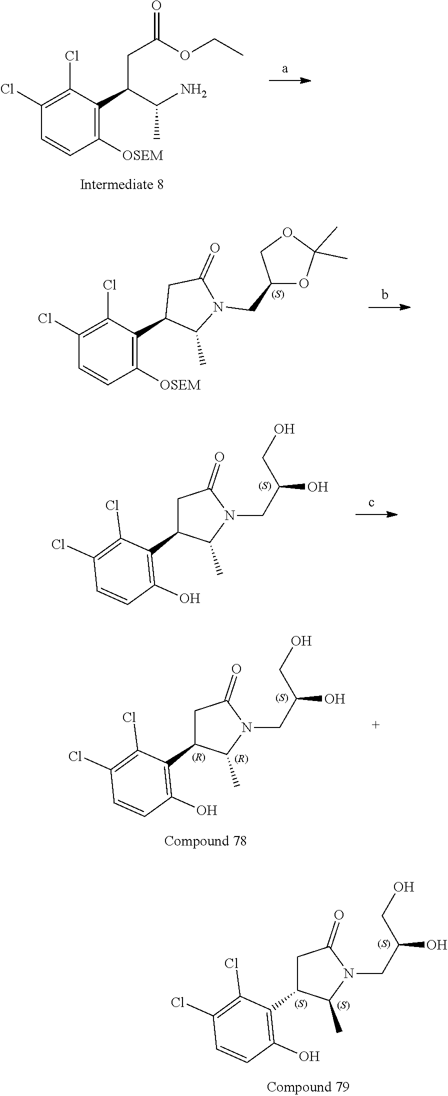

Step a:

A solution of ethyl-(3R,4R)-rel-4-amino-3-(2,3-dichloro-6-[[2-(trimethylsilyl)ethoxy]methoxy]phenyl)pentanoate (Intermediate 8, Example 8) (0.300 g, 0.56 mmol), (4R)-2,2-dimethyl-1,3-dioxolane-4-carbaldehyde (88.0 mg, 0.67 mmol), and TEA (0.110 g, 1.12 mmol) in DCE (5 mL) was stirred at room temperature for 30 min and NaBH(AcO)₃ (0.240 g, 1.12 mmol) was added. The resulting reaction mixture was stirred for 2 h, quenched with saturated aqueous NH₄Cl (20 mL), and extracted with EA (2×20 mL). The combined organic layers were washed with brine (2×20 mL) and dried over anhydrous Na₂SO₄. After filtration, the filtrate was concentrated under reduced pressure. The residue was purified by reverse phase chromatography, eluting with 65% ACN in water (plus 0.05% TFA) to afford (4R,5R)-rel-4-(2,3-dichloro-6-[[2-(trimethylsilyl)ethoxy]methoxy]phenyl)-1-[[(4S)-2,2-dimethyl-1,3-dioxolan-4-yl]methyl]-5-methylpyrrolidin-2-one as a light-yellow oil (0.150 g, 53%): LCMS (ESI) calc'd for C₂₃H₃₅Cl₂NO₅Si [M+H]⁺: 504, 506 (3:2) found 504, 506 (3:2); ¹H NMR (300 MHz, CDCl₃) δ 7.35 (d, J=9.0 Hz, 1H), 7.13 (d, J=9.0 Hz, 1H), 5.28-5.20 (m, 2H), 4.37-4.25 (m, 1H), 4.22-3.88 (m, 3H), 3.80-3.65 (m, 4H), 3.44-3.35 (m, 1H), 3.11-2.85 (m, 1H), 2.85-2.66 (m, 1H), 1.42 (d, J=2.3 Hz, 3H), 1.38-1.31 (m, 6H), 1.02-0.88 (m, 2H), 0.02 (s, 9H).

Step b:

A solution of (4R,5R)-rel-4-(2,3-dichloro-6-[[2-(trimethylsilyl)ethoxy]methoxy]phenyl)-1-[[(4S)-2,2-dimethyl-1,3-dioxolan-4-yl]methyl]-5-methylpyrrolidin-2-one (0.150 g, 0.297 mmol) and aqueous HCl (6 M, 1 mL) in MeOH (1 mL) was stirred at room temperature for 1 h. The resulting mixture was concentrated under reduced pressure. The residue was purified by reverse phase chromatography, eluting with 30% ACN in water (plus 0.05% TFA) to afford (4R,5R)-rel-4-(2,3-dichloro-6-hydroxyphenyl)-1-[(2S)-2,3-dihydroxypropyl]-5-methylpyrrolidin-2-one as a light-yellow oil (90.0 mg, 90%): LCMS (ESI) calc'd for C₁₄H₁₇Cl₂NO₄ [M+H]⁺: 334, 336 (3:2) found 334, 336 (3:2); ¹H NMR (300 MHz, CD₃OD) δ 7.28 (d, J=8.8 Hz, 1H), 6.79 (d, J=8.7 Hz, 1H), 4.19-4.16 (m, 1H), 4.02-3.70 (m, 3H), 3.64-3.48 (m, 2H), 3.19-2.89 (m, 2H), 2.71-2.57 (m, 1H), 1.33 (dd, J=6.4, 4.2 Hz, 3H).

Step c:

(4R,5R)-rel-4-(2,3-dichloro-6-hydroxyphenyl)-1-[(2S)-2,3-dihydroxypropyl]-5-methylpyrrolidin-2-one (90.0 mg, 0.27 mmol) was separated by Prep Chiral HPLC with the following conditions: Column: CHIRALPAK IC, 2×25 cm, 5 μm; Mobile Phase A: Hex (plus 0.3% IPA)-HPLC, Mobile Phase B: EtOH-HPLC; Flow rate: 20 mL/min; Gradient: 10% B to 10% B in 23 min; Detector: UV 220/254 nm; Retention Time 1: 14.16 min; Retention Time 2: 20.27 min. The faster-eluting isomer at 14.16 min was obtained as Compound 78 ((4R,5R)-4-(2,3-dichloro-6-hydroxyphenyl)-1-[(2S)-2,3-dihydroxypropyl]-5-methylpyrrolidin-2-one) as an off-white solid (11.3 mg, 12%): LCMS (ESI) calc'd for C₁₄H₁₇Cl₂NO₄ [M+H]⁺: 334, 336 (3:2) found 334, 336 (3:2); ¹H NMR (300 MHz, CD₃OD) δ 7.28 (d, J=8.8 Hz, 1H), 6.79 (d, J=8.8 Hz, 1H), 4.22-4.08 (m, 1H), 3.99-3.82 (m, 2H), 3.65-3.50 (m, 4H), 3.01 (dd, J=17.0, 8.7 Hz, 1H), 2.62 (dd, J=17.0, 10.6 Hz, 1H), 1.33 (d, J=6.3 Hz, 3H). The slower-eluting isomer at 20.27 min was obtained as Compound 79 ((4S,5S)-4-(2,3-dichloro-6-hydroxyphenyl)-1-[(2S)-2,3-dihydroxypropyl]-5-methylpyrrolidin-2-one) as a yellow solid (21.7 mg, 23%): LCMS (ESI) calc'd for C₁₄H₁₇Cl₂NO₄ [M+H]⁺: 334, 336 (3:2) found 334, 336 (3:2); ¹H NMR (300 MHz, CD₃OD) δ 7.28 (d, J=8.8 Hz, 1H), 6.79 (d, J=8.8 Hz, 1H), 4.25-4.11 (m, 1H), 3.99-3.69 (m, 3H), 3.60-3.46 (m, 2H), 3.14 (dd, J=13.9, 6.6 Hz, 1H), 2.96 (dd, J=17.0, 8.4 Hz, 1H), 2.65 (dd, J=17.0, 10.7 Hz, 1H), 1.34 (d, J=6.3 Hz, 3H).

### Example 21. Compound 80 ((4R,5R)-4-(2,3-dichloro-6-hydroxyphenyl)-1-(2-hydroxyethyl)-5-methylpyrrolidin-2-one) and Compound 81 ((4S,5S)-4-(2,3-dichloro-6-hydroxyphenyl)-1-(2-hydroxyethyl)-5-methylpyrrolidin-2-one)

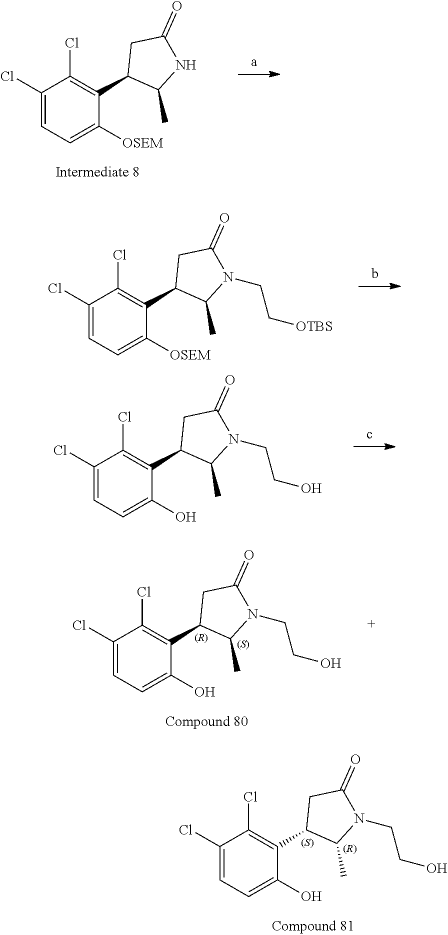

Step a:

To a stirred solution of (4R,5R)-rel-4-(2,3-dichloro-6-[[2-(trimethylsilyl)ethoxy]methoxy]phenyl)-5-methyl pyrrolidin-2-one (0.150 g, 0.38 mmol) and (2-bromoethoxy)(tert-butyl)dimethylsilane (0.140 g, 0.58 mmol) in DMF (2 mL) was added NaH (46.0 mg, 1.15 mmol, 60% in oil) at 0° C. under nitrogen atmosphere. The reaction mixture was stirred at room temperature for 3 h under nitrogen atmosphere. The resulting mixture was quenched with water (20 mL) and extracted with EA (3×30 mL). The combined organic layers were washed with brine (2×20 mL) and dried over anhydrous Na₂SO₄. After filtration, the filtrate was concentrated under reduced pressure. The residue was purified by reverse phase chromatography, eluted 95% ACN in water (plus 0.05% TFA) to afford (4R,5R)-rel-1-[2-[(tert-butyldimethylsilyl)oxy]ethyl]-4-(2,3-dichloro-6-[[2-(trimethylsilyl)ethoxy]methoxy]phenyl)-5-methylpyrrolidin-2-one as a yellow oil (0.130 g, 62%): LCMS (ESI) calc'd for C₂₅H₄₃Cl₂NO₄Si₂ [M+H]⁺: 548, 550 (3:2) found 548, 550 (3:2).

Step b:

To a stirred solution of (4R,5R)-rel-1-[2-[(tert-butyldimethylsilyl)oxy]ethyl]-4-(2,3-dichloro-6-[[2-(trimethylsilyl)ethoxy]methoxy]phenyl)-5-methylpyrrolidin-2-one (0.130 g, 0.24 mmol) in 1,4-dioxane (1 mL) was added aqueous HCl (6 M, 1 mL) at room temperature. The reaction solution was stirred for 2 h and concentrated under reduced pressure. The residue was purified by Prep-HPLC with the following conditions: Column: X Bridge Prep Phenyl OBD Column, 19×150 mm 5 μm 13 nm; Mobile Phase A: Water (plus 10 mM NH₄HCO₃), Mobile Phase B: ACN; Flow rate: 20 mL/min; Gradient: 30% to 40% in 5.3 min; Detector: UV 254/220 nm; Retention time: 5.2 min. The fractions containing the desired product were collected and concentrated under reduced pressure to afford (4R,5R)-rel-4-(2,3-dichloro-6-hydroxyphenyl)-1-(2-hydroxyethyl)-5-methylpyrrolidin-2-one as an off-white solid. (27.2 mg, 37%): LCMS (ESI) calc'd for C₁₃H₁₅Cl₂NO₃ [M+H]⁺: 304, 306 (3:2) found 304, 306 (3:2); ¹H NMR (400 MHz, CD₃OD) δ 7.27 (d, J=8.8 Hz, 1H), 6.78 (d, J=8.7 Hz, 1H), 4.18-4.06 (m, 1H), 3.99-3.87 (m, 1H), 3.72-3.66 (m, 2H), 3.65-3.61 (m, 1H), 3.31-3.26 (m, 1H), 2.94 (dd, J=17.0, 8.5 Hz, 1H), 2.63 (dd, J=16.9, 10.6 Hz, 1H), 1.32 (d, J=6.3 Hz, 3H).

Step c:

(4R,5R)-rel-4-(2,3-dichloro-6-hydroxyphenyl)-1-(2-hydroxyethyl)-5-methylpyrrolidin-2-one (27.0 mg, 0.09 mmol) was purified by Prep Chiral HPLC with the following conditions: Column: CHIRALPAK IG, 2×25 cm, 5 μm; Mobile Phase A: Hex (plus 0.5% 2 M NH₃-MeOH)-HPLC, Mobile Phase B: EtOH-HPLC; Flow rate: 20 mL/min; Gradient: 10% to 10% in 11 min; Detector: UV 254/220 nm; Retention time 1: 7.24 min, Retention time 2: 8.56 min. The faster-eluting enantiomer at 7.24 min was obtained as Compound 80 ((4R,5R)-4-(2,3-dichloro-6-hydroxyphenyl)-1-(2-hydroxyethyl)-5-methylpyrrolidin-2-one) as an off-white solid (7.40 mg, 27%): LCMS (ESI) calc'd for C₁₃H₁₅Cl₂NO₃ [M+H]⁺: 304, 306 (3:2) found 304, 306 (3:2); ¹H NMR (300 MHz, CD₃OD) δ 7.27 (d, J=8.8 Hz, 1H), 6.78 (d, J=8.8 Hz, 1H), 4.19-4.05 (m, 1H), 4.01-3.87 (m, 1H), 3.74-3.52 (m, 3H), 3.30-3.21 (m, 1H), 2.94 (dd, J=16.9, 8.5 Hz, 1H), 2.63 (dd, J=16.9, 10.5 Hz, 1H), 1.32 (d, J=6.3 Hz, 3H). The slower-eluting enantiomer at 8.56 min was obtained as Compound 81 ((4S,5S)-4-(2,3-dichloro-6-hydroxyphenyl)-1-(2-hydroxyethyl)-5-methylpyrrolidin-2-one) as an off-white solid (7.70 mg, 28%): LCMS (ESI) calc'd for C₁₃H₁₅Cl₂NO₃ [M+H]⁺: 304, 306 (3:2) found 304, 306 (3:2); ¹H NMR (300 MHz, CD₃OD) δ 7.27 (d, J=8.8 Hz, 1H), 6.78 (d, J=8.8 Hz, 1H), 4.19-4.07 (m, 1H), 3.99-3.85 (m, 1H), 3.74-3.52 (m, 3H), 3.30-3.21 (m, 1H), 2.94 (dd, J=16.9, 8.5 Hz, 1H), 2.63 (dd, J=17.0, 10.5 Hz, 1H), 1.32 (d, J=6.3 Hz, 3H).

The compounds in Table F below were prepared in analogous fashion to Compounds 80 and 81.

TABLE F

| Compound | | | |
|---|---|---|---|
| Number | Structure | Chemical Name | MS: (M + H)⁺ & ¹H NMR |
| 82 | 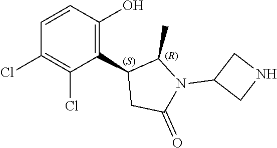 | (4S,5R)-1-(azetidin-3-yl)- 4-(2,3-dichloro-6- hydroxyphenyl)-5- methylpyrrolidin-2-one | [M + H]⁺: 315, 317 (3:2); ¹H NMR (400 MHz, CD₃OD) δ 7.29 (d, J = 8.8 Hz, 1H), 6.79 (d, J = 8.8 Hz, 1H), 4.76-4.68 (m, 2H), 4.48-4.33 (m, 3H), 4.04-3.91 (m, 2H), 2.89 (dd, J = 17.3, 7.4 Hz, 1H), 2.79 (dd, J = 17.2, 10.5 Hz, 1H), 1.30 (d, J = 6.1 Hz, 3H). |
| 83 | 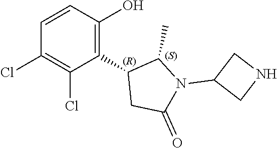 | (4R,5S)-1-(azetidin-3-yl)- 4-(2,3-dichloro-6- hydroxyphenyl)-5- methylpyrrolidin-2-one | [M + H]⁺: 315, 317 (3:2); ¹H NMR (400 MHz, CD₃OD) δ 7.28 (d, J = 8.8 Hz, 1H), 6.79 (d, J = 8.8 Hz, 1H), 4.76-4.68 (m, 2H), 4.48-4.33 (m, 3H), 4.04-3.91 (m, 2H), 2.88 (dd, J = 17.2, 7.3 Hz, 1H), 2.79 (dd, J = 17.2, 10.4 Hz, 1H), 1.29 (d, J = 6.1 Hz, 3H). |
| 84 | 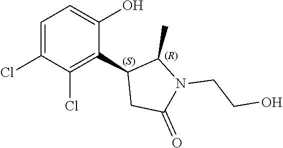 | (4S,5R)-4-(2,3-dichloro-6- hydroxyphenyl)-1-(2- hydroxyethyl)-5- methylpyrrolidin-2-one | [M + H]⁺: 304, 306 (3:2) found 304, 306 (3:2); ¹H NMR (400 MHz, CD₃OD) δ 7.27 (d, J = 8.8 Hz, 1H), 6.76 (d, J = 8.8 Hz, 1H), 4.56-4.45 (m, 1H), 4.36-4.24 (m, 1H), 3.77-3.62 (m, 3H), 3.26-3.08 (m, 2H), 2.71 (dd, J = 17.0, 10.6 Hz, 1H), 1.00 (d, J = 6.7 Hz, 3H). |
| 85 | 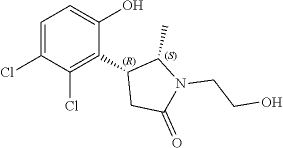 | (4R,5S)-4-(2,3-dichloro-6- hydroxyphenyl)-1-(2- hydroxyethyl)-5- methylpyrrolidin-2-one | [M + H]⁺: 304, 306 (3:2) found 304, 306 (3:2); ¹H NMR (400 MHz, CD₃OD) δ 7.28 (d, J = 8.8 Hz, 1H), 6.76 (d, J = 8.7 Hz, 1H), 4.54-4.46 (m, 1H), 4.36-4.24 (m, 1H), 3.77-3.64 (m, 3H), 3.26-3.09 (m, 2H), 2.71 (dd, J = 17.0, 10.6 Hz, 1H), 1.00 (d, J = 6.7 Hz, 3H). |
| 86 | 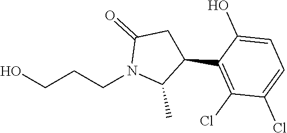 [SMILES: C[C@H]1[C@H](c2c(O)ccc(Cl)c2Cl)CC(=O)N1CCCO] | (4S,5S)-4-(2,3-dichloro-6- hydroxyphenyl)-1-(3- hydroxypropyl)-5- methylpyrrolidin-2-one | [M + H]⁺: 318, 320 (3:2); ¹H NMR (400 MHz, CD₃OD) δ 7.28 (d, J = 8.8 Hz, 1H), 6.78 (d, J = 8.8 Hz, 1H), 4.09-4.02 (m, 1H), 3.95-3.89 (m, 1H), 3.75-3.67 (m, 1H), 3.66- 3.57 (m, 2H), 3.25-3.17 (m, 1H), 2.93 (dd, J = 17.0, 8.3 Hz, 1H), 2.65 (dd, J = 17.0, 10.7 Hz, 1H), 1.86- |
| | | | 1.70 (m, 2H), 1.32 (d, J = 6.2 Hz, |
| | | | 3H). |

### Example 22. Compound 87 (1-(azetidin-3-yl)-4-(2,3-dichloro-6-hydroxyphenyl)-3-methylpyrrolidin-2-one isomer 1), Compound 88 (1-(azetidin-3-yl)-4-(2,3-dichloro-6-hydroxyphenyl)-3-methylpyrrolidin-2-one isomer 2), Compound 89 (1-(azetidin-3-yl)-4-(2,3-dichloro-6-hydroxyphenyl)-3-methylpyrrolidin-2-one isomer 3), and Compound 90 (1-(azetidin-3-yl)-4-(2,3-dichloro-6-hydroxyphenyl)-3-methylpyrrolidin-2-one isomer 4)

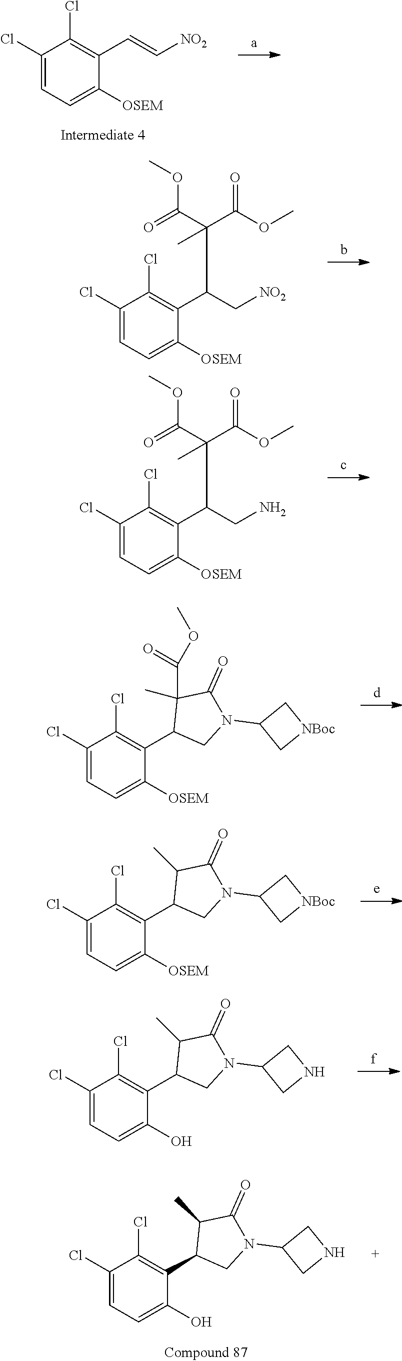

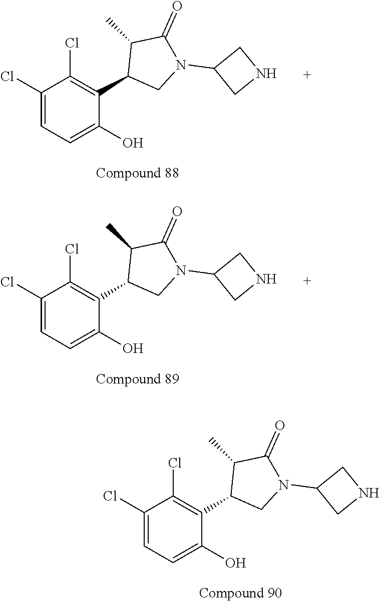 [SMILES: C[C@@H]1C(=O)N(C2CNC2)C[C@H]1c1c(O)ccc(Cl)c1Cl ; C[C@H]1C(=O)N(C2CNC2)C[C@@H]1c1c(O)ccc(Cl)c1Cl ; C[C@@H]1C(=O)N(C2CNC2)C[C@@H]1c1c(O)ccc(Cl)c1Cl]

Step a:

To a solution of (2-[3,4-dichloro-2-[(E)-2-nitroethenyl]phenoxymethoxy]ethyl)trimethylsilane (Intermediate 4, Example 4) (1.00 g, 2.75 mmol) in DMF (10 mL) were added 1,3-dimethyl 2-methylpropanedioate (0.800 g, 5.49 mmol) and K₂CO₃ (0.76 g, 5.49 mmol) at room temperature. The reaction mixture was stirred for 1 h, diluted with water (50 mL), and extracted with EA (3×30 mL). The combined organic layers were washed with brine (4×30 mL) and dried over anhydrous Na₂SO₄. After filtration, the filtrate was concentrated under reduced pressure. The residue was purified by silica gel column chromatography, eluting with PE/EA (10/1) to afford 1,3-dimethyl 2-[1-(2,3-dichloro-6-[[2-(trimethylsilyl)ethoxy]methoxy]phenyl)-2-nitroethyl]-2-methylpropanedioate as a yellow oil (1.05 g, 75%): ¹H NMR (400 MHz, CDCl₃) δ 7.37 (d, J=9.0 Hz, 1H), 7.11 (d, J=9.1 Hz, 1H), 5.42 (dd, J=10.9, 3.7 Hz, 1H), 5.27 (dd, J=13.0, 10.9 Hz, 1H), 5.19 (d, J=7.2 Hz, 1H), 5.10-5.03 (m, 2H), 3.83 (s, 3H), 3.81 (s, 3H), 3.79-3.72 (m, 2H), 1.32 (s, 3H), 1.01-0.94 (m, 2H), 0.04 (s, 9H).

Step b:

To a solution of 1,3-dimethyl 2-[1-(2,3-dichloro-6-[[2-(trimethylsilyl)ethoxy]methoxy]phenyl)-2-nitroethyl]-2-methylpropanedioate (0.850 g, 1.67 mmol) in AcOH (10 mL) was added Zn (1.09 g, 16.65 mmol) at room temperature. The reaction mixture was stirred for 16 h and filtered. The filter cake was washed with MeOH (2×10 mL) and the filtrate concentrated under reduced pressure. The residue was purified by reverse phase chromatography, eluting with 46% ACN in water (plus 0.05% TFA) to afford 1,3-dimethyl-2-[2-amino-1-(2,3-dichloro-6-[[2-(trimethylsilyl)ethoxy]methoxy]phenyl)ethyl]-2-methylpropanedioate as a yellow oil (0.560 g, 85%): LCMS (ESI) calc'd for C₂₀H₃₁Cl₂NO₆Si [M+H]⁺: 480, 482 (3:2) found 480, 482 (3:2); ¹H NMR (400 MHz, CDCl₃) δ 7.40 (d, J=9.1 Hz, 1H), 7.16 (d, J=9.0 Hz, 1H), 5.10 (s, 2H), 4.77 (t, J=6.5 Hz, 1H), 3.86 (s, 3H), 3.84-3.80 (m, 4H), 3.79-3.62 (m, 2H), 3.53-3.44 (m, 1H), 1.28 (s, 3H), 0.99-0.92 (m, 2H), 0.03 (s, 9H).

Step c:

To a solution of 1,3-dimethyl-2-[2-amino-1-(2,3-dichloro-6-[[2-(trimethylsilyl)ethoxy]methoxy]phenyl)ethyl]-2-methylpropanedioate (0.560 g, 1.17 mmol) and tert-butyl 3-oxoazetidine-1-carboxylate (0.300 g, 1.75 mmol) in DCE (10 mL) were added TEA (0.360 g, 3.50 mmol) and NaBH(AcO)₃ (0.740 g, 3.50 mmol) at room temperature. The reaction mixture was stirred at 60° C. for 1 h. The resulting mixture was diluted with water (20 mL) and extracted with EA (3×30 mL). The combined organic layers were washed with brine (2×30 mL) and dried over anhydrous Na₂SO₄. After filtration, the filtrate was concentrated under reduced pressure. The residue was purified by reverse phase chromatography, eluting with 90% ACN in water (plus 10 mM NH₄HCO₃) to afford methyl 1-[1-(tert-butoxycarbonyl)azetidin-3-yl]-4-(2,3-dichloro-6-[[2-(trimethylsilyl)ethoxy]methoxy]phenyl)-3-methyl-2-oxopyrrolidine-3-carboxylate as a yellow oil (0.500 g, 71%): LCMS (ESI) calc'd for C₂₇H₄₀Cl₂N₂O₇Si [M+H]⁺: 603, 605 (3:2) found 603, 605 (3:2); ¹H NMR (400 MHz, CDCl₃) δ 7.36 (d, J=9.0 Hz, 1H), 7.11 (d, J=9.0 Hz, 1H), 5.17-5.09 (m, 2H), 5.06 (d, J=7.1 Hz, 1H), 4.42 (dd, J=8.7, 6.4 Hz, 1H), 4.26-4.19 (m, 2H), 4.17-4.07 (m, 2H), 4.03 (dd, J=9.4, 5.5 Hz, 1H), 3.83-3.73 (m, 2H), 3.73-3.64 (m, 1H), 3.40 (s, 3H), 1.63 (s, 3H), 1.45 (s, 9H), 0.97 (t, J=8.1 Hz, 2H), 0.04 (s, 9H).

Step d:

To a stirred solution of methyl 1-[1-(tert-butoxycarbonyl)azetidin-3-yl]-4-(2,3-dichloro-6-[[2-(trimethylsilyl)ethoxy]methoxy]phenyl)-3-methyl-2-oxopyrrolidine-3-carboxylate (0.500 g, 0.830 mmol) in MeOH (6 mL) and H₂O (2 mL) was added LiOH H₂O (0.100 g, 2.37 mmol) at room temperature. The reaction mixture was stirred at 80° C. for 16 h. After cooling to room temperature, the mixture was acidified to pH 3 with citric acid and extracted with EA (30×30 mL). The combined organic layers were concentrated under reduced pressure. The residue was dissolved in toluene (8 mL) and stirred at 110° C. for 16 h and concentrated under reduced pressure to afford 1-[1-(tert-butoxycarbonyl)azetidin-3-yl]-4-(2,3-dichloro-6-[[2-(trimethylsilyl)ethoxy]methoxy]phenyl)-3-methyl-2-oxopyrrolidine-3-carboxylic acid as a brown-yellow oil (0.300 g, crude), which was used directly in the next step without purification: LCMS (ESI) calc'd for C₂₅H₃₈Cl₂N₂O₅Si [M+Na]⁺: 567, 569 (3:2) found 567, 569 (3:2).

Step e:

To a stirred solution of tert-butyl 3-[4-(2,3-dichloro-6-[[2-(trimethylsilyl)ethoxy]methoxy]phenyl)-3-methyl-2-oxopyrrolidin-1-yl]azetidine-1-carboxylate (0.300 g, 0.55 mmol) in DCM (3 mL) was added TFA (3 mL) at room temperature. The reaction mixture was stirred for 2 h and concentrated under reduced pressure. The residue was purified by Prep HPLC with the following conditions: Column: Sun Fire Prep C18 OBD Column, 19×150 mm 5 μm, 10 nm; Mobile Phase A: Water (plus 0.05% TFA), Mobile Phase B: ACN; Flow rate: 20 mL/min; Gradient: 25% B to 60% B in 4.3 min; Detector: UV 220/254 nm; Retention time: 4.20 min. The fractions containing the desired product were collected and concentrated under reduced pressure to afford 1-(azetidin-3-yl)-4-(2,3-dichloro-6-hydroxyphenyl)-3-methylpyrrolidin-2-one as an off-white solid (20.0 mg, 6% overall two steps): LCMS (ESI) calc'd for C₁₄H₁₆Cl₂N₂O₂ [M+H]⁺: 315, 317(3:2) found 315, 317 (3:2); ¹H NMR (400 MHz, CD₃OD) δ 7.30 (dd, J=8.8, 5.3 Hz, 1H), 6.78 (dd, J=19.7, 8.8 Hz, 1H), 4.99-4.92 (m, 1H), 4.69-4.45 (m, 3H), 4.35-4.20 (m, 2H), 4.13-3.89 (m, 1H), 3.76-3.64 (m, 1H), 3.24-3.01 (m, 1H), 1.03 (dd, J=137.2, 7.3 Hz, 3H).

Step f:

1-(azetidin-3-yl)-4-(2,3-dichloro-6-hydroxyphenyl)-3-methylpyrrolidin-2-one (20.0 mg, 0.06 mmol) was separated by Prep Chiral HPLC with the following conditions: Column: CHIRALPAK IG, 2×25 cm, 5 μm; Mobile Phase A: Hex (plus 0.5% 2M NH₃-MeOH)-HPLC, Mobile Phase B: EtOH-HPLC; Flow rate: 20 mL/min; Gradient: 10% B to 10% B in 27 min; Detector: UV 220/254 nm; Retention time 1: 9.87 min; Retention time 2: 17.13 min. The faster-eluting peak at 9.87 min was obtained two isomers as an off-white solid (5.00 mg, 25%). The slower-eluting peak at 17.13 min was obtained the other two isomers as an off-white solid (4.00 mg, 20%). The isomers from peak 1 (5.00 mg, 0.02 mmol) were separated by Prep Chiral HPLC with the following conditions: Column: CHIRALPAK IC, 2×25 cm, 5 μm; Mobile Phase A: Hex (plus 0.3% IPA)-HPLC, Mobile Phase B: EtOH-HPLC; Flow rate: 20 mL/min; Gradient: 15% B to 15% B in 18 min; Detector: UV 220/254 nm; Retention time 1: 11.79 min; Retention time 2: 15.07 min. The faster-eluting isomer at 11.79 min was obtained as Compound 87 (1-(azetidin-3-yl)-4-(2,3-dichloro-6-hydroxyphenyl)-3-methylpyrrolidin-2-one isomer 1) as a white solid (0.800 mg, 16%): LCMS (ESI) calc'd for C₁₄H₁₆Cl₂N₂O₂ [M+H]⁺: 315, 317(3:2) found 315, 317 (3:2); ¹H NMR (400 MHz, CD₃OD) δ 7.31 (d, J=8.8 Hz, 1H), 6.81 (d, J=8.8 Hz, 1H), 4.99-4.91 (m, 1H), 4.57-4.45 (m, 2H), 4.34-4.25 (m, 2H), 4.10-4.06 (m, 1H), 3.96-3.92 (m, 1H), 3.75-3.71 (m, 1H), 3.24-3.16 (m, 1H), 1.20 (d, J=7.2 Hz, 3H). The slower-eluting isomer at 15.07 min was obtained as Compound 88 (1-(azetidin-3-yl)-4-(2,3-dichloro-6-hydroxyphenyl)-3-methylpyrrolidin-2-one isomer 2) as a white solid (0.800 mg, 16%): LCMS (ESI) calc'd for C₁₄H₁₆Cl₂N₂O₂ [M+H]⁺: 315, 317(3:2) found 315, 317 (3:2); ¹H NMR (400 MHz, CD₃OD) δ 7.30 (d, J=8.7 Hz, 1H), 6.76 (d, J=8.8 Hz, 1H), 5.01-4.91 (m, 1H), 4.67-4.45 (m, 3H), 4.34-4.20 (m, 2H), 4.03-3.97 (m, 1H), 3.67 (dd, J=9.9, 2.7 Hz, 1H), 3.13-3.00 (m, 1H), 0.86 (d, J=7.4 Hz, 3H). The isomers from peak 2 (4.00 mg, 0.02 mmol) were separated by Prep Chiral HPLC with the following conditions: Column: CHIRALPAK AD-H, 2×25 cm, 5 μm; Mobile Phase A: Hex (plus 0.3% IPA)-HPLC, Mobile Phase B: EtOH-HPLC; Flow rate: 20 mL/min; Gradient: 15% B to 15% B in 20 min; Detector: UV 220/254 nm; Retention time 1: 12.76 min; Retention time 2: 18.47 min. The faster-eluting isomer at 12.76 min was obtained as Compound 89 (1-(azetidin-3-yl)-4-(2,3-dichloro-6-hydroxyphenyl)-3-methylpyrrolidin-2-one isomer 3) as a white solid (1.80 mg, 45%): LCMS (ESI) calc'd for C₁₄H₁₆Cl₂N₂O₂ [M+H]⁺: 315, 317(3:2) found 315, 317 (3:2); ¹H NMR (400 MHz, CD₃OD) δ 7.29 (d, J=8.8 Hz, 1H), 6.76 (d, J=8.8 Hz, 1H), 5.00-4.91 (m, 1H), 4.67-4.49 (m, 3H), 4.33-4.20 (m, 2H), 4.03-3.97 (m, 1H), 3.67 (dd, J=9.8, 2.6 Hz, 1H), 3.11-3.03 (m, 1H), 0.88-0.83 (d, J=7.2 Hz, 3H). The slower-eluting isomer at 18.47 min was obtained as Compound 90 (1-(azetidin-3-yl)-4-(2,3-dichloro-6-hydroxyphenyl)-3-methylpyrrolidin-2-one isomer 4) as a white solid (0.900 mg, 22%): LCMS (ESI) calc'd for C₁₄H₁₆Cl₂N₂O₂ [M+H]⁺: 315, 317(3:2) found 315, 317 (3:2); ¹H NMR (400 MHz, CD₃OD) δ 7.31 (d, J=8.8 Hz, 1H), 6.81 (d, J=8.7 Hz, 1H), 5.00-4.92 (m, 1H), 4.58-4.46 (m, 2H), 4.36-4.24 (m, 2H), 4.13-4.03 (m, 1H), 3.97-3.92 (m, 1H), 3.75-3.70 (m, 1H), 3.24-3.15 (m, 1H), 1.20 (d, J=7.2 Hz, 3H).

### Example 23. Compound 91 ((3S,4R)-3-amino-1-(azetidin-3-yl)-4-(2,3-dichloro-6-hydroxyphenyl)pyrrolidin-2-one) and Compound 92 ((3R,4S)-3-amino-1-(azetidin-3-yl)-4-(2,3-dichloro-6-hydroxyphenyl)pyrrolidin-2-one)

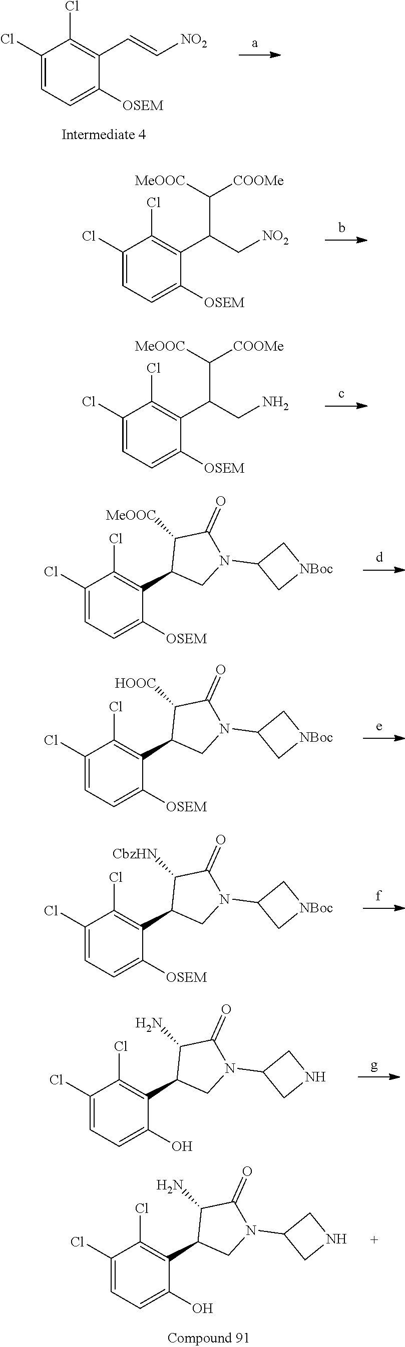

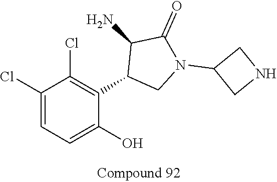 [SMILES: N[C@H]1C(=O)N(C2CNC2)C[C@@H]1c1c(O)ccc(Cl)c1Cl]

Step a:

To a mixture of (2-[3,4-dichloro-2-[(E)-2-nitroethenyl]phenoxymethoxy]ethyl)trimethylsilane (Intermediate 4, Example 4) (1.20 g, 3.29 mmol) and K₂CO₃ (1.37 g, 9.88 mmol) in DMF (6 mL) was added 1,3-dimethyl propanedioate (0.650 g, 4.94 mmol) at room temperature. The reaction mixture was stirred for 2 h, diluted with water (50 mL), and extracted with EA (3×30 mL). The combined organic layers were washed with brine (2×30 mL) and dried over anhydrous Na₂SO₄. After filtration, the filtrate was concentrated under reduced pressure. The residue was purified by reverse phase chromatography, eluting with 76% ACN in water (plus 0.05% TFA) to afford 1,3-dimethyl-2-[1-(2,3-dichloro-6-[[2-(trimethylsilyl)ethoxy]methoxy]phenyl)-2-nitroethyl]propanedioate as a yellow oil (1.20 g, 73%): LCMS (ESI) calc'd for C₁₉H₂₇Cl₂NO₈Si [M−H]⁻: 494, 496 (3:2) found 494, 496 (3:2); ¹H NMR (400 MHz, CDCl₃) δ 7.36 (d, J=9.0 Hz, 1H), 7.11 (d, J=9.0 Hz, 1H), 5.28 (s, 2H), 5.22-5.11 (m, 1H), 5.09-4.99 (m, 1H), 4.91 (dd, J=12.7, 4.9 Hz, 1H), 4.26 (d, J=10.8 Hz, 1H), 3.87-3.79 (m, 5H), 3.50 (s, 3H), 1.04-0.98 (m, 2H), 0.05 (s, 9H).

Step b:

To a solution of 1,3-dimethyl-2-[1-(2,3-dichloro-6-[[2-(trimethylsilyl)ethoxy]methoxy]phenyl)-2-nitroethyl]propanedioate (1.20 g, 2.41 mmol) in AcOH (20 mL) was added Zn (4.74 g, 72.5 mmol) at room temperature. The reaction mixture was stirred for 1 h and filtered. The filter cake was washed with EA (3×10 mL) and the filtrate was concentrated under reduced pressure followed by purification by reverse phase chromatography, eluting with 53% ACN in water (plus 0.05% TFA) to afford 1,3-dimethyl-2-[2-amino-1-(2,3-dichloro-6-[[2-(trimethylsilyl)ethoxy]methoxy]phenyl)ethyl]propanedioate as a yellow oil (1.04 g, 92%): LCMS (ESI) calc'd for C₁₉H₂₉Cl₂NO₆Si [M+H]⁺: 466, 468 (3:2) found 466, 468 (3:2); ¹H NMR (400 MHz, CDCl₃) δ 7.37 (d, J=9.0 Hz, 1H), 7.12 (d, J=9.0 Hz, 1H), 5.34-5.21 (m, 2H), 4.57-4.45 (m, 1H), 4.32 (d, J=10.0 Hz, 1H), 3.89-3.72 (m, 5H), 3.68-3.56 (m, 1H), 3.53-3.41 (m, 4H), 0.99 (t, J=8.2 Hz, 2H), 0.04 (s, 9H).

Step c:

To a stirred solution of 1,3-dimethyl-2-[2-amino-1-(2,3-dichloro-6-[[2-(trimethylsilyl)ethoxy]methoxy]phenyl)ethyl]propanedioate (1.04 g, 2.23 mmol) and tert-butyl 3-oxoazetidine-1-carboxylate (0.460 g, 2.67 mmol) in DCE (6 mL) were added TEA (0.270 g, 2.67 mmol) and NaBH(OAc)₃ (1.42 g, 6.68 mmol) at room temperature. The reaction mixture was stirred at 60° C. for 1 h. After cooling to room temperature, the mixture was quenched with water (50 mL) at room temperature and extracted with EA (3×30 mL). The combined organic layers were washed with brine (2×30 mL) and dried over anhydrous Na₂SO₄. After filtration, the filtrate was concentrated under reduced pressure. The residue was purified by reverse phase chromatography, eluting with 80% ACN in water (plus 10 mM NH₄HCO₃) to afford methyl (3S,4R)-rel-1-[1-(tert-butoxycarbonyl)azetidin-3-yl]-4-(2,3-dichloro-6-[[2-(trimethylsilyl)ethoxy]methoxy]phenyl)-2-oxopyrrolidine-3-carboxylate as a light-yellow solid (0.770 g, 58%): LCMS (ESI) calc'd for C₂₆H₃₈Cl₂N₂O₇Si [M+H]⁺: 589, 591 (3:2) found 589, 591 (3:2); ¹H NMR (400 MHz, CDCl₃) δ 7.38 (d, J=9.0 Hz, 1H), 7.11 (d, J=9.1 Hz, 1H), 5.29-5.18 (m, 2H), 5.12-5.00 (m, 1H), 4.96-4.86 (m, 1H), 4.26-4.15 (m, 2H), 4.09-3.96 (m, 3H), 3.95-3.88 (m, 1H), 3.78 (s, 3H), 3.76-3.67 (m, 3H), 1.45 (s, 9H), 0.99-0.91 (m, 2H), 0.03 (s, 9H).

Step d:

To a solution of methyl (3S,4R)-rel-1-[1-(tert-butoxycarbonyl)azetidin-3-yl]-4-(2,3-dichloro-6-[[2-(trimethylsilyl)ethoxy]methoxy]phenyl)-2-oxopyrrolidine-3-carboxylate (0.770 g, 1.30 mmol) in MeOH (6 mL) and H₂O (2 mL) was added LiOH H₂O (0.150 g, 6.53 mmol) at room temperature. The reaction mixture was stirred for 1 h, acidified to pH 6 with citric acid, diluted with water (20 mL), extracted with EA (2×50 mL), and dried over anhydrous Na₂SO₄. After filtration, the filtrate was concentrated under reduced pressure to afford (3S,4R)-rel-1-[1-(tert-butoxycarbonyl)azetidin-3-yl]-4-(2,3-dichloro-6-[[2-(trimethylsilyl)ethoxy]methoxy]phenyl)-2-oxopyrrolidine-3-carboxylic acid as a yellow solid (0.780 g, crude), which was used in the next step directly without further purification: LCMS (ESI) calc'd for C₂₅H₃₆Cl₂N₂O₇Si [M+H]⁺: 575, 577 (3:2) found 575, 577 (3:2); ¹H NMR (400 MHz, CDCl₃) δ 7.39 (d, J=9.0 Hz, 1H), 7.11 (d, J=9.0 Hz, 1H), 5.29-5.25 (m, 2H), 5.10-5.00 (m, 1H), 4.89-4.85 (m, 1H), 4.29-4.19 (m, 3H), 4.08-3.98 (m, 2H), 3.89 (t, J=9.6 Hz, 1H), 3.80-3.70 (m, 3H), 1.45 (s, 9H), 1.00-0.93 (m, 2H), 0.03 (s, 9H).

Step e:

A mixture of (3S,4R)-rel-1-[1-(tert-butoxycarbonyl)azetidin-3-yl]-4-(2,3-dichloro-6-[[2-(trimethylsilyl)ethoxy]methoxy]phenyl)-2-oxopyrrolidine-3-carboxylic acid (0.780 g, 1.35 mmol), DPPA (0.560 g, 2.03 mmol), and TEA (0.200 g, 2.03 mmol) in toluene (4 mL) was firstly stirred at room temperature for 1 h and then at 80° C. for 30 min. After cooling to room temperature, benzyl alcohol (0.660 g, 6.09 mmol) was added. The reaction mixture was stirred at 110° C. for 1 h and concentrated under reduced pressure. The residue was purified by reverse phase chromatography, eluting with 80% ACN in water (plus 10 mM NH₄HCO₃) to afford tert-butyl 3-[(3S,4R)-rel-3-[[(benzyloxy)carbonyl]amino]-4-(2,3-dichloro-6-[[2-(trimethylsilyl)ethoxy]methoxy]phenyl)-2-oxopyrrolidin-1-yl]azetidine-1-carboxylate as an off-white solid (0.570 g, 61%): LCMS (ESI) calc'd for C₃₂H₄₃Cl₂N₃O₇Si [M+H]⁺: 680, 682 (3:2) found 680, 682 (3:2); ¹H NMR (400 MHz, CDCl₃) δ 7.41-7.31 (m, 6H), 7.17-7.08 (m, 1H), 5.31-5.15 (m, 2H), 5.15-5.07 (m, 2H), 5.04 (d, J=12.2 Hz, 2H), 4.95 (t, J=8.5 Hz, 1H), 4.53-4.38 (m, 1H), 4.28-4.15 (m, 2H), 4.12-3.95 (m, 2H), 3.86-3.64 (m, 4H), 1.45 (s, 9H), 0.96 (t, J=8.3 Hz, 2H), 0.03 (d, J=1.1 Hz, 9H).

Step f:

A mixture of tert-butyl 3-[(3S,4R)-rel-3-[[(benzyloxy)carbonyl]amino]-4-(2,3-dichloro-6-[[2-(trimethylsilyl)ethoxy]methoxy]phenyl)-2-oxopyrrolidin-1-yl]azetidine-1-carboxylate (0.570 g, 0.830 mmol) in HBr (2.5 mL, 33% in AcOH) was stirred at room temperature for 1 h. The reaction mixture was diluted with water (10 mL), basified to pH 7 with saturated aqueous NaHCO₃, and concentrated under reduced pressure. The residue was purified by reverse phase chromatography, eluting with 4% ACN in water (plus 0.05% TFA) to afford the crude product. The crude product was purified by Prep-HPLC with the following conditions: Column: Atlantis Prep T3 OBD Column, 19×250 mm, 10 μm; Mobile Phase A: Water (plus 0.05% TFA), Mobile Phase B: ACN; Flow rate: 20 mL/min; Gradient: 0% B to 40% B in 6 min; Detector: UV 210/254 nm; Retention time: 5.56 min. The fractions containing the desired product were collected and concentrated under reduced pressure to afford (3S,4R)-rel-3-amino-1-(azetidin-3-yl)-4-(2,3-dichloro-6-hydroxyphenyl)pyrrolidin-2-one as an off-white solid (95.0 mg, 27%): LCMS (ESI) calc'd for C₁₃H₁₅Cl₂N₃O₂ [M+H]⁺: 316, 318 (3:2) found 316, 318 (3:2); ¹H NMR (300 MHz, DMSO-d₆+D₂O) δ 7.44 (d, J=8.9 Hz, 1H), 6.91 (d, J=8.9 Hz, 1H), 5.04-4.88 (m, 1H), 4.60 (d, J=9.6 Hz, 1H), 4.39-4.04 (m, 5H), 3.89-3.85 (m, 1H), 3.75-3.68 (m, 1H).

Step g:

(3S,4R)-rel-3-amino-1-(azetidin-3-yl)-4-(2,3-dichloro-6-hydroxyphenyl)pyrrolidin-2-one (60.0 mg, 0.14 mmol) was purified by Prep Chiral HPLC with the following conditions: Column: CHIRALPAK IG, 2.0 cm I.D×25 cm, 5 μm; Mobile Phase A: Hex (plus 0.1% IPA), Mobile Phase B: EtOH; Flow rate: 20 mL/min; Gradient: 25% B to 25% B in 15 min; Detector: UV 220/254 nm; Retention time 1: 7.77 min; Retention time 2: 11.93 min. The faster-eluting enantiomer at 7.77 min was obtained (3S,4R)-3-amino-1-(azetidin-3-yl)-4-(2,3-dichloro-6-hydroxyphenyl)pyrrolidin-2-one. The product was purified by Prep-HPLC with the following conditions: Column: Sun Fire Prep C18 OBD Column, 19×150 mm 5 μm 10 nm; Mobile Phase A: Water (plus 0.05% TFA), Mobile Phase B: ACN; Flow rate: 20 mL/min; Gradient: 10% B to 40% B in 4.3 min; Detector: UV 254/210 nm; Retention Time: 4.20 min. The fractions containing the desired product were collected and concentrated under reduced pressure to afford Compound 91 ((3S,4R)-3-amino-1-(azetidin-3-yl)-4-(2,3-dichloro-6-hydroxyphenyl)pyrrolidin-2-one) as an off-white solid (15.0 mg, 19%): LCMS (ESI) calc'd for C₁₃H₁₅Cl₂N₃O₂ [M+H]⁺: 316, 318 (3:2) found 316, 318 (3:2); ¹H NMR (300 MHz, DMSO-d₆+D₂O) δ 7.45 (d, J=8.9 Hz, 1H), 6.92 (d, J=8.9 Hz, 1H), 5.04-4.89 (m, 1H), 4.61 (d, J=9.5 Hz, 1H), 4.33 (dd, J=11.4, 7.6 Hz, 1H), 4.29-4.09 (m, 4H), 3.89-3.85 (m, 1H), 3.75-3.70 (m, 1H). The slower-eluting enantiomer at 11.93 min was obtained (3R,4S)-3-amino-1-(azetidin-3-yl)-4-(2,3-dichloro-6-hydroxyphenyl)pyrrolidin-2-one. The product was purified by Prep-HPLC with the following conditions: Column: Sun Fire Prep C18 OBD Column, 19×150 mm 5 μm, 10 nm; Mobile Phase A: Water (plus 0.05% TFA), Mobile Phase B: ACN; Flow rate: 20 mL/min; Gradient: 5% B to 30% B in 4.3 min; Detector: UV 254/210 nm; Retention Time: 4.20 min. The fractions containing the desired product were collected and concentrated under reduced pressure to afford Compound 92 ((3R,4S)-3-amino-1-(azetidin-3-yl)-4-(2,3-dichloro-6-hydroxyphenyl)pyrrolidin-2-one) as a purple solid (13.0 mg, 17%): LCMS (ESI) calc'd for C₁₃H₁₅Cl₂N₃O₂ [M+H]⁺: 316, 318 (3:2) found 316, 318 (3:2); ¹H NMR (300 MHz, DMSO-d₆+D₂O) δ 7.45 (d, J=8.9 Hz, 1H), 6.92 (d, J=8.9 Hz, 1H), 5.02-4.90 (m, 1H), 4.61 (d, J=9.6 Hz, 1H), 4.33 (dd, J=11.3, 7.6 Hz, 1H), 4.29-4.08 (m, 4H), 3.89-3.85 (m, 1H), 3.75-3.71 (m, 1H).

### Example 24. Compound 93 (5-(2,3-dichloro-6-hydroxyphenyl)-3-(2-hydroxyethyl)oxazolidin-2-one isomer 1) and Compound 94 (5-(2,3-dichloro-6-hydroxyphenyl)-3-(2-hydroxyethyl)oxazolidin-2-one isomer 2)

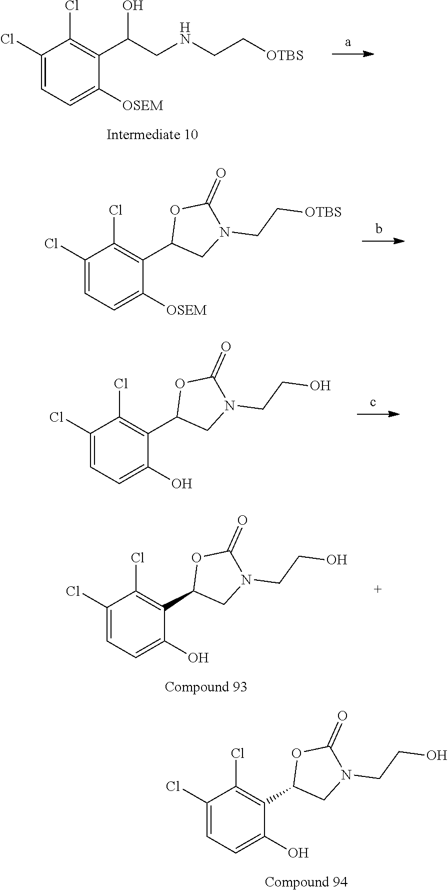

Step a:

To a stirred solution of 2-([2-[(tert-butyldimethylsilyl)oxy]ethyl]amino)-1-(2,3-dichloro-6-[[2-(trimethylsilyl) ethoxy]methoxy]phenyl)ethanol (Intermediate 10, Example 9) (0.250 g, 0.49 mmol) and TEA (0.100 g, 0.98 mmol) in DCM (2 mL) was added CDI (0.160 g, 0.98 mmol) at room temperature. The reaction solution was stirred for 1 h, diluted with water (20 mL), and extracted with EA (3×20 mL). The combined organic layers were washed with brine (3×20 mL) and dried over anhydrous Na₂SO₄. After filtration, the filtrate was concentrated under reduced pressure. The residue was purified by reverse phase chromatography, eluted 85% ACN in water (plus 10 mM NH₄HCO₃) to afford 3-[2-[(tert-butyldimethylsilyl)oxy]ethyl]-5-(2,3-dichloro-6-[[2-(trimethylsilyl)ethoxy]methoxy]phenyl)-1,3-oxazolidin-2-one as a colorless oil (0.150 g, 57%): LCMS (ESI) calc'd for C₂₃H₃₉Cl₂NO₅Si₂ [M+H]⁺: 536, 538 (3:2) found 536, 538 (3:2); ¹H NMR (300 MHz, CDCl₃) δ 7.42 (d, J=9.0 Hz, 1H), 7.11 (d, J=9.0 Hz, 1H), 6.17 (dd, J=10.0, 7.8 Hz, 1H), 5.28-5.18 (m, 2H), 4.04 (dd, J=10.1, 8.4 Hz, 1H), 3.85-3.80 (m, 2H), 3.79-3.69 (m, 3H), 3.69-3.60 (m, 1H), 3.32-3.22 (m, 1H), 0.98-0.91 (m, 2H), 0.90 (s, 9H), 0.08 (d, J=3.2 Hz, 6H), 0.02 (s, 9H).

Step b:

To a stirred solution of 3-[2-[(tert-butyldimethylsilyl)oxy]ethyl]-5-(2,3-dichloro-6-[[2-(trimethylsilyl)ethoxy]methoxy]phenyl)-1,3-oxazolidin-2-one (0.150 g, 0.28 mmol) in 1,4-dioxane (1.50 mL) was added aqueous HCl (6 M, 1.50 mL) at room temperature. The reaction mixture was stirred for 1 h and concentrated under reduced pressure. The residue was purified by Prep-HPLC with the following conditions: Column: X Bridge Prep Phenyl OBD Column, 19×150 mm, 5 μm, 13 nm; Mobile Phase A: Water (plus 10 mM NH₄HCO₃), Mobile Phase B: ACN; Flow rate: 20 mL/min; Gradient: 30% to 50% in 4.3 min; Detector: UV 254/220 nm; Retention time: 4.2 min. The fractions containing the desired product were collected and concentrated under reduced pressure to afford 5-(2,3-dichloro-6-hydroxyphenyl)-3-(2-hydroxyethyl)-1,3-oxazolidin-2-one as an off-white solid (55.7 mg, 68%): LCMS (ESI) calc'd for C₁₁H₁₁Cl₂NO₄ [M+H]⁺: 292, 294 (3:2) found 292, 294 (3:2); ¹H NMR (300 MHz, CD₃OD) δ 7.39 (d, J=8.9 Hz, 1H), 6.84 (d, J=8.9 Hz, 1H), 6.26 (dd, J=10.1, 7.8 Hz, 1H), 4.08 (dd, J=10.2, 8.4 Hz, 1H), 3.84-3.70 (m, 3H), 3.65-3.51 (m, 1H), 3.28 (t, J=5.4 Hz, 1H).

Step c:

The product 5-(2,3-dichloro-6-hydroxyphenyl)-3-(2-hydroxyethyl)-1,3-oxazolidin-2-one (55.0 mg, 0.19 mmol) was separated by Prep Chiral HPLC with the following conditions: Column: CHIRALPAK AD-H, 2×25 cm, 5 μm; Mobile Phase A: CO₂, Mobile Phase B: MeOH-Preparative; Flow rate: 50 mL/min; Gradient: 50% B; Detector: UV 254/220 nm; Retention time 1: 2.04 min; Retention time 2: 2.74 min. The faster-eluting enantiomer at 2.04 min was obtained as Compound 93 (5-(2,3-dichloro-6-hydroxyphenyl)-3-(2-hydroxyethyl)-1,3-oxazolidin-2-one isomer 1) as an off-white solid (11.1 mg, 20%): LCMS (ESI) calc'd for C₁₁H₁₁Cl₂NO₄ [M+H]⁺: 292, 294 (3:2) found 292, 294 (3:2); ¹H NMR (400 MHz, CD₃OD) δ 7.39 (d, J=8.8 Hz, 1H), 6.84 (d, J=8.9 Hz, 1H), 6.26 (dd, J=10.1, 7.8 Hz, 1H), 4.08 (dd, J=10.2, 8.4 Hz, 1H), 3.80 (t, J=8.1 Hz, 1H), 3.77-3.72 (m, 2H), 3.62-3.54 (m, 1H), 3.31-3.27 (m, 1H). The slower-eluting enantiomer at 2.74 min was obtained as Compound 94 (5-(2,3-dichloro-6-hydroxyphenyl)-3-(2-hydroxyethyl)-1,3-oxazolidin-2-one isomer 2) as an off-white solid (11.0 mg, 19.58%): LCMS (ESI) calc'd for C₁₁H₁₁Cl₂NO₄ [M+H]⁺: 292, 294 (3:2) found 292, 294 (3:2): ¹H NMR (400 MHz, CD₃OD) δ 7.39 (d, J=8.8 Hz, 1H), 6.84 (d, J=8.9 Hz, 1H), 6.26 (dd, J=10.2, 7.8 Hz, 1H), 4.08 (dd, J=10.2, 8.4 Hz, 1H), 3.80 (t, J=8.1 Hz, 1H), 3.79-3.73 (m, 2H), 3.62-3.54 (m, 1H), 3.31-3.27 (m, 1H).

### Example 25. Compound 95 (6-(2,3-dichloro-6-hydroxyphenyl)-4-(2-hydroxyethyl)morpholin-3-one isomer 1) and Compound 96 (6-(2,3-dichloro-6-hydroxyphenyl)-4-(2-hydroxyethyl)morpholin-3-one isomer 2)

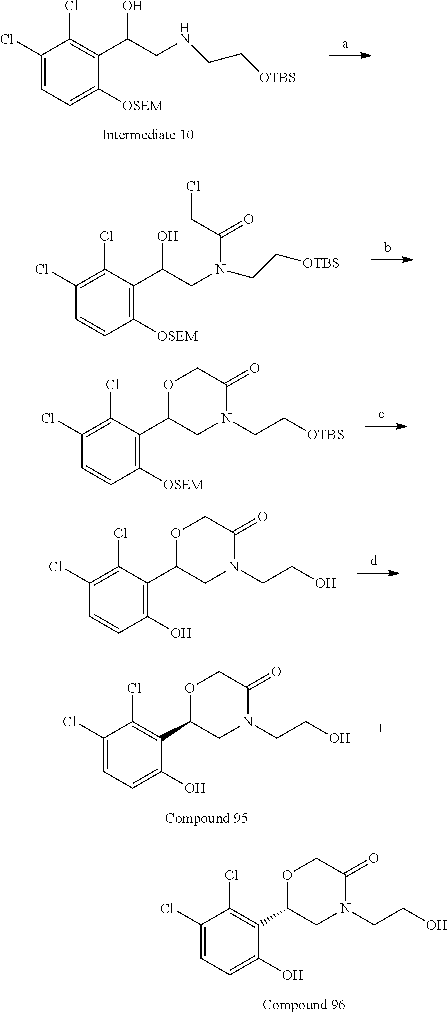

Step a:

To a stirred solution of 2-([2-[(tert-butyldimethylsilyl)oxy]ethyl]amino)-1-(2,3-dichloro-6-[[2-(trimethylsilyl) ethoxy]methoxy]phenyl)ethanol (Intermediate 10, Example 9) (0.250 g, 0.49 mmol) and TEA (0.100 g, 0.98 mmol) in DCM (3 mL) was added chloroacetyl chloride (0.110 g, 0.98 mmol) at 0° C. The reaction mixture was stirred at room temperature for 1 h, diluted with water (20 mL), and extracted with EA (3×20 mL). The combined organic layers were washed with brine (3×20 mL) and dried over anhydrous Na₂SO₄. After filtration, the filtrate was concentrated under reduced pressure to afford N-[2-[(tert-butyldimethylsilyl)oxy]ethyl]-2-chloro-N-[2-(2,3-dichloro-6-[[2-(trimethylsilyl)ethoxy]methoxy]phenyl)-2-hydroxyethyl]acetamide as a yellow oil (0.250 g, crude), which was used directly in the next step without purification: LCMS (ESI) calc'd for C₂₄H₄₂Cl₃NO₅Si₂ [M+H]⁺: 586, 588, 590 (3:3:1) found 586, 588, 590 (3:3:1).

Step b:

To a stirred solution of N-[2-[(tert-butyldimethylsilyl)oxy]ethyl]-2-chloro-N-[2-(2,3-dichloro-6-[[2-(trimethylsilyl)ethoxy] methoxy]phenyl)-2-hydroxyethyl]acetamide (0.250 g, 0.43 mmol) in i-PrOH (3 mL) was added KOH (48.0 mg, 0.85 mmol) at room temperature. The reaction mixture was stirred for 1 h, diluted with water (20 mL), and extracted with EA (3×20 mL). The combined organic layers were washed with brine (3×20 mL) and dried over anhydrous Na₂SO₄. After filtration, the filtrate was concentrated under reduced pressure. The residue was purified by reverse phase chromatography, eluting with 85% ACN in water (plus 0.05% TFA) to afford 4-[2-[(tert-butyldimethylsilyl)oxy]ethyl]-6-(2,3-dichloro-6-[[2-(trimethylsilyl)ethoxy]methoxy]phenyl)morpholin-3-one as a yellow oil (0.130 g, 52% overall two steps): LCMS (ESI) calc'd for C₂₄H₄₁Cl₂NO₅Si₂ [M+H]⁺: 550, 552 (3:2) found 550, 552 (3:2); ¹H NMR (300 MHz, CDCl₃) δ 7.43 (d, J=9.0 Hz, 1H), 7.11 (d, J=9.1 Hz, 1H), 5.61-5.52 (m, 1H), 5.31-5.23 (m, 2H), 4.53-4.26 (m, 3H), 3.93 (dd, J=5.9, 4.1 Hz, 2H), 3.83-3.73 (m, 2H), 3.60-3.49 (m, 2H), 3.36-3.28 (m, 1H), 1.01-0.87 (m, 11H), 0.06-0.01 (m, 15H).

Step c:

To a stirred solution of 4-[2-[(tert-butyldimethylsilyl)oxy]ethyl]-6-(2,3-dichloro-6-[[2-(trimethylsilyl) ethoxy]methoxy]phenyl)morpholin-3-one (0.130 g, 0.24 mmol) in 1,4-dioxane (1 mL) was added aqueous HCl (6 M, 1 mL) at room temperature. The reaction mixture was stirred for 1 h and concentrated under reduced pressure. The residue was purified by Prep HPLC with the following conditions: Column: X Bridge Prep Phenyl OBD Column, 19×150 mm, 5 μm, 13 nm; Mobile Phase A: Water (plus 10 mM NH₄HCO₃), Mobile Phase B: ACN; Flow rate: 20 mL/min; Gradient: 30% to 50% in 4.3 min; Detector: UV 254/220 nm; Retention time: 4.2 min. The fractions containing the desired product were collected and concentrated under reduced pressure to afford 6-(2,3-dichloro-6-hydroxyphenyl)-4-(2-hydroxyethyl)morpholin-3-one as a yellow solid (35.2 mg, 42.21%): LCMS (ESI) calc'd for C₁₂H₁₃Cl₂NO₄ [M+H]⁺: 306, 308 (3:2) found 306, 308 (3:2); ¹H NMR (400 MHz, CD₃OD) δ 7.37 (d, J=8.9 Hz, 1H), 6.83 (d, J=8.9 Hz, 1H), 5.62 (dd, J=10.9, 3.5 Hz, 1H), 4.46-4.32 (m, 2H), 4.12 (dd, J=12.4, 11.0 Hz, 1H), 3.77 (t, J=5.7 Hz, 2H), 3.64-3.47 (m, 3H).

Step d:

The product 6-(2,3-dichloro-6-hydroxyphenyl)-4-(2-hydroxyethyl)morpholin-3-one (50.0 mg, 0.16 mmol) was purified by Prep Chiral HPLC with the following conditions: Column: CHIRALPAK IG, 30 mm×250 mm, 5 μm; Mobile Phase A: CO₂, Mobile Phase B: MeOH (plus 0.1% 2M NH₃-MeOH); Flow rate: 70 mL/min; Gradient: 50% B; Detector: UV 254/220 nm; Retention time 1: 4.24 min; Retention time 2: 7.92 min. The faster-eluting enantiomer at 4.24 min was obtained as Compound 95 (6-(2,3-dichloro-6-hydroxyphenyl)-4-(2-hydroxyethyl)morpholin-3-one isomer 1) as a brown solid (20.4 mg, 40%) LCMS (ESI) calc'd for C₁₂H₁₃Cl₂NO₄ [M+H]⁺: 306, 308 (3:2) found 306, 308 (3:2); ¹H NMR (400 MHz, CD₃OD) δ 7.37 (d, J=8.9 Hz, 1H), 6.83 (d, J=8.8 Hz, 1H), 5.62 (dd, J=10.9, 3.5 Hz, 1H), 4.49-4.33 (m, 2H), 4.12 (dd, J=12.5, 11.0 Hz, 1H), 3.77 (t, J=5.5 Hz, 2H), 3.65-3.48 (m, 3H). The slower-eluting enantiomer at 7.92 min was obtained as Compound 96 (6-(2,3-dichloro-6-hydroxyphenyl)-4-(2-hydroxyethyl)morpholin-3-one isomer 2) as a brown solid (23.2 mg, 45%) LCMS (ESI) calc'd for C₁₂H₁₃Cl₂NO₄ [M+H]⁺: 306, 308 (3:2) found 306, 308 (3:2); ¹H NMR (400 MHz, CD₃OD) δ 7.37 (d, J=8.9 Hz, 1H), 6.83 (d, J=8.9 Hz, 1H), 5.62 (dd, J=10.9, 3.5 Hz, 1H), 4.46-4.31 (m, 2H), 4.12 (dd, J=12.4, 11.0 Hz, 1H), 3.77 (t, J=5.5 Hz, 2H), 3.65-3.48 (m, 3H).

### Example 26. Compound 97 (1-(azetidin-3-yl)-5-(2,3-dichloro-6-hydroxyphenyl)piperazin-2-one isomer 1) and Compound 98 (1-(azetidin-3-yl)-5-(2,3-dichloro-6-hydroxyphenyl)piperazin-2-one isomer 2)

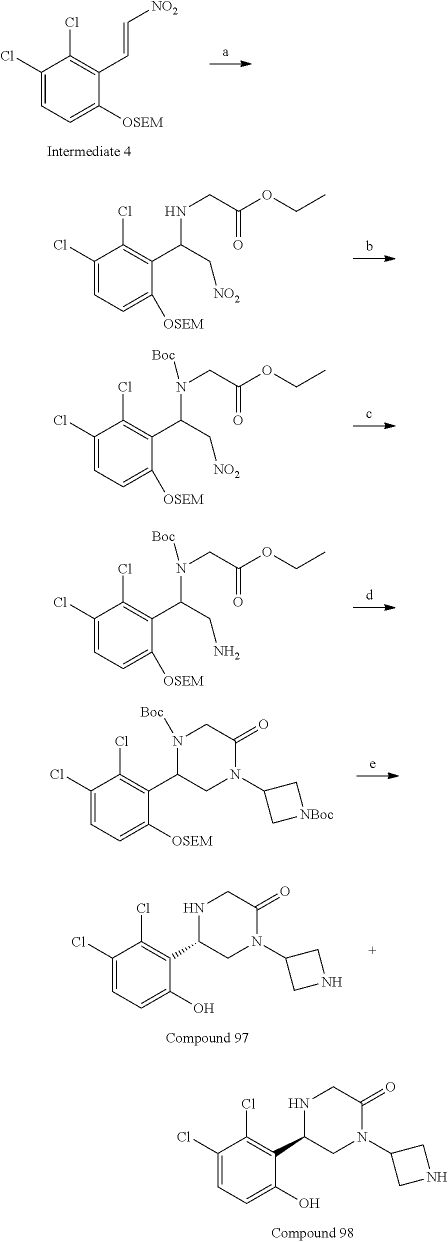

Step a:

To a stirred solution of (2-[3,4-dichloro-2-[(E)-2-nitroethenyl]phenoxymethoxy]ethyl)trimethylsilane (Intermediate 4, Example 4) (1.00 g, 2.74 mmol) and ethyl glycinate hydrochloride (0.770 g, 5.49 mmol) in ACN (10 mL) was added DIEA (1.43 mL, 11.1 mmol) at room temperature. The reaction mixture was stirred at 60° C. for 2 h. The resulting mixture was diluted with water (50 mL) and extracted with EA (3×30 mL). The combined organic layers were washed with brine (3×20 mL) and dried over anhydrous Na₂SO₄. After filtration, the filtrate was concentrated under reduced pressure. The residue was purified by reverse phase chromatography, eluting with 70% ACN in water (plus 0.05% TFA) to afford ethyl 2-[[1-(2,3-dichloro-6-[[2-(trimethylsilyl)ethoxy]methoxy]phenyl)-2-nitroethyl]amino]acetate as a light-yellow solid (0.670 g, 47%): LCMS (ESI) calc'd for C₁₈H₂₈Cl₂N₂O₆Si [M+H]⁺: 467, 469 (3:2) found 467, 469 (3:2); ¹H NMR (400 MHz, CDCl₃) δ 7.38 (d, J=9.0 Hz, 1H), 7.12 (d, J=9.0 Hz, 1H), 5.34 (s, 2H), 5.31-5.26 (m, 1H), 5.00-4.96 (m, 1H), 4.63 (dd, J=12.4, 5.7 Hz, 1H), 4.10-3.99 (m, 2H), 3.85-3.79 (m, 2H), 3.48 (d, J=16.9 Hz, 1H), 3.26 (d, J=16.9 Hz, 1H), 1.20 (t, J=7.2 Hz, 3H), 1.03-0.96 (m, 2H), 0.04 (s, 9H).

Step b:

To a stirred solution of ethyl 2-[[1-(2,3-dichloro-6-[[2-(trimethylsilyl)ethoxy]methoxy]phenyl)-2-nitroethyl]amino]acetate (0.670 g, 1.44 mmol) in 1,4-dioxane (7 mL) was added Boc₂O (1.57 g, 7.20 mmol) at room temperature. The reaction mixture was stirred at 80° C. for 16 h. The resulting mixture was diluted with water (50 mL) and extracted with EA (3×20 mL). The combined organic layers were washed with brine (2×20 mL) and dried over anhydrous Na₂SO₄. After filtration, the filtrate was concentrated under reduced pressure. The residue was purified by reverse phase chromatography, eluting with 75% ACN in water (plus 0.05% TFA) to afford ethyl 2-[(tert-butoxycarbonyl)[1-(2,3-dichloro-6-[[2-(trimethylsilyl)ethoxy]methoxy]phenyl)-2-nitroethyl]amino]acetate as a light-yellow oil (0.580 g, 64%): LCMS (ESI) calc'd for C₂₃H₃₆Cl₂N₂O₈Si [M+Na]⁺: 589, 591 (3:2) found 589, 591 (3:2); ¹H NMR (400 MHz, CDCl₃) δ 7.45-7.39 (m, 1H), 7.18-7.13 (m, 1H), 6.65-6.61 (m, 1H), 5.32-5.17 (m, 3H), 5.13-5.00 (m, 1H), 4.23-3.92 (m, 2H), 3.92-3.53 (m, 4H), 1.50 (d, J=35.0 Hz, 9H), 1.32-1.18 (m, 3H), 1.02-0.93 (m, 2H), 0.04 (s, 9H).

Step c:

To a stirred solution of ethyl 2-[(tert-butoxycarbonyl)[1-(2,3-dichloro-6-[[2-(trimethylsilyl)ethoxy]methoxy]phenyl)-2-nitroethyl]amino]acetate (0.570 g, 1.00 mmol) in AcOH (6 mL) was added Zn (1.31 g, 20.08 mmol) at room temperature. The reaction mixture was stirred for 1 h and filtered. The filtrate was concentrated under reduced pressure. The residue was purified by reverse phase chromatography, eluting with 50% ACN in water (plus 0.05% TFA) to afford ethyl 2-[[2-amino-1-(2,3-dichloro-6-[[2-(trimethylsilyl)ethoxy]methoxy]phenyl)ethyl](tert-butoxycarbonyl)amino] acetate as a light-yellow oil (0.310 g, 47%): LCMS (ESI) calc'd for C₂₃H₃₈Cl₂N₂O₆Si [M+H]⁺: 537, 539 (3:2) found 537, 539 (3:2); ¹H NMR (300 MHz, CDCl₃) δ 7.51-7.41 (m, 1H), 7.24-7.14 (m, 1H), 6.21-6.07 (m, 1H), 5.34-5.20 (m, 2H), 4.38-4.13 (m, 3H), 4.04-3.92 (m, 1H), 3.82-3.64 (m, 2H), 3.58-3.50 (m, 2H), 1.47 (s, 9H), 1.36-1.26 (m, 3H), 1.00-0.91 (m, 2H), 0.04 (s, 9H).

Step d:

To a stirred mixture of ethyl 2-[[2-amino-1-(2,3-dichloro-6-[[2-(trimethylsilyl)ethoxy]methoxy]phenyl)ethyl](tert-butoxycarbonyl)amino]acetate trifluoroacetic acid (0.300 g, 0.46 mmol) and tert-butyl 3-oxoazetidine-1-carboxylate (0.120 g, 0.69 mmol) in DCE (5 mL) were added NaOAc (75.5 mg, 0.92 mmol) and NaBH(AcO)₃ (0.290 g, 1.38 mmol) at room temperature. The reaction mixture was stirred for 16 h, quenched with saturated aqueous NH₄Cl (20 mL), and extracted with EA (3×20 mL). The combined organic layers were washed with brine (2×20 mL) and dried over anhydrous Na₂SO₄. After filtration, the filtrate was concentrated under reduced pressure. The residue was purified by reverse phase chromatography, eluting with 55% ACN in water (plus 10 mM NH₄HCO₃) to afford tert-butyl 4-[1-(tert-butoxycarbonyl)azetidin-3-yl]-2-(2,3-dichloro-6-[[2-(trimethylsilyl)ethoxy]methoxy]phenyl)-5-oxopiperazine-1-carboxylate as a light-yellow oil (0.240 g, 73%): LCMS (ESI) calc'd for C₂₉H₄₅Cl₂N₃O₇Si [M+H]⁺: 646, 648 (3:2) found 646, 648 (3:2); ¹H NMR (400 MHz, CDCL₃) δ 7.40 (d, J=8.7 Hz, 1H), 7.15 (d, J=9.0 Hz, 1H), 5.33 (s, 2H), 5.26-5.15 (m, 1H), 4.58-4.41 (m, 1H), 4.26 (t, J=9.0 Hz, 1H), 4.21-4.01 (m, 1H), 3.99-3.86 (m, 1H), 3.83-3.62 (m, 3H), 3.62-3.41 (m, 4H), 1.45 (s, 9H), 1.19 (s, 9H), 0.95 (t, J=8.2 Hz, 2H), 0.05 (s, 9H).

Step e:

To a stirred solution of tert-butyl 4-[1-(tert-butoxycarbonyl)azetidin-3-yl]-2-(2,3-dichloro-6-[[2-(trimethylsilyl)ethoxy]methoxy]phenyl)-5-oxopiperazine-1-carboxylate (0.150 g, 0.23 mmol) in 1,4-dioxane (1 mL) was added HCl (6 M, 1 mL) at room temperature. The reaction solution was stirred for 1 h and concentrated under reduced pressure. The residue was purified by Prep-HPLC with the following conditions: Column: Sun Fire Prep C18 OBD Column, 19×150 mm, 5 μm, 10 nm; Mobile Phase A: water (plus 0.05% TFA), Mobile Phase B: ACN; Flow rate: 20 mL/min; Gradient: 5% B to 30% B in 4.3 min; Detector: UV 210 nm; Retention time: 4.20 min. The fractions containing the desired product were collected and concentrated under reduced pressure. The product was separated by Prep Chiral HPLC with the following conditions: Column: CHIRALPAK IG UL001, 20×250 mm, 5 μm; Mobile Phase A: Hex (plus 0.2% IPA)-HPLC, Mobile Phase B: EtOH-HPLC; Flow rate: 20 mL/min; Gradient: 25% B to 25% B in 25 min; Detector: UV 220/254 nm; Retention time 1: 12.35 min; Retention time 2: 20.55 min. The faster-eluting enantiomer at 12.35 min was concentrated under reduced pressure. The residue was purified by Prep HPLC with the following conditions: Column: Sun Fire Prep C18 OBD Column, 19×150 mm 5 μm, 10 nm; Mobile Phase A: Water (plus 0.05% TFA), Mobile Phase B: ACN; Flow rate: 20 mL/min; Gradient: 25% B to 45% B in 4.3 min; Detector: UV 254/210 nm; Retention Time: 4.23 min. The fractions containing the desired product were collected and concentrated under reduced pressure to afford Compound 97 (1-(azetidin-3-yl)-5-(2,3-dichloro-6-hydroxyphenyl)piperazin-2-one isomer 1) as a purple solid (18.0 mg, 14%): LCMS (ESI) calc'd for C₁₃H₁₅Cl₂N₃O₂ [M+H]⁺: 316, 318 (3:2) found 316, 318 (3:2); ¹H NMR (400 MHz, CD₃OD) δ 7.52 (d, J=8.9 Hz, 1H), 6.95 (d, J=8.9 Hz, 1H), 5.35 (dd, J=11.6, 4.5 Hz, 1H), 4.63-4.53 (m, 3H), 4.43-4.31 (m, 2H), 4.19-4.06 (m, 2H), 3.96 (d, J=16.6 Hz, 1H), 3.65 (dd, J=12.7, 4.5 Hz, 1H). The slower-eluting enantiomer at 20.55 min was concentrated under reduced pressure. The residue was purified by Prep HPLC with the following conditions: Column: Sun Fire Prep C18 OBD Column, 19×150 mm, 5 μm, 10 nm; Mobile Phase A: Water (plus 0.05% TFA), Mobile Phase B: ACN; Flow rate: 20 mL/min; Gradient: 20% B to 45% B in 4.3 min; Detector: UV 254/210 nm; Retention Time: 4.23 min. The fractions containing the desired product were collected and concentrated under reduced pressure to afford Compound 98 (1-(azetidin-3-yl)-5-(2,3-dichloro-6-hydroxyphenyl)piperazin-2-one isomer 2) as a purple solid (17.4 mg, 14%): LCMS (ESI) calc'd for C₁₃H₁₅Cl₂N₃O₂ [M+H]⁺: 316, 318 (3:2) found 316, 318 (3:2); ¹H NMR (400 MHz, CD₃OD) δ 7.51 (d, J=8.9 Hz, 1H), 6.94 (d, J=8.9 Hz, 1H), 5.33 (dd, J=11.5, 4.5 Hz, 1H), 4.63-4.51 (m, 3H), 4.42-4.32 (m, 2H), 4.15-4.04 (m, 2H), 3.95 (d, J=16.6 Hz, 1H), 3.64 (dd, J=12.7, 4.5 Hz, 1H).

### Example 27. Compound 99 ((4S)-4-(2,3-dichloro-6-hydroxyphenyl)-1-(2-hydroxyethyl)imidazolidin-2-one)

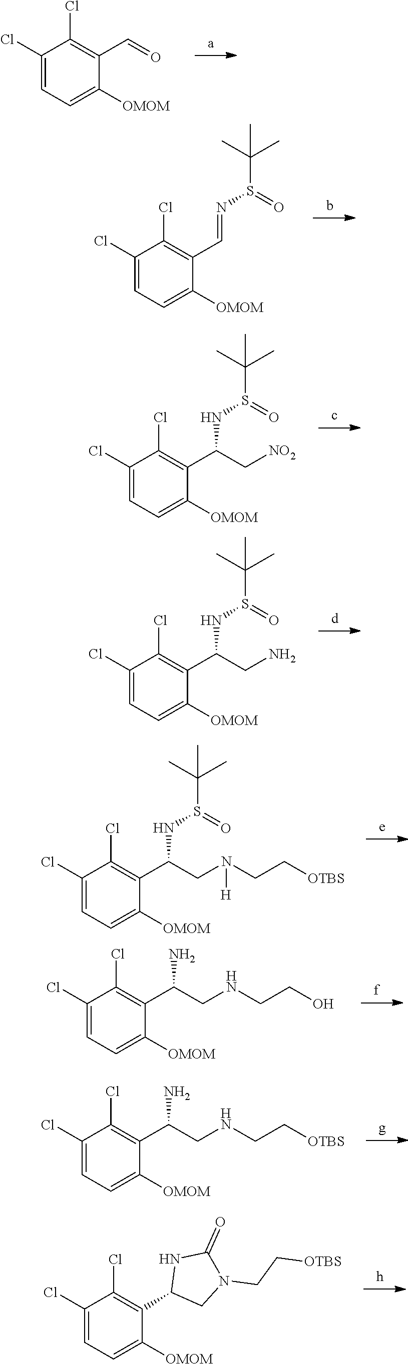

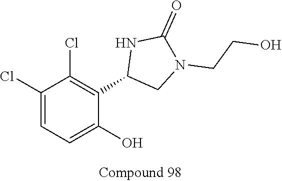 [SMILES: O=C1N[C@@H](c2c(O)ccc(Cl)c2Cl)CN1CCO]

Step a:

To a stirred solution of 2,3-dichloro-6-(methoxymethoxy)benzaldehyde (2.00 g, 8.50 mmol) and (S)-2-methylpropane-2-sulfinamide (1.55 g, 12.8 mmol) in THE (20 mL) was added Ti(OEt)₄ (5.82 g, 25.5 mmol) dropwise at room temperature under nitrogen atmosphere. The reaction mixture was stirred for 16 h, quenched with saturated aqueous NaHCO₃(30 mL), and filtered. The filter cake was washed with EA (5×20 mL) and the filtrate was extracted with EA (2×20 mL). The combined organic layers were washed with brine (3×20 mL) and dried over anhydrous Na₂SO₄. After filtration, the filtrate was concentrated under reduced pressure to afford (S)—N-[[2,3-dichloro-6-(methoxymethoxy)phenyl]methylidene]-2-methylpropane-2-sulfinamide as a light-yellow oil (2.60 g, 81%): LCMS (ESI) calc'd for C₁₃H₁₇Cl₂NO₃S [M+H]⁺: 338, 340 (3:2) found 338, 340 (3:2); ¹H NMR (400 MHz, CDCl₃) δ 8.92 (s, 1H), 7.50 (d, J=9.0 Hz, 1H), 7.14 (d, J=9.0 Hz, 1H), 5.24 (s, 2H), 3.49 (s, 3H), 1.32 (s, 9H).

Step b:

To a stirred solution of (S)—N-[[2,3-dichloro-6-(methoxymethoxy)phenyl]methylidene]-2-methylpropane-2-sulfinamide (1.00 g, 2.95 mmol) in nitromethane (10 mL) was added K₂CO₃ (1.02 g, 7.39 mmol) at room temperature. The reaction mixture was stirred at 60° C. for 16 h. After cooling to room temperature, the mixture was diluted with water (20 mL) and extracted with EA (3×30 mL). The combined organic layers were washed with brine (2×20 mL) and dried over anhydrous Na₂SO₄. After filtration, the filtrate was concentrated under reduced pressure to afford (S)—N-[(1S)-1-[2,3-dichloro-6-(methoxymethoxy)phenyl]-2-nitroethyl]-2-methylpropane-2-sulfinamide as a light-yellow oil (1.29 g, crude), which was used directly in the next step without purification: LCMS (ESI) calc'd for C₁₄H₂₀Cl₂N₂O₅S [M+H]⁺: 399, 401 (3:2) found 399, 401 (3:2); ¹H NMR (300 MHz, CDCl₃) δ 7.40 (d, J=9.0 Hz, 1H), 7.10 (d, J=9.0 Hz, 1H), 5.32-5.22 (m, 2H), 5.13 (dd, J=12.5, 6.7 Hz, 1H), 4.98 (dd, J=12.6, 7.6 Hz, 1H), 4.66 (d, J=10.7 Hz, 1H), 3.53 (s, 3H), 1.17 (s, 9H).

Step c:

To a stirred mixture of (S)—N-[(1S)-1-[2,3-dichloro-6-(methoxymethoxy)phenyl]-2-nitroethyl]-2-methylpropane-2-sulfinamide (1.29 g, 3.23 mmol) in AcOH (13 mL) was added Zn (4.23 g, 64.6 mmol) in portions at room temperature. The reaction mixture was stirred for 1 h and filtered. The filter cake was washed with DCM (3×10 mL) and the filtrate concentrated under reduced pressure. The residue was purified by reverse phase chromatography, eluting with 40% ACN in water (plus 0.05% TFA) to afford (S)—N-[(1S)-2-amino-1-[2,3-dichloro-6-(methoxymethoxy)phenyl]ethyl]-2-methylpropane-2-sulfinamide as a light-yellow oil (0.520 g, 48% overall two steps): LCMS (ESI) calc'd for C₁₄H₂₂Cl₂N₂O₃S [M+H]⁺: 369, 371 (3:2) found 369, 371 (3:2); ¹H NMR (300 MHz, CDCL₃) δ 7.35 (d, J=9.0 Hz, 1H), 7.08 (d, J=9.0 Hz, 1H), 5.30-5.17 (m, 2H), 5.09-4.93 (m, 1H), 3.51 (s, 3H), 3.31-3.11 (m, 1H), 3.06-2.91 (m, 1H), 1.18 (s, 9H).

Step d:

To a stirred mixture of (S)—N-[(1S)-2-amino-1-[2,3-dichloro-6-(methoxymethoxy)phenyl]ethyl]-2-methylpropane-2-sulfinamide (0.500 g, 1.35 mmol) and 2-[(tert-butyldimethylsilyl)oxy]acetaldehyde (0.210 g, 1.22 mmol) in DCM (5 mL) was added NaBH₃CN (0.170 g, 2.70 mmol) in portions at room temperature. The reaction mixture was stirred for 2 h, quenched with water (20 mL), and extracted with EA (3×20 mL). The combined organic layers were washed with brine (2×20 mL) and dried over anhydrous Na₂SO₄. After filtration, the filtrate was concentrated under reduced pressure. The residue was purified by reverse phase chromatography, eluting with 45% ACN in water (plus 0.05% TFA) to afford (S)—N-[(1S)-2-([2-[(tert-butyldimethylsilyl)oxy]ethyl]amino)-1-[2,3-dichloro-6-(methoxymethoxy)phenyl]ethyl]-2-methylpropane-2-sulfinamide as a light-yellow oil (0.400 g, 34%): LCMS (ESI) calc'd for C₂₂H₄₀Cl₂N₂O₄SSi [M+H]⁺: 527, 529 (3:2) found 527, 529 (3:2).

Step e:

To a stirred solution of (S)—N-[(1S)-2-([2-[(tert-butyldimethylsilyl)oxy]ethyl]amino)-1-[2,3-dichloro-6-(methoxymethoxy)phenyl]ethyl]-2-methylpropane-2-sulfinamide (0.400 g, 0.45 mmol) in MeOH (2.4 mL) was added HCl (1.2 mL, 2 M) dropwise at room temperature. The reaction mixture was stirred for 16 h, diluted with water (15 mL), and extracted with EA (2×10 mL). The aqueous layer was basified to pH 8 with saturated aqueous NaHCO₃ and the mixture was concentrated under reduced pressure to afford 2-[[(2S)-2-amino-2-[2,3-dichloro-6-(methoxymethoxy)phenyl]ethyl]amino]ethanol as a light-yellow oil (0.150 g, crude): LCMS (ESI) calc'd for C₁₂H₁₈Cl₂N₂O₃ [M+H]⁺: 309, 311 (3:2) found 309, 311 (3:2).

Step f:

To a stirred mixture of 2-[[(2S)-2-amino-2-[2,3-dichloro-6-(methoxymethoxy)phenyl]ethyl]amino]ethanol (0.150 g, 0.48 mmol) and imidazole (0.100 g, 1.45 mmol) in DCM (2 mL) was added TBSCl (0.150 g, 0.97 mmol) in portions at room temperature. The reaction mixture was stirred for 16 h, diluted with water (30 mL), and extracted with EA (3×20 mL). The combined organic layers were washed with brine (2×20 mL) and dried over anhydrous Na₂SO₄. After filtration, the filtrate was concentrated under reduced pressure. The residue was purified by reverse phase chromatography, eluting with 40% ACN in water (plus 10 mM NH₄HCO₃) to afford [(2S)-2-amino-2-[2,3-dichloro-6-(methoxymethoxy)phenyl]ethyl]([2-[(tert-butyldimethylsilyl)oxy]ethyl])amine as a light-yellow oil (70 mg, 31%): LCMS (ESI) calc'd for C₁₈H₃₂Cl₂N₂O₃Si [M+H]⁺: 423, 425 (3:2) found 423, 425 (3:2).

Step g:

To a stirred mixture of [(2S)-2-amino-2-[2,3-dichloro-6-(methoxymethoxy)phenyl]ethyl]([2-[(tert-butyldimethylsilyl)oxy]ethyl])amine (70.0 mg, 0.16 mmol) and CDI (0.270 g, 0.16 mmol) in THE (1 mL) was added TEA (42.0 mg, 0.41 mmol) at room temperature. The reaction mixture was stirred at 60° C. for 1 h under nitrogen atmosphere and concentrated under reduced pressure. The residue was purified by reverse phase chromatography eluting with 60% ACN in water (plus 0.05% TFA) to afford (4S)-1-[2-[(tert-butyldimethylsilyl)oxy]ethyl]-4-[2,3-dichloro-6-(methoxymethoxy)phenyl]imidazolidin-2-one as a light-yellow oil (40.0 mg, 48%): LCMS (ESI) calc'd for C₁₉H₃₀Cl₂N₂O₄Si [M+H]⁺: 449, 451 (3:2) found 449, 451 (3:2).

Step h:

To a stirred mixture of (4S)-1-[2-[(tert-butyldimethylsilyl)oxy]ethyl]-4-[2,3-dichloro-6-(methoxymethoxy)phenyl]imidazolidin-2-one (40.0 mg, 0.09 mmol) in DCM (2 mL) was added BBr₃ (0.2 mL) dropwise at room temperature. The reaction mixture was stirred for 0.5 h, quenched with MeOH (0.2 mL) at 0° C., and concentrated under reduced pressure. The residue was purified by Prep-HPLC with the following conditions: Column: Atlantis HILIC OBD Column, 19×150 mm, 5 μm; Mobile Phase A: water (plus 10 mM NH₄HCO₃), Mobile Phase B: ACN; Flow rate: 20 mL/min; Gradient: 25% B to 50% B in 5.5 min; Detector: UV 210 nm; Retention time: 4.50 min. The fractions containing the desired product were collected and concentrated under reduced pressure to afford Compound 99 ((4S)-4-(2,3-dichloro-6-hydroxyphenyl)-1-(2-hydroxyethyl)imidazolidin-2-one) as an off-white solid (14.7 mg, 57%): LCMS (ESI) calc'd for C₁₁H₁₂Cl₂N₂O₃ [M+H]⁺: 291, 293 (3:2) found 291, 293 (3:2); ¹H NMR (400 MHz, CD₃OD) δ 7.30 (d, J=8.8 Hz, 1H), 6.79 (d, J=8.8 Hz, 1H), 5.61 (dd, J=10.7, 7.2 Hz, 1H), 4.06-3.99 (m, 1H), 3.71 (td, J=5.6, 1.1 Hz, 2H), 3.63 (dd, J=8.9, 7.2 Hz, 1H), 3.58-3.49 (m, 1H), 3.25-3.17 (m, 1H).

The compounds in Table G below were prepared in analogous fashion to Compound 99.

TABLE G

| Compound | | | |
|---|---|---|---|
| Number | Structure | Chemical Name | MS: (M + H)⁺ & ¹H NMR |
| 100 | 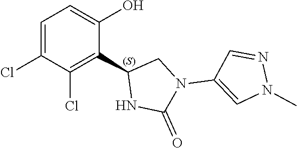 | (4S)-4-(2,3-dichloro-6- hydroxyphenyl)-1-(1- methylpyrazol-4- yl)imidazolidin-2-one | [M + H]⁺: 327, 329 (3:2); ¹H NMR (400 MHz, CD₃OD) δ 7.70 (s, 1H), 7.55 (s, 1H), 7.33 (d, J = 8.8 Hz, 1H), 6.80 (d, J = 8.8 Hz, 1H), 5.76 (dd, J = 10.9, 6.4 Hz, 1H), 4.19 (dd, J = 10.9, 8.9 Hz, 1H), 3.89 (dd, J = 8.7, 6.3 Hz, 1H), 3.88 (s, 3H). |
| 101 | 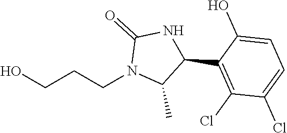 [SMILES: C[C@H]1[C@H](c2c(O)ccc(Cl)c2Cl)NC(=O)N1CCCO] | (4S,5S)-rel-4-(2,3-dichloro- 6-hydroxyphenyl)-1-(3- hydroxypropyl)-5- methylimidazolidin-2-one | [M + H]⁺: 319, 321 (3:2); ¹H NMR (400 MHz, CD₃OD) δ 7.32 (d, J = 8.8 Hz, 1H), 6.80 (d, J = 8.8 Hz, 1H), 5.14 (d, J = 7.2 Hz, 1H), 4.05-3.95 (m, 1H), 3.65 (t, J = 6.3 Hz, 2H), 3.51-3.43 (m, 1H), 3.31-3.23 (m, 1H), 1.82-1.75 (m, 2H), 1.36 (d, J = 6.2 Hz, 3H). |
| 102 | 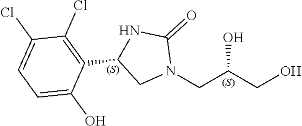 | (4S)-4-(2,3-dichloro-6- hydroxyphenyl)-1-[(2S)- 2,3- dihydroxypropyl]imidazoli- din-2-one | [M + H]⁺: 321, 323 (3:2); ¹H NMR (400 MHz, CD₃OD) δ 7.31 (d, J = 8.8 Hz, 1H), 6.80 (d, J = 8.8 Hz, 1H), 5.62 (dd, J = 10.8, 7.1 Hz, 1H), 4.01 (dd, J = 10.8, 8.9 Hz, 1H), 3.87-3.77 (m, 1H), 3.66 (dd, J = 8.9, 7.1 Hz, 1H), 3.65-3.52 (m, 2H), 3.43 (dd, J = |
| | | | 14.3, 6.4 Hz, 1H), 3.27 (dd, J = |
| | | | 14.3, 4.9 Hz, 1H). |
| 103 | 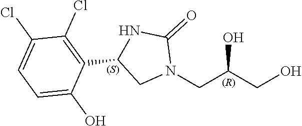 | (4S)-4-(2,3-dichloro-6- hydroxyphenyl)-1-[(2R)- 2,3- dihydroxypropyl]imidazoli- din-2-one | [M + H]⁺: 321, 323 (3:2); ¹H NMR (400 MHz, CD₃OD) δ 7.31 (d, J = 8.8 Hz, 1H), 6.80 (d, J = 8.8 Hz, 1H), 5.62 (dd, J = 10.8, 7.1 Hz, 1H), 4.05 (dd, J = 10.8, 9.0 Hz, 1H), 3.85-3.79 (m, 1H), 3.66 (dd, J = 9.0, 7.1 Hz, 1H), 3.63-3.48 (m, 3H), 3.13 (dd, J = |
| | | | 14.4, 6.7 Hz, 1H). |
| 104 | 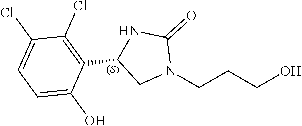 | (4S)-4-(2,3-dichloro-6- hydroxyphenyl)-1-(3- hydroxypropyl)imidazolidin- 2-one | [M + H]⁺: 305, 307 (3:2); H NMR (400 MHz, CD₃OD) δ 7.31 (d, J = 8.8 Hz, 1H), 6.79 (d, J = 8.8 Hz, 1H), 5.61 (dd, J = 10.6, 6.9 Hz, 1H), 3.90 (t, J = 9.8 Hz, 1H), 3.69-3.58 (m, 3H), 3.43-3.35 (m, 2H), 1.85-1.72 (m, 2H). |

### Example 28. Evaluation of Kv1.3 Potassium Channel Blocker Activities

This assay is used to evaluate the disclosed compounds' activities as Kv1.3 potassium channel blockers.

### Cell Culture

CHO-K₁ cells stably expressing Kv1.3 were grown in DMEM containing 10% heat-inactivated FBS, 1 mM sodium pyruvate, 2 mM L-glutamine and G418 (500 μg/ml). Cells were grown in culture flasks at 37° C. in a 5% CO₂-humidified incubator.

### Solutions

The cells were bathed in an extracellular solution containing 140 mM NaCl, 4 mM KCl, 2 mM CaCl₂), 1 mM MgCl₂, 5 mM glucose, 10 mM HEPES; pH adjusted to 7.4 with NaOH; 295-305 mOsm. The internal solution contained 50 mM KCl, 10 mM NaCl, 60 mM KF, 20 mM EGTA, 10 mM HEPES; pH adjusted to 7.2 with KOH; 285 mOsm. All compounds were dissolved in DMSO at 30 mM. Compound stock solutions were freshly diluted with external solution to concentrations of 30 nM, 100 nM, 300 nM, 1 μM, 3 μM, 10 μM, 30 μM and 100 μM. The highest content of DMSO (0.3%) was present in 100 μM.

### Voltage Protocol

The currents were evoked by applying 100 ms depolarizing pulses from −90 mV (holding potential) to +40 mV were applied with 0.1 Hz frequency. Control (compound-free) and compound pulse trains for each compound concentration applied contained 20 pulses. 10-second breaks were used between pulse trains (see Table H below).

TABLE H
Voltage protocol.

| 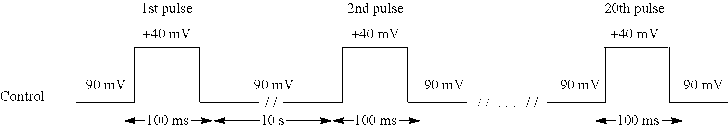 | | |
|---|---|---|
| | //10 s | Compound |
| | | application |
| | | 1st concentration |
| 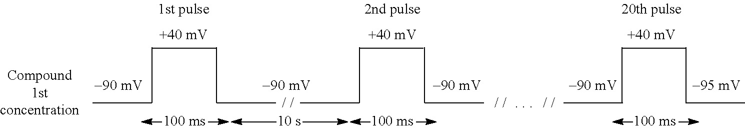 | | |
| | //10 s | Compound |
| | | application |
| | | 2nd concentration |
| 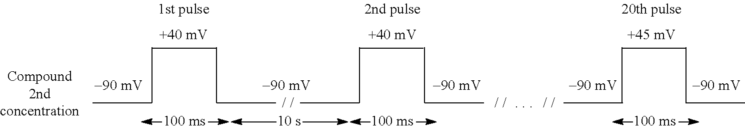 | | |
| . | . | Compound |
| . | . | application |
| . | . | Nth concentration |

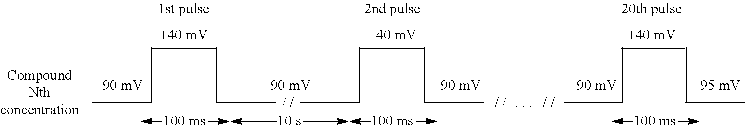

### Patch Clamp Recordings and Compound Application

Whole-cell current recordings and compound application were enabled by means of an automated patch clamp platform Patchliner (Nanion Technologies GmbH). EPC 10 patch clamp amplifier (HEKA Elektronik Dr. Schulze GmbH) along with Patchmaster software (HEKA Elektronik Dr. Schulze GmbH) was used for data acquisition. Data were sampled at 10 kHz without filtering. Passive leak currents were subtracted online using a P/4 procedure (HEKA Elektronik Dr. Schulze GmbH). Increasing compound concentrations were applied consecutively to the same cell without washouts in between. Total compound incubation time before the next pulse train was not longer than 10 seconds. Peak current inhibition was observed during compound equilibration.

### Data Analysis

AUC and peak values were obtained with Patchmaster (HEKA Elektronik Dr. Schulze GmbH). To determine IC₅₀, the last single pulse in the pulse train corresponding to a given compound concentration was used. Obtained AUC and peak values in the presence of compound were normalized to control values in the absence of compound. Using Origin (OridinLab), IC₅₀ was derived from data fit to Hill equation: I_{compound}/I_{control}=(100−A)/(1+([compound]/IC₅₀)nH)+A, where IC₅₀ value is the concentration at which current inhibition is half-maximal, [compound] is the applied compound concentration, A is the fraction of current that is not blocked and nH is the Hill coefficient.

### Example 29. Evaluation of hERG Activities

This assay is used to evaluate the disclosed compounds' inhibition activities against the hERG channel.

hERG Electrophysiology

This assay is used to evaluate the disclosed compounds' inhibition activities against the hERG channel.

### Cell Culture

CHO-K₁ cells stably expressing hERG were grown in Ham's F-12 Medium with glutamine containing 10% heat-inactivated FBS, 1% penicillin/streptomycin, hygromycin (100 μg/ml) and G418 (100 μg/ml). Cells were grown in culture flasks at 37° C. in a 5% CO₂-humidified incubator.

### Solutions

The cells were bathed in an extracellular solution containing 140 mM NaCl, 4 mM KCl, 2 mM CaCl₂), 1 mM MgCl₂, 5 mM Glucose, 10 mM HEPES; pH adjusted to 7.4 with NaOH; 295-305 mOsm. The internal solution contained 50 mM KCl, 10 mM NaCl, 60 mM KF, 20 mM EGTA, 10 mM HEPES; pH adjusted to 7.2 with KOH; 285 mOsm. All compounds were dissolved in DMSO at 30 mM. Compound stock solutions were freshly diluted with external solution to concentrations of 30 nM, 100 nM, 300 nM, 1 μM, 3 μM, 10 μM, 30 μM and 100 μM. The highest content of DMSO (0.3%) was present in 100 μM.

### Voltage Protocol

The voltage protocol (see Table I) was designed to simulate voltage changes during a cardiac action potential with a 300 ms depolarization to +20 mV (analogous to the plateau phase of the cardiac action potential), a repolarization for 300 ms to −50 mV (inducing a tail current) and a final step to the holding potential of −80 mV. The pulse frequency was 0.3 Hz. Control (compound-free) and compound pulse trains for each compound concentration applied contained 70 pulses.

TABLE I
hERG voltage protocol.

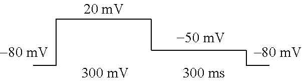

### Patch Clamp Recordings and Compound Application

Whole-cell current recordings and compound application were enabled by means of an automated patch clamp platform Patchliner (Nanion). EPC 10 patch clamp amplifier (HEKA) along with Patchmaster software (HEKA Elektronik Dr. Schulze GmbH) was used for data acquisition. Data were sampled at 10 kHz without filtering. Increasing compound concentrations were applied consecutively to the same cell without washouts in between.

### Data Analysis

AUC and PEAK values were obtained with Patchmaster (HEKA Elektronik Dr. Schulze GmbH). To determine IC₅₀ the last single pulse in the pulse train corresponding to a given compound concentration was used. Obtained AUC and PEAK values in the presence of compound were normalized to control values in the absence of compound. Using Origin (OridinLab), IC₅₀ was derived from data fit to Hill equation: I_{compound}/I_{control}=(100−A)/(1+([compound]/IC₅₀)nH)+A, where IC₅₀ is the concentration at which current inhibition is half-maximal, [compound] is the applied compound concentration, A is the fraction of current that is not blocked and nH is the Hill coefficient.

Table 1 provides a summary of the inhibition activities of certain selected compounds of the instant invention against Kv1.3 potassium channel and hERG channel.

TABLE 1
IC₅₀ (μM) values of certain exemplified compounds against the Kv1.3 potassium
channel and the hERG channel.

| Compound | | Kv1.3 | hERG |
|---|---|---|---|
| Number | Structure | IC₅₀ | IC₅₀ |
| 1 | | <0.1 | >30 |
| 2 | 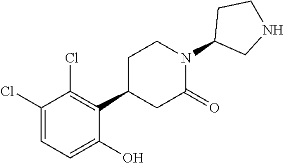 [SMILES: O=C1C[C@@H](c2c(O)ccc(Cl)c2Cl)CCN1[C@H]1CCNC1] | <0.1 | >30 |
| 3 | 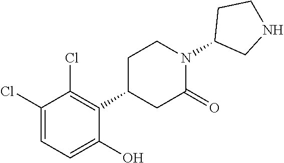 [SMILES: O=C1C[C@H](c2c(O)ccc(Cl)c2Cl)CCN1[C@@H]1CCNC1] | <1 | * |
| 4 | 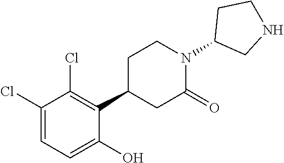 [SMILES: O=C1C[C@@H](c2c(O)ccc(Cl)c2Cl)CCN1[C@@H]1CCNC1] | <1 | * |
| 5 | 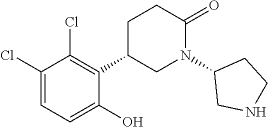 [SMILES: O=C1CC[C@@H](c2c(O)ccc(Cl)c2Cl)CN1[C@@H]1CCNC1] | <0.1 | >30 |
| 6 | 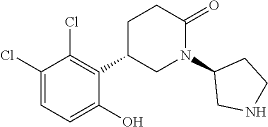 [SMILES: O=C1CC[C@@H](c2c(O)ccc(Cl)c2Cl)CN1[C@H]1CCNC1] | <1 | * |
| 7 | 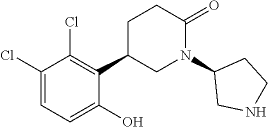 [SMILES: O=C1CC[C@H](c2c(O)ccc(Cl)c2Cl)CN1[C@H]1CCNC1] | <0.1 | >30 |
| 8 | 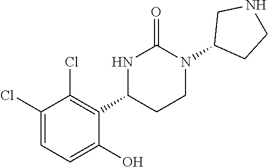 [SMILES: O=C1N[C@@H](c2c(O)ccc(Cl)c2Cl)CCN1[C@H]1CCNC1] | <1 | * |
| 9 | 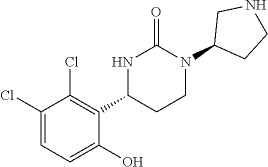 [SMILES: O=C1N[C@@H](c2c(O)ccc(Cl)c2Cl)CCN1[C@@H]1CCNC1] | <1 | >10 |
| 10 | 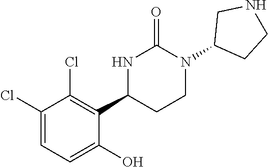 [SMILES: O=C1N[C@H](c2c(O)ccc(Cl)c2Cl)CCN1[C@H]1CCNC1] | <1 | * |
| 11 | 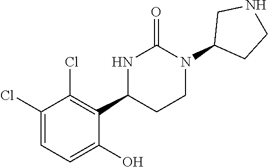 [SMILES: O=C1N[C@H](c2c(O)ccc(Cl)c2Cl)CCN1[C@@H]1CCNC1] | <1 | >10 |
| 12 | 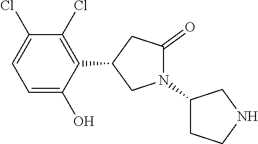 [SMILES: O=C1C[C@@H](c2c(O)ccc(Cl)c2Cl)CN1[C@H]1CCNC1] | <1 | >30 |
| 13 | 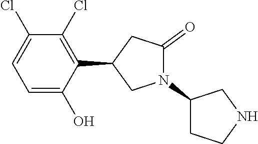 [SMILES: O=C1C[C@H](c2c(O)ccc(Cl)c2Cl)CN1[C@@H]1CCNC1] | <0.1 | >10 |
| 14 | 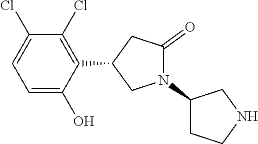 [SMILES: O=C1C[C@@H](c2c(O)ccc(Cl)c2Cl)CN1[C@@H]1CCNC1] | <1 | >30 |
| 15 | 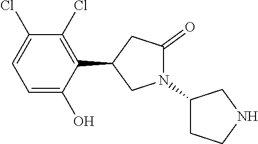 [SMILES: O=C1C[C@H](c2c(O)ccc(Cl)c2Cl)CN1[C@H]1CCNC1] | <0.1 | >30 |
| 16 | 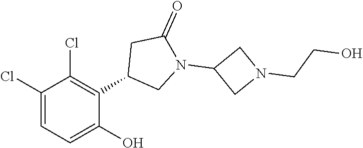 [SMILES: O=C1C[C@@H](c2c(O)ccc(Cl)c2Cl)CN1C1CN(CCO)C1] | <0.1 | >10 |
| 17 | 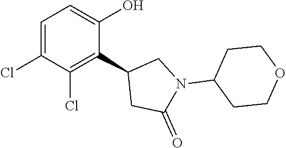 [SMILES: O=C1C[C@@H](c2c(O)ccc(Cl)c2Cl)CN1C1CCOCC1] | <1 | >30 |
| 18 | 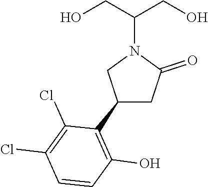 [SMILES: O=C1C[C@@H](c2c(O)ccc(Cl)c2Cl)CN1C(CO)CO] | <1 | * |
| 19 | 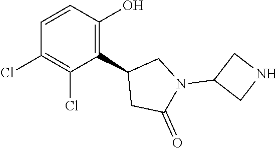 [SMILES: O=C1C[C@@H](c2c(O)ccc(Cl)c2Cl)CN1C1CNC1] | <0.1 | >100 |
| 20 | 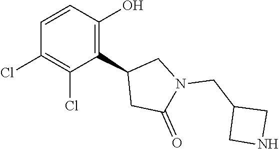 [SMILES: O=C1C[C@@H](c2c(O)ccc(Cl)c2Cl)CN1CC1CNC1] | <0.1 | >100 |
| 21 | 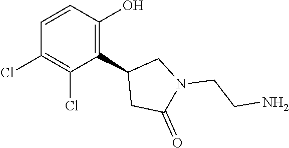 [SMILES: NCCN1C[C@H](c2c(O)ccc(Cl)c2Cl)CC1=O] | <0.1 | >100 |
| 22 | 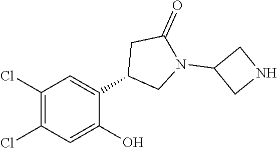 [SMILES: O=C1C[C@@H](c2cc(Cl)c(Cl)cc2O)CN1C1CNC1] | <1 | >100 |
| 23 | 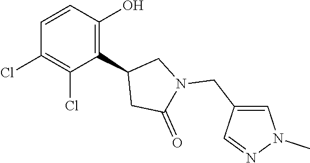 [SMILES: Cn1cc(CN2C[C@H](c3c(O)ccc(Cl)c3Cl)CC2=O)cn1] | <1 | >30 |
| 24 | 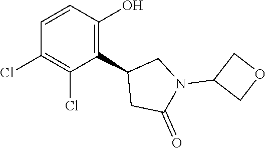 [SMILES: O=C1C[C@@H](c2c(O)ccc(Cl)c2Cl)CN1C1COC1] | <0.1 | >30 |
| 25 | 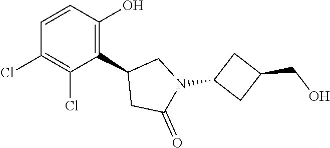 [SMILES: O=C1C[C@@H](c2c(O)ccc(Cl)c2Cl)CN1[C@H]1C[C@H](CO)C1] | <0.1 | >30 |
| 26 | 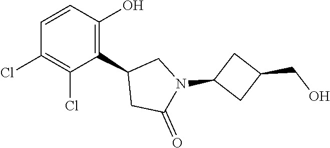 [SMILES: O=C1C[C@@H](c2c(O)ccc(Cl)c2Cl)CN1[C@H]1C[C@@H](CO)C1] | <0.1 | >30 |
| 27 | 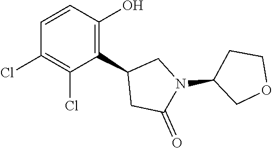 [SMILES: O=C1C[C@@H](c2c(O)ccc(Cl)c2Cl)CN1[C@H]1CCOC1] | <1 | * |
| 28 | 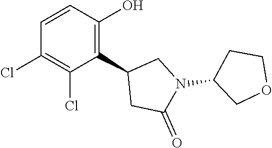 [SMILES: O=C1C[C@@H](c2c(O)ccc(Cl)c2Cl)CN1[C@@H]1CCOC1] | <1 | * |
| 29 | 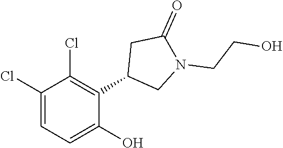 [SMILES: O=C1C[C@@H](c2c(O)ccc(Cl)c2Cl)CN1CCO] | <0.1 | >30 |
| 30 | 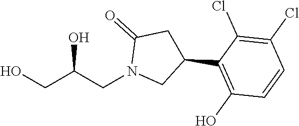 [SMILES: O=C1C[C@@H](c2c(O)ccc(Cl)c2Cl)CN1C[C@H](O)CO] | <0.1 | >100 |
| 31 | 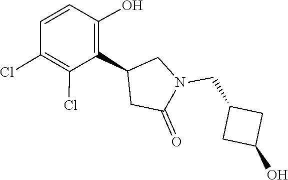 [SMILES: O=C1C[C@@H](c2c(O)ccc(Cl)c2Cl)CN1C[C@H]1C[C@H](O)C1] | <0.1 | >30 |
| 32 | 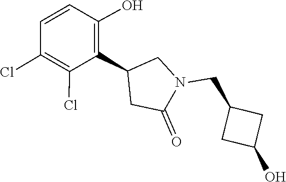 [SMILES: O=C1C[C@@H](c2c(O)ccc(Cl)c2Cl)CN1C[C@H]1C[C@@H](O)C1] | <0.1 | >30 |
| 33 | 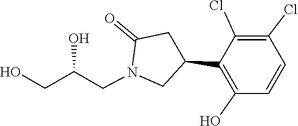 [SMILES: O=C1C[C@@H](c2c(O)ccc(Cl)c2Cl)CN1C[C@@H](O)CO] | <0.1 | >100 |
| 34 | 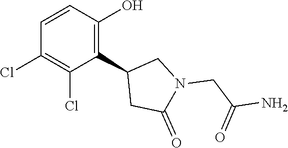 [SMILES: NC(=O)CN1C[C@H](c2c(O)ccc(Cl)c2Cl)CC1=O] | <0.1 | >100 |
| 35 | 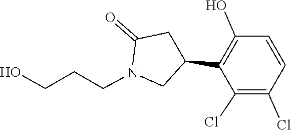 [SMILES: O=C1C[C@@H](c2c(O)ccc(Cl)c2Cl)CN1CCCO] | <0.01 | >30 |
| 36 | 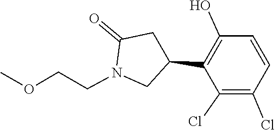 [SMILES: COCCN1C[C@H](c2c(O)ccc(Cl)c2Cl)CC1=O] | <0.1 | >30 |
| 37 | 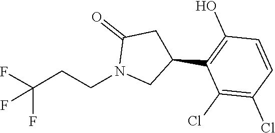 [SMILES: O=C1C[C@@H](c2c(O)ccc(Cl)c2Cl)CN1CCC(F)(F)F] | <1 | >100 |
| 38 | 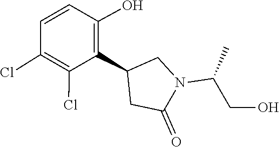 [SMILES: C[C@H](CO)N1C[C@H](c2c(O)ccc(Cl)c2Cl)CC1=O] | <1 | >30 |
| 39 | 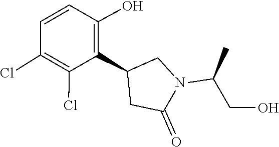 [SMILES: C[C@@H](CO)N1C[C@H](c2c(O)ccc(Cl)c2Cl)CC1=O] | <1 | >100 |
| 40 | 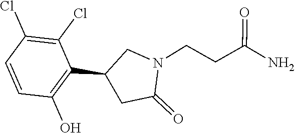 [SMILES: NC(=O)CCN1C[C@H](c2c(O)ccc(Cl)c2Cl)CC1=O] | <0.1 | >100 |
| 41 | 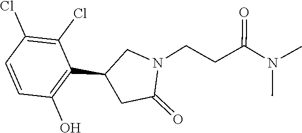 [SMILES: CN(C)C(=O)CCN1C[C@H](c2c(O)ccc(Cl)c2Cl)CC1=O] | <0.1 | >100 |
| 42 | 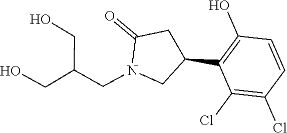 [SMILES: O=C1C[C@@H](c2c(O)ccc(Cl)c2Cl)CN1CC(CO)CO] | <0.1 | >30 |
| 43 | 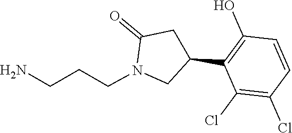 [SMILES: NCCCN1C[C@H](c2c(O)ccc(Cl)c2Cl)CC1=O] | <0.01 | >30 |
| 44 | 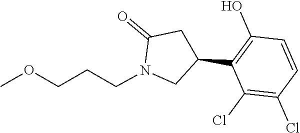 [SMILES: COCCCN1C[C@H](c2c(O)ccc(Cl)c2Cl)CC1=O] | <0.1 | >10 |
| 45 | 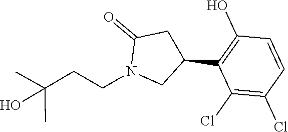 [SMILES: CC(C)(O)CCN1C[C@H](c2c(O)ccc(Cl)c2Cl)CC1=O] | <1 | * |
| 46 | 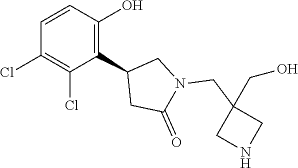 [SMILES: O=C1C[C@@H](c2c(O)ccc(Cl)c2Cl)CN1CC1(CO)CNC1] | <0.1 | >100 |
| 47 | 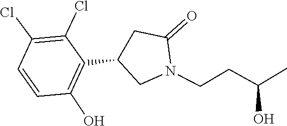 [SMILES: C[C@@H](O)CCN1C[C@H](c2c(O)ccc(Cl)c2Cl)CC1=O] | <0.1 | * |
| 48 | 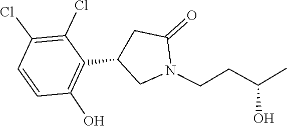 [SMILES: C[C@H](O)CCN1C[C@H](c2c(O)ccc(Cl)c2Cl)CC1=O] | <0.1 | * |
| 49 | 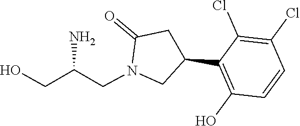 [SMILES: N[C@@H](CO)CN1C[C@H](c2c(O)ccc(Cl)c2Cl)CC1=O] | <0.1 | * |
| 50 | 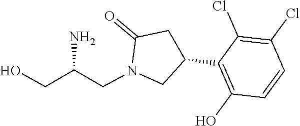 [SMILES: N[C@@H](CO)CN1C[C@@H](c2c(O)ccc(Cl)c2Cl)CC1=O] | <0.1 | * |
| 51 | 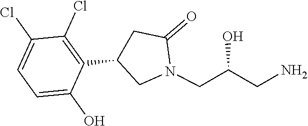 [SMILES: NC[C@@H](O)CN1C[C@H](c2c(O)ccc(Cl)c2Cl)CC1=O] | <0.1 | >100 |
| 52 | 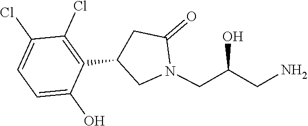 [SMILES: NC[C@H](O)CN1C[C@H](c2c(O)ccc(Cl)c2Cl)CC1=O] | <0.1 | >100 |
| 53 | 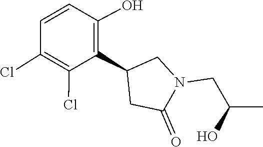 [SMILES: C[C@@H](O)CN1C[C@H](c2c(O)ccc(Cl)c2Cl)CC1=O] | <0.1 | >30 |
| 54 | 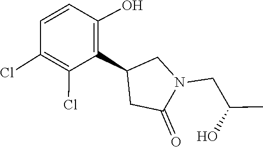 [SMILES: C[C@H](O)CN1C[C@H](c2c(O)ccc(Cl)c2Cl)CC1=O] | <0.1 | >30 |
| 55 | 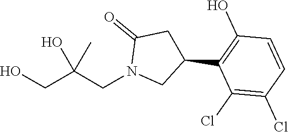 [SMILES: CC(O)(CO)CN1C[C@H](c2c(O)ccc(Cl)c2Cl)CC1=O] | <1 | >100 |
| 56 | 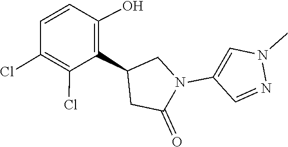 [SMILES: Cn1cc(N2C[C@H](c3c(O)ccc(Cl)c3Cl)CC2=O)cn1] | <0.1 | >100 |
| 57 | 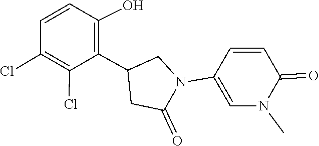 [SMILES: Cn1cc(N2CC(c3c(O)ccc(Cl)c3Cl)CC2=O)ccc1=O] | <0.1 | >10 |
| 58 | 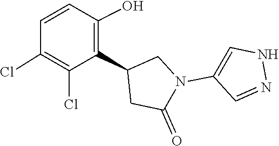 [SMILES: O=C1C[C@@H](c2c(O)ccc(Cl)c2Cl)CN1c1cn[nH]c1] | <0.1 | >30 |
| 59 | 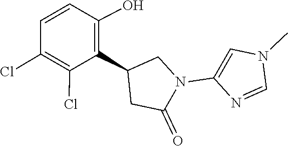 [SMILES: Cn1cnc(N2C[C@H](c3c(O)ccc(Cl)c3Cl)CC2=O)c1] | <1 | * |
| 60 | 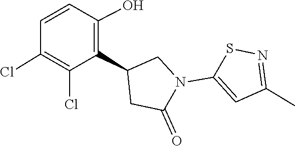 [SMILES: Cc1cc(N2C[C@H](c3c(O)ccc(Cl)c3Cl)CC2=O)sn1] | <1 | * |
| 61 | 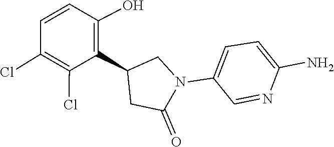 [SMILES: Nc1ccc(N2C[C@H](c3c(O)ccc(Cl)c3Cl)CC2=O)cn1] | <0.1 | >10 |
| 62 | 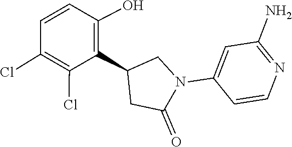 [SMILES: Nc1cc(N2C[C@H](c3c(O)ccc(Cl)c3Cl)CC2=O)ccn1] | <0.1 | >1 |
| 63 | 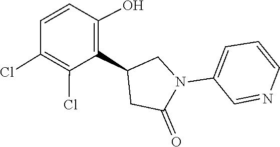 [SMILES: O=C1C[C@@H](c2c(O)ccc(Cl)c2Cl)CN1c1cccnc1] | <1 | * |
| 64 | 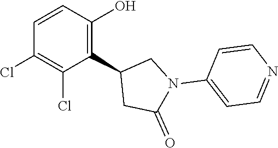 [SMILES: O=C1C[C@@H](c2c(O)ccc(Cl)c2Cl)CN1c1ccncc1] | <0.1 | >10 |
| 65 | 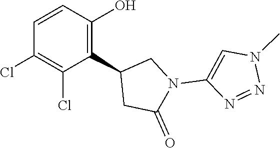 [SMILES: Cn1cc(N2C[C@H](c3c(O)ccc(Cl)c3Cl)CC2=O)nn1] | <1 | >10 |
| 66 | 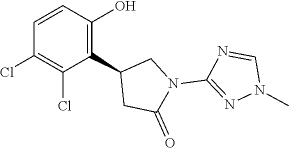 [SMILES: Cn1cnc(N2C[C@H](c3c(O)ccc(Cl)c3Cl)CC2=O)n1] | <1 | >100 |
| 67 | 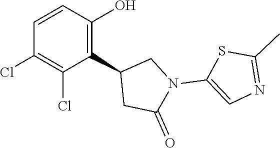 [SMILES: Cc1ncc(N2C[C@H](c3c(O)ccc(Cl)c3Cl)CC2=O)s1] | <0.1 | >100 |
| 68 | 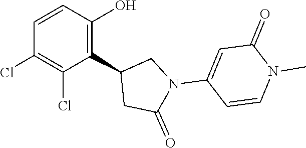 [SMILES: Cn1ccc(N2C[C@H](c3c(O)ccc(Cl)c3Cl)CC2=O)cc1=O] | <1 | >100 |
| 69 | 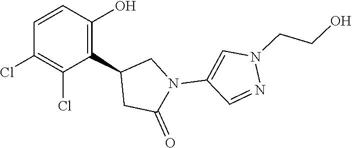 [SMILES: O=C1C[C@@H](c2c(O)ccc(Cl)c2Cl)CN1c1cnn(CCO)c1] | <0.1 | * |
| 70 | 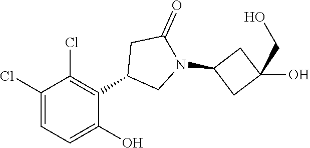 [SMILES: O=C1C[C@@H](c2c(O)ccc(Cl)c2Cl)CN1[C@H]1C[C@@](O)(CO)C1] | <0.1 | >100 |
| 71 | 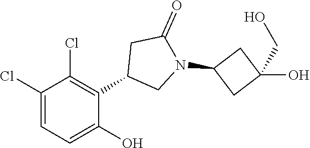 [SMILES: O=C1C[C@@H](c2c(O)ccc(Cl)c2Cl)CN1[C@H]1C[C@](O)(CO)C1] | <0.1 | >100 |
| 72 | 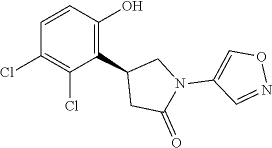 [SMILES: O=C1C[C@@H](c2c(O)ccc(Cl)c2Cl)CN1c1cnoc1] | <1 | >100 |
| 73 | 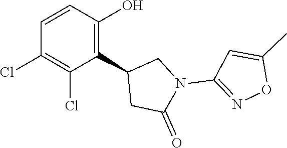 [SMILES: Cc1cc(N2C[C@H](c3c(O)ccc(Cl)c3Cl)CC2=O)no1] | <10 | * |
| 74 | 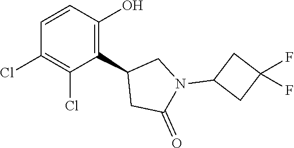 [SMILES: O=C1C[C@@H](c2c(O)ccc(Cl)c2Cl)CN1C1CC(F)(F)C1] | <1 | >30 |
| 75 | 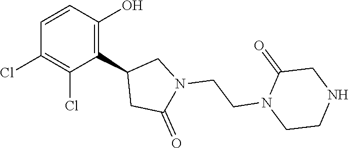 [SMILES: O=C1CNCCN1CCN1C[C@H](c2c(O)ccc(Cl)c2Cl)CC1=O] | <0.1 | >30 |
| 76 | 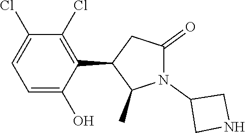 [SMILES: C[C@H]1[C@@H](c2c(O)ccc(Cl)c2Cl)CC(=O)N1C1CNC1] | <1 | >100 |
| 77 | 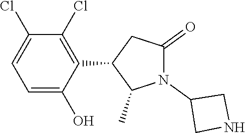 [SMILES: C[C@@H]1[C@H](c2c(O)ccc(Cl)c2Cl)CC(=O)N1C1CNC1] | <0.1 | >30 |
| 78 | 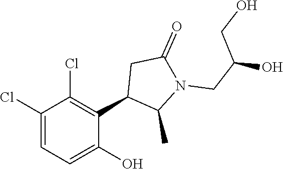 [SMILES: C[C@H]1[C@@H](c2c(O)ccc(Cl)c2Cl)CC(=O)N1C[C@H](O)CO] | <10 | * |
| 79 | 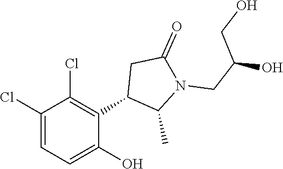 [SMILES: C[C@@H]1[C@H](c2c(O)ccc(Cl)c2Cl)CC(=O)N1C[C@H](O)CO] | <0.1 | >30 |
| 80 | 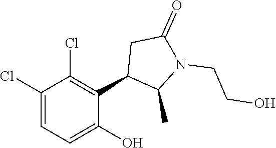 [SMILES: C[C@H]1[C@@H](c2c(O)ccc(Cl)c2Cl)CC(=O)N1CCO] | <10 | * |
| 81 | 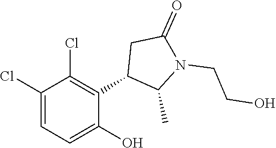 [SMILES: C[C@@H]1[C@H](c2c(O)ccc(Cl)c2Cl)CC(=O)N1CCO] | <0.1 | >30 |
| 82 | 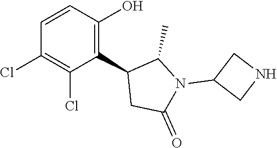 [SMILES: C[C@H]1[C@H](c2c(O)ccc(Cl)c2Cl)CC(=O)N1C1CNC1] | <0.1 | >30 |
| 83 | 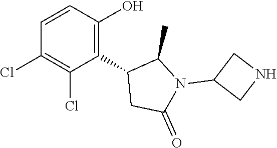 [SMILES: C[C@@H]1[C@@H](c2c(O)ccc(Cl)c2Cl)CC(=O)N1C1CNC1] | <10 | * |
| 84 | 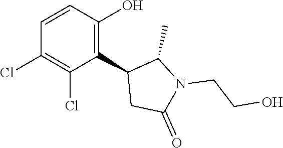 [SMILES: C[C@H]1[C@H](c2c(O)ccc(Cl)c2Cl)CC(=O)N1CCO] | <1 | * |
| 85 | 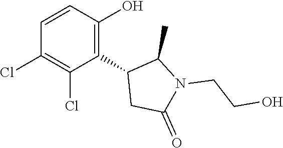 [SMILES: C[C@@H]1[C@@H](c2c(O)ccc(Cl)c2Cl)CC(=O)N1CCO] | <30 | * |
| 86 | 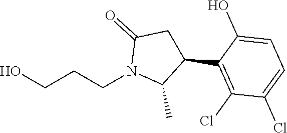 [SMILES: C[C@H]1[C@H](c2c(O)ccc(Cl)c2Cl)CC(=O)N1CCCO] | <0.1 | >30 |
| 87 | 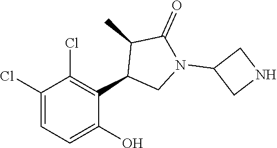 [SMILES: C[C@H]1C(=O)N(C2CNC2)C[C@H]1c1c(O)ccc(Cl)c1Cl] | <1 | * |
| 88 | 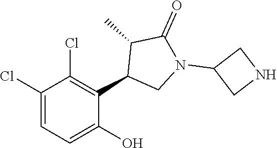 [SMILES: C[C@@H]1C(=O)N(C2CNC2)C[C@H]1c1c(O)ccc(Cl)c1Cl] | <10 | * |
| 89 | 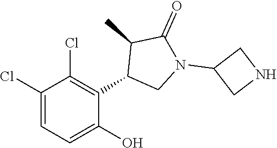 [SMILES: C[C@H]1C(=O)N(C2CNC2)C[C@@H]1c1c(O)ccc(Cl)c1Cl] | <10 | * |
| 90 | 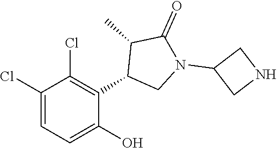 [SMILES: C[C@@H]1C(=O)N(C2CNC2)C[C@@H]1c1c(O)ccc(Cl)c1Cl] | <1 | >100 |
| 91 | 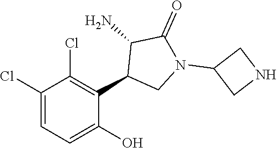 [SMILES: N[C@@H]1C(=O)N(C2CNC2)C[C@H]1c1c(O)ccc(Cl)c1Cl] | <10 | * |
| 92 | 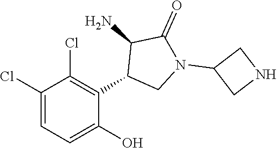 [SMILES: N[C@H]1C(=O)N(C2CNC2)C[C@@H]1c1c(O)ccc(Cl)c1Cl] | <1 | >100 |
| 93 | 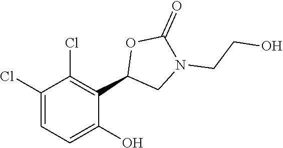 [SMILES: O=C1O[C@H](c2c(O)ccc(Cl)c2Cl)CN1CCO] | <1 | >100 |
| 94 | 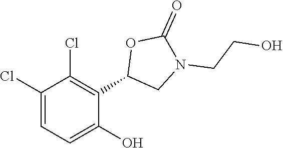 [SMILES: O=C1O[C@@H](c2c(O)ccc(Cl)c2Cl)CN1CCO] | <1 | >100 |
| 95 | 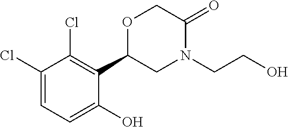 [SMILES: O=C1CO[C@H](c2c(O)ccc(Cl)c2Cl)CN1CCO] | <1 | >100 |
| 96 | 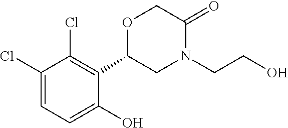 [SMILES: O=C1CO[C@@H](c2c(O)ccc(Cl)c2Cl)CN1CCO] | <30 | * |
| 97 | 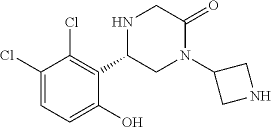 [SMILES: O=C1CN[C@@H](c2c(O)ccc(Cl)c2Cl)CN1C1CNC1] | <1 | * |
| 98 | 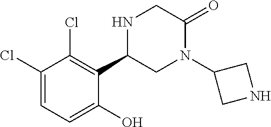 [SMILES: O=C1CN[C@H](c2c(O)ccc(Cl)c2Cl)CN1C1CNC1] | <30 | * |
| 99 | 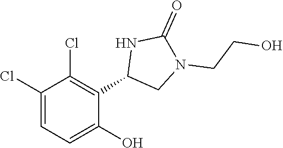 [SMILES: O=C1N[C@@H](c2c(O)ccc(Cl)c2Cl)CN1CCO] | <0.1 | >100 |
| 100 | 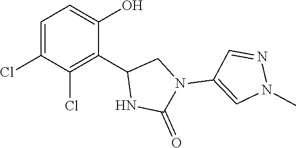 [SMILES: Cn1cc(N2CC(c3c(O)ccc(Cl)c3Cl)NC2=O)cn1] | <0.1 | >30 |
| 101 | 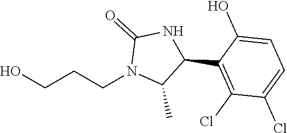 [SMILES: C[C@H]1[C@H](c2c(O)ccc(Cl)c2Cl)NC(=O)N1CCCO] | <0.1 | >30 |
| 102 | 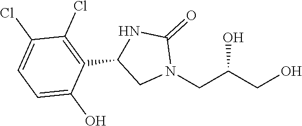 [SMILES: O=C1N[C@@H](c2c(O)ccc(Cl)c2Cl)CN1C[C@H](O)CO] | <0.1 | >100 |
| 103 | 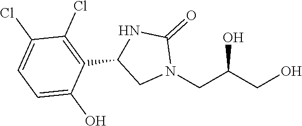 [SMILES: O=C1N[C@@H](c2c(O)ccc(Cl)c2Cl)CN1C[C@@H](O)CO] | <1 | >100 |
| 104 | 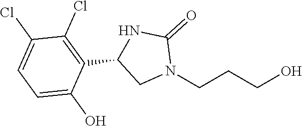 [SMILES: O=C1N[C@@H](c2c(O)ccc(Cl)c2Cl)CN1CCCO] | <0.1 | >100 |
| 105 | 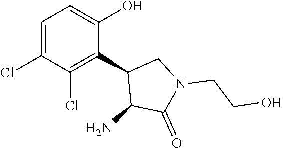 [SMILES: N[C@@H]1C(=O)N(CCO)C[C@@H]1c1c(O)ccc(Cl)c1Cl] | <1 | >100 |

*Not tested.

1. A compound of Formula I or a pharmaceutically acceptable salt thereof: 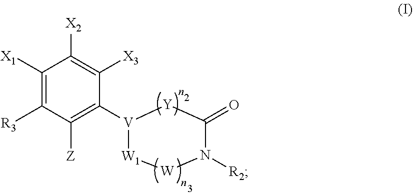
wherein:
X₁, X₂, and X₃ are each independently H, halogen, CN, alkyl, cycloalkyl, halogenated alkyl, or halogenated cycloalkyl;
or alternatively X₁ and X₂ and the carbon atoms they are connected to taken together form an optionally substituted 5- or 6-membered aryl;
or alternatively X₂ and X₃ and the carbon atoms they are connected to taken together form an optionally substituted 5- or 6-membered aryl;
Z is ORₐ;
R₃ is H, halogen, alkyl, cycloalkyl, saturated heterocycle, aryl, heteroaryl, CN, CF₃, OCF₃, ORₐ, SRₐ, NRₐR_{b}, or NRₐ(C═O)R_{b};
V is CR₁;
W₁ is CHR₁, O, or NR₄;
each occurrence of W is independently CHR₁ or NR₅;
each occurrence of Y is independently CHR₁, O, or NR₆;
each occurrence of R₁ is independently H, alkyl, halogen, or (CR₇R₈)ₚNRₐ,R_{b};
each occurrence of R₄, R₅, and R₆ is independently H, alkyl, heteroalkyl, cycloalkyl, cycloheteroalkyl, alkylaryl, aryl, or heteroaryl;
R₂ is H, alkyl, (CR₇R₈)ₚcycloalkyl, (CR₇R₈)ₚheteroalkyl, (CR₇R₈)ₚcycloheteroalkyl, (CR₇R₈)ₚaryl, (CR₇R₈)ₚheteroaryl, (CR₇R₈)ₚORₐ, (CR₇R₈)ₚNRₐR_{b}, (CR₇R₈)ₚ(C═O)ORₐ, (CR₇R₈)ₚNRₐ(C═O)R_{b}, or (CR₇R₈)ₚ(C═O)NRₐR_{b};
each occurrence of R₇ and R₈ is independently H, alkyl, cycloalkyl, aryl, or heteroaryl;
each occurrence of Rₐ and R_{b} is independently H, alkyl, cycloalkyl, heterocycle, aryl, or heteroaryl;
or alternatively Rₐ and R_{b} together with the atom that they are connected to form a 3-7-membered optionally substituted carbocycle or heterocycle;
the alkyl, cycloalkyl, heteroalkyl, cycloheteroalkyl, aryl, heteroaryl, carbocycle, and heterocycle of X₁, X₂, X₃, R₁, R₂, R₃, R₄, R₅, R₆, R₇, R₈, Rₐ, and R_{b}, where applicable, are each independently and optionally substituted by 1-4 substituents each independently selected from the group consisting of alkyl, cycloalkyl, halogenated alkyl, halogenated cycloalkyl, halogen, CN, R_{c}, (CR_{c}R_{d})ₚOR_{c}, (CR_{c}R_{d})ₚ(C═O)OR_{c}, (CR_{c}R_{d})ₚNR_{c}R_{d}, (CR_{c}R_{d})ₚ(C═O)NR_{c}R_{d}, (CR_{c}R_{d})ₚNR_{c}(C═O)R_{d}, and oxo where valence permits;
each occurrence of R_{c} and R_{d} is independently H, alkyl, cycloalkyl, heterocycle, aryl, or heteroaryl;
each heterocycle comprises 1-3 heteroatoms each independently selected from the group consisting of O, S, and N;
n₂ is an integer from 0-2;
n₃ is an integer from 0-2;
wherein the sum of n₂ and n₃ is 1 or 2; and
each occurrence of p is independently an integer from 0-4.

2. The compound of claim 1, wherein W₁ is CHR₁ or NR₄.

3. (canceled)

4. The compound of claim 1, wherein each occurrence of W is independently CHR₁.

5-6. (canceled)

7. The compound of claim 1, wherein each occurrence of Y is independently CHR₁ or NR₆.

8-9. (canceled)

10. The compound of claim 1, wherein each occurrence of R₁ is H, alkyl, cycloalkyl, or (CR₇R₈)ₚNRₐR_{b}.

11-13. (canceled)

14. The compound of claim 1, wherein each occurrence of R₁ is independently H, CH₃, CH₂CH₃, NH₂, NHCH₃, or N(CH₃)₂.

15. The compound of claim 1, wherein each occurrence of R₄, R₅, and R₆ is independently H, alkyl, heteroalkyl, cycloalkyl, or cycloheteroalkyl.

16. (canceled)

17. The compound of claim 1, wherein each occurrence of R₄, R₅, and R₆ is independently H or alkyl.

18. (canceled)

19. The compound of claim 1, wherein R₂ is H, alkyl, (CR₇R₈)ₚcycloalkyl, (CR₇R₈)ₚheteroalkyl, (CR₇R₈)ₚcycloheteroalkyl, (CR₇R₈)ₚaryl, (CR₇R₈)ₚheteroaryl, (CR₇R₈)ₚORₐ, (CR₇R₈)ₚNRₐR_{b}, (CR₇R₈)ₚ(C═O)ORₐ, or (CR₇R₈)ₚNRₐ(C═O)R_{b}.

20. The compound of claim 19, wherein the cycloalkyl is selected from the group consisting of a cyclopropyl, cyclobutyl, and cyclopentyl group,
wherein the cycloheteroalkyl is selected from the group consisting of an azetidinyl, oxetanyl, pyrrolidinyl, tetrahydrofuranyl, tetrahydropyranyl, piperazinyl, piperazinonyl, and pyridinonyl group; and
wherein the heteroaryl is selected from the group consisting of an isoxazolyl, isothiazolyl, pyridinyl, imidazolyl, thiazolyl, pyrazolyl, and triazolyl group.

21-26. (canceled)

27. The compound of claim 19, wherein each occurrence of R₇ and R₈ is independently H, cycloalkyl, aryl, heteroaryl, or alkyl; and
wherein each occurrence of Rₐ and R_{b} is independently H, alkyl, cycloalkyl, heterocycle, aryl, or heteroaryl.

28-30. (canceled)

31. The compound of claim 1, wherein at least one occurrence of p is 0, 1, or 2.

32. The compound of claim 1, wherein at least one occurrence of p is 3 or 4.

33. The compound of claim 1, wherein V is CH and the structural moiety 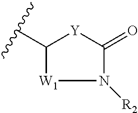
has the structure of

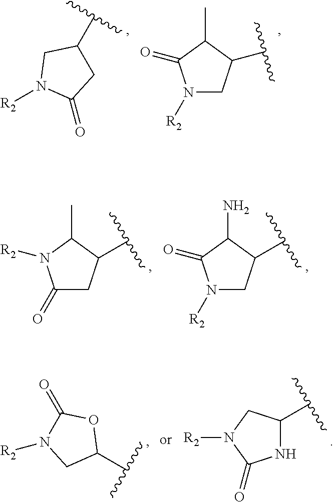

34. The compound of claim 1, wherein V is CH and the structural moiety 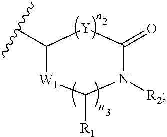
has the structure of

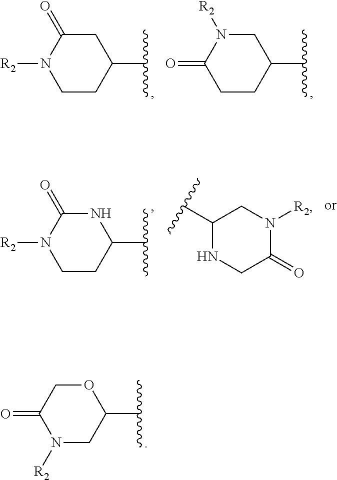

35. The compound of claim 1, wherein R₂ is

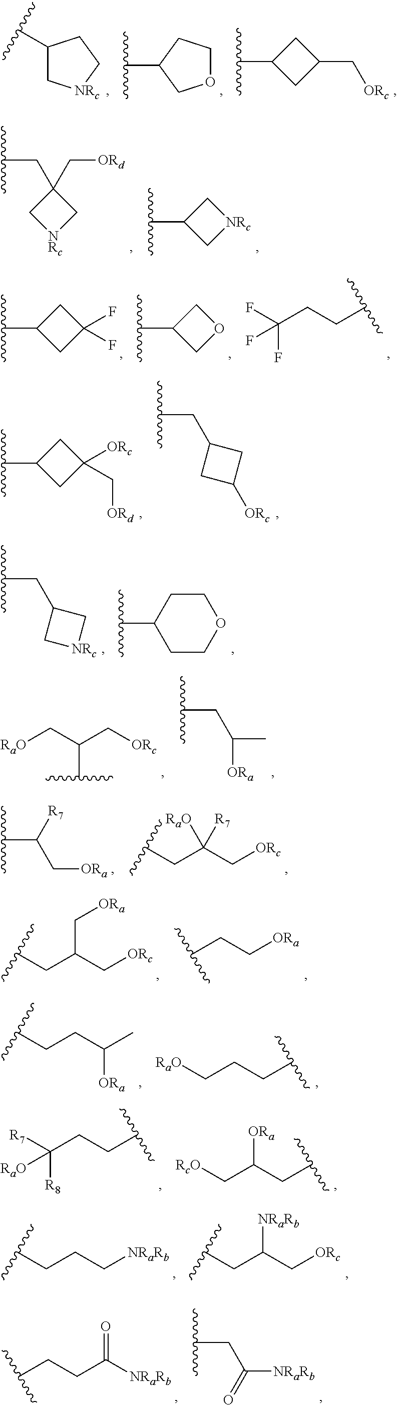

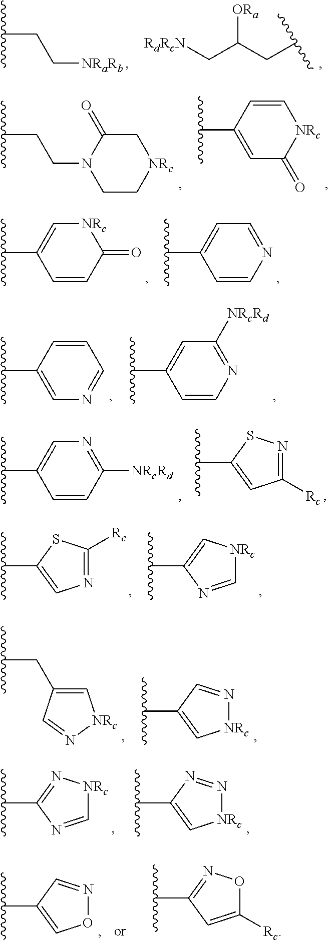

36. The compound of claim 1, wherein R₂ is

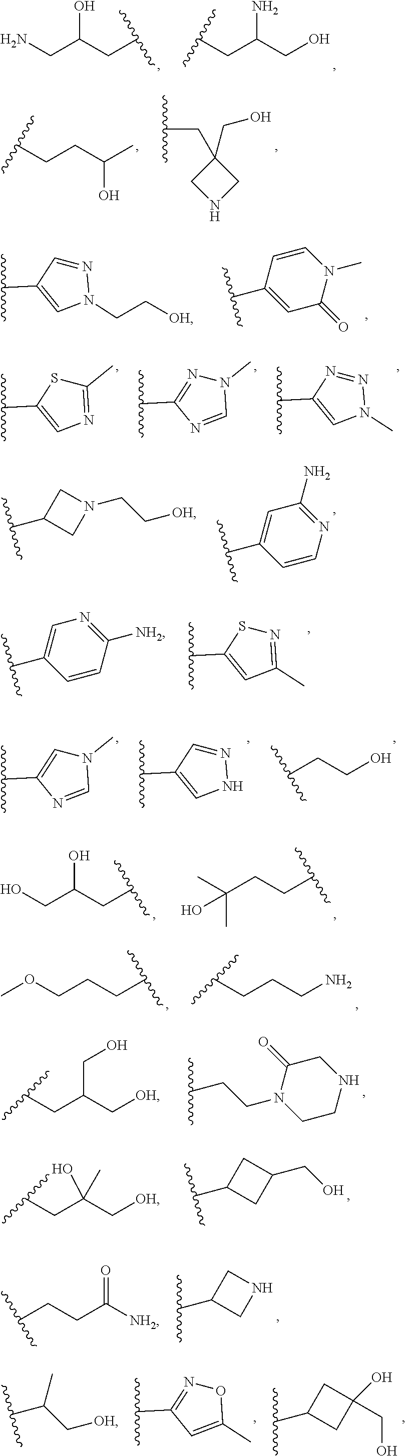

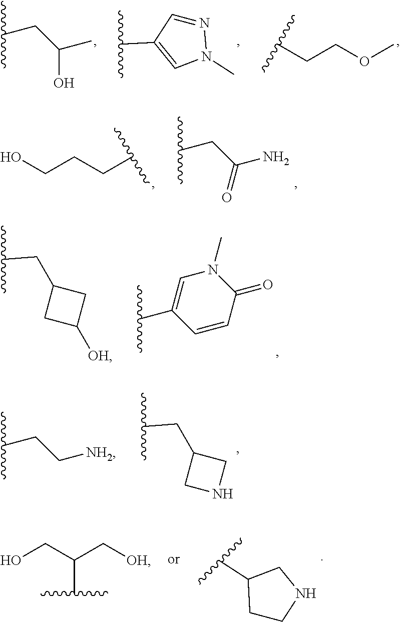

37. The compound of claim 1, wherein the structural moiety 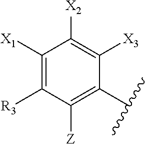
has the structure of

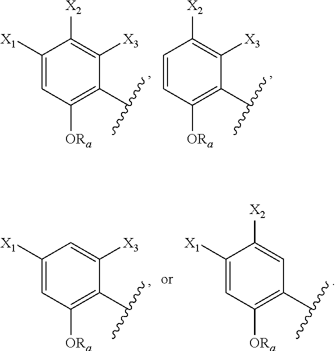

38. The compound of claim 1, wherein X₁, X₂, and X₃ are each independently H, halogen, alkyl, halogenated alkyl, CN, cycloalkyl, or halogenated cycloalkyl.

39. (canceled)

40. The compound of claim 1, wherein X₁, X₂, and X₃ are each independently H, F, Cl, Br, CH₃, CH₂F, CHF₂, or CF₃.

41. The compound of claim 1, wherein X₁, X₂, and X₃ are each independently H or Cl.

42. The compound of claim 1, wherein Z is OH or O(C₁₋₄ alkyl).

43. (canceled)

44. The compound of claim 1, wherein R₃ is H, halogen, alkyl, cycloalkyl, saturated heterocycle, aryl, heteroaryl, CN, CF₃, OCF₃, ORₐ, NRₐR_{b}, or NRₐ(C═O)R_{b}.

45-47. (canceled)

48. The compound of claim 44, wherein each occurrence of Rₐ and R_{b} is independently H or alkyl.

49. The compound of claim 1, wherein R₃ is H, F, Cl, Br, C₁₋₄ alkyl, or CF₃.

50. (canceled)

51. The compound of claim 1, wherein the structural moiety 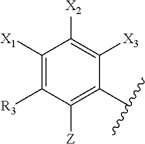
has the structure of

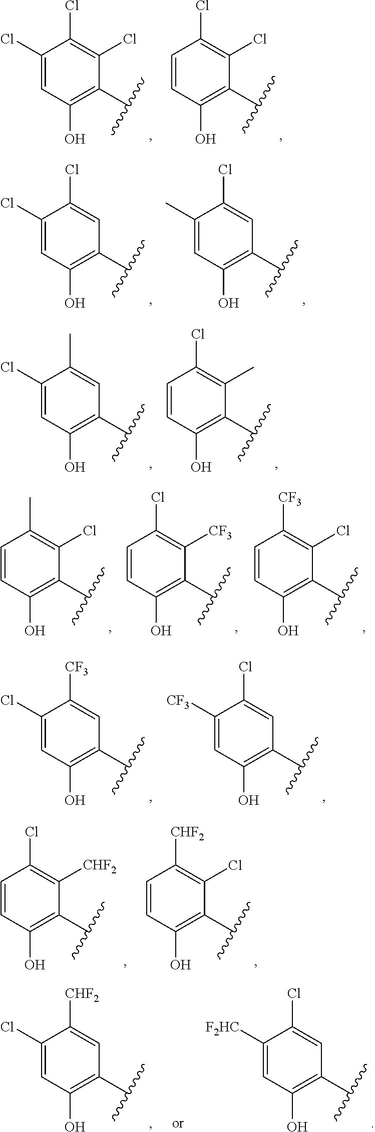

52. The compound of claim 1, wherein the structural moiety 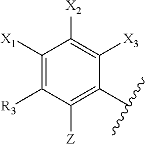
has the structure of

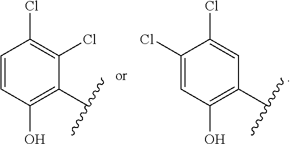 [SMILES: *c1c(O)ccc(Cl)c1Cl ; "or" ; *c1cc(Cl)c(Cl)cc1O ; "."]

53-66. (canceled)
